(19) United States
(12) Patent Application Publication
(10) Pub. No.: US 2022/0372080 A1
(43) Pub. Date: Nov. 24, 2022

(54) SARS-COV-2 SUBUNIT AND VARIANT VACCINES

(71) Applicant: iBio, Inc., Bryan, TX (US)

(72) Inventors: Peter Kipp, Bryan, TX (US); Brian Berquist, Bryan, TX (US); Sreenath Palle, Bryan, TX (US)

(21) Appl. No.: 17/726,110

(22) Filed: Apr. 21, 2022

Related U.S. Application Data
(60) Provisional application No. 63/178,443, filed on Apr. 22, 2021.
(60) Provisional application No. 63/217,364, filed on Jul. 1, 2021.
(60) Provisional application No. 63/222,358, filed on Jul. 15, 2021.

(51) Int. Cl. C07K 14/005; A61K 39/215; C12N 15/82

(52) U.S. Cl. CPC C07K 14/005; A61K 39/215; C12N 15/8258; C12N 2770/20022; C12N 2770/20034; A61K 2039/6031

(57) ABSTRACT

The present invention includes an immunogenic protein, constructs, vectors, and methods of making, comprising at least 90% amino acid identity to at least one antigenic peptide selected from: a coronavirus Receptor Binding Domain (RBD), coronavirus a Receptor Binding Motif (RBM) of a coronavirus spike protein, a coronavirus spike protein N-terminus, a nucleocapsid protein, one or more T cell epitopes from a coronavirus spike protein, or one or more T cell epitopes from a coronavirus nucleocapsid protein, or combination thereof. In one example, the at least one antigenic peptide is positioned at, at least one of, the N-terminus, the C-terminus, or in a loop region of the carrier protein or peptide tag.

### CROSS-REFERENCE TO RELATED APPLICATIONS

This application claims priority to U.S. Provisional Application Ser. No. 63/178,443, filed Apr. 22, 2021, U.S. Provisional Application Ser. No. 63/217,364, filed Jul. 1, 2021, and U.S. Provisional Application Ser. No. 63/222,358 filed Jul. 15, 2021, the entire contents of which are incorporated herein by reference.

### TECHNICAL FIELD OF THE INVENTION

The present invention relates in general to the field of coronaviruses, and more particularly, to novel SARS-COV-2 subunit and variant vaccines and methods for using the same.

### STATEMENT OF FEDERALLY FUNDED RESEARCH

Not applicable.

### INCORPORATION-BY-REFERENCE OF MATERIALS FILED ON COMPACT DISC

The present application includes a Sequence Listing which has been submitted in ASCII format via EFS-Web and is hereby incorporated by reference in its entirety. Said ASCII copy, created on Apr. 25, 2022, is named IBIO2013WO_ST25.txt and is 345,820 bytes in size.

### BACKGROUND OF THE INVENTION

Without limiting the scope of the invention, its background is described in connection with anti-viral agents.

Vaccines are a very effective means for preventing and even eliminating infectious diseases. Although there are a number of efficacious vaccines based on full pathogens, development of safer more potent and cost-effective vaccines based on portions of pathogen (subunit vaccines) is important. During the last two decades several approaches to the expression (bacterial, yeast, mammalian cell culture and plant) and delivery (DNA, live virus vectors, purified proteins, plant virus particles) of vaccine antigens have been developed. All these approaches have significant impact on the development and testing of newly developed candidate vaccines. However, there is a need for improving expression and delivery systems to create more efficacious but safer vaccines with fewer side effects. Some of the desired features or future vaccines are (a) to be highly efficacious (stimulates both arms of immune system), (b) to have known and controlled genetic composition, (c) to have time efficiency of the system, (d) to be suitable for expression of both small size peptides and large size polypeptides, (e) to be suitable for expression in different systems (bacteria, yeast, mammalian cell cultures, live virus vectors, DNA vectors, transgenic plants and transient expression vectors), and (f) to be capable of forming structures such as aggregates or virus like particles that are easy to recover and are immunogenic.

What is needed is an immunization that that be developed quickly, has an enhanced immune response, and can be produced rapidly and effectively.

### SUMMARY OF THE INVENTION

In one embodiment, the present invention includes an immunogenic protein comprising at least 90% amino acid identity to an amino acid sequence of at least one antigenic peptide selected from: a coronavirus Receptor Binding Domain (RBD), coronavirus a Receptor Binding Motif (RBM) of a coronavirus spike protein, a coronavirus spike protein N-terminus, a nucleocapsid protein, one or more T cell epitopes from a coronavirus spike protein, or one or more T cell epitopes from a coronavirus nucleocapsid protein, or combination thereof. In one aspect, the immunogenic protein further comprises a carrier protein or peptide tag, wherein the at least one antigenic peptide is positioned at, at least one of, the N-terminus, the C-terminus, or in a loop region of the carrier protein. In another aspect, the carrier protein is selected from a modified thermostable lichenase (LicKM), a human hepatitis core antigen (HBcAg), or a truncated woodchuck hepatitis core antigen (WHcAg). In another aspect, the immunogenic protein is formulated into an immunization. In one aspect, the at least one antigenic peptide is a fusion protein is selected from at least one of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 79, 81, 83, 85, 87, 91, 93, 95, 97, 99, 101, 103, 105, 137, 139, 141, 43, 145, 147, 149, 151, 153, 155, 157, 158, 159, 160, or 161. In another aspect, the immunogenic protein is encoded by a nucleic acid selected from at least one of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 107, 108, 109, 110, 111, 112, 113, 114, 138, 140, 142, 144, 146, 148, 150, 152, 154, or 156. In another aspect, the immunogenic protein further comprises an adjuvant selected from at least one of alum, aluminum hydroxide, aluminum phosphate, calcium phosphate hydroxide, cytosine-guanosine oligonucleotide (CpG-ODN) sequence, granulocyte macrophage colony stimulating factor (GM-CSF), monophosphoryl lipid A (MPL), poly(I:C), MF59, Quil A, N-acetyl muramyl-L-alanyl-D-isoglutamine (MDP), FIA, montanide, poly (DL-lactide-coglycolide), squalene, glucopyranosyl lipid adjuvant (GLA), GLA-Alum, 3M-052, a glucopyranosyl lipid adjuvant GLA emulsion with squalene (GLA-SE), virosome, AS03, ASO4, IL-1, IL-2, IL-3, IL-4, IL-5, IL-6, IL-7, IL-8, IL-10, IL-12, IL-15, IL-17, IL-18, STING, CD40L, pathogen-associated molecular patterns (PAMPs), damage-associated molecular pattern molecules (DAMPs), Freund's complete adjuvant, Freund's incomplete adjuvant, transforming growth factor (TGF)-beta antibody or antagonists, A2aR antagonists, lipopolysaccharides (LPS), Fas ligand, Trail, lymphotactin, Mannan (M-FP), APG-2, Hsp70 and Hsp90, pattern recognition receptor ligands, TLR3 ligands, TLR4 ligands, TLR5 ligands, TLR7/8 ligands, or TLR9 ligands. In another aspect, the immunogenic protein is further modified to include one or more engineered glycosylation sites, or less disulfide forming residues. In another aspect, the coronavirus is SARS, MERS, 229E (alpha), NL63 (alpha), OC43 (beta), HKU1 (beta), or SARS-CoV-2 variants including the Wuhan parental sequence with or without the D614G mutation, Alpha (B.1.1.7 and Q lineages), Beta (B.1.351 and descendent lineages), Gamma (P.1 and descendent lineages), Epsilon (B.1.427 and B.1.429), Eta (B.1.525), Iota (B.1.526), Kappa (B.1.617.1), Mu (B.1.621, B.1.621.1), Zeta (P.2), Delta (B.1.617.2 and AY lineages), and Omicron (B.1.1.529) at least one of variants BA.1, BA.2, or BA.3.

In another embodiment, the present invention includes a method of stimulating an immune response in an animal comprising administering to the animal a composition comprising a protein that has at least 90% amino acid identity at least one antigenic peptide selected from: a coronavirus Receptor Binding Domain (RBD), coronavirus a Receptor Binding Motif (RBM) of a coronavirus spike protein, a coronavirus spike protein N-terminus, a nucleocapsid protein, one or more T cell epitopes from a coronavirus spike protein, or one or more T cell epitopes from a coronavirus nucleocapsid protein, or combination thereof. In one aspect, the method further comprises adding a carrier protein or peptide tag, wherein the at least one antigenic peptide is positioned at, at least one of, the N-terminus, the C-terminus, or in a loop region of the carrier protein. In one aspect, the carrier protein is selected from a modified thermostable lichenase (LicKM), a human hepatitis core antigen (HBcAg), or a truncated woodchuck hepatitis core antigen (WHcAg). In another aspect, the immunogenic protein is formulated into an immunization. In another aspect, the at least one antigenic peptide is a fusion protein selected from at least one of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 79, 81, 83, 85, 87, 91, 93, 95, 97, 99, 101, 103, 105, 137, 139, 141, 43, 145, 147, 149, 151, 153, 155, 157, 158, 159, 160, or 161. In another aspect, the immunogenic protein is encoded by a nucleic acid selected from at least one of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 107, 108, 109, 110, 111, 112, 113, 114, 138, 140, 142, 144, 146, 148, 150, 152, 154, or 156. In one aspect, the immune response is at least one of: a humoral immune response, a cellular immune response, or an innate immune response. In one aspect, the coronavirus is SARS, MERS, 229E (alpha), NL63 (alpha), OC43 (beta), HKU1 (beta), or SARS-CoV-2 variants including the Wuhan parental sequence with or without the D614G mutation, Alpha (B.1.1.7 and Q lineages), Beta (B.1.351 and descendent lineages), Gamma (P.1 and descendent lineages), Epsilon (B.1.427 and B.1.429), Eta (B.1.525), Iota (B.1.526), Kappa (B.1.617.1), Mu (B.1.621, B.1.621.1), Zeta (P.2), Delta (B.1.617.2 and AY lineages), and Omicron (B.1.1.529) at least one of variants BA.1, BA.2, or BA.3.

In another embodiment, the present invention includes a method for production of a carrier protein in a plant comprising: (a) providing a plant containing an expression cassette having a nucleic acid encoding an immunogenic protein that has at least 90% amino acid identity to at least one antigenic peptide selected from: a coronavirus Receptor Binding Domain (RBD), coronavirus a Receptor Binding Motif (RBM) of a coronavirus spike protein, a coronavirus spike protein N-terminus, a nucleocapsid protein, one or more T cell epitopes from a coronavirus spike protein, or one or more T cell epitopes from a coronavirus nucleocapsid protein, or combination thereof; and (b) growing the plant under conditions in which the nucleic acid is expressed and the immunogenic protein is produced. In one aspect, the antigenic protein further comprises a carrier protein or peptide tag, wherein the at least one immunogenic protein is positioned at, at least one of, the N-terminus, the C-terminus, or in a loop region of the carrier protein or peptide tag. In another aspect, the method further comprises the step of recovering the immunogenic protein. In another aspect, a promoter is selected from the group consisting of plant constitutive promoters and plant tissue specific promoters. In another aspect, the immunogenic protein is expressed in leaf, root, fruit, tubercle or seed of a plant. In another aspect, a plant is a Nicotiana sp. plant. In another aspect, the carrier protein is selected from a modified thermostable lichenase (LicKM), a human hepatitis core antigen (HBcAg), or a truncated woodchuck hepatitis core antigen (WHcAg). In another aspect, the immunogenic protein is formulated into an immunization. In another aspect, the at least one antigenic peptide selected from at least one of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 79, 81, 83, 85, 87, 91, 93, 95, 97, 99, 101, 103, 105, 137, 139, 141, 43, 145, 147, 149, 151, 153, 155, 157, 158, 159, 160, or 161. In another aspect, the immunogenic protein is encoded by a nucleic acid selected from at least one of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 107, 108, 109, 110, 111, 112, 113, 114, 138, 140, 142, 144, 146, 148, 150, 152, 154, or 156. In another aspect, the coronavirus is MERS, SARS, SARS-CoV-2 or variants thereof. In another aspect, the adjuvant is selected from at least one of alum, aluminum hydroxide, aluminum phosphate, calcium phosphate hydroxide, cytosine-guanosine oligonucleotide (CpG-ODN) sequence, granulocyte macrophage colony stimulating factor (GM-CSF), monophosphoryl lipid A (MPL), poly(I:C), MF59, Quil A, N-acetyl muramyl-L-alanyl-D-isoglutamine (MDP), FIA, montanide, poly (DL-lactide-coglycolide), squalene, glucopyranosyl lipid adjuvant (GLA), GLA-Alum, 3M-052, a glucopyranosyl lipid adjuvant GLA emulsion with squalene (GLA-SE), virosome, AS03, AS04, IL-1, IL-2, IL-3, IL-4, IL-5, IL-6, IL-7, IL-8, IL-10, IL-12, IL-15, IL-17, IL-18, STING, CD40L, pathogen-associated molecular patterns (PAMPs), damage-associated molecular pattern molecules (DAMPs), Freund's complete adjuvant, Freund's incomplete adjuvant, transforming growth factor (TGF)-beta antibody or antagonists, A2aR antagonists, lipopolysaccharides (LPS), Fas ligand, Trail, lymphotactin, Mannan (M-FP), APG-2, Hsp70 and Hsp90, pattern recognition receptor ligands, TLR3 ligands, TLR4 ligands, TLR5 ligands, TLR7/8 ligands, or TLR9 ligands. In another aspect, the coronavirus is SARS, MERS, 229E (alpha), NL63 (alpha), OC43 (beta), HKU1 (beta), or SARS-CoV-2 variants including the Wuhan parental sequence with or without the D614G mutation, Alpha (B.1.1.7 and Q lineages), Beta (B.1.351 and descendent lineages), Gamma (P.1 and descendent lineages), Epsilon (B.1.427 and B.1.429), Eta (B.1.525), Iota (B.1.526), Kappa (B.1.617.1), Mu (B.1.621, B.1.621.1), Zeta (P.2), Delta (B.1.617.2 and AY lineages), and Omicron (B.1.1.529) at least one of variants BA.1, BA.2, or BA.3.

In another embodiment, the present invention includes a nucleic acid encoding a protein comprising: an immunogenic fusion protein that has at least 90% amino acid identity to at least one antigenic peptide selected from: a coronavirus Receptor Binding Domain (RBD), coronavirus a Receptor Binding Motif (RBM) of a coronavirus spike protein, a coronavirus spike protein N-terminus, a nucleocapsid protein, one or more T cell epitopes from a coronavirus spike protein, or one or more T cell epitopes from a coronavirus nucleocapsid protein, or combination thereof. In one aspect, the nucleic acid further comprises a carrier protein or peptide tag, wherein the at least one antigenic peptide is positioned at, at least one of, the N-terminus, the C-terminus, or in a loop region of the carrier protein. In another aspect, the nucleic acid further comprises a promoter for plant cell expression. In another aspect, the nucleic acid further comprises a plant promoter selected from one or more plant constitutive promoters, and one or more plant tissue specific promoters. In another aspect, the at least one antigenic peptide is expressed in a leaf, root, fruit, tubercle or seed of a plant. In another aspect, the at least one antigenic peptide is inserted into a recombinant RNA viral vector has a recombinant genomic component of a tobamovirus, an alfalfa mosaic virus, an ilarvirus, a cucumovirus or a closterovirus. In another aspect, a host plant is a dicotyledon or a monocotyledon. In another aspect, t the coronavirus is SARS, MERS, 229E (alpha), NL63 (alpha), OC43 (beta), HKU1 (beta), or SARS-CoV-2 variants including the Wuhan parental sequence with or without the D614G mutation, Alpha (B.1.1.7 and Q lineages), Beta (B.1.351 and descendent lineages), Gamma (P.1 and descendent lineages), Epsilon (B.1.427 and B.1.429), Eta (B.1.525), Iota (B.1.526), Kappa (B.1.617.1), Mu (B.1.621, B.1.621.1), Zeta (P.2), Delta (B.1.617.2 and AY lineages), and Omicron (B.1.1.529) at least one of variants BA.1, BA.2, or BA.3. In another aspect, the nucleic acid is selected from at least one of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 107, 108, 109, 110, 111, 112, 113, 114, 138, 140, 142, 144, 146, 148, 150, 152, 154, or 156. In another aspect, the nucleic acid encodes a protein selected from at least one of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 79, 81, 83, 85, 87, 91, 93, 95, 97, 99, 101, 103, 105, 137, 139, 141, 43, 145, 147, 149, 151, 153, 155, 157, 158, 159, 160, or 161.

In another embodiment, the present invention includes a vector that comprises a nucleic acid that encodes an immunogenic protein that has at least 90% amino acid identity to at least one antigenic peptide selected from: a coronavirus Receptor Binding Domain (RBD), coronavirus a Receptor Binding Motif (RBM) of a coronavirus spike protein, a coronavirus spike protein N-terminus, a nucleocapsid protein, one or more T cell epitopes from a coronavirus spike protein, or one or more T cell epitopes from a coronavirus nucleocapsid protein, or combination thereof. In one aspect, the at least one immunogenic protein or peptide tag is positioned at, at least one of, the N-terminus, the C-terminus, or in a loop region of a carrier protein or peptide tag.

In another embodiment, the present invention includes a host cell that comprises a vector that expresses an immunogenic protein that has at least 90% amino acid identity to at least one antigenic peptide selected from: a coronavirus Receptor Binding Domain (RBD), coronavirus a Receptor Binding Motif (RBM) of a coronavirus spike protein, a coronavirus spike protein N-terminus, a nucleocapsid protein, one or more T cell epitopes from a coronavirus spike protein, or one or more T cell epitopes from a coronavirus nucleocapsid protein, or combination thereof. In one aspect, the at least one immunogenic protein is positioned at, at least one of, the N-terminus, the C-terminus, or in a loop region of a carrier protein or peptide tag.

In another embodiment, the present invention includes a pan-coronavirus booster comprising: an immunogenic protein comprising at least 90% amino acid identity to an amino acid sequence of a coronavirus nucleocapsid protein and adjuvant that triggers a Th1 immune response. In one aspect, the booster is adapted for injected or intranasal administration. In another aspect, the booster triggers a Th1 immune response. In another aspect, the Th1 immune response shows a high secretion of IFN and low secretion of IL-13, IL-5, or both when compared to a non-immunized subject or a subject with a TH2 immune response. In another aspect, the coronavirus is SARS, MERS, 229E (alpha), NL63 (alpha), OC43 (beta), HKU1 (beta), or SARS-CoV-2 variants including the Wuhan parental sequence with or without the D614G mutation, Alpha (B.1.1.7 and Q lineages), Beta (B.1.351 and descendent lineages), Gamma (P.1 and descendent lineages), Epsilon (B.1.427 and B.1.429), Eta (B.1.525), Iota (B.1.526), Kappa (B.1.617.1), Mu (B.1.621, B.1.621.1), Zeta (P.2), Delta (B.1.617.2 and AY lineages), and Omicron (B.1.1.529) at least one of variants BA.1, BA.2, or BA.3. In another aspect, the immunogenic protein only triggers a T cell response when administered intranasally without an adjuvant. In another aspect, the immunogenic protein is administered intramuscularly with an adjuvant and intranasally without an adjuvant. In another aspect, the immunogenic protein is administered with an adjuvant that triggers a Th1 immune response. In another aspect, the immunogenic protein is administered to a subject previously immunized with a coronavirus vaccine.

### BRIEF DESCRIPTION OF THE DRAWINGS

For a more complete understanding of the features and advantages of the present invention, reference is now made to the detailed description of the invention along with the accompanying figures and in which:

FIG. 1 shows a Western blot analyses results for the listed constructs, Lane 1, iBio201 40 1 (39.5 kDa); Lane 2, iBio201 40 2 (39.5 kDa); Lane 3, iBio200 41 (14.4 kDa); Lane 4, iBio201 42 (14.4 kDa); Lane 5, iBio201 43 (14.6 kDa); Lane 6, iBio201 44 (14.6 kDa); Lane 7, SARS CoV2 N with His tag Positive control 3 ug; Lane 8, LicKM with His tag Positive control 4 ug; Lane M, Novex pre stained protein ladder; Lane 9, Mock; Lane 10, IL 6 with His tag Positive control 3 ug; Antibody Direct blot with Anti His Antibody HRP conjugated, all samples are heated and reduced.

FIG. 2 shows the expression of 8HIS-CoV-41 antigen, purified.

FIG. 3 is a chart with the intact mass determination for purified 8HIS-CoV-41 by mass spectrometry.

FIG. 4 is a gel that shows the expression of the purified 8HIS-CoV-41.

FIG. 5 is a graph that shows the results from mouse immunizations with CoV-41 and the immunological skew at day 42 (D42) with the different groups.

FIG. 6 are graphs that show the results from mouse immunizations with CoV-41 and show the T cell priming by ELIspot.

FIG. 7 are graphs that show the results from mouse immunizations with CoV-41 and show the T cell priming by ELIspot.

FIG. 8 are graphs that show the results from mouse immunizations with CoV-41 and show the T cell priming by ELIspot.

FIG. 9 are graphs that show the results from mouse immunizations with CoV-41 and show the T cell priming by ELIspot.

FIG. 10 are graphs that show the results from a naïve mouse and mice immunized with CoV-41 and show the T cell priming by ELIspot.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the term “antigen” refers to a molecule containing one or more epitopes (either linear, conformational or both) that will stimulate a host's immune-system to make a humoral and/or cellular antigen-specific response. The term is used interchangeably with the term “immunogen.” Normally, a B-cell epitope will include at least about 5 amino acids but can be as small as 3-4 amino acids. A T-cell epitope, such as a CTL epitope, will include at least about 7-9 amino acids, and a helper T-cell epitope at least about 12-20 amino acids. Normally, an epitope will include between about 7 and 15 amino acids, such as, 9, 10, 12 or 15 amino acids. The term includes polypeptides, which include modifications, such as deletions, additions and substitutions (generally conservative in nature) as compared to a native sequence, so long as the protein maintains the ability to elicit an immunological response, as defined herein. These modifications may be deliberate, as through site-directed mutagenesis, or may be accidental, such as through mutations of hosts, which produce the antigens.

As used herein, the term “immunological response” refers to an antigen or composition is the development in a subject of a humoral and/or a cellular immune response to an antigen present in the composition of interest. For purposes of the present disclosure, a “humoral immune response” refers to an immune response mediated by antibody molecules, while a “cellular immune response” is one mediated by T-lymphocytes and/or other white blood cells. One important aspect of cellular immunity involves an antigen-specific response by cytolytic T-cells (CTLs). CTLs have specificity for peptide antigens that are presented in association with proteins encoded by the major histocompatibility complex (MHC) and expressed on the surfaces of cells. CTLs help induce and promote the destruction of intracellular microbes, or the lysis of cells infected with such microbes. Another aspect of cellular immunity involves an antigen-specific response by helper T-cells. Helper T-cells act to help stimulate the function, and focus the activity of, nonspecific effector cells against cells displaying peptide antigens in association with MHC molecules on their surface. A “cellular immune response” also refers to the production of cytokines, chemokines and other such molecules produced by activated T-cells and/or other white blood cells, including those derived from CD4+ and CD8+ T-cells. Hence, an immunological response may include one or more of the following effects: the production of antibodies by B-cells; and/or the activation of suppressor T-cells and/or gamma-delta T-cells directed specifically to an antigen or antigens present in the composition or vaccine of interest. These responses may serve to neutralize infectivity, and/or mediate antibody-complement, or antibody dependent cell cytotoxicity (ADCC) to provide protection to an immunized host. Such responses can be determined using standard immunoassays and neutralization assays, well known in the art.

As used herein, the term an “immunogenic composition” refers to a composition that comprises an antigenic molecule where administration of the composition to a subject results in the development in the subject of a humoral and/or a cellular immune response to the antigenic molecule of interest.

As used herein, the term “substantially purified” refers to isolation of a substance (compound, polynucleotide, protein, polypeptide, polypeptide composition) such that the substance comprises the majority percent of the sample in which it resides. Typically, in a sample a substantially purified component comprises 50%, preferably 80%-85%, more preferably 90-95% of the sample. Techniques for purifying polynucleotides and polypeptides of interest are well-known in the art and include, for example, ion-exchange chromatography, affinity chromatography and sedimentation according to density.

As used herein, the term “high-mannose” refers to carbohydrate chains or glycans that contain unsubstituted terminal mannose sugars, and typically contain between five and nine mannose residues, often attached to a chitobiose (GlcNAc₂) core. The name abbreviations are indicative of the total number of mannose residues in the structure, and the position on the carbohydrate of attachment, for example, alpha1,6 is attachment of a mannose in an alpha configuration between carbons 1 and 6, while beta 1,4 is a beta attachment between carbons 1 and 4. The skilled artisan will recognize that the carbohydrates may be high mannose, complex or hybrid, as will beknown to those of skill in the art.

Signal sequences for delivering the proteins of the present invention to different cellular compartments and/or export out of the cell are well-known to the skilled artisan, such as those taught in U.S. Pat. No. 10,577,403, relevant sequences incorporated herein by reference.

As used herein, the term a “coding sequence” or a sequence which “encodes” a selected polypeptide, refers to a nucleic acid molecule that is transcribed (in the case of DNA) and translated (in the case of mRNA) into a polypeptide in vivo when placed under the control of appropriate regulatory sequences (or “control elements”). The boundaries of the coding sequence are determined by a start codon at the 5′ (amino) terminus and a translation stop codon at the 3′ (carboxy) terminus. A coding sequence can include, but is not limited to, cDNA from viral, prokaryotic or eukaryotic mRNA, genomic DNA sequences from viral or prokaryotic DNA, and even synthetic DNA sequences. A transcription termination sequence may be located 3′ to the coding sequence.

As used herein, the term “control elements”, includes, but is not limited to, transcription promoters, transcription enhancer elements, transcription termination signals, polyadenylation sequences (located 3′ to the translation stop codon), sequences for optimization of initiation of translation (located 5′ to the coding sequence), and translation termination sequences, and/or sequence elements controlling an open chromatin structure see e.g., McCaughan et al. (1995) PNAS USA 92:5431-5435; Kochetov et al (1998) FEBS Letts. 440:351-355.

As used herein, the term “nucleic acid” includes, but is not limited to, prokaryotic sequences, eukaryotic mRNA, cDNA from eukaryotic mRNA, genomic DNA sequences from eukaryotic (e.g., mammalian) DNA, and even synthetic DNA sequences. The term also captures sequences that include any of the known base analogs of DNA and RNA.

As used herein, the term “operably linked” refers to an arrangement of elements wherein the components so described are configured so as to perform their usual function. Thus, a given promoter operably linked to a coding sequence is capable of effecting the expression of the coding sequence when active. The promoter need not be contiguous with the coding sequence, so long as it functions to direct the expression thereof. Thus, for example, intervening untranslated yet transcribed sequences can be present between the promoter sequence and the coding sequence and the promoter sequence can still be considered “operably linked” to the coding sequence.

As used herein, the term “recombinant” refers to a polynucleotide of genomic, cDNA, semisynthetic, or synthetic origin which, by virtue of its origin or manipulation: (1) is not associated with all or a portion of the polynucleotide with which it is associated in nature; and/or (2) is linked to a polynucleotide other than that to which it is linked in nature. The term “recombinant” as used with respect to a protein or polypeptide means a polypeptide produced by expression of a recombinant polynucleotide. “Recombinant host cells,” “host cells,” “cells,” “cell lines,” “cell cultures,” and other such terms denoting prokaryotic microorganisms or eukaryotic cell lines cultured as unicellular entities, are used interchangeably, and refer to cells which can be, or have been, used as recipients for recombinant vectors or other transfer DNA, and include the progeny of the original cell which has been transfected. It is understood that the progeny of a single parental cell may not necessarily be completely identical in morphology or in genomic or total DNA complement to the original parent, due to accidental or deliberate mutation. Progeny of the parental cell which are sufficiently similar to the parent to be characterized by the relevant property, such as the presence of a nucleotide sequence encoding a desired peptide, are included in the progeny intended by this definition, and are covered by the above terms.

Techniques for determining amino acid sequence “similarity” are well known in the art. In general, “similarity” means the exact amino acid to amino acid comparison of two or more polypeptides at the appropriate place, where amino acids are identical or possess similar chemical and/or physical properties such as charge or hydrophobicity. A so-termed “percent similarity” then can be determined between the compared polypeptide sequences. Techniques for determining nucleic acid and amino acid sequence identity also are well known in the art and include determining the nucleotide sequence of the mRNA for that gene (usually via a cDNA intermediate) and determining the amino acid sequence encoded thereby and comparing this to a second amino acid sequence. In general, “identity” refers to an exact nucleotide to nucleotide or amino acid to amino acid correspondence of two polynucleotides or polypeptide sequences, respectively.

Two or more polynucleotide sequences can be compared by determining their “percent identity.” Two or more amino acid sequences likewise can be compared by determining their “percent identity.” The percent identity of two sequences, whether nucleic acid or peptide sequences, is generally described as the number of exact matches between two aligned sequences divided by the length of the shorter sequence and multiplied by 100. An approximate alignment for nucleic acid sequences is provided by the local homology algorithm of Smith and Waterman, Advances in Applied Mathematics 2:482-489 (1981). This algorithm can be extended to use with peptide sequences using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986), relevant portion incorporated herein by reference. Suitable programs for calculating the percent identity or similarity between sequences are generally known in the art.

As used herein, a polypeptide or peptide “variant” has at least 90%, 91%, 92%, 93%, 94%, 95%, 96%, 97%, 98% or 99% sequence identity with the amino acid sequence set forth in any one of SEQ ID NOS of the amino acid sequences disclosed herein. The polypeptide or peptide “variant” disclosed herein may have one or more amino acids deleted or substituted by different amino acids. It is well understood in the art that some amino acids may be substituted or deleted without changing biological activity of the peptide (conservative substitutions). Suitably, the variant has at least 90%, 91%, 92%, 93%, 94%, 95%, 96%, 97%, 98% or 99% of the biological activity of the isolated polypeptide or peptide of any one of SEQ ID NOS of the amino acid sequences disclosed herein. In particular embodiments, the variant comprises, or is capable of forming antigenic proteins or polypeptides capable of triggering an immune response, whether humoral and/or cellular.

Terms used generally herein to describe sequence relationships between respective proteins and nucleic acids include “comparison window”, “sequence identity”, “percentage of sequence identity” and “substantial identity”. Because respective nucleic acids/proteins may each comprise (1) only one or more portions of a complete nucleic acid/protein sequence that are shared by the nucleic acids/proteins, and (2) one or more portions which are divergent between the nucleic acids/proteins, sequence comparisons are typically performed by comparing sequences over a “comparison window” to identify and compare local regions of sequence similarity. A “comparison window” refers to a conceptual segment of typically 6, 9 or 12 contiguous residues that is compared to a reference sequence. The comparison window may comprise additions or deletions (i.e., gaps) of about 20% or less as compared to the reference sequence for optimal alignment of the respective sequences. Optimal alignment of sequences for aligning a comparison window may be conducted by computerized implementations of algorithms (Geneworks program by Intelligenetics; GAP, BESTFIT, FASTA, and TFASTA in the Wisconsin Genetics Software Package Release 7.0, Genetics Computer Group, 575 Science Drive Madison, Wis., USA, incorporated herein by reference) or by inspection and the best alignment (i.e. resulting in the highest percentage homology over the comparison window) generated by any of the various methods selected. Reference also may be made to the BLAST family of programs as for example disclosed by Altschul et al., 1997, Nucl. Acids Res. 25 3389, which is incorporated herein by reference. A detailed discussion of sequence analysis can be found in Unit 19.3 of CURRENT PROTOCOLS IN MOLECULAR BIOLOGY Eds. Ausubel et al. (John Wiley & Sons Inc NY, 1995-2015), relevant portions incorporated herein by reference.

The term “sequence identity” is used herein in its broadest sense to include the number of exact nucleotide or amino acid matches having regard to an appropriate alignment using a standard algorithm, having regard to the extent that sequences are identical over a window of comparison. Thus, a “percentage of sequence identity” is calculated by comparing two optimally aligned sequences over the window of comparison, determining the number of positions at which the identical nucleic acid base (e.g., A, T, C, G, I) occurs in both sequences to yield the number of matched positions, dividing the number of matched positions by the total number of positions in the window of comparison (i.e., the window size), and multiplying the result by 100 to yield the percentage of sequence identity. For example, “sequence identity” may be understood to mean the “match percentage” calculated by the DNASIS or equivalent computer program (Version 2.5 for windows; available from Hitachi Software engineering Co., Ltd., South San Francisco, Calif., USA), relevant portions incorporated herein by reference.

The invention also provides fragments of the isolated peptide disclosed herein. In some embodiments, fragments may comprise, consist essentially of, or consist of 50%, 55%, 60%, 65%, 70%, 75%, 80%, 85%, 90%, 91%, 92%, 93%, 94%, 95%, 96%, 97%, 98% or 99% identity with any one of the amino acid sequences disclosed herein. In particular embodiments, the fragments comprise, or are capable of forming antigenic proteins or polypeptides capable of triggering an immune response, whether humoral and/or cellular.

Suitably, the fragments are antigenic proteins or polypeptides capable of triggering an immune response, whether humoral and/or cellular. Preferably, the fragment has at least 50%, 55%, 60%, 65%, 70%, 75%, 80%, 85%, 90%, 91%, 92%, 93%, 94%, 95%, 96%, 97%, 98% or 99% of the biological activity of the isolated peptide of any one of the amino acid sequences disclosed herein.

Derivatives of the isolated peptide disclosed herein are also provided. As used herein, “derivative” proteins or peptides have been altered, for example by conjugation or complexing with other chemical moieties, by post-translational modification (e.g. phosphorylation, ubiquitination, glycosylation), chemical modification (e.g. cross-linking, acetylation, biotinylation, oxidation or reduction and the like), conjugation with labels (e.g. fluorophores, enzymes, radioactive isotopes) and/or inclusion of additional amino acid sequences as would be understood in the art.

In this regard, the skilled person is referred to Chapter 15 of CURRENT PROTOCOLS IN PROTEIN SCIENCE, Eds. Coligan et al. (John Wiley & Sons NY 1995-2015), or equivalent, for more extensive methodology relating to chemical modification of proteins, relevant portions incorporated herein by reference. Additional amino acid sequences may include fusion partner amino acid sequences which create a fusion protein. By way of example, fusion partner amino acid sequences may assist in detection and/or purification of the isolated fusion protein. Non-limiting examples include metal-binding (e.g., polyhistidine) fusion partners, maltose binding protein (MBP), Protein A, glutathione S-transferase (GST), green fluorescent protein sequences (e.g., GFP), epitope tags such as myc, FLAG and haemagglutinin tags.

The isolated peptides, variant and/or derivatives of the present invention may be produced by any method known in the art, including but not limited to, chemical synthesis and recombinant DNA technology. Chemical synthesis is inclusive of solid phase and solution phase synthesis. Such methods are well known in the art, although reference is made to examples of chemical synthesis techniques as provided in Chapter 9 of SYNTHETIC VACCINES Ed. Nicholson (Blackwell Scientific Publications) and Chapter 15 of CURRENT PROTOCOLS IN PROTEIN SCIENCE Eds. Coligan et al., (John Wiley & Sons, Inc. NY USA 1995-2008). In this regard, reference is also made to International Publication WO 99/02550 and International Publication WO 97/45444. Recombinant proteins may be conveniently prepared by a person skilled in the art using standard protocols as for example described in Sambrook et al., MOLECULAR CLONING. A Laboratory Manual (Cold Spring Harbor Press, 1989), in particular Sections 16 and 17; CURRENT PROTOCOLS IN MOLECULAR BIOLOGY Eds. Ausubel et al., (John Wiley & Sons, Inc. NY USA 1995-2008), in particular Chapters 10 and 16; and CURRENT PROTOCOLS IN PROTEIN SCIENCE Eds. Coligan et al., (John Wiley & Sons, Inc. NY USA 1995-2008), in particular Chapters 1, 5 and 6, relevant portions incorporated herein by reference.

As used herein, the term a “vector” refers to a nucleic acid capable of transferring gene sequences to target cells (e.g., bacterial plasmid vectors, viral vectors, non-viral vectors, particulate carriers, and liposomes). Typically, “vector construct,” “expression vector,” and “gene transfer vector,” refers to any nucleic acid construct capable of directing the expression of one or more sequences of interest in a host cell. Thus, the term includes cloning and expression vehicles, as well as viral vectors. The term is used interchangeable with the terms “nucleic acid expression vector” and “expression cassette.”

Many suitable expression systems are commercially available, including, for example, the following: Plant Molecular Biology Manual A3:1-19 (1988); Miki, B. L. A., et al., pp. 249-265, and others in Plant DNA Infectious Agents (Hohn, T., et al., eds.) Springer-Verlag, Wien, Austria, (1987); Plant Molecular Biology: Essential Techniques, P. G. Jones and J. M. Sutton, New York, J. Wiley, 1997; Miglani, Gurbachan Dictionary of Plant Genetics and Molecular Biology, New York, Food Products Press, 1998; Henry, R. J., Practical Applications of Plant Molecular Biology, New York, Chapman & Hall, 1997), baculovirus expression (Reilly, P. R., et al., BACULOVIRUS EXPRESSION VECTORS: A LABORATORY MANUAL (1992); Beames, et al., Biotechniques 11:378 (1991); Pharmingen; Clontech, Palo Alto, Calif.)), vaccinia expression systems (Earl, P. L., et al., “Expression of proteins in mammalian cells using vaccinia” In Current Protocols in Molecular Biology (F. M. Ausubel, et al. Eds.), Greene Publishing Associates & Wiley Interscience, New York (1991); Moss, B., et al., U.S. Pat. No. 5,135,855, issued Aug. 4, 1992), expression in bacteria (Ausubel, F. M., et al., CURRENT PROTOCOLS IN MOLECULAR BIOLOGY, John Wiley and Sons, Inc., Media Pa.; Clontech), expression in yeast (Rosenberg, S. and Tekamp-Olson, P., U.S. Pat. No. RE35,749, issued, Mar. 17, 1998, herein incorporated by reference; Shuster, J. R., U.S. Pat. No. 5,629,203, issued May 13, 1997, herein incorporated by reference; Gellissen, G., et al., Antonie Van Leeuwenhoek, 62(1-2):79-93 (1992); Romanos, M. A., et al., Yeast 8(6):423-488 (1992); Goeddel, D. V., Methods in Enzymology 185 (1990); Guthrie, C., and G. R. Fink, Methods in Enzymology 194 (1991)), expression in mammalian cells (Clontech; Gibco-BRL, Ground Island, N.Y.; e.g., Chinese hamster ovary (CHO) cell lines (Haynes, J., et al., Nuc. Acid. Res. 11:687-706 (1983); 1983, Lau, Y. F., et al., Mol. Cell. Biol. 4:1469-1475 (1984); Kaufman, R. J., “Selection and coamplification of heterologous genes in mammalian cells,” in Methods in Enzymology, vol. 185, pp 537-566. Academic Press, Inc., San Diego Calif. (1991)), and expression in plant cells (plant cloning vectors, Clontech Laboratories, Inc., Palo-Alto, Calif., and Pharmacia LKB Biotechnology, Inc., Pistcataway, N.J.; Hood, E., et al., J. Bacteriol. 168:1291-1301 (1986); Nagel, R., et al., FEMS Microbiol. Lett. 67:325 (1990); An, et al., “Binary Vectors”, and others in relevant portion incorporated herein by reference.

As used herein, the term “subject” refers to any chordates, including, but not limited to, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The term does not denote a particular age. Thus, both adult and newborn individuals are intended to be covered. The system described herein is intended for use in any of the above vertebrate species, since the immune systems of all of these vertebrates operate similarly.

As used herein, the terms “pharmaceutically acceptable” or “pharmacologically acceptable” refer to a material which is not biologically or otherwise undesirable, i.e., the material may be administered to an individual in a formulation or composition without causing any unacceptable biological effects or interacting in a deleterious manner with any of the components of the composition in which it is contained.

As used herein, the term “treatment” refers to any of (i) the prevention of infection or reinfection, as in a traditional vaccine, (ii) the reduction or elimination of symptoms, and (iii) the substantial or complete elimination of the pathogen in question. Treatment may be effected prophylactically (prior to infection) or therapeutically (following infection).

As used herein, the term “adjuvant” refers to a substance that non-specifically changes or enhances an antigen-specific immune response of an organism to the antigen. Generally, adjuvants are non-toxic, have high-purity, are degradable, and are stable. The recombinant adjuvant of the present invention meets all of these requirements; it is non-toxic, highly-pure, degradable, and stable. Adjuvants are often included as one component in a vaccine or therapeutic composition that increases the specific immune response to the antigen. However, the present invention includes a novel adjuvant that does not have to be concurrently administered with the antigen to enhance an immune response, e.g., a humoral immune response. Unlike the common principle of action of other immunologic adjuvants, such as: (1) increasing surface area of an antigen to improve the immunogenicity thereof; (2) causing slow-release of the antigen to extend the retention time of the antigen in tissue; or (3) promoting an inflammatory reaction to stimulate active immune response, the present invention targets the B cells directly to enhance the production of antibodies. Non-limiting examples of adjuvant for use with the present invention includes one or more adjuvants selected from alum, aluminum hydroxide, aluminum phosphate, calcium phosphate hydroxide, cytosine-guanosine oligonucleotide (CpG-ODN) sequence, granulocyte macrophage colony stimulating factor (GM-CSF), monophosphoryl lipid A (MPL), poly(I:C), MF59, Quil A, N-acetyl muramyl-L-alanyl-D-isoglutamine (MDP), FIA, montanide, poly (DL-lactide-coglycolide), squalene, glucopyranosyl lipid adjuvant (GLA), GLA-Alum, 3M-052, a glucopyranosyl lipid adjuvant GLA emulsion with squalene (GLA-SE), virosome, AS03, ASO4, IL-1, IL-2, IL-3, IL-4, IL-5, IL-6, IL-7, IL-8, IL-10, IL-12, IL-15, IL-17, IL-18, STING, CD40L, pathogen-associated molecular patterns (PAMPs), damage-associated molecular pattern molecules (DAMPs), Freund's complete adjuvant, Freund's incomplete adjuvant, transforming growth factor (TGF)-beta antibody or antagonists, A2aR antagonists, lipopolysaccharides (LPS), Fas ligand, Trail, lymphotactin, Mannan (M-FP), APG-2, Hsp70 and Hsp90, pattern recognition receptor ligands, TLR3 ligands, TLR4 ligands, TLR5 ligands, TLR7/8 ligands, or TLR9 ligands.

As used herein, the term “effective dose” refers to that amount of an immunogenic peptide or fusion protein that includes the coronavirus antigens described herein. Further, the immunogenic peptide can be fused with another protein to express and/or display the antigenic epitope or to provide a fusion protein that is processed by antigen presenting cells for display in the context of MHC Class I and/or Class II protein. As described herein, the antigenic peptide can be fused to an N-terminal, C-terminal, and/or a loop formed between amino acid 74 and 82 to form a fusion protein that includes, e.g., a coronavirus Receptor Binding Motif (RBM) of the spike protein (S protein), a nucleocapsid protein (N protein), or both such as a SARS-CoV-2 spike protein, of the present invention sufficient to induce immunity, to prevent and/or ameliorate an infection or to reduce at least one symptom of an infection and/or to enhance the efficacy of another dose of a coronavirus. The coronavirus can be SARS, MERS, 229E (alpha), NL63 (alpha), OC43 (beta), or HKU1 (beta), B.1.617.2 (Delta), and P.1 (Gamma), SARS-CoV-2, or variants thereof. An effective dose may refer to the amount of the fusion protein sufficient to delay or minimize the onset of an infection. An effective dose may also refer to the fusion protein in an amount that provides a therapeutic benefit in the treatment or management of an infection. Further, an effective dose is the amount with respect to the fusion protein of the invention alone, or in combination with other therapies, that provides a therapeutic benefit in the treatment or management of an infection. An effective dose may also be the amount sufficient to enhance a subject's (e.g., a human's) own immune response against a subsequent exposure to an infectious agent. Levels of immunity can be monitored, e.g., by measuring amounts of neutralizing secretory and/or serum antibodies, e.g., by plaque neutralization, complement fixation, enzyme-linked immunosorbent, or microneutralization assay. In the case of a vaccine, an “effective dose” is one that prevents disease and/or reduces the severity of symptoms.

As used herein, the term “carrier protein” refers to a polypeptide chain into which an antigenic peptide or polypeptide is inserted in the form of a fusion protein. In certain embodiments, the present invention includes carrier proteins such as a modified thermostable lichenase (LicKM) polypeptide-antigen fusion proteins that have multiple antigenic proteins against multiple types, variants, or strains of coronavirus; a human hepatitis core antigen (HBcAg) polypeptide-antigen fusion proteins or VLPs that have one or more antigenic peptides, domains, or proteins against multiple types, variants, or strains of coronavirus; and/or a truncated woodchuck hepatitis core antigen (WHcAg) polypeptide-antigen fusion proteins or VLPs that have one or more antigenic peptides, domains, or proteins against multiple types, variants, or strains of coronavirus.

As used herein, the term “immune stimulator” refers to a compound that enhances an immune response via the body's own chemical messengers (cytokines). These molecules comprise various cytokines, lymphokines and chemokines with immunostimulatory, immunopotentiating, and pro-inflammatory activities, such as interferons, interleukins (e.g., IL-1, IL-2, IL-3, IL-4, IL-12, IL-13); growth factors (e.g., granulocyte-macrophage (GM)-colony stimulating factor (CSF)); and other immunostimulatory molecules, such as macrophage inflammatory factor, Flt3 ligand, B7.1; B7.2, etc. The immune stimulator molecules can be administered in the same formulation as the HBcAg-RBM fusion protein of the present invention, or can be administered separately. Either the protein or an expression vector encoding the protein can be administered to produce an immunostimulatory effect.

As used herein, the term “innate immune response stimulator” refers to agents that trigger the innate or non-specific immune response. The innate immune response is a nonspecific defense mechanism is able to act immediately (or within hours) of an antigen's appearance in the body and the response to which is non-specific, that is, it responds to an entire class of agents (such as oligosaccharides, lipopolysaccharides, nucleic acids such as the CpG motif, etc.) and does not generate an adaptive response, that is, they do not cause immune memory to the antigen. Pathogen-associated immune stimulants act through the Complement cascade, Toll-like Receptors, and other membrane bound receptors to trigger phagocytes to directly kill the perceived pathogen via phagocytosis and/or the expression of immune cell stimulating cytokines and chemokines to stimulate both the innate and adaptive immune responses. The present inventors take advantage of the innate immune response to help enhance the adaptive immune response by glycosylating to the antigens taught herein, thus enhancing antigen presentation and generation of both T and B cell-drive immune responses. Glycosylation sites can also be added to enhance the glycosylation of the antigens taught herein, in particular, those that are incorporated in plant cells.

As used herein, the term “protective immune response” or “protective response” refers to an immune response mediated by antibodies or effector cells against an infectious agent, which is exhibited by a vertebrate (e.g., a human), which prevents or ameliorates an infection or reduces at least one symptom thereof. The LicKM-, HBcAg-, WHcAg-antigen fusion protein of the invention can stimulate the production of antibodies that, for example, neutralize infectious agents, blocks infectious agents from entering cells, blocks replication of said infectious agents, and/or protect host cells from infection and destruction. The term can also refer to an immune response that is mediated by T-lymphocytes and/or other white blood cells against an infectious agent, exhibited by a vertebrate (e.g., a human), that prevents or ameliorates coronavirus infection or reduces at least one symptom thereof.

As used herein, the term “antigenic formulation” or “antigenic composition” refers to a preparation which, when administered to a vertebrate, e.g., a mammal, will induce an immune response.

As used herein, the terms “immunization” or “vaccine” are used interchangeably to refer to a formulation which contains the LicKM-, HBcAg-, WHcAg-antigen fusion proteins of the present invention, which is in a form that is capable of being administered to a vertebrate and which induces a protective immune response sufficient to induce immunity to prevent and/or ameliorate an infection and/or to reduce at least one symptom of an infection and/or to enhance the efficacy of another dose or exposure to the coronavirus. Typically, the vaccine comprises a conventional saline or buffered aqueous solution medium in which the composition of the present invention is suspended or dissolved. In this form, the composition of the present invention can be used conveniently to prevent, ameliorate, or otherwise treat an infection. Upon introduction into a host, the vaccine is able to provoke an immune response including, but not limited to, the production of antibodies and/or cytokines and/or the activation of cytotoxic T cells, antigen presenting cells, helper T cells, dendritic cells and/or other cellular responses.

The practice of the present invention employs, unless otherwise indicated, conventional methods of chemistry, biochemistry, molecular biology, immunology and pharmacology, within the skill of the art. Such techniques are explained fully in the literature. See, e.g., Remington's Pharmaceutical Sciences, 18th Edition (Easton, Pa.: Mack Publishing Company, 1990); Methods In Enzymology (S. Colowick and N. Kaplan, eds., Academic Press, Inc.); and Handbook of Experimental Immunology, Vols. I-IV (D. M. Weir and C. C. Blackwell, eds., 1986, Blackwell Scientific Publications); Sambrook, et al., Molecular Cloning: A Laboratory Manual (2nd Edition, 1989); Short Protocols in Molecular Biology, 4th ed. (Ausubel et al. eds., 1999, John Wiley & Sons); Molecular Biology Techniques: An Intensive Laboratory Course, (Ream et al., eds., 1998, Academic Press); PCR (Introduction to Biotechniques Series), 2nd ed. (Newton & Graham eds., 1997, Springer Verlag); Fundamental Virology, Second Edition (Fields & Knipe eds., 1991, Raven Press, New York), relevant portion incorporated herein by reference.

The capability of single plant cell to regenerate and give rise to whole plant with all genetic features of the parent and ii) transfer of foreign genes into a plant genome by a plant-infecting bacterium, Agrobacterium tumefaciens (A. tumefaciens) enabled workers in this field to develop new procedures for crop improvement and stable expression of foreign proteins in plants.

In addition to transgenic plants, with the advances made in molecular plant virology, plant viruses have also emerged as promising tools. Plant viruses have features that range from detrimental to potentially beneficial. The substantial crop losses world-wide due to viral infections have prompted the molecular plant virologists to develop genetic systems that allow manipulation of the virus for management of plant diseases. These genetic systems have also led to the use of viruses as tools, since small plus-sense single-stranded RNA viruses that commonly infect higher plants can be used rapidly amplify virus-related RNAs and produce large amounts of protein.

Both transgenic plants and engineered plant viruses have been used in producing foreign proteins in plant. In the early 1990, transgenic plant technology moved to a new arena as a heterologous expression system for antigens from mammalian pathogens. Since then, a variety of medically important antigens have been expressed in transgenic plants, including hepatitis B surface antigen (HBsAg) E. coli heat-labile enterotoxin, rabies virus glycoprotein, and Norwalk virus capsid protein.

A number of inducible promoters that may allow control over the expression of target genes in transgenic plants have been described. Based on their specificity to a particular class of inducers these promoters could be divided into three groups: i) promoters that are induced at different developmental stages (flowering, senescence, etc.) in different organs (roots, flowers, seeds, etc.), ii) promoters that respond to particular environmental signals (heat-shock, nutritional status, pathogen attack or mechanical wounding), iii) promoters that are induced by chemicals of non-plant origin (tetracycline-, glucocorticoid-, ecdysteroid-, copper- and ethanol-inducible promoters). The latter generally utilize non-plant transcription factors that require chemical inducers for activation. Compared to the first two groups of promoters, chemical-inducible systems have much greater potential for a strict temporal and spatial control of the expression of the target gene expression in transgenic plants. Unfortunately, current inducible plant expression systems have some shortcomings, including leaky promoters or commercially unfeasible manufacturing conditions.

An alternative system for the expression of foreign proteins in plants is based on plant virus vectors. Although plant virus vector-based expression systems have a number of advantages (time, efficient engineering and production, level of target protein expression, environmental safety, etc.) compared to that of transgenic plants, they have some limitations as well. For example, the stability and systemic movement of the recombinant virus may be affected by the size of the target gene. Virus-based vectors are probably less applicable in projects that require coordinated expression of multi-subunit proteins, such as antibodies and enzyme complexes.

The present invention provides vectors and methods for expression of foreign sequences (peptides, polypeptides, and RNA) in plants. Specifically, the present invention relates to vectors and methods for activation of silenced or inactive foreign nucleic acid sequence(s) or gene(s) of interest in plant and animal cells for production of peptides, polypeptides, and RNA in such cells. The vectors used for the activation of silenced or inactive sequence(s) are viral vectors.

The activation of silenced or inactive foreign nucleic acid sequence(s) or gene(s) in plant or animal cells is achieved, in trans, by a factor (e.g., a protein or polypeptide) encoded by a nucleic acid sequence located on the viral vector after the cells are infected with the viral vector. In other words, delivery of activator gene via infection with transient gene delivery viral vector into plant or animal cell activates and results in the expression of target sequence(s). Thus, in the present invention, the activation of silenced or inactive foreign nucleic acid sequence(s) or gene(s) in plant or animal cells is transactivation. It is transactivation because the factor(s) are encoded by nucleic acid sequences that are remotely located, i.e., on the viral vectors, and the factor(s) are free to migrate or diffuse through the cell to their sites of action.

The antigenic portion of the coronavirus may be fused with other sequences that facilitate expression, transport across the cell membranes, tissues and/or systemic delivery. See, for example, U.S. Pat. No. 6,051,239 for sequences which can be fused to the target gene of interest. As part of creating the first component of the transactivation system, a nucleic acid construct is introduced into the plant cell or a plant via a genetic transformation procedure. The nucleic acid construct can be a circular construct such as a plasmid construct or a phagemid construct or cosmid vector or a linear nucleic acid construct including, but not limited to, PCR products. Regardless of the form, the nucleic acid construct introduced is a cassette (also referred to herein as a transfer cassette or an expression cassette) having elements such as promoter(s) and/or enhancer(s) elements besides target gene(s) or the desired coding sequence, among other things. Expression of the target gene, however, depends on transactivation provided by the second component of the invention described further below.

The transactivation system can be a recombinase-based transactivation system or a transcription factor type (with activation and binding domains) based transactivation system. In the recombinase-based transactivation system, the gene of interest (target gene or TG) is cloned into a transfer cassette (or a transformation plasmid) for integration into the plant genome and stable transformation. The target gene in the transformation plasmid is made non-functional by placing a blocking sequence between the promoter (and other regulatory sequences) for driving the expression of the target gene and the target gene. The resulting transfer cassette (or transgenic DNA) is said to have, among other things, the following structure: promoter-blocking sequence-TG.

Different promoters may be used with the present invention, such as, ubiquitous or constitutive (e.g., Cauliflower Mosaic Virus 35S promoter), or tissue specific promoters (e.g., potato protease inhibitor II (pin2) gene promoter, promoters from a number of nodule genes). A number of such promoters are known in the art. Inducible promoters that specifically respond to certain chemicals (copper etc.,) or heat-shock (HSP) are also contemplated. Numerous tissue specific and inducible promoters have been described from plants. The blocking sequence contains a selectable marker element or any other nucleic acid sequence (referred to herein as stuffer) flanked on each side by a recombinase target site (e.g., “FRT” site) with a defined 5′ to 3′ orientation. The FRT refers to a nucleic acid sequence at which the product of the FLP gene, i.e., FLP recombinase, can catalyze the site-specific recombination.

A selectable marker element or stuffer is generally an open reading frame of a gene or alternatively of a length sufficient enough to prevent readthrough. When a suitable recombinase is provided by the second component of the transactivation system to the cells of the transgenic plant containing the transgenic DNA or expression cassette, the recombinase protein can bind to the two target sites on the transgenic DNA, join its two target sequences together and excise the DNA between them so that the target gene is attached to a promoter and/or an enhancer in operable linkage. The recombinase is provided in cells by a viral vector and the recombinase activates the expression of the target gene in cells where it is otherwise silenced or not usually expressed because of the blocking sequence.

It should be noted that the type of recombinase, which is provided to the plant cells in the present invention, would depend upon the recombination target sites in the transgenic DNA (or more specifically in the targeting cassette). For example, if FRT sites are used, the FLP recombinase is provided in the plant cells. Similarly, where lox sites are used, the Cre recombinase is provided in the plant cells. If the non-identical sites are used, for example both an FRT and a lox site, then both the FLP and Cre are provided in the plant cells.

The recombinases used herein are sequence-specific recombinases. These are enzymes that recognize and bind to a short nucleic acid site or a target sequence and catalyze the recombination events. A number of sequence-specific recombinases and their corresponding target sequences are known in the art. For example, the FLP recombinase protein and its target sequence, FRT, are well-characterized and known to one skilled in the art. Briefly, the FLP is a 48 kDa protein encoded by the plasmid of the yeast, Saccharomyces cerevisiae. The FLP recombinase function is to amplify the copy number of the plasmid in the yeast. The FLP recombinase mediates site-specific recombination between a pair of nucleotide sequences, FLP Recognition Targets (FRY s). The FRT is a site for the 48 kDa FLP recombinase. The FRT site is a three repeated DNA sequences of 13 bp each; two repeats in a direct orientation and one in an inverted to the other two. The repeats are separated by the 8 bp spacer region that determine the orientation of the FRT recombination site. Depending of the orientation of the FRT sites FLP-mediated DNA excision or inversion occurs. FRT and FLP sequences can be either wild type or mutant sequences as long as they retain their ability to interact and catalyze the specific excision. Transposases and integrases and their recognition sequences may also be used.

A transfer cassette system may also be used. A viral replicon (e.g., V-BEC) is placed upstream of a target gene. The viral replicon is a viral nucleic acid sequence that allows for the extrachromosomal replication of a nucleic acid construct in a host cell expressing the appropriate replication factors. The replication factor may be provided by a viral vector or a transgenic plant carrying a replicase transgene. Such transgenic plants are known in the art. See, for example, PCT International Publication WO 00/46350. The constructs of the present invention containing a viral origin of replication, once transcribed, replicate to a high copy number in cells that express the appropriate replication factors. The transfer cassette may contain more than one target gene each linked to a promoter and other elements. Each of the target genes may be transactivated by factors provided by a specific viral vector in a host cell.

In the transcription factor type (for example with activation and binding domains) based transactivation system, the gene of interest (target gene or TG) is cloned into a transfer cassette (or a transformation plasmid) for integration into the host genome (animal or plant) and stable transformation. The target gene will only be expressed when a suitable transcription factor activity is available. This can happen when a fusion protein containing a DNA-binding domain and an activation domain interacts with certain regulatory sequences cloned into the transfer cassette that is integrated into the host genome.

Viral vectors may also be used to deliver factors for transactivation of inactive or silenced target genes in transgenic host cells or organisms. The viral vectors can be RNA type and do not integrate into host genome and the expression is extrachromosomal (transient or in the cytoplasm). Recombinant plant viruses are used in the case of transgenic plant cells or plants. The use of plant viral vectors for expression of recombinases in plants provides a means to have high levels of gene expression within a short time. The autonomously replicating viruses offer several advantages for use as gene delivery vehicles for transient expression of foreign genes, including their characteristic high levels of multiplication and transient gene expression. The recombinant viral vectors used in the present invention are also capable of infecting a suitable host plant and systemically transcribing or expressing foreign sequences or polypeptides in the host plant. Systemic infection or the ability to spread systemically of a virus is an ability of the virus to spread from cell to cell and from infected areas to uninfected distant areas of the infected plant, and to replicate and express in most of the cells of the plant. Thus, this ability of plant viruses to spread to the rest of the plant and their rapid replication would aid in delivery of factors for transactivation throughout the plant and the consequent large-scale production of polypeptides of interest in a short time.

Therefore, the invention also includes the construction of recombinant viral vectors by manipulating the genomic component of the wild-type viruses. Viruses include RNA containing plant viruses. Although many plant viruses have RNA genomes, it is well known that organization of genetic information differs among groups. Thus, a virus can be a mono-, bi-, tri-partite virus. “Genome” refers to the total genetic material of the virus. “RNA genome” states that as present in virions (virus particles), the genome is in RNA form.

[1] Some of the viruses which meet this requirement, and are therefore suitable, include Alfalfa Mosaic Virus (Al MV), ilarviruses, cucumoviruses such as Cucumber Green Mottle Mosaic virus (CGMMV), closteroviruses or tobamaviruses (tobacco mosaic virus group) such as Tobacco Mosaic virus (TMV), Tobacco Etch Virus (TEV), Cowpea Mosaic virus (CMV), and viruses from the brome mosaic virus group such as Brome Mosaic virus (BMV), broad bean mottle virus and cowpea chlorotic mottle virus. Additional suitable viruses include Rice Necrosis virus (RNV), and geminiviruses such as tomato golden mosaic virus (TGMV), Cassava latent virus (CLV) and maize streak virus (MSV). Each of these groups of suitable viruses are well characterized and are well known to the skilled artisans in the field. A number of recombinant viral vectors have been used by those skilled in the art to transiently express various polypeptides in plants. See, for example, U.S. Pat. Nos. 5,316,931 and 6,042,832; and PCT International Publications WO 00/46350, WO 96/12028 and WO 00/25574, the contents of which are incorporated herein by reference. Thus, the methods already known in the art can be used as a guidance to develop recombinant viral vectors of the present invention to deliver transacting factors.

The recombinant viral vector used in the present invention can be heterologous virus vectors. The heterologous virus vectors as referred to herein are those having a recombinant genomic component of a given class of virus (for example TMV) with a movement protein encoding nucleic acid sequence of the given class of virus but coat protein (either a full-length or truncated but functional) nucleic acid sequence of a different class of virus (for example AIMV) in place of the native coat protein nucleic acid sequence of the given class of virus. Likewise native movement protein nucleic acid sequence instead of the coat protein sequence is replaced by heterologous (i.e., not native) movement protein from another class of virus. For example, a TMV genomic component having an AlMV coat protein is one such heterologous vector. Similarly, an AlMV genomic component having a TMV coat protein is another such heterologous vector. The vectors are designed such that these vectors, upon infection, are capable of replicating in the host cell and transiently activating genes of interest in transgenic plants. Such vectors are known in the art, for example, as described in PCT International Publication WO 00/46350.

In accordance with the present invention, the host plants included within the scope of the present invention are all species of higher and lower plants of the Plant Kingdom. Mature plants, seedlings, and seeds are included in the scope of the invention. A mature plant includes a plant at any stage in development beyond the seedling. A seedling is a very young, immature plant in the early stages of development. Specifically, plants that can be used as hosts to produce foreign sequences and polypeptides include and are not limited to Angiosperms, Bryophytes such as Hepaticae (liverworts) and Musci (mosses); Pteridophytes such as ferns, horsetails, and lycopods; Gymnosperms such as conifers, cycads, Ginkgo, and Gnetales; and Algae including Chlorophyceae, Phaeophpyceae, Rhodophyceae, Myxophyceae, Xanthophyceae, and Euglenophyceae.

Host plants used for transactivation of genes can be grown either in vivo and/or in vitro depending on the type of the selected plant and the geographic location. It is important that the selected plant is amenable to cultivation under the appropriate field conditions and/or in vitro conditions. The conditions for the growth of the plants are described in various basic books on botany, Agronomy, Taxonomy and Plant Tissue Culture, and are known to a skilled artisan in these fields.

Among angiosperms, the use of crop and/or crop-related members of the families are particularly contemplated. The plant members used in the present methods also include interspecific and/or intergeneric hybrids, mutagenized and/or genetically engineered plants. These families include and not limited to Leguminosae (Fabaceae) including pea, alfalfa, and soybean; Gramineae (Poaceae) including rice, corn, wheat; Solanaceae particularly of the genus Lycopersicon, particularly the species esculentum (tomato), the genus Solanum, particularly the species tuberosum (potato) and melongena (eggplant), the genus Capsicum, particularly the species annum (pepper), tobacco, and the like; Umbelliferae, particularly of the genera Daucus, particularly the species carota (carrot) and Apium, particularly the species graveolens duke, (celery) and the like; Rutaceae, particularly of the genera Citrus (oranges) and the like; Compositae, particularly the genus Lactuca, and the species sativa (lettuce), and the like and the family Cruciferae, particularly of the genera Brassica and Sinapis. Examples of “vegetative” crop members of the family Brassicaceae include, but are not limited to, digenomic tetraploids such as Brassica juncea (L.) Czern. (mustard), B. carinata Braun (ethopian mustard), and monogenomic diploids such as B. oleracea (L.) (cole crops), B. nigra (L.) Koch (black mustard), B. campestris (L.) (turnip rape) and Raphanus sativus (L.) (radish). Examples of “oil-seed” crop members of the family Brassicaceae include, but are not limited to, B. napus (L.) (rapeseed), B. campestris (L.), B. juncea (L.) Czem. and B. tournifortii and Sinapis alba (L.) (white mustard). Flax plants are also contemplated.

Particularly preferred host plants are those that can be infected by AlMV. For example, it is known in the art that alfalfa mosaic virus has full host range. Other species that are known to be susceptible to the virus are: Abelmoschus esculentus, Ageratum conyzoides, Amaranthus caudatus, Amaranthus retroflexus, Antirrhinum majus, Apium graveolens, Apium graveolens var. rapaceum, Arachis hypogaea, Astragalus glycyphyllos, Beta vulgaris, Brassica campestris ssp. raga, Calendula officinalis, Capsicum annuum, Capsicumfrutescens, Caryopteris incana, Catharanthus roseus, Celosia argentea, Cheiranthus cheiri, Chenopodium album, Chenopodium amaranticol, Chenopodium murale, Chenopodium quinoa, Cicer arietinum, Cichium endiva, Ciandrum sativum, Crotalaria spectabilis, Cucumis melo, Cucumis sativus, Cucurbita pepo, Cyamopsis tetragonoloba, Daucus carota (var. sativa), Dianthus barbatus, Dianthus caryophyllus, Emilia sagittata, Fagopyrum esculentum, Glycine max, Gomphrena globosa, Helianthus annuus, Lablab purpureus, Lactuca sativa, Lathyrus odatus, Lens culinaris, Linum usitatissimum, Lupinus a/bus, Lycopersicon esculentum, Macroptilium lathyroides, Malva parvifla, Matthiola incana, Medicago hispida, Medicago sativa, Melilotus albus, Nicotiana bigelovii, Nicotiana clevelandii, Nicotiana debneyi, Nicotiana glutinosa, Nicotiana megalosiphon, Nicotiana rustica, Nicotiana sylvestris, Nicotiana tabacum, Ocimum basilicum, Petunia x hybrida, Phaseolus lunatus, Phaseolus vulgaris, Philadelphus, Physalis flidana, Physalis peruviana, Phytolacca americana, Pisum sativum, Solanum demissum, Solanum melongena, Solanum nigrum, Solanum nodiflum, Solanum rostratum, Solanum tuberosum, Sonchus oleraceus, Spinacia oleracea, Ste/Zaria media, Tetragonia tetragonioides, Trifolium dubium, Trifolium hybridum, Trifolium incarnatum, Trifolium pratense, Trifolium repens, Trifolium subterraneum, Tropaeolum majus, Viburnum opulus, Viciafaba, Vigna radiata, Vigna unguiculata, Vigna unguiculata ssp. sesquipedalis, and Zinnia elegans.

A plant virus vector (Av or AlMV) is engineered to express FLP recombinase. The gene for this protein is cloned under subgenomic promoter for coat protein, movement protein or artificial subgenomic promoter. The target gene is cloned into an agrobacterial vector and introduced into nuclear genome to obtain transgenic plants. The target gene is placed under a strong promoter (ubiquitin, dub35, super). However, the expression is silenced by the introduction of NPT or stuffer sequence flanked by FRT (blocking sequence). Target gene is activated by removing the blocking sequences. There can be more than one target gene in a transfer cassette. The target gene(s) is (are) cloned into an agrobacterial vector and introduced into nuclear genome or chloroplast genome. These transformation procedures are well known in the art. Target gene is placed under strong promoter (ubiquitin, dub35, super). However, the expression is silenced by the introduction NPT or stuffer sequences flanked by recombinase recognition sites (e.g., FRT or lox) between the promoter and the TG. The target gene is activated by removing sequences between the promoter and the TG. There could be more than one target gene. The virus vector capable of expressing recombinase in plant cells and transgenic plants (nuclear or chloroplast) that are so made can readily be used to produce target proteins. Transgenic plants are infected with virus containing gene for recombinase.

Inoculation of plants; sprouts, leaves, roots, or stems is done using infectious RNA transcripts, infectious cDNA clones or pregenerated virus material. See, PCT International Publication, WO 00/46350 for guidance on infectious RNA transcripts and procedures for viral infection. Because the time span for target protein production according to the present invention is short (up to 15 days) the expression may not be affected by the gene silencing machinery within the host.

Vaccines. The present invention contemplates immunization for use in both active and passive immunization embodiments. Immunogenic compositions, proposed to be suitable for use as a vaccine, may be prepared most readily directly from immunogenic LicKM-, HBcAg-, WHcAg-antigen fusion protein prepared in a manner disclosed herein. Often, the antigenic material is extensively processed to remove undesired contaminants, such as, small molecular weight molecules, incomplete proteins, or when manufactured in plant cells, plant components such as cell walls, plant proteins, and the like. Often, these immunizations are lyophilized for ease of transport and/or to increase shelf-life and can then be more readily dissolved in a desired vehicle, such as saline.

The present inventors used two different approaches to identify novel antigens for immunization. An in silico immunogenicity study was conducted to identify T cell epitopes in S and N proteins. The novel S and N T cell epitopes were incorporated into new, diversely organized LicKM and VLPs constructs for accumulation testing. Multiple N epitopes were combined with S sequences in VLP and LicKM constructs.

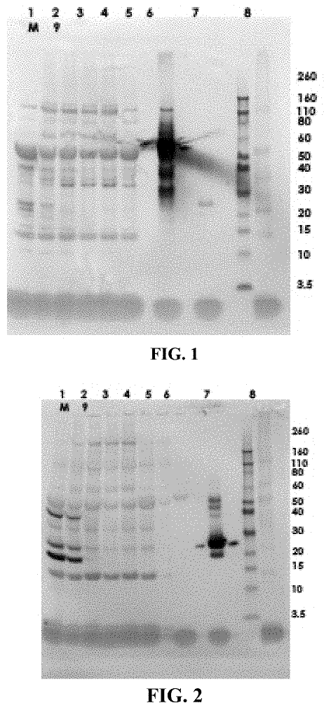

FIG. 1 shows a Western blot analyses results for the listed constructs, Lane 1, iBio201 40 1 (39.5 kDa); Lane 2, iBio201 40 2 (39.5 kDa); Lane 3, iBio200 41 (14.4 kDa); Lane 4, iBio201 42 (14.4 kDa); Lane 5, iBio201 43 (14.6 kDa); Lane 6, iBio201 44 (14.6 kDa); Lane 7, SARS CoV2 N with His tag Positive control 3 ug; Lane 8, LicKM with His tag Positive control 4 ug; Lane M, Novex pre stained protein ladder; Lane 9, Mock; Lane 10, IL 6 with His tag Positive control 3 ug; Antibody Direct blot with Anti His Antibody HRP conjugated, all samples are heated and reduced.

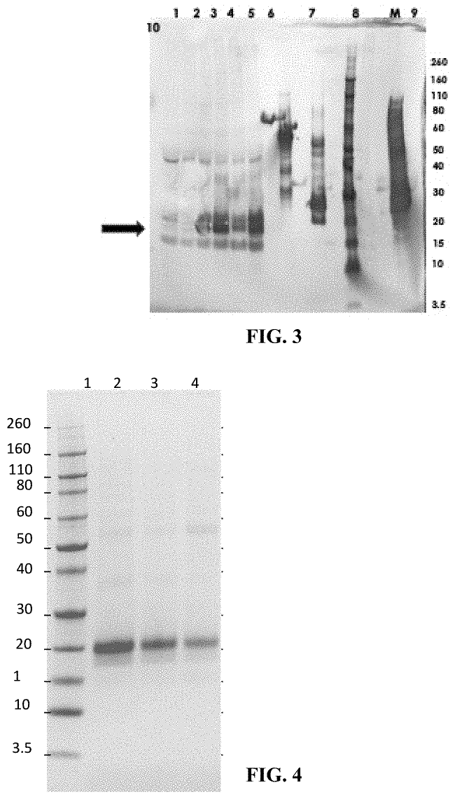

FIG. 2 shows the expression of 8HIS-CoV-41 antigen, purified.

FIG. 3 is a chart with the intact mass determination for purified 8HIS-CoV-41 by mass spectrometry.

FIG. 4 is a gel that shows the expression of the purified 8HIS-CoV-41.

TABLE 1
Fusion Proteins

| | |
|---|---|
| CoV-1 | HB 144-CoV-RBM: HBcAg core 144—Linker—RBM end |
| CoV-2 | HB78-CoV-RBM: HBcAg core 149—RBM loop |
| CoV-3 | WH148-CoV-RBM: Extensin SP—WHeAg core—Linker— |
| | RBM |
| CoV-4 | WH78-CoV-RBMee: Extensin SP—WHeAg core RBM |
| | Position 78 with linker |
| CoV-5 | WH78-CoV-RBM: Extensin SP—WHeAg core RBM |
| | Position 78 |
| CoV-6 | WH74-CoV-RBMee: Extensin SP—WHeAg core RBM |
| | Position 74 with linker |
| CoV-7 | LicKM-CoV-RBD-loop |
| CoV-8 | LicKM-CoV-RBM-loop |
| CoV-9 | LicKM-CoV-RBD end |
| CoV-10 | LicKM-CoV-RBM end |
| CoV-11 | PR1a SP-LicKM-CoV-RBD-loop |
| CoV-12 | PR1a SP-LicKM-CoV-RBM-loop |
| CoV-13 | PR1a SP-LicKM-CoV-RBD end |
| CoV-14 | PR1a SP-LicKM-CoV-RBM end |
| CoV-15 | HB78-CoV-RBM Epi 1 loop |
| CoV-16 | HB78-CoV-RBM Epi 2 loop |
| CoV-17 | HB78-CoV-RBM Epi l & 2 loop |
| CoV-18 | PR1a-LicKM-CoV-RBD end No His tag |
| CoV-19 | HBcAg—RBM with long linker loop |
| CoV-20 | HBcAg—RBD with long linker loop |
| CoV-21 | HBcAg—Grifoni RBD epitope 1 with long linker loop |
| CoV-22 | HBcAg—Grifoni RBD epitopes 1 & 2 with long linker loop |
| CoV-23 | HBcAg—RBD with long linker end |
| CoV-24 | LicKM-CoV-RBD end No His tag |
| CoV25: | PR1a-LicKM-Spike 319-684 c-term/66 kDa |
| CoV26: | PR1a-LicKM-Spike 319-684 Loop/66 kDa |
| CoV27: | Spike 227-684 c-term/76.2 kDa |
| CoV28: | Spike 227-684 Loop/76.2 kDa |
| CoV29: | Spike 319-684 c-term + T-cell epitope/72.7 kDa |
| CoV30: | Spike 319-598 c-term + T-cell epitope 63.5 kDa |
| CoV-31: | HBcAg S1-93/S1-105/S2-78 25 kDa |
| CoV-32: | Spike 524-598 HBcAg loop/27 kDa |
| CoV-33: | Spike 601-640 HBcAg loop/23 kDa |
| CoV-34: | Spike 530-684 HBcAg dual core/58 kDa |
| CoV-35: | Spike 437-508 HBcAg dual core [RBM] |
| CoV-36: | LicKM-N protein T-cell epitope mix no cysteines + RBM/44 |
| | kDa |
| CoV-37: | HB144-CoV-Ntcell: HBcAg core 144—Linker—N protein |
| | T-cell epitopes/26 kDa |
| CoV-38 | PR1a-LicKM-N protein T-cell epitope mix no cysteines—35 |
| | kDa |
| CoV-39 | PR1a-LicKM-N protein T-cell epitope mix no cysteines and |
| | RBM at C terminus—43.5 kDa |
| CoV-40 | Ext-LicKM-Linker-CoV N 247-365 |
| CoV-41 | Ext-8HIS-CoV N 247-365 |
| CoV-42 | Ext-CoV N 247-365-8HIS |
| CoV-43 | Ext-8HIS-CoV N 245-365 |
| CoV-44 | Ext-CoV N 245-365-8HIS |
| CoV-45 | IBIO201 S-NTD (19-310)_N-CTD (245-370) Long-Long |
| CoV-46 | IBIO201 S-NTD (19-310)_N-CTD (248-366) Long-Short |
| CoV-47 | IBIO201 S-NTD (19-290)_N-CTD (245-370) Short-Long |
| CoV-48 | IBIO201S-NTD(19-290)_N-CTD(248-366)Short-Short |
| CoV-49 | IBIO201S-RBD(319-554)_N-CTD(245-370)Long-Long |
| CoV-50 | IBIO201S-RBD(319-554)_N-CTD(248-366)Long-Short |
| CoV-51 | IBIO201S-RBD(348-523)_N-CTD(245-370)Short-Long |
| CoV-52 | IBIO201S-RBD(348-523)_N-CTD(248-366)Short-Short |

For each of the following sequences, the bolded, underlined, or italicized portions are as set forth in the description for each construct. For example, in the example of construct, CoV-1: 11B144-CoV-RBM: describes a fusion protein that includes the HBcAg core sequence to residue 144, then a linker in bolded letter (Linker) followed by the receptor binding domain in italics (RBM), which is a fusion protein with a molecular weight of 24.76 kDa.

CoV-1 HB144-CoV-RBM: HBcAg core 144 - Linker - RBM (24.76 kDa),
(SEQ ID NO: 1)
MDIDPYKEFGATVELLSFLPSDFFPSVRDLLDTASALYREALESPEHCSPHHTALRQAILCW
GELMTLATWVGANLEDPASRDLVVNYVNTNMGLKIRQLLWFHISCLTFGRETVLEYLVSF
GVWIRTPPAYRPPNAPILSTLPSGGSNSNNLDSKVGGNYNYLYRLFRKSNLKPFERDISTEIYQA
GSTPCNGVEGFNCYFPLQSYGFQPTNGVGYQPY
(SEQ ID NO: 2)
TtaattaaATGGACATCGATCCGTACAAAGAATTTGGCGCGACCGTCGAGCTGCTGAGCTTC
CTGCCGAGCGATTTTTTCCCGAGCGTGCGTGACCTGCTGGACACCGCGAGCGCACTGTA
TCGTGAAGCACTGGAAAGCCCAGAGCACTGTAGCCCGCACCACACCGCCCTGCGCCAG
GCGATTCTGTGCTGGGGTGAACTGATGACCCTGGCCACCTGGGTGGGTGCTAACCTTGA
GGATCCGGCGAGCCGTGATCTGGTCGTCAACTATGTGAATACCAACATGGGTCTGAAA
ATTCGTCAGCTGCTGTGGTTTCATATTAGCTGCCTGACCTTCGGTCGTGAAACCGTGCT
GGAGTATCTGGTGAGCTTCGGTGTGTGGATTCGCACCCCGCCGGCGTATCGTCCGCCGA
ACGCGCCAATTCTGAGCACGCTGCCGTCCGGAGGTAGCAACTCTAACAACCTGGACTCT
AAGGTTGGCGGCAACTACAACTACCTCTACAGGCTGTTCCGGAAGTCCAACCTTAAGCCTTTC
GAGAGGGATATCAGCACCGAGATCTATCAGGCTGGTTCTACTCCTTGCAACGGTGTTGAGGG
TTTCAACTGCTACTTCCCGCTTCAGTCTTACGGTTTCCAGCCTACTAATGGTGTGGGCTACCA
GCCTTATTAGctcgag
CoV-2 HB78-CoV-RBM: HBcAg core 149 - RBM
(SEQ ID NO: 3)
MDIDPYKEFGATVELLSFLPSDFFPSVRDLLDTASALYREALESPEHCSPHHTALRQAILCW
GELMYLAYWVGANLEDPNSNNLDSKFGGNYNYLYRLFRKSNLKPFERDISTEIYQAGSTPCNGV
EGFNCYFPLQSYGFQPTNGVGYQPYASRDLVVNYVNTNMGLKIRQLLWFHISCLTFGRETVL
EYLVSFGVWIRTPPAYRPPNAPILSTLPETTVV
(SEQ ID NO: 4)
TtaattaaATGGACATCGATCCGTACAAAGAATTTGGCGCGACCGTCGAGCTGCTGAGCTTC
CTGCCGAGCGATTTTTTCCCGAGCGTGCGTGACCTGCTGGACACCGCGAGCGCACTGTA
TCGTGAAGCACTGGAAAGCCCAGAGCACTGTAGCCCGCACCACACCGCCCTGCGCCAG
GCGATTCTGTGCTGGGGTGAACTGATGACCCTGGCCACCTGGGTGGGTGCTAACCTTGA
GGATCCGAACTCTAACAACCTGGACTCTAAGGTTGGCGGCAACTACAACTACCTCTACAGGC
TGTTCCGGAAGTCCAACCTTAAGCCTTTCGAGAGGGATATCAGCACCGAGATCTATCAGGCT
GGTTCTACTCCTTGCAACGGTGTTGAGGGTTTCAACTGCTACTTCCCGCTTCAGTCTTACGGT
TTCCAGCCTACTAATGGTGTGGGCTACCAGCCTTATGCTAGCCGTGATCTGGTCGTCAACT
ATGTGAATACCAACATGGGTCTGAAAATTCGTCAGCTGCTGTGGTTTCATATTAGCTGC
CTGACCTTCGGTCGTGAAACCGTGCTGGAGTATCTGGTGAGCTTCGGTGTGTGGATTCG
CACCCCGCCGGCGTATCGTCCGCCGAACGCGCCAATTCTGAGCACGCTGCCGGAGACC
ACCGTGGTTTAGctcgag
BamHI and NheI sites respectively before and after the RBM
CoV-3 WH148-CoV-RBM: Extensin Signal peptide - WHcAg core - Linker - RBM
(25.46 kDa + 1.6 kDa)
(SEQ ID NO: 5)
MGKMASLFATFLVVLVSLSLASESSADIDPYKEFGSSYQLLNFLPLDFFPDLNALVDTATAL
YEEELTGREHCSPHHTAIRQALVCWDELTKLIAWMSSNITSEQVRTIIVNHVNDTWGLKVR
QSLWFHLSCLTFGQHTVQEFLVSFGVWIRTPAPYRPPNAPILSTLPEHTVISGGSNSNNLDSK
VGGNYNYLYRLFRKSNLKPFERDISTEIYQAGSTPCNGVEGFNCYFPLQSYGFQPTNGVGYQPY
(SEQ ID NO: 6)
ttaattaaATGGGAAAAATGGCTTCTCTTTTTGCTACTTTCCTTGTTGTGTTGGTTAGTCTTTC
TCTAGCTAGTGAGAGTAGTGCTGATATCGATCCTTATAAGGAATTCGGTTCTTCATACC
AACTTTTGAATTTTCTTCCTTTGGATTTCTTTCCAGATCTTAATGCTTTGGTTGATACTGC
TACAGCTCTTTATGAAGAGGAATTGACTGGAAGAGAGCATTGTTCTCCACATCATACA
GCTATTAGGCAAGCTTTGGTTTGCTGGGATGAGCTTACTAAGTTGATTGCTTGGATGTC
TTCAAACATCACTTCAGAACAAGTTAGAACAATTATTGTTAACCATGTTAATGATACAT
GGGGTCTTAAAGTTAGGCAATCTTTGTGGTTCCATCTTTCATGTTTGACTTTTGGACAAC
ATACAGTTCAAGAGTTTCTTGTTTCTTTTGGTGTTTGGATTAGAACTCCAGCTCCTTATA
GGCCACCTAATGCTCCTATTCTTTCTACTTTGCCAGAACATACAGTTATTTCCGGAGGT
AGCAACTCTAACAACCTGGACTCTAAGGTTGGCGGCAACTACAACTACCTCTACAGGCTGTT
CCGGAAGTCCAACCTTAAGCCTTTCGAGAGGGATATCAGCACCGAGATCTATCAGGCTGGTT
CTACTCCTTGCAACGGTGTTGAGGGTTTCAACTGCTACTTCCCGCTTCAGTCTTACGGTTTCC
AGCCTACTAATGGTGTGGGCTACCAGCCTTATTAGctcgag
CoV-4 WH78-CoV-RBMee: Extensin Signal peptide - WHcAg core RBM Position
78 (25.7 kDa)
(SEQ ID NO: 7)
MGKMASLFATFLVVLVSLSLASESSADIDPYKEFGSSYQLLNFLPLDFFPDLNALVDTATAL
YEEELTGREHCSPHHTAIRQALVCWDELTKLIAWMSSNITSEENSNNLDSKVGGNYNYLYRLF
RKSNLKPFERDISTEIYQAGSTPCNGVEGFNCYFPLQSYGFQPTNGVGYQPYEEEQVRTIIVNHV
NDTWGLKVRQSLWFHLSCLTFGQHTVQEFLVSFGVWIRTPAPYRPPNAPILSTLPEHTVI
(SEQ ID NO: 8)
TtaattaaATGGGAAAAATGGCTTCTCTTTTTGCTACTTTCCTTGTTGTGTTGGTTAGTCTTTC
TCTAGCTAGTGAGAGTAGTGCTGATATCGATCCTTATAAGGAATTCGGTTCTTCATACC
AACTTTTGAATTTTCTTCCTTTGGATTTCTTTCCAGATCTTAATGCTTTGGTTGATACTGC
TACAGCTCTTTATGAAGAGGAATTGACTGGAAGAGAGCATTGTTCTCCACATCATACA
GCTATTAGGCAAGCTTTGGTTTGCTGGGATGAGCTTACTAAGTTGATTGCTTGGATGTC
TTCAAACATCACTTCAgaagagAACTCTAACAACCTGGACTCTAAGGTTGGCGGCAACTACAA
CTACCTCTACAGGCTGTTCCGGAAGTCCAACCTTAAGCCTTTCGAGAGGGATATCAGCACCG
AGATCTATCAGGCTGGTTCTACTCCTTGCAACGGTGTTGAGGGTTTCAACTGCTACTTCCCGC
TTCAGTCTTACGGTTTCCAGCCTACTAATGGTGTGGGCTACCAGCCTTATgaagagGAACAAGT
TAGAACAATTATTGTTAACCATGTTAATGATACATGGGGTCTTAAAGTTAGGCAATCTT
TGTGGTTCCATCTTTCATGTTTGACTTTTGGACAACATACAGTTCAAGAGTTTCTTGTTT
CTTTTGGTGTTTGGATTAGAACTCCAGCTCCTTATAGGCCACCTAATGCTCCTATTCTTT
CTACTTTGCCAGAACATACAGTTATTTAGctcgag
CoV-5 WH78-CoV-RBM: Extensin Signal peptide - WHcAg core RBM Position 78
(25.17 kDa)
(SEQ ID NO: 9)
MGKMASLFATFLVVLVSLSLASESSADIDPYKEFGSSYQLLNFLPLDFFPDLNALVDTATAL
YEEELTGREHCSPHHTAIRQALVCWDELTKLIAWMSSNITSNSNNLDSKVGGNYNYLYRLFRK
SNLKPFERDISTEIYQAGSTPCNGVEGFNCYFPLQSYGFQPTNGVGYQPYEQVRTIIVNHVNDT
WGLKVRQSLWFHLSCLTFGQHTVQEFLVSFGVWIRTPAPYRPPNAPILSTLPEHTVI
(SEQ ID NO: 10)
TtaattaaATGGGAAAAATGGCTTCTCTTTTTGCTACTTTCCTTGTTGTGTTGGTTAGTCTTTC
TCTAGCTAGTGAGAGTAGTGCTGATATCGATCCTTATAAGGAATTCGGTTCTTCATACC
AACTTTTGAATTTTCTTCCTTTGGATTTCTTTCCAGATCTTAATGCTTTGGTTGATACTGC
TACAGCTCTTTATGAAGAGGAATTGACTGGAAGAGAGCATTGTTCTCCACATCATACA
GCTATTAGGCAAGCTTTGGTTTGCTGGGATGAGCTTACTAAGTTGATTGCTTGGATGTC
TTCAAACATCACTTCAAACTCTAACAACCTGGACTCTAAGGTTGGCGGCAACTACAACTACC
TCTACAGGCTGTTCCGGAAGTCCAACCTTAAGCCTTTCGAGAGGGATATCAGCACCGAGATC
TATCAGGCTGGTTCTACTCCTTGCAACGGTGTTGAGGGTTTCAACTGCTACTTCCCGCTTCAG
TCTTACGGTTTCCAGCCTACTAATGGTGTGGGCTACCAGCCTTATGAACAAGTTAGAACAA
TTATTGTTAACCATGTTAATGATACATGGGGTCTTAAAGTTAGGCAATCTTTGTGGTTC
CATCTTTCATGTTTGACTTTTGGACAACATACAGTTCAAGAGTTTCTTGTTTCTTTTGGT
GTTTGGATTAGAACTCCAGCTCCTTATAGGCCACCTAATGCTCCTATTCTTTCTACTTTG
CCAGAACATACAGTTATTTAGctcgag
CoV-6 WH74-CoV-RBMee: Extensin Signal peptide - WHcAg core RBM Position
74 (25.69 kDa)
(SEQ ID NO: 11)
MGKMASLFATFLVVLVSLSLASESSADIDPYKEFGSSYQLLNFLPLDFFPDLNALVDTATAL
YEEELTGREHCSPHHTAIRQALVCWDELTKLIAWMSSEENSNNLDSKVGGNYNYLYRLFRKS
NLKPFERDISTEIYQAGSTPCNGVEGFNCYFPLQSYGFQPTNGVGYQPYEENITSEQVRTIIVNH
VNDTWGLKVRQSLWFHLSCLTFGQHTVQEFLVSFGVWIRTPAPYRPPNAPILSTLPEHTVI
(SEQ ID NO: 12)
TtaattaaATGGGAAAAATGGCTTCTCTTTTTGCTACTTTCCTTGTTGTGTTGGTTAGTCTTTC
TCTAGCTAGTGAGAGTAGTGCTGATATCGATCCTTATAAGGAATTCGGTTCTTCATACC
AACTTTTGAATTTTCTTCCTTTGGATTTCTTTCCAGATCTTAATGCTTTGGTTGATACTGC
TACAGCTCTTTATGAAGAGGAATTGACTGGAAGAGAGCATTGTTCTCCACATCATACA
GCTATTAGGCAAGCTTTGGTTTGCTGGGATGAGCTTACTAAGTTGATTGCTTGGATGTC
TTCAgaagagAACTCTAACAACCTGGACTCTAAGGTTGGCGGCAACTACAACTACCTCTACAGG
CTGTTCCGGAAGTCCAACCTTAAGCCTTTCGAGAGGGATATCAGCACCGAGATCTATCAGGC
TGGTTCTACTCCTTGCAACGGTGTTGAGGGTTTCAACTGCTACTTCCCGCTTCAGTCTTACGG
TTTCCAGCCTACTAATGGTGTGGGCTACCAGCCTTATgaagagAACAYCACYYCAGAACAAGY
TAGAACAATTATTGTTAACCATGTTAATGATACATGGGGTCTTAAAGTTAGGCAATCTT
TGTGGTTCCATCTTTCATGTTTGACTTTTGGACAACATACAGTTCAAGAGTTTCTTGTTT
CTTTTGGTGTTTGGATTAGAACTCCAGCTCCTTATAGGCCACCTAATGCTCCTATTCTTT
CTACTTTGCCAGAACATACAGTTATTTAGctcgag
CoV-7 LicKM-CoV-RBD-loop (50.33 kDa)
(SEQ ID NO: 13)
MHHHHHHHHGGSYPYKSGEYRTKSFFGYGYYEVRMKAAKNVGIVSSFFTYTGPSDNNPW
DEIDIEFLGKDTTKVQFNWYKNGVGGNEYLHNLGFDASQDFHTYGFEWRPDYIDFYVDG
KKVYRGTRNIPVTPGKIMMNLWPGIGVDEWLGRYDGRTPLQAEYEYVKYYPNGRSRVQP
TESIVRFPNITNLCPFGEVFNATRFASVYAWNRKRISNCVADYSVLYNSASFSTFKCYGVSPTKLND
LCFTNVYADSFVIRGDEVRQIAPGQTGKIADYNYKLPDDFTGCVIAWNSNNLDSKVGGNYNYLYR
LFRKSNLKPFERDISTEIYQAGSTPCNGVEGFNCYFPLQSYGFQPTNGVGYQPYRVWLSFELLH
APATVCGPKKFKLVVNTPFVAVFSNFDSSQWEKADWANGSVFNCVWKPSQVTFSNGKMIL
TLDREY
(SEQ ID NO: 14)
ttaattaaATGCATCACCATCACCACCATCATCATGGCGGCTCTTACCCTTATAAGAGCGGT
GAGTACCGGACCAAGAGCTTCTTTGGTTACGGTTACTACGAGGTGCGGATGAAGGCTG
CTAAGAACGTGGGTATCGTGTCCAGCTTCTTTACCTACACCGGGCCATCTGATAACAAC
CCTTGGGATGAGATCGACATCGAGTTCCTTGGTAAGGACACTACCAAGGTGCAGTTCA
ACTGGTACAAGAACGGTGTTGGTGGCAACGAGTACCTTCACAACCTTGGCTTTGATGC
CAGCCAGGATTTCCACACTTACGGTTTTGAATGGCGGCCTGACTACATCGACTTCTACG
TGGACGGTAAGAAGGTGTACAGGGGCACCAGAAATATCCCTGTGACTCCTGGCAAGAT
CATGATGAACCTTTGGCCTGGTATCGGTGTGGATGAGTGGCTTGGTAGATACGATGGT
AGGACTCCTCTGCAGGCTGAGTACGAGTACGTTAAGTACTACCCTAACGGCAGATCTA
GGGTTCAGCCTACTGAGTCTATTGTGCGGTTCCCGAACATCACCAACTTGTGCCCTTTTGGC
GAGGTGTTCAATGCTACCAGGTTCGCTTCTGTGTACGCCTGGAATCGGAAGCGGATTTCTAA
CTGCGTGGCCGATTACAGCGTGCTGTACAACTCTGCTAGCTTCAGCACCTTCAAGTGCTACG
GTGTGTCTCCTACCAAGCTGAACGATCTGTGCTTCACCAACGTGTACGCTGACTCTTTCGTGA
TCAGGGGTGATGAGGTTAGGCAGATTGCTCCTGGTCAGACCGGTAAGATCGCTGACTACAA
CTACAAGCTGCCTGATGACTTCACCGGTTGCGTGATCGCTTGGAACTCTAACAACCTGGACT
CTAAGGTTGGCGGCAATTACAACTACCTCTACCGGCTGTTCCGGAAGTCTAACCTTAAGCCTT
TCGAGCGGGATATCAGCACCGAGATCTATCAGGCTGGTTCTACTCCTTGCAATGGCGTTGAG
GGTTTCAACTGCTACTTCCCGCTTCAGTCTTACGGATTCCAGCCTACTAATGGTGTGGGCTAC
CAGCCTTACAGAGTGGTGGTTTTGTCTTTCGAGCTTCTGCATGCTCCTGCTACTGTTTGCGGT
CCGAAGAAGTTCAAGCTTGTCGTTAATACCCCTTTCGTGGCCGTGTTCAGCAACTTCGAT
TCTAGCCAGTGGGAGAAAGCTGATTGGGCTAACGGTTCTGTGTTCAACTGCGTGTGGA
AGCCTTCTCAGGTGACCTTCTCTAACGGCAAGATGATTCTGACCCTGGACCGTGAGTAT
TAGctcgag
CoV-8 LicKM-CoV-RBM-loop (34.78 kDa)
(SEQ ID NO: 15)
MHHHHHHHHGGSYPYKSGEYRTKSFFGYGYYEVRMKAAKNVGIVSSFFTYTGPSDNNPW
DEIDIEFLGKDTTKVQFNWYKNGVGGNEYLHNLGFDASQDFHTYGFEWRPDYIDFYVDG
KKVYRGTRNIPVTPGKIMMNLWPGIGVDEWLGRYDGRTPLQAEYEYVKYYPNGRSNSNN
LDSKVGGNYNYLYRLFRKSNLKPFERDISTEIYQAGSTPCNGVEGFNCYFPLQSYGFQPTNGVGY
QPYFKLVVNTPFVAVFSNFDSSQWEKADWANGSVFNCVWKPSQVTFSNGKMILTLDREY
(SEQ ID NO: 16)
TTAATTAAATGCATCACCATCACCACCATCATCATGGCGGCTCTTACCCTTATAAGAGC
GGTGAGTACCGGACCAAGAGCTTCTTTGGTTACGGTTACTACGAGGTGCGGATGAAGG
CTGCTAAGAACGTGGGTATCGTGTCCAGCTTCTTTACCTACACCGGGCCATCTGATAAC
AACCCTTGGGATGAGATCGACATCGAGTTCCTTGGTAAGGACACTACCAAGGTGCAGT
TCAACTGGTACAAGAACGGTGTTGGTGGCAACGAGTACCTTCACAACCTTGGCTTTGA
TGCCAGCCAGGATTTCCACACTTACGGTTTTGAATGGCGGCCTGACTACATCGACTTCT
ACGTGGACGGTAAGAAGGTGTACAGGGGCACCAGAAATATCCCTGTGACTCCTGGCA
AGATCATGATGAACCTTTGGCCTGGTATCGGTGTGGATGAGTGGCTTGGTAGATACGA
TGGTAGGACTCCTCTGCAGGCTGAGTACGAGTACGTTAAGTACTACCCTAACGGCAGA
TCTAACAGCAACAACCTGGATTCTAAGGTTGGCGGCAACTACAACTACCTCTACAGGCTGTT
CCGGAAGTCCAACCTTAAGCCTTTCGAGAGGGATATCAGCACCGAGATCTATCAGGCTGGTT
CTACTCCTTGCAATGGCGTTGAGGGTTTCAACTGCTACTTCCCGCTTCAGTCTTACGGATTCC
AGCCTACTAATGGTGTTGGCTACCAGCCGTACTTCAAGCTTGTGGTGAATACCCCTTTCGT
GGCCGTGTTCAGCAACTTCGATTCTAGCCAGTGGGAGAAAGCTGATTGGGCTAACGGT
TCTGTGTTCAACTGCGTGTGGAAGCCTTCTCAGGTGACCTTCTCTAACGGCAAGATGAT
TCTGACCCTGGACCGTGAGTATTAGCTCGAG
CoV-9 LicKM-CoV-RBD (50.46 kDa)
(SEQ ID NO: 17)
MHHHHHHHHGGSYPYKSGEYRTKSFFGYGYYEVRMKAAKNVGIVSSFFTYTGPSDNNPW
DEIDIEFLGKDTTKVPFNWYKNGVGGNEYLHNLGFDASPDFHTYGFEWRPDYIDFYVDG
KKVYRGTRNIPVTPGKIMMNLWPGIGVDEWLGRYDGRTPLPAEYEYVKYYPNGRSEFKL
VVNTPFVAVFSNFDSSPWEKADWANGSVFNCVWKPSPVTFSNGKMILTLDREYRVQPTES
IVRFPNITNLCPFGEVFNATRFASVYAWNRKRISNCVADYSVLYNSASFSTFKCYGVSPTKLNDLC
FTNVYADSFVIRGDEVRQIAPGQTGKIADYNYKLPDDFTGCVIAWNSNNLDSKVGGNYNYLYRLF
RKSNLKPFERDISTEIYQAGSTPCNGVEGFNCYFPLQSYGFQPTNGVGYQPYRVWLSFELLHAP
ATVCGPKK
(SEQ ID NO: 18)
TTAATTAAATGCATCACCATCACCACCATCATCATGGCGGCTCTTACCCTTATAAGAGC
GGTGAGTACCGGACCAAGAGCTTCTTTGGTTACGGTTACTACGAGGTGCGGATGAAGG
CTGCTAAGAACGTGGGTATCGTGTCCAGCTTCTTTACCTACACCGGGCCATCTGATAAC
AACCCTTGGGATGAGATCGACATCGAGTTCCTTGGTAAGGACACTACCAAGGTGCAGT
TCAACTGGTACAAGAACGGTGTTGGTGGCAACGAGTACCTTCACAACCTTGGCTTTGA
TGCCAGCCAGGATTTCCACACTTACGGTTTTGAATGGCGGCCTGACTACATCGACTTCT
ACGTGGACGGTAAGAAGGTGTACAGGGGCACCAGAAATATCCCTGTGACTCCTGGCA
AGATCATGATGAACCTTTGGCCTGGTATCGGTGTGGATGAGTGGCTTGGTAGATACGA
TGGTAGGACTCCTCTGCAGGCTGAGTACGAGTACGTTAAGTACTACCCTAACGGCAGA
TCTGAATTCAAGCTTGTGGTGAATACTCCTTTCGTGGCCGTGTTCAGCAACTTCGATTCT
AGCCAGTGGGAGAAAGCTGATTGGGCTAACGGTTCTGTGTTCAACTGCGTGTGGAAGCCTTC
TCAGGTGACCTTCTCTAACGGCAAGATGATTCTGACCCTGGACCGTGAGTATAGGGTTCAGC
CTACTGAGTCTATCGTGCGGTTCCCTAACATCACCAACTTGTGCCCTTTCGGCGAGGTGTTC
AATGCTACTAGGTTCGCTTCTGTGTACGCCTGGAACCGGAAGAGGATTTCTAACTGCGTGGC
CGATTACAGCGTGCTGTACAACTCTGCTAGCTTCAGCACCTTCAAGTGCTACGGTGTGTCTC
CTACCAAGCTGAACGATCTGTGCTTCACCAACGTGTACGCTGACTCTTTCGTGATCAGGGGT
GATGAGGTTAGGCAGATTGCTCCTGGTCAGACCGGTAAGATCGCTGACTACAACTACAAGCT
GCCTGATGACTTCACCGGTTGCGTGATCGCTTGGAACTCTAACAACCTGGACTCTAAGGTTG
GCGGCAATTACAACTACCTCTACCGGCTGTTCCGGAAGTCTAACCTTAAGCCTTTCGAGCGG
GATATCAGCACCGAGATCTATCAGGCTGGTTCTACTCCTTGCAATGGCGTTGAGGGTTTCAA
CTGCTACTTCCCGCTTCAGTCTTACGGATTCCAGCCTACTAATGGTGTGGGCTACCAGCCTTA
CAGAGTGGTGGTTTTGTCTTTCGAGCTTCTGCATGCTCCTGCTACTGTTTGCGGTCCTAAGAA
GTAGCTCGAG
CoV-10 - LicKM-CoV-RBM (34.91 kDa)
(SEQ ID NO: 19)
MHHHHHHHHGGSYPYKSGEYRTKSFFGYGYYEVRMKAAKNVGIVSSFFTYTGPSDNNPW
DEIDIEFLGKDTTKVPFNWYKNGVGGNEYLHNLGFDASPDFHTYGFEWRPDYIDFYVDG
KKVYRGTRNIPVTPGKIMMNLWPGIGVDEWLGRYDGRTPLPAEYEYVKYYPNGRSEFKL
VVNTPFVAVFSNFDSSQWEKADWANGSVFNCVWKPSQVTFSNGKMILTLDREYNSNNLDS
KVGGNYNYLYRLFRKSNLKPFERDISTEIYQAGSTPCNGVEGFNCYFPLQSYGFQPTNGVGYQPY
(SEQ ID NO: 20)
TTAATTAAATGCATCACCATCACCACCATCATCATGGCGGCTCTTACCCTTATAAGAGC
GGTGAGTACCGGACCAAGAGCTTCTTTGGTTACGGTTACTACGAGGTGCGGATGAAGG
CTGCTAAGAACGTGGGTATCGTGTCCAGCTTCTTTACCTACACCGGGCCATCTGATAAC
AACCCTTGGGATGAGATCGACATCGAGTTCCTTGGTAAGGACACTACCAAGGTGCAGT
TCAACTGGTACAAGAACGGTGTTGGTGGCAACGAGTACCTTCACAACCTTGGCTTTGA
TGCCAGCCAGGATTTCCACACTTACGGTTTTGAATGGCGGCCTGACTACATCGACTTCT
ACGTGGACGGTAAGAAGGTGTACAGGGGCACCAGAAATATCCCTGTGACTCCTGGCA
AGATCATGATGAACCTTTGGCCTGGTATCGGTGTGGATGAGTGGCTTGGTAGATACGA
TGGTAGGACTCCTCTGCAGGCTGAGTACGAGTACGTTAAGTACTACCCTAACGGCAGA
TCTGAATTCAAGCTTGTGGTGAATACTCCTTTCGTGGCCGTGTTCAGCAACTTCGATT
CTAGCCAGTGGGAGAAAGCTGATTGGGCTAACGGTTCTGTGTTCAACTGCGTGTGGAA
GCCTTCTCAGGTGACCTTCTCTAACGGCAAGATGATTCTGACCCTGGACCGTGAGTACA
ACAGCAACAACCTGGATTCTAAGGTCGGCGGCAACTACAACTACCTCTACAGGCTGTTCCGG
AAGTCCAACCTTAAGCCTTTCGAGAGGGATATCAGCACCGAGATCTATCAGGCTGGTTCTAC
TCCTTGCAATGGCGTTGAGGGTTTCAACTGCTACTTCCCGCTTCAGTCTTACGGATTCCAGCC
TACTAATGGTGTGGGCTACCAGCCTTATTAGCTCGAG
CoV-11 PR1a-LicKM-CoV-RBD-loop (50.33 kDa)
(SEQ ID NO: 21)
MGFVLFSQLPSFLLVSTLLLFLVISHSCRAHHHHHHHHGGSYPYKSGEYRTKSFFGYGYYE
VRMKAAKNVGIVSSFFTYTGPSDNNPWDEIDIEFLGKDTTKVQFNWYKNGVGGNEYLHN
LGFDASQDFHTYGFEWRPDYIDFYVDGKKVYRGTRNIPVTPGKIMMNLWPGIGVDEWLG
RYDGRTPLQAEYEYVKYYPNGRSRVQPTESIVRFPNITNLCPFGEVFNATRFASVYAWNRKRIS
NCVADYSVLYNSASFSTFKCYGVSPTKLNDLCFTNVYADSFVIRGDEVRQIAPGQTGKIADYNYKL
PDDFTGCVIAWNSNNLDSKVGGNYNYLYRLFRKSNLKPFERDISTEIYQAGSTPCNGVEGFNCYF
PLQSYGFQPTNGVGYQPYRVVVLSFELLHAPATVCGPKKFKLVVNTPFVAVFSNFDSSQWEK
ADWANGSVFNCVWKPSQVTFSNGKMILTLDREY
(SEQ ID NO: 22)
ACGTCAttaattaaATGGGATTCGTTTTGTTTTCTCAATTGCCTTCATTTCTTTTGGTTTCTAC
TCTTTTGCTTTTTCTTGTTATTTCTCATTCATGTAGAGCTCATCACCATCACCACCATCA
TCATGGCGGCTCTTACCCTTATAAGAGCGGTGAGTACCGGACCAAGAGCTTCTTTGGTT
ACGGTTACTACGAGGTGCGGATGAAGGCTGCTAAGAACGTGGGTATCGTGTCCAGCTT
CTTTACCTACACCGGGCCATCTGATAACAACCCTTGGGATGAGATCGACATCGAGTTC
CTTGGTAAGGACACTACCAAGGTGCAGTTCAACTGGTACAAGAACGGTGTTGGTGGCA
ACGAGTACCTTCACAACCTTGGCTTTGATGCCAGCCAGGATTTCCACACTTACGGTTTT
GAATGGCGGCCTGACTACATCGACTTCTACGTGGACGGTAAGAAGGTGTACAGGGGCA
CCAGAAATATCCCTGTGACTCCTGGCAAGATCATGATGAACCTTTGGCCTGGTATCGGT
GTGGATGAGTGGCTTGGTAGATACGATGGTAGGACTCCTCTGCAGGCTGAGTACGAGT
ACGTTAAGTACTACCCTAACGGCAGATCTAGGGTTCAGCCTACTGAGTCTATTGTGCGGT
TCCCGAACATCACCAACTTGTGCCCTTTTGGCGAGGTGTTCAATGCTACCAGGTTCGCTTCT
GTGTACGCCTGGAATCGGAAGCGGATTTCTAACTGCGTGGCCGATTACAGCGTGCTGTACAA
CTCTGCTAGCTTCAGCACCTTCAAGTGCTACGGTGTGTCTCCTACCAAGCTGAACGATCTGT
GCTTCACCAACGTGTACGCTGACTCTTTCGTGATCAGGGGTGATGAGGTTAGGCAGATTGCT
CCTGGTCAGACCGGTAAGATCGCTGACTACAACTACAAGCTGCCTGATGACTTCACCGGTTG
CGTGATCGCTTGGAACTCTAACAACCTGGACTCTAAGGTTGGCGGCAATTACAACTACCTCTA
CCGGCTGTTCCGGAAGTCTAACCTTAAGCCTTTCGAGCGGGATATCAGCACCGAGATCTATC
AGGCTGGTTCTACTCCTTGCAATGGCGTTGAGGGTTTCAACTGCTACTTCCCGCTTCAGTCTT
ACGGATTCCAGCCTACTAATGGTGTGGGCTACCAGCCTTACAGAGTGGTGGTTTTGTCTTTC
GAGCTTCTGCATGCTCCTGCTACTGTTTGCGGTCCGAAGAAGTTCAAGCTTGTCGTTAATA
CCCCTTTCGTGGCCGTGTTCAGCAACTTCGATTCTAGCCAGTGGGAGAAAGCTGATTGG
GCTAACGGTTCTGTGTTCAACTGCGTGTGGAAGCCTTCTCAGGTGACCTTCTCTAACGG
CAAGATGATTCTGACCCTGGACCGTGAGTATTAGctcgagACGAAG
CoV-12 PR1a-LicKM-CoV-RBM-loop (34.78 kDa)
(SEQ ID NO: 23)
MGFVLFSQLPSFLLVSTLLLFLVISHSCRAHHHHHHHHGGSYPYKSGEYRTKSFFGYGYYE
VRMKAAKNVGIVSSFFTYTGPSDNNPWDEIDIEFLGKDTTKVQFNWYKNGVGGNEYLHN
LGFDASQDFHTYGFEWRPDYIDFYVDGKKVYRGTRNIPVTPGKIMMNLWPGIGVDEWLG
RYDGRTPLGAEYEYVRYYPNGRSNSNNLDSKVGGNYNYLYRLFRKSNLKPFERDISTEIYQAG
STPCNGVEGFNCYFPLQSYGFQPTNGVGYQPYFKLVVNTPFVAVFSNFDSSQWEKADWANG
SVFNCVWKPSQVTFSNGKMILTLDREY
(SEQ ID NO: 24)
ACGTCATTAATTAAATGGGATTCGTTTTGTTTTCTCAATTGCCTTCATTTCTTTTGGTTT
CTACTCTTTTGCTTTTTCTTGTTATTTCTCATTCATGTAGAGCTCATCACCATCACCACC
ATCATCATGGCGGCTCTTACCCTTATAAGAGCGGTGAGTACCGGACCAAGAGCTTCTTT
GGTTACGGTTACTACGAGGTGCGGATGAAGGCTGCTAAGAACGTGGGTATCGTGTCCA
GCTTCTTTACCTACACCGGGCCATCTGATAACAACCCTTGGGATGAGATCGACATCGA
GTTCCTTGGTAAGGACACTACCAAGGTGCAGTTCAACTGGTACAAGAACGGTGTTGGT
GGCAACGAGTACCTTCACAACCTTGGCTTTGATGCCAGCCAGGATTTCCACACTTACG
GTTTTGAATGGCGGCCTGACTACATCGACTTCTACGTGGACGGTAAGAAGGTGTACAG
GGGCACCAGAAATATCCCTGTGACTCCTGGCAAGATCATGATGAACCTTTGGCCTGGT
ATCGGTGTGGATGAGTGGCTTGGTAGATACGATGGTAGGACTCCTCTGCAGGCTGAGT
ACGAGTACGTTAAGTACTACCCTAACGGCAGATCTAACAGCAACAACCTGGATTCTAAGG
TTGGCGGCAACTACAACTACCTCTACAGGCTGTTCCGGAAGTCCAACCTTAAGCCTTTCGAG
AGGGATATCAGCACCGAGATCTATCAGGCTGGTTCTACTCCTTGCAATGGCGTTGAGGGTTT
CAACTGCTACTTCCCGCTTCAGTCTTACGGATTCCAGCCTACTAATGGTGTTGGCTACCAGCC
GTACTTCAAGCTTGTGGTGAATACCCCTTTCGTGGCCGTGTTCAGCAACTTCGATTCTAG
CCAGTGGGAGAAAGCTGATTGGGCTAACGGTTCTGTGTTCAACTGCGTGTGGAAGCCT
TCTCAGGTGACCTTCTCTAACGGCAAGATGATTCTGACCCTGGACCGTGAGTATTAGCT
CGAGACGAAG
CoV-13 PR1a-LicKM-CoV-RBD (50.46 kDa)
(SEQ ID NO: 25)
MGFVLFSQLPSFLLVSTLLLFLVISHSCRAHHHHHHHHGGSYPYKSGEYRTKSFFGYGYYE
VRMKAAKNVGIVSSFFTYTGPSDNNPWDEIDIEFLGKDTTKVQFNWYKNGVGGNEYLHN
LGFDASQDFHTYGFEWRPDYIDFYVDGKKVYRGTRNIPVTPGKIMMNLWPGIGVDEWLG
RYDGRTPLQAEYEYVKYYPNGRSEFKLVVNTPFVAVFSNFDSSQWEKADWANGSVFNCV
WKPSQVYYSNGKM1EYEDREYRVQPTESIVRFPNITNLCPFGEVFNATRFASVYAWNRKRISNC
VADYSVLYNSASFSTFKCYGVSPTKLNDLCFTNVYADSFVIRGDEVRQIAPGQTGKIADYNYKLPD
DFTGCVIAWNSNNLDSKVGGNYNYLYRLFRKSNLKPFERDISTEIYQAGSTPCNGVEGFNCYFPL
QSYGFQPTNGVGYQPYRVVVLSFELLHAPATVCGPKK
(SEQ ID NO: 26)
ACGTCATTAATTAAATGGGATTCGTTTTGTTTTCTCAATTGCCTTCATTTCTTTTGGTTT
CTACTCTTTTGCTTTTTCTTGTTATTTCTCATTCATGTAGAGCTCATCACCATCACCACC
ATCATCATGGCGGCTCTTACCCTTATAAGAGCGGTGAGTACCGGACCAAGAGCTTCTTT
GGTTACGGTTACTACGAGGTGCGGATGAAGGCTGCTAAGAACGTGGGTATCGTGTCCA
GCTTCTTTACCTACACCGGGCCATCTGATAACAACCCTTGGGATGAGATCGACATCGA
GTTCCTTGGTAAGGACACTACCAAGGTGCAGTTCAACTGGTACAAGAACGGTGTTGGT
GGCAACGAGTACCTTCACAACCTTGGCTTTGATGCCAGCCAGGATTTCCACACTTACG
GTTTTGAATGGCGGCCTGACTACATCGACTTCTACGTGGACGGTAAGAAGGTGTACAG
GGGCACCAGAAATATCCCTGTGACTCCTGGCAAGATCATGATGAACCTTTGGCCTGGT
ATCGGTGTGGATGAGTGGCTTGGTAGATACGATGGTAGGACTCCTCTGCAGGCTGAGT
ACGAGTACGTTAAGTACTACCCTAACGGCAGATCTGAATTCAAGCTTGTGGTGAATA
CTCCTTTCGTGGCCGTGTTCAGCAACTTCGATTCTAGCCAGTGGGAGAAAGCTGATTGGGCT
AACGGTTCTGTGTTCAACTGCGTGTGGAAGCCTTCTCAGGTGACCTTCTCTAACGGCAAGAT
GATTCTGACCCTGGACCGTGAGTATAGGGTTCAGCCTACTGAGTCTATCGTGCGGTTCCCTA
ACATCACCAACTTGTGCCCTTTCGGCGAGGTGTTCAATGCTACTAGGTTCGCTTCTGTGTACG
CCTGGAACCGGAAGAGGATTTCTAACTGCGTGGCCGATTACAGCGTGCTGTACAACTCTGCT
AGCTTCAGCACCTTCAAGTGCTACGGTGTGTCTCCTACCAAGCTGAACGATCTGTGCTTCAC
CAACGTGTACGCTGACTCTTTCGTGATCAGGGGTGATGAGGTTAGGCAGATTGCTCCTGGTC
AGACCGGTAAGATCGCTGACTACAACTACAAGCTGCCTGATGACTTCACCGGTTGCGTGATC
GCTTGGAACTCTAACAACCTGGACTCTAAGGTTGGCGGCAATTACAACTACCTCTACCGGCT
GTTCCGGAAGTCTAACCTTAAGCCTTTCGAGCGGGATATCAGCACCGAGATCTATCAGGCTG
GTTCTACTCCTTGCAATGGCGTTGAGGGTTTCAACTGCTACTTCCCGCTTCAGTCTTACGGAT
TCCAGCCTACTAATGGTGTGGGCTACCAGCCTTACAGAGTGGTGGTTTTGTCTTTCGAGCTT
CTGCATGCTCCTGCTACTGTTTGCGGTCCTAAGAAGTAGCTCGAGACGAAG
CoV-14 PR1a-LicKM-CoV-RBM (34.91 kDa)
(SEQ ID NO: 27)
MGFVLFSQLPSFLLVSTLLLFLVISHSCRAHHHHHHHHGGSYPYKSGEYRTKSFFGYGYYE
VRMKAAKNVGIVSSFFTYTGPSDNNPWDEIDIEFLGKDTTKVQFNWYKNGVGGNEYLHN
LGFDASQDFHTYGFEWRPDYIDFYVDGKKVYRGTRNIPVTPGKIMMNLWPGIGVDEWLG
RYDGRTPLQAEYEYVKYYPNGRSEFKLVVNTPFVAVFSNFDSSQWEKADWANGSVFNCV
WKPSQNTYSNGKMILTGDREYNSNNLDSKVGGNYNYLYRLFRKSNLKPFERDISTEIYQAGSTP
CNGVEGFNCYFPLQSYGFQPTNGVGYQPY
(SEQ ID NO: 28)
ACGTCATTAATTAAATGGGATTCGTTTTGTTTTCTCAATTGCCTTCATTTCTTTTGGTTT
CTACTCTTTTGCTTTTTCTTGTTATTTCTCATTCATGTAGAGCTCATCACCATCACCACC
ATCATCATGGCGGCTCTTACCCTTATAAGAGCGGTGAGTACCGGACCAAGAGCTTCTTT
GGTTACGGTTACTACGAGGTGCGGATGAAGGCTGCTAAGAACGTGGGTATCGTGTCCA
GCTTCTTTACCTACACCGGGCCATCTGATAACAACCCTTGGGATGAGATCGACATCGA
GTTCCTTGGTAAGGACACTACCAAGGTGCAGTTCAACTGGTACAAGAACGGTGTTGGT
GGCAACGAGTACCTTCACAACCTTGGCTTTGATGCCAGCCAGGATTTCCACACTTACG
GTTTTGAATGGCGGCCTGACTACATCGACTTCTACGTGGACGGTAAGAAGGTGTACAG
GGGCACCAGAAATATCCCTGTGACTCCTGGCAAGATCATGATGAACCTTTGGCCTGGT
ATCGGTGTGGATGAGTGGCTTGGTAGATACGATGGTAGGACTCCTCTGCAGGCTGAGT
ACGAGTACGTTAAGTACTACCCTAACGGCAGATCTGAATTCAAGCTTGTGGTGAAT
ACTCCTTTCGTGGCCGTGTTCAGCAACTTCGATTCTAGCCAGTGGGAGAAAGCTGATTG
GGCTAACGGTTCTGTGTTCAACTGCGTGTGGAAGCCTTCTCAGGTGACCTTCTCTAACG
GCAAGATGATTCTGACCCTGGACCGTGAGTACAACAGCAACAACCTGGATTCTAAGGTCG
GCGGCAACTACAACTACCTCTACAGGCTGTTCCGGAAGTCCAACCTTAAGCCTTTCGAGAGG
GATATCAGCACCGAGATCTATCAGGCTGGTTCTACTCCTTGCAATGGCGTTGAGGGTTTCAA
CTGCTACTTCCCGCTTCAGTCTTACGGATTCCAGCCTACTAATGGTGTGGGCTACCAGCCTTA
TTAGCTCGAGACGAAG
CoV RBD Epitope constructs
CoV-15 HB78-CoV-RBM Epi 1: linker - HBcAg core 149 - RBM epitope 1 (25 kDa)
(SEQ ID NO: 29)
MDIDPYKEFGATVELLSFLPSDFFPSVRDLLDTASALYREALESPEHCSPHHTALRQAILCW
GELMTLATWVGANLEDPSGGSVCGPKKSTNLVKNKCVNFNFNGLTGTGVLTESNKKFLPFQ
QFGRDIADTTDAFRDPQTLEILDITPCSFGGVSVISGGSASRDLVVNAVNTNMGLKIRQLLWF
HISCLTFGRETVLEYLVSFGVWIRTPPAYRPPNAPILSTLPETTVV
(SEQ ID NO: 30)
ATGGACATCGATCCGTACAAAGAATTTGGCGCGACCGTCGAGCTGCTGAGCTTCCTGC
CGAGCGATTTTTTCCCGAGCGTGCGTGACCTGCTGGACACCGCGAGCGCACTGTATCGT
GAAGCACTGGAAAGCCCAGAGCACTGTAGCCCGCACCACACCGCCCTGCGCCAGGCGA
TTCTGTGCTGGGGTGAACTGATGACCCTGGCCACCTGGGTGGGTGCTAACCTTGAggatcc
gTCCGGAGGTAGCGTTTGTGGTCCTAAGAAAAGTACCAATCTCGTGAAGAACAAATGCGTC
AATTTCAACTTCAATGGGTTAACTGGAACTGGAGTTCTAACAGAGTCCAATAAGAAATTCCTTC
CATTTCAGCAATTTGGCAGGGATATTGCAGACACAACAGATGCTGTTAGAGATCCACAAACGT
TGGAAATTCTGGACATAACTCCCTGTTCTTTTGGTGGAGTATCAGTGATCTCCGGAGGTAG
CgctagcCGTGATCTGGTCGTCAACTATGTGAATACCAACATGGGTCTGAAAATTCGTCAG
CTGCTGTGGTTTCATATTAGCTGCCTGACCTTCGGTCGTGAAACCGTGCTGGAGTATCT
GGTGAGCTTCGGTGTGTGGATTCGCACCCCGCCGGCGTATCGTCCGCCGAACGCGCCA
ATTCTGAGCACGCTGCCGGAGACCACCGTGGTTTAG
CoV-16 HB78-CoV-RBM Epi 2: - linker - HBcAg core 149 - RBM epitope 2
(22 kDa)
(SEQ ID NO: 31)
MDIDPYKEFGATVELLSFLPSDFFPSVRDLLDTASALYREALESPEHCSPHHTALRPAILCW
GELMTLATWVGANLEDPSGGSGTNTSNQVAVLYQDVNCTEVPVAIHADQLTPTWRVYSTGSS
GGSASRDLVVNYVNTNMGLKIRPLLWFHISCLTFGRETVLEYLVSFGVWIRTPPAYRPPNA
PILSTLPETTVV
(SEQ ID NO: 32)
ATGGACATCGATCCGTACAAAGAATTTGGCGCGACCGTCGAGCTGCTGAGCTTCCTGC
CGAGCGATTTTTTCCCGAGCGTGCGTGACCTGCTGGACACCGCGAGCGCACTGTATCGT
GAAGCACTGGAAAGCCCAGAGCACTGTAGCCCGCACCACACCGCCCTGCGCCAGGCGA
TTCTGTGCTGGGGTGAACTGATGACCCTGGCCACCTGGGTGGGTGCTAACCTTGAggatcc
gTCCGGAGGTAGCGGTACGAATACCTCAAATCAGGTTGCTGTGTTGTATCAAGACGTGAAC
TGTACTGAAGTTCCTGTAGCCATTCATGCAGATCAACTTACACCAACTTGGAGAGTCTACTCTA
CAGGAAGTTCCGGAGGTAGCgctagcCGTGATCTGGTCGTCAACTATGTGAATACCAACA
TGGGTCTGAAAATTCGTCAGCTGCTGTGGTTTCATATTAGCTGCCTGACCTTCGGTCGT
GAAACCGTGCTGGAGTATCTGGTGAGCTTCGGTGTGTGGATTCGCACCCCGCCGGCGT
ATCGTCCGCCGAACGCGCCAATTCTGAGCACGCTGCCGGAGACCACCGTGGTTTAG
CoV-17 HB78-CoV-RBM Epi 1&2: linker - HBcAg core 149 - RBM epitope 1&2
(30 kDa)
(SEQ ID NO: 33)
MDIDPYKEFGATVELLSFLPSDFFPSVRDLLDTASALYREALESPEHCSPHHTALRPAILCW
GELMTLAYWVGANLEDPSGGSVCGPKKSTNLVKNKCVNFNFNGLTGTGVLTESNKKFLPFQ
QFGRDIADTTDAVRDPQTLEILDITPCSFGGVSVITPGTNTSNQVAVLYQDVNCTEVPVAIHADQL
TPTWRVYSTGSSGGSASRDLVVNYVNTNMGLKIRQLLWFHISCLTFGRETVLEYLVSFGVWI
RTPPAYRPPNAPILSTLPETTVV
(SEQ ID NO: 34)
ATGGACATCGATCCGTACAAAGAATTTGGCGCGACCGTCGAGCTGCTGAGCTTCCTGC
CGAGCGATTTTTTCCCGAGCGTGCGTGACCTGCTGGACACCGCGAGCGCACTGTATCGT
GAAGCACTGGAAAGCCCAGAGCACTGTAGCCCGCACCACACCGCCCTGCGCCAGGCGA
TTCTGTGCTGGGGTGAACTGATGACCCTGGCCACCTGGGTGGGTGCTAACCTTGAggatcc
gTCCGGAGGTAGCGTCTGTGGTCCCAAGAAAAGCACCAATCTGGTGAAGAACAAATGCGTG
AATTTCAACTTCAATGGCTTAACAGGAACAGGTGTTCTAACGGAGTCTAACAAGAAATTCCTTC
CGTTTCAACAGTTTGGCAGAGACATTGCTGATACCACAGATGCAGTTCGTGATCCTCAAACTC
TCGAAATCTTGGACATTACTCCATGTAGTTTTGGAGGTGTTTCTGTGATAACTCCAGGAACTAA
CACCTCCAATCAAGTTGCTGTGTTGTATCAGGATGTCAATTGCACTGAAGTACCTGTTGCCAT
TCATGCAGATCAGCTTACTCCAACATGGAGGGTATACTCAACAGGGTCATCCGGAGGTAGC
gctagcCGTGATCTGGTCGTCAACTATGTGAATACCAACATGGGTCTGAAAATTCGTCAGC
TGCTGTGGTTTCATATTAGCTGCCTGACCTTCGGTCGTGAAACCGTGCTGGAGTATCTG
GTGAGCTTCGGTGTGTGGATTCGCACCCCGCCGGCGTATCGTCCGCCGAACGCGCCAAT
TCTGAGCACGCTGCCGGAGACCACCGTGGTTTAG
CoV-18 PR1a-LicKM-CoV-RBD No His tag (49.4 kDa)
(SEQ ID NO: 35)
MGFVLFSQLPSFLLVSTLLLFLVISHSCRAGGSYPYKSGEYRTKSFFGYGYYEVRMKAAKN
VGIVSSFFTYTGPSDNNPWDEIDIEFLGKDTTKVQFNWYKNGVGGNEYLHNLGFDASQDF
HTYGFEWRPDYIDFYVDGKKVYRGTRNIPVTPGKIMMNLWPGIGVDEWLGRYDGRTPLQ
AEYEYVKYYPNGRSEFKLVVNTPFVAVFSNFDSSQWEKADWANGSVFNCVWKPSQVTFS
NGKMILTLDREYRVQPTESIVRFPNITNLCPFGEVFNATRFASVYAWNRKRISNCVADYSVLYNS
ASFSTFKCYGVSPTKLNDLCFTNVYADSFVIRGDEVRQIAPGQTGKIADYNYKLPDDFTGCVIAW
NSNNLDSKVGGNYNYLYRLFRKSNLKPFERDISTEIYQAGSTPCNGVEGFNCYFPLQSYGFQPTN
GVGYQPYRVWLSFELLHAPATVCGPKK
(SEQ ID NO: 36)
ACGTCATTAATTAAATGGGATTCGTTTTGTTTTCTCAATTGCCTTCATTTCTTTTGGTTT
CTACTCTTTTGCTTTTTCTTGTTATTTCTCATTCATGTAGAGCTGGCGGCTCTTACCCTTA
TAAGAGCGGTGAGTACCGGACCAAGAGCTTCTTTGGTTACGGTTACTACGAGGTGCGG
ATGAAGGCTGCTAAGAACGTGGGTATCGTGTCCAGCTTCTTTACCTACACCGGGCCAT
CTGATAACAACCCTTGGGATGAGATCGACATCGAGTTCCTTGGTAAGGACACTACCAA
GGTGCAGTTCAACTGGTACAAGAACGGTGTTGGTGGCAACGAGTACCTTCACAACCTT
GGCTTTGATGCCAGCCAGGATTTCCACACTTACGGTTTTGAATGGCGGCCTGACTACAT
CGACTTCTACGTGGACGGTAAGAAGGTGTACAGGGGCACCAGAAATATCCCTGTGACT
CCTGGCAAGATCATGATGAACCTTTGGCCTGGTATCGGTGTGGATGAGTGGCTTGGTA
GATACGATGGTAGGACTCCTCTGCAGGCTGAGTACGAGTACGTTAAGTACTACCCTAA
CGGCAGATCTGAATTCAAGCTTGTGGTGAATACTCCTTTCGTGGCCGTGTTCAGCAACT
TCGATTCTAGCCAGTGGGAGAAAGCTGATTGGGCTAACGGTTCTGTGTTCAACTGCGTGTGG
AAGCCTTCTCAGGTGACCTTCTCTAACGGCAAGATGATTCTGACCCTGGACCGTGAGTATAG
GGTTCAGCCTACTGAGTCTATCGTGCGGTTCCCTAACATCACCAACTTGTGCCCTTTCGGCG
AGGTGTTCAATGCTACTAGGTTCGCTTCTGTGTACGCCTGGAACCGGAAGAGGATTTCTAAC
TGCGTGGCCGATTACAGCGTGCTGTACAACTCTGCTAGCTTCAGCACCTTCAAGTGCTACGG
TGTGTCTCCTACCAAGCTGAACGATCTGTGCTTCACCAACGTGTACGCTGACTCTTTCGTGAT
CAGGGGTGATGAGGTTAGGCAGATTGCTCCTGGTCAGACCGGTAAGATCGCTGACTACAAC
TACAAGCTGCCTGATGACTTCACCGGTTGCGTGATCGCTTGGAACTCTAACAACCTGGACTC
TAAGGTTGGCGGCAATTACAACTACCTCTACCGGCTGTTCCGGAAGTCTAACCTTAAGCCTTT
CGAGCGGGATATCAGCACCGAGATCTATCAGGCTGGTTCTACTCCTTGCAATGGCGTTGAGG
GTTTCAACTGCTACTTCCCGCTTCAGTCTTACGGATTCCAGCCTACTAATGGTGTGGGCTACC
AGCCTTACAGAGTGGTGGTTTTGTCTTTCGAGCTTCTGCATGCTCCTGCTACTGTTTGCGGTC
CTAAGAAGTAGCTCGAGACGAAG
HB78-CoV-RBM: HBcAg core 149 - RBM
(SEQ ID NO: 37)
MDIDPYKEFGATVELLSFLPSDFFPSVRDLLDTASALYREALESPEHCSPHHTALRQAILCW
GELMTLATWVGANLEDPNSNNLDSKVGGNYNYLYRLFRKSNLKPFERDISTEIYQAGSTPCNGV
EGFNCYFPLQSYGFQPTNGVGYQPYASRDLVVNYVNTNMGLKIRQLLWFHISCLTFGRETVL
EYLVSFGVWIRTPPAYRPPNAPILSTLPETTVV
HB78-CoV-RBD: HBcAg core 149 - RBD
(SEQ ID NO: 38)
MDIDPYKEFGATVELLSFLPSDFFPSVRDLLDTASALYREALESPEHCSPHHTALRQAILCW
GELMTLAYWVGANLEDPRVQPTESIVRFPNITNLCPFGEVFNATRFASVYAWNRKRISNCVADY
SVLYNSASFSTFKCYGVSPTKLNDLCFTNVYADSFVIRGDEVRQIAPGQTGKIADYNYKLPDDFTG
CVIAWNSNNLDSKVGGNYNYLYRLFRKSNLKPFERDISTEIYQAGSTPCNGVEGFNCYFPLQSYG
FQPTNGVGYQPYRVVVLSFELLHAPATVCGPKKASRDLVVNYVNTNMGLKIRQLLWFHISCL
TFGRETVLEYLVSFGVWIRTPPAYRPPNAPILSTLPETTVV
CoV-19 - HBcAg - Just RBM with long linker
(SEQ ID NO: 39)
MDIDPYKEFGATVELLSFLPSDFFPSVRDLLDTASALYREALESPEHCSPHHTALRQAILCW
GELMTLAYWNGANLEDGGGGSGGGGTNSNNLDSKVGGNYNYLYRLFRKSNLKPFERDISTEI
YQAGSTPCNGVEGFNCYFPLQSYGFQPTNGVGYQPYGGGGSGGGGSRDLVVNYVNTNMGL
KIRQLLWFHISCLTFGRETVLEYLVSFGVWIRTPPAYRPPNAPILSTLPETTVV-
(SEQ ID NO: 40)
ATGGATATTGATCCATACAAGGAATTCGGTGCAACTGTGGAGCTTCTGTCATTCTTGCC
AAGCGACTTTTTCCCGTCAGTTCGAGATCTGCTTGATACGGCTTCTGCACTTTACAGGG
AAGCGTTGGAGAGTCCAGAGCATTGCTCTCCTCACCATACTGCACTAAGACAAGCAAT
ACTTTGTTGGGGTGAATTAATGACACTCGCTACATGGGTTGGAGCCAACCTCGAAGAT
GGAGGAGGCGGCTCTGGTGGGGGCGGAACTAATTCCAATAATTTGGACTCTAAAGTAG
GTGGTAACTATAATTACCTGTATCGTTTATTTAGAAAATCGAATCTCAAACCATTTGAG
AGAGACATCTCAACTGAGATATACCAAGCTGGATCAACCCCATGCAACGGTGTGGAAG
GGTTCAATTGTTATTTTCCGTTACAATCCTATGGTTTTCAACCTACAAATGGTGTTGGTT
ATCAGCCTTACGGCGGCGGAGGGAGTGGAGGTGGAGGGAGTAGAGATTTGGTTGTCAA
CTATGTCAATACCAACATGGGATTGAAGATTAGGCAGCTTTTATGGTTTCATATTTCTT
GTTTGACATTCGGGCGGGAAACAGTACTTGAATATCTAGTAAGCTTTGGAGTGTGGATC
CGCACCCCCCCTGCTTATAGGCCCCCAAATGCTCCTATTCTTTCAACTCTTCCTGAAACT
ACAGTTGTTtga
CoV-20 - HBcAg - RBD - extra sequence with long linker
(SEQ ID NO: 41)
MDIDPYKEFGATVELLSFLPSDFFPSVRDLLDTASALYREALESPEHCSPHHTALRQAILCW
GELMTLATWVGANLEDGGGGSGGGGTRVQPTESIVRFPNITNLCPFGEVFNATRFASVYAW
NRKRISNCVADYSVLYNSASFSTFKCYGVSPTKLNDLCFTNVYADSFVIRGDEVRQIAPGQTGKIA
DYNYKLPDDFTGCVIAWNSNNLDSKVGGNYNYLYRLFRKSNLKPFERDISTEIYQAGSTPCNGVE
GFNCYFPLQSYGFQPTNGVGYQPYRVWLSFELLHAPATVCGPKKSTNLVKNKGGGGSGGGG
SRDLVVNYVNTNMGLKIRQLLWFHISCLTFGRETVLEYLVSFGVWIRTPPAYRPPNAPILST
LPETTVV
(SEQ ID NO: 42)
ATGGATATTGATCCTTATAAAGAATTTGGGGCTACCGTCGAACTACTATCTTTCTTGCC
GTCTGATTTTTTCCCATCCGTCAGAGACCTTCTTGACACTGCATCTGCTTTATACAGAGA
GGCTCTGGAATCTCCTGAGCATTGTTCACCACACCACACAGCTCTGAGACAAGCGATAT
TGTGTTGGGGTGAATTGATGACACTTGCTACATGGGTTGGAGCAAATCTGGAAGATGG
AGGGGGTGGTTCAGGTGGTGGGGGCACTCGGGTTCAACCAACAGAGAGCATTGTTAGG
TTCCCTAATATTACCAACTTGTGCCCTTTTGGAGAGGTTTTCAATGCAACCCGTTTCGCC
AGCGTCTATGCATGGAACCGCAAGAGGATCTCCAACTGTGTAGCAGATTATTCTGTACT
ATACAATTCCGCCAGTTTTTCAACTTTCAAATGTTATGGTGTAAGTCCCACTAAGCTTA
ATGATTTATGTTTTACGAATGTGTATGCTGATTCGTTTGTAATCAGAGGTGATGAAGTG
AGACAGATAGCACCTGGGCAAACAGGAAAAATAGCAGACTACAATTACAAGTTGCCT
GATGATTTCACTGGTTGTGTTATTGCTTGGAATAGTAACAACCTTGATAGTAAGGTAGG
TGGAAACTACAACTATTTGTACAGGCTGTTTCGTAAATCCAATTTAAAGCCGTTTGAGA
GAGATATTAGCACAGAGATTTATCAAGCTGGATCAACTCCTTGCAATGGCGTAGAAGG
ATTTAATTGCTATTTTCCCTTGCAGTCATACGGTTTTCAACCTACTAACGGAGTCGGCTA
CCAGCCATATAGGGTGGTTGTGCTCTCTTTCGAGTTGCTTCATGCACCAGCGACTGTTT
GTGGACCAAAGAAATCGACAAATCTAGTGAAGAATAAAGGAGGAGGCGGCTCAGGGG
GTGGCGGGTCACGAGACTTGGTTGTGAATTATGTTAATACAAACATGGGACTGAAAAT
CCGGCAGCTATTATGGTTTCATATTAGTTGCCTCACTTTCGGAAGGGAAACTGTTTTAG
AATATCTTGTCTCTTTTGGTGTTTGGATTAGAACGCCTCCCGCCTATCGACCACCAAAT
GCTCCAATACTTTCTACTCTCCCTGAAACAACCGTTGTGtga
CoV-21 - HBcAg - New RBD with extra sequence and long linker
(SEQ ID NO: 43)
MDIDPYKEFGATVELLSFLPSDFFPSVRDLLDTASALYREALESPEHCSPHHTALRQAILCW
GELMTLATWVGANLEDGGGGSGGGGTRVQPTESIVRFPNITNLCPFGEVFNATRFASVYAW
NRKRISNCVADYSVLYNSASFSTFKCYGVSPTKLNDLCFTNVYADSFVIRGDEVRQIAPGQTGKIA
DYNYKLPDDFTGCVIAWNSNNLDSKVGGNYNYLYRLFRKSNLKPFERDISTEIYQAGSTPCNGVE
GFNCYFPLQSYGFQPTNGVGYQPYRVWLSFELLHAPATVCGPKKSTNLVKNKCVNFNFNGLTG
TGVLTESNKKFLPFQQFGRDIADTWAVRDPQTLEILDITPCSFGGVSVIGGGGSGGGGSRDLV
VNYVNTNMGLKIRQLLWFHISCLTFGRETVLEYLVSFGVWIRTPPAYRPPNAPILSTLPETT
VV
(SEQ ID NO: 44)
ATGGATATTGATCCTTACAAAGAATTTGGTGCTACCGTGGAGTTACTCAGTTTCCTACC
CTCTGATTTTTTCCCTAGTGTCAGGGACCTATTGGACACCGCCTCCGCTCTTTACCGAGA
AGCTTTAGAGTCTCCAGAACACTGTTCTCCTCATCATACAGCCTTGAGACAAGCTATTT
TGTGTTGGGGAGAGCTGATGACTCTTGCCACATGGGTTGGGGCAAATCTTGAAGATGG
TGGAGGCGGCTCAGGTGGTGGAGGTACCCGTGTTCAACCAACTGAGTCAATTGTTAGG
TTTCCTAATATTACAAATTTATGCCCATTTGGCGAAGTTTTTAATGCAACGAGATTTGCT
AGCGTGTATGCCTGGAATCGGAAGAGAATCAGCAATTGTGTAGCTGATTATTCAGTGC
TCTATAATTCGGCGTCCTTTTCTACGTTCAAGTGCTATGGCGTGTCGCCAACAAAACTT
AATGATCTTTGCTTCACTAATGTTTATGCAGACTCTTTTGTGATCAGAGGAGATGAAGT
GCGACAGATAGCCCCGGGACAAACTGGGAAAATTGCAGATTACAATTACAAGTTGCCT
GATGATTTCACAGGTTGTGTAATAGCATGGAACTCGAATAACTTGGATTCAAAAGTTG
GTGGTAACTACAACTATTTATATCGTCTCTTCAGGAAATCCAATCTGAAGCCTTTTGAA
AGAGATATCTCAACTGAAATATATCAAGCAGGGTCTACTCCATGCAATGGAGTTGAAG
GATTTAACTGTTATTTTCCACTGCAATCTTATGGTTTTCAACCTACGAACGGGGTAGGA
TATCAGCCCTACAGAGTTGTGGTACTAAGTTTTGAGCTTTTACACGCTCCTGCAACTGT
TTGTGGTCCAAAGAAATCAACCAACCTAGTGAAAAATAAATGTGTCAACTTCAATTTC
AATGGTTTGACTGGAACTGGTGTCCTGACAGAGAGCAACAAGAAGTTTCTGCCCTTTCA
GCAATTCGGGAGAGATATAGCTGACACTACTGATGCTGTTCGAGACCCACAGACCTTG
GAGATTCTAGACATAACACCTTGTAGTTTCGGGGGAGTAAGTGTTATCGGCGGTGGAG
GCAGTGGAGGGGGCGGGTCCCGTGATCTCGTTGTCAATTATGTTAATACAAACATGGG
ATTGAAGATTAGGCAGCTTCTTTGGTTTCATATTTCTTGCTTAACATTCGGTCGGGAAA
CTGTCTTGGAATATTTGGTTTCATTTGGAGTTTGGATTCGCACCCCACCGGCATACAGG
CCTCCAAATGCTCCAATTCTTTCAACTTTACCTGAGACAACAGTTGTAtga
CoV-22 - HBcAg - New RBD with extra sequence and long linker
(SEQ ID NO: 45)
MDIDPYKEFGATVELLSFLPSDFFPSVRDLLDTASALYREALESPEHCSPHHTALRQAILCW
GELMTLATWVGANLEDGGGGSGGGGTRVQPTESIVRFPNITNLCPFGEVFNATRFASVYAW
NRKRISNCVADYSVLYNSASFSTFKCYGVSPTKLNDLCFTNVYADSFVIRGDEVRQIAPGQTGKIA
DYNYKLPDDFTGCVIAWNSNNLDSKVGGNYNYLYRLFRKSNLKPFERDISTEIYQAGSTPCNGVE
GFNCYFPLQSYGFQPTNGVGYQPYRVWLSFELLHAPATVCGPKKSTNLVKNKCVNFNFNGLTG
TGVLTESNKKFLPFQQFGRDIADTTDAVRDPQTLEILDITPCSFGGVSVITPGTNTSNQVAVLYQD
VNCTEVPVAIHADQLTPTWRVYSTGSGGGGSGGGGSRDLVVNYVNTNMGLKIRQLLWFHIS
CLTFGRETVLEYLVSFGVWIRTPPAYRPPNAPILSTLPETTVV-
(SEQ ID NO: 46)
ATGGATATCGATCCTTACAAAGAATTTGGTGCAACCGTCGAGCTTCTATCCTTTCTTCCT
TCTGATTTTTTCCCGAGTGTACGTGACCTACTTGACACTGCTTCAGCTCTTTACAGGGAA
GCATTAGAGTCTCCCGAGCATTGTAGCCCTCATCATACAGCTCTTAGGCAAGCAATACT
CTGTTGGGGTGAGTTGATGACCCTTGCAACTTGGGTAGGAGCGAATTTGGAAGATGGT
GGAGGAGGCAGTGGTGGAGGTGGCACCAGAGTTCAACCAACAGAAAGCATCGTTCGG
TTCCCCAATATTACTAACCTCTGCCCATTCGGAGAAGTTTTTAATGCTACTAGGTTTGCT
TCTGTATATGCTTGGAACAGAAAAAGAATCAGCAACTGTGTCGCCGATTATAGTGTTTT
ATACAACTCTGCCTCCTTCTCCACTTTCAAGTGCTATGGAGTTTCGCCCACTAAATTAA
ATGATTTATGCTTCACAAATGTTTATGCAGATTCTTTTGTGATACGAGGTGATGAAGTA
CGGCAAATTGCCCCTGGTCAAACTGGAAAGATTGCTGACTACAATTACAAGCTGCCTG
ATGATTTCACTGGTTGTGTTATAGCATGGAATTCTAATAATCTTGATTCCAAAGTCGGA
GGAAATTATAACTATTTGTACCGACTCTTTAGGAAGTCTAATCTAAAACCATTTGAGAG
GGACATTAGTACTGAAATATATCAGGCTGGTAGTACACCTTGCAATGGTGTTGAAGGA
TTTAATTGTTATTTTCCATTGCAGAGTTATGGGTTTCAGCCAACTAATGGGGTTGGTTAC
CAGCCTTATCGTGTGGTGGTACTAAGCTTTGAATTATTGCACGCGCCCGCAACTGTGTG
TGGACCAAAGAAGTCTACAAATCTTGTGAAGAATAAATGCGTGAATTTCAACTTCAAC
GGGCTTACGGGTACAGGTGTCTTAACCGAGTCAAACAAGAAATTTCTCCCGTTTCAACA
ATTTGGCAGAGATATAGCCGACACAACTGATGCTGTACGCGATCCACAAACATTGGAG
ATTTTGGACATTACGCCATGTTCATTCGGAGGCGTGTCTGTAATCACTCCTGGGACCAA
CACATCCAATCAAGTTGCTGTTCTGTATCAAGATGTTAACTGCACAGAGGTTCCAGTCG
CGATTCATGCTGATCAGTTGACCCCAACATGGAGAGTCTATTCTACAGGATCGGGTGGC
GGTGGGTCAGGCGGCGGGGGGTCTAGGGATTTGGTAGTGAATTATGTTAATACTAACA
TGGGACTGAAAATTCGTCAGCTCTTATGGTTTCACATTTCATGTTTGACATTTGGGAGA
GAAACAGTGCTTGAATATCTGGTCTCATTCGGAGTTTGGATTAGAACTCCACCTGCATA
CAGACCTCCTAATGCACCGATCTTATCAACGCTGCCTGAGACTACTGTGGTTtga
CoV-23 HBcAg-CoV RBD
(SEQ ID NO: 47)
MDIDPYKEFGATVELLSFLPSDFFPSVRDLLDTASALYREALESPEHCSPHHTALRQAILCW
GELMTLATWVGANLEDPASRDLVVNYVNTNMGLKIRQLLWFHISCLTFGRETVLEYLVSF
GVWIRTPPAYRPPNAPILSTLPGGGGSGGGGTRVQPTESIVRFPNITNLCPFGEVFNATRFAS
VYAWNRKRISNCVADYSVLYNSASFSTFKCYGVSPTKLNDLCFTNVYADSFVIRGDEVRQIAPGQT
GKIADYNYKLPDDFTGCVIAWNSNNLDSKVGGNYNYLYRLFRKSNLKPFERDISTEIYQAGSTPC
NGVEGFNCYFPLQSYGFQPTNGVGYQPYRVWLSFELLHAPATVCGPKKSTNLVKNK
(SEQ ID NO: 48)
ATGGATATTGATCCATACAAGGAATTTGGTGCAACTGTGGAGCTGTTAAGTTTCTTACC
TTCTGATTTCTTCCCTAGTGTGCGTGATTTGCTGGACACTGCGTCGGCTCTTTATCGTGA
GGCTCTGGAGAGTCCTGAACATTGCAGTCCACACCACACAGCTTTGAGACAGGCAATT
TTGTGTTGGGGTGAACTTATGACTCTTGCTACCTGGGTTGGAGCTAACTTAGAAGATCC
TGCATCGAGAGACCTCGTTGTTAATTATGTGAATACTAACATGGGACTCAAAATACGA
CAACTTTTGTGGTTTCATATTAGCTGTCTAACATTTGGGCGGGAGACAGTGCTTGAGTA
CCTAGTATCATTTGGAGTTTGGATTCGTACTCCTCCAGCCTATAGGCCACCAAATGCTC
CTATCCTTTCTACCCTACCCGGTGGAGGAGGTTCTGGGGGCGGCGGTACGAGGGTTCA
ACCCACAGAATCTATTGTGCGCTTCCCAAATATCACTAATCTCTGCCCCTTTGGGGAAG
TTTTCAATGCAACAAGATTTGCCTCAGTTTATGCATGGAATAGAAAAAGAATATCTAA
TTGTGTTGCAGACTACTCCGTCCTTTACAACTCAGCCTCCTTCTCTACTTTCAAGTGTTA
TGGTGTTTCACCAACTAAATTGAATGATCTTTGCTTTACCAATGTATATGCTGACAGCT
TTGTTATCCGGGGAGATGAGGTGAGGCAAATAGCACCAGGACAGACGGGTAAGATAG
CAGATTACAATTACAAACTGCCTGATGATTTTACAGGGTGCGTCATTGCTTGGAACAGT
AATAATTTGGACAGCAAGGTTGGTGGCAACTATAACTACTTGTATAGATTGTTCAGGA
AATCAAACTTGAAGCCTTTTGAAAGGGATATTTCAACTGAAATTTATCAAGCTGGCTCC
ACACCCTGTAATGGTGTAGAAGGGTTTAATTGCTATTTTCCTTTACAGTCATATGGATT
CCAACCAACAAATGGAGTGGGTTATCAGCCTTATAGAGTCGTAGTATTATCTTTTGAGC
TCCTTCATGCTCCGGCCACTGTTTGTGGGCCGAAGAAAAGTACCAACCTAGTCAAGAA
TAAAtag
CoV-24-_LicKM-CoV-RBD No His tag (49.4 kDa)
(SEQ ID NO: 49)
MGGSYPYKSGEYRTKSFFGYGYYEVRMKAAKNVGIVSSFFTYTGPSDNNPWDEIDIEFLG
KDTTKVQFNWYKNGVGGNEYLHNLGFDASQDFHTYGFEWRPDYIDFYVDGKKVYRGTR
NIPVTPGKIMMNLWPGIGVDEWLGRYDGRTPLQAEYEYVKYYPNGRSEFKLVVNTPFVA
VFSNFDSSQWEKADWANGSVFNCVWKPSQVTFSNGKMILTLDREYRVQPTESIVRFPNITN
LCPFGEVFNATRFASVYAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYNYLYRLFRKSNLKPFE
RDISTEIYQAGSTPCNGVEGFNCYFPLQSYGFQPTNGVGYQPYRVWLSFELLHAPATVCGPKK
(SEQ ID NO: 50)
ACGTCATTAATTAAATGGGCGGCTCTTACCCTTATAAGAGCGGTGAGTACCGGACCAA
GAGCTTCTTTGGTTACGGTTACTACGAGGTGCGGATGAAGGCTGCTAAGAACGTGGGT
ATCGTGTCCAGCTTCTTTACCTACACCGGGCCATCTGATAACAACCCTTGGGATGAGAT
CGACATCGAGTTCCTTGGTAAGGACACTACCAAGGTGCAGTTCAACTGGTACAAGAAC
GGTGTTGGTGGCAACGAGTACCTTCACAACCTTGGCTTTGATGCCAGCCAGGATTTCCA
CACTTACGGTTTTGAATGGCGGCCTGACTACATCGACTTCTACGTGGACGGTAAGAAG
GTGTACAGGGGCACCAGAAATATCCCTGTGACTCCTGGCAAGATCATGATGAACCTTT
GGCCTGGTATCGGTGTGGATGAGTGGCTTGGTAGATACGATGGTAGGACTCCTCTGCA
GGCTGAGTACGAGTACGTTAAGTACTACCCTAACGGCAGATCTGAATTCAAGCTTG
TGGTGAATACTCCTTTCGTGGCCGTGTTCAGCAACTTCGATTCTAGCCAGTGGGAGAAA
GCTGATTGGGCTAACGGTTCTGTGTTCAACTGCGTGTGGAAGCCTTCTCAGGTGACCTT
CTCTAACGGCAAGATGATTCTGACCCTGGACCGTGAGTATAGGGTTCAGCCTACTGAG
TCTATCGTGCGGTTCCCTAACATCACCAACTTGTGCCCTTTCGGCGAGGTGTTCAATGC
TACTAGGTTCGCTTCTGTGTACGCCTGGAACCGGAAGAGGATTTCTAACTGCGTGGCC
GATTACAGCGTGCTGTACAACTCTGCTAGCTTCAGCACCTTCAAGTGCTACGGTGTGTC
TCCTACCAAGCTGAACGATCTGTGCTTCACCAACGTGTACGCTGACTCTTTCGTGATCA
GGGGTGATGAGGTTAGGCAGATTGCTCCTGGTCAGACCGGTAAGATCGCTGACTACAA
CTACAAGCTGCCTGATGACTTCACCGGTTGCGTGATCGCTTGGAACTCTAACAACCTGG
ACTCTAAGGTTGGCGGCAATTACAACTACCTCTACCGGCTGTTCCGGAAGTCTAACCTT
AAGCCTTTCGAGCGGGATATCAGCACCGAGATCTATCAGGCTGGTTCTACTCCTTGCA
ATGGCGTTGAGGGTTTCAACTGCTACTTCCCGCTTCAGTCTTACGGATTCCAGCCTACT
AATGGTGTGGGCTACCAGCCTTACAGAGTGGTGGTTTTGTCTTTCGAGCTTCTGCATGC
TCCTGCTACTGTTTGCGGTCCTAAGAAGTAGCTCGAGACGAAG
CoV25: LicKM- Spike 319-684 c-term/66 kDa
(SEQ ID NO: 51)
MGFVLFSQLPSFLLVSTLLLFLVISHSCRAGGSYPYKSGEYRTKSFFGYGYYEVRMKAAKN
VGIVSSFFTYTGPSDNNPWDEIDIEFLGKDTTKVQFNWYKNGVGGNEYLHNLGFDASQDF
HTYGFEWRPDYIDFYVDGKKVYRGTRNIPVTPGKIMMNLWPGIGVDEWLGRYDGRTPLQ
AEYEYVKYYPNGRSEFKLVVNTPFVAVFSNFDSSQWEKADWANGSVFNCVWKPSQVTFS
NGKMILTLDREYRVQPTESIVRFPNITNLCPFGEVFNATRFASVYAWNRKRISNCVADYSVLYNS
ASFSTFKCYGVSPTKLNDLCFTNVYADSFVIRGDEVRQIAPGQTGKIADYNYKLPDDFTGCVIAW
NSNNLDSKVGGNYNYLYRLFRKSNLKPFERDISTEIYQAGSTPCNGVEGFNCYFPLQSYGFQPTN
GVGYQPYRVVVLSFELLHAPATVCGPKKSTNLVKNKCVNFNFNGLTGTGVLTESNKKFLPFQ
QFGRDIADTTDAVRDPQTLEILDITPCSFGGVSVITPGTNTSNQVAVLYQDVNCTEVPVAIH
ADQLTPTWRVYSTGSNVFQTRAGCLIGAEHVNNSYECDIPIGAGICASYQTQTNSPRRA
(SEQ ID NO: 52)
ACGTCATTAATTAAATGGGATTCGTTTTGTTTTCTCAATTGCCTTCATTTCTTTTGGTTT
CTACTCTTTTGCTTTTTCTTGTTATTTCTCATTCATGTAGAGCTGGCGGCTCTTACCCTTA
TAAGAGCGGTGAGTACCGGACCAAGAGCTTCTTTGGTTACGGTTACTACGAGGTGCGG
ATGAAGGCTGCTAAGAACGTGGGTATCGTGTCCAGCTTCTTTACCTACACCGGGCCAT
CTGATAACAACCCTTGGGATGAGATCGACATCGAGTTCCTTGGTAAGGACACTACCAA
GGTGCAGTTCAACTGGTACAAGAACGGTGTTGGTGGCAACGAGTACCTTCACAACCTT
GGCTTTGATGCCAGCCAGGATTTCCACACTTACGGTTTTGAATGGCGGCCTGACTACAT
CGACTTCTACGTGGACGGTAAGAAGGTGTACAGGGGCACCAGAAATATCCCTGTGACT
CCTGGCAAGATCATGATGAACCTTTGGCCTGGTATCGGTGTGGATGAGTGGCTTGGTA
GATACGATGGTAGGACTCCTCTGCAGGCTGAGTACGAGTACGTTAAGTACTACCCTAA
CGGCAGATCTGAATTCAAGCTTGTGGTGAATACTCCTTTCGTGGCCGTGTTCAGCAA
CTTCGATTCTAGCCAGTGGGAGAAAGCTGATTGGGCTAACGGTTCTGTGTTCAACTGC
GTGTGGAAGCCTTCTCAGGTGACCTTCTCTAACGGCAAGATGATTCTGACCCTGGACC
GTGAGTATAGGGTTCAACCAACTGAGTCAATCGTTAGGTTCCCAAACATCACAAATTTGTGT
CCTTTCGGTGAAGTTTTTAATGCTACTAGATTCGCTTCTGTTTACGCTTGGAACAGAAAAAGG
ATCTCAAATTGCGTTGCTGATTACTCTGTTCTTTACAACTCTGCTTCTTTTTCTACTTTTAAGTG
TTACGGTGTTTCACCAACTAAGTTGAACGATCTTTGCTTCACAAACGTTTACGCTGATTCTTTC
GTTATTAGAGGAGATGAGGTTAGGCAAATTGCTCCTGGACAAACTGGAAAGATTGCTGATTAC
AACTACAAATTGCCAGATGATTTCACAGGATGCGTTATCGCTTGGAACTCAAATAACCTTGATT
CTAAGGTTGGAGGTAATTATAACTACTTGTACAGACTTTTTAGGAAGTCAAATTTGAAGCCTTT
CGAAAGGGATATCTCAACTGAGATCTATCAAGCTGGTTCTACACCATGTAATGGTGTTGAAGG
TTTTAATTGCTACTTCCCACTTCAATCTTATGGATTTCAACCTACTAATGGTGTTGGTTACCAAC
CTACAGAGTTGTTGTTTTGTCATTCGAGTTGCTTCATGCTCCAGCTACTGTTTGTGGTCCTAA
GAAATCTACAAATCTTGTTAAGAATAAGTGCGTTAACTTCAACTTCAATGGTTTGACTGGAACA
GGTGTTCTTACTGAATCAAATAAGAAGTTCTTGCCTTTCCAACAATTCGGTAGAGATATTGCTG
ATACTACAGATGCTGTTAGGGATCCTCAAACTTTGGAGATTCTTGATATTACACCATGTTCATT
TGGAGGTGTTTCTGTTATTACTCCAGGAACTAACACATCTAACCAAGTTGCTGTTTTGTACCAA
GATGTTAATTGCACAGAAGTTCCTGTTGCTATTCATGCTGATCAACTTACTCCAACATGGAGA
GTTTACTCAACTGGATCTAACGTTTTCCAAACAAGGGCTGGATGTCTTATTGGTGCTGAACAT
GTTAATAACTCTTACGAGTGTGATATTCCTATTGGAGCTGGTATTTGCGCTTCATATCAAACTC
AAACAAATTCTCCAAGAAGGGCTTAGCTCGAGACGAAG
CoV26: LicKM-Spike 319-684 Loop/66 kDa
(SEQ ID NO: 53)
MGFVLFSQLPSFLLVSTLLLFLVISHSCRAGGSYPYKSGEYRTKSFFGYGYYEVRMKAAKN
VGIVSSFFTYTGPSDNNPWDEIDIEFLGKDTTKVQFNWYKNGVGGNEYLHNLGFDASQDF
HTYGFEWRPDYIDFYVDGKKVYRGTRNIPVTPGKIMMNLWPGIGVDEWLGRYDGRTPLQ
AEYEYVKYYPNGRSRVQPTESIVRFPNITNLCPFGEVFNATRFASVYAWNRKRISNCVADYSVLY
NSASFSTFKCYGVSPTKLNDLCFTNVYADSFVIRGDEVRQIAPGQTGKIADYNYKLPDDFTGCVI
AWNSNNLDSKVGGNYNYLYRLFRKSNLKPFERDISTEIYQAGSTPCNGVEGFNCYFPLQSYGFQP
TNGVGYQPYRVVVLSFELLHAPATVCGPKKSTNLVKNKCVNFNFNGLTGTGVLTESNKKFLP
FQQFGRDIADTTDAVRDPQTLEILDITPCSFGGVSVITPGTNTSNQVAVLYQDVNCTEVPVA
IHADQLTPTWRVYSTGSNVFQTRAGCLIGAEHVNNSYECDIPIGAGICASYQTQTNSPRRAE
FKLVVNTPFVAVFSNFDSSQWEKADWANGSVFNCVWKPSQVTFSNGKMILTLDREY
(SEQ ID NO: 54)
ACGTCATTAATTAAATGGGATTCGTTTTGTTTTCTCAATTGCCTTCATTTCTTTTGGTTTC
TACTCTTTTGCTTTTTCTTGTTATTTCTCATTCATGTAGAGCTGGCGGCTCTTACCCTTAT
AAGAGCGGTGAGTACCGGACCAAGAGCTTCTTTGGTTACGGTTACTACGAGGTGCGGA
TGAAGGCTGCTAAGAACGTGGGTATCGTGTCCAGCTTCTTTACCTACACCGGGCCATCT
GATAACAACCCTTGGGATGAGATCGACATCGAGTTCCTTGGTAAGGACACTACCAAGG
TGCAGTTCAACTGGTACAAGAACGGTGTTGGTGGCAACGAGTACCTTCACAACCTTGG
CTTTGATGCCAGCCAGGATTTCCACACTTACGGTTTTGAATGGCGGCCTGACTACATCG
ACTTCTACGTGGACGGTAAGAAGGTGTACAGGGGCACCAGAAATATCCCTGTGACTCC
TGGCAAGATCATGATGAACCTTTGGCCTGGTATCGGTGTGGATGAGTGGCTTGGTAGAT
ACGATGGTAGGACTCCTCTGCAGGCTGAGTACGAGTACGTTAAGTACTACCCTAACGG
CAGATCTAGGGTTCAACCTACTGAATCTATCGTTAGGTTCCCAAACATCACAAATTTGTGTCC
TTTCGGAGAGGTTTTTAATGCTACTAGATTCGCTTCTGTTTATGCTTGGAATAGAAAGAGGATT
TCAAATTGCGTTGCTGATTACTCTGTTCTTTACAACTCTGCTTCTTTTTCTACTTTTAAGTGTTA
CGGTGTTTCACCAACTAAGTTGAACGATCTTTGCTTCACAAACGTTTACGCTGATTCTTTCGTT
ATTAGAGGAGATGAGGTTAGGCAAATTGCTCCTGGACAAACTGGAAAGATTGCTGATTACAAC
TAGAAATTGCCAGATGATTTCACAGGTTGTGTTATCGCTTGGAACTCAAATAACCTTGATTCTA
AGGTTGGAGGTAATTATAACTACTTGTACAGACTTTTTAGGAAGTCAAATTTGAAGCCTTTCGA
AAGGGATATCTCAACTGAGATCTATCAAGCTGGTTCTACACCATGTAATGGTGTTGAAGGTTTT
AATTGCTACTTCCCACTTCAATCTTATGGATTTCAACCTACTAATGGTGTTGGTTACCAACCAT
ACAGAGTTGTTGTTTTGTCATTCGAGTTGCTTCATGCTCCAGCTACTGTTTGTGGTCCTAAGAA
ATCTACAAATCTTGTTAAGAATAAGTGCGTTAACTTCAACTTCAATGGTTTGACTGGAACAGGT
GTTCTTACAGAATCAAATAAGAAGTTCCTTCCTTTCCAACAATTCGGTAGAGATATTGCTGATA
CTACAGATGCTGTTAGGGATCCTCAAACTTTGGAGATTCTTGATATTACACCATGTTCATTTGG
AGGTGTTTCTGTTATTACTCCAGGAACTAACACATCTAACCAAGTTGCTGTTTTGTACCAAGAT
GTTAATTGCACAGAAGTTCCTGTTGCTATTCATGCTGATCAACTTACTCCAACATGGAGAGTTT
ACTCAACTGGATCTAACGTTTTCCAAACAAGGGCTGGATGTTTGATTGGTGCTGAACATGTTA
ATAACTCTTACGAGTGTGATATTCCTATTGGAGCTGGTATTTGCGCTTCATATCAAACTCAAAC
AAATTCTCCTAGAAGGGCTGAATTCAAGCTTGTGGTGAATACTCCTTTCGTGGCCGTGT
TCAGCAACTTCGATTCTAGCCAGTGGGAGAAAGCTGATTGGGCTAACGGTTCTGTGTTC
AACTGCGTGTGGAAGCCTTCTCAGGTGACCTTCTCTAACGGCAAGATGATTCTGACCCT
GGACCGTGAGTATTAGCTCGAGACGAAG
CoV27: Spike 227-684 c-term/76.2 kDa
(SEQ ID NO: 55)
MGFVLFSQLPSFLLVSTLLLFLVISHSCRAGGSYPYKSGEYRTKSFFGYGYYEVRMKAAKN
VGIVSSFFTYTGPSDNNPWDEIDIEFLGKDTTKVQFNWYKNGVGGNEYLHNLGFDASQDF
HTYGFEWRPDYIDFYVDGKKVYRGTRNIPVTPGKIMMNLWPGIGVDEWLGRYDGRTPLQ
AEYEYVKYYPNGRSEFKLVVNTPFVAVFSNFDSSQWEKADWANGSVFNCVWKPSQVTFS
NGKMILTLDREYVDLPIGINITRFQTLLALHRSYLTPGDSSSGWTAGAAAYYVGYLQPRTF
LLKYNENGTITDAVDCALDPLSETKCTLKSFTVEKGIYQTSNFRVQPTESIVRFPNITNLCPFG
EVFNATRFASVYAWNRKRISNCVADYSVLYNSASFSTFKCYGVSPTKLNDLCFTNVYADSFVIRGD
EVRQIAPGQTGKIADYNYKLPDDFTGCVIAWNSNNLDSKVGGNYNYLYRLFRKSNLKPFERDIST
EIYQAGSTPCNGVEGFNCYFPLQSYGFQPTNGVGYQPYRVWLSFELLHAPATVCGPKKSTNLV
KNKCVNFNFNGLTGTGVLTESNKKFLPFQQFGRDIADTTDAVRDPQTLEILDITPCSFGGVS
VITPGTNTSNQVAVLYQDVNCTEVPVAIHADQLTPTWRVYSTGSNVFQTRAGCLIGAEHV
NNSYECDIPIGAGICASYQTQTNSPRRA
(SEQ ID NO: 56)
ACGTCATTAATTAAATGGGATTCGTTTTGTTTTCTCAATTGCCTTCATTTCTTTTGGTTT
CTACTCTTTTGCTTTTTCTTGTTATTTCTCATTCATGTAGAGCTGGCGGCTCTTACCCTTA
TAAGAGCGGTGAGTACCGGACCAAGAGCTTCTTTGGTTACGGTTACTACGAGGTGCGG
ATGAAGGCTGCTAAGAACGTGGGTATCGTGTCCAGCTTCTTTACCTACACCGGGCCAT
CTGATAACAACCCTTGGGATGAGATCGACATCGAGTTCCTTGGTAAGGACACTACCAA
GGTGCAGTTCAACTGGTACAAGAACGGTGTTGGTGGCAACGAGTACCTTCACAACCTT
GGCTTTGATGCCAGCCAGGATTTCCACACTTACGGTTTTGAATGGCGGCCTGACTACAT
CGACTTCTACGTGGACGGTAAGAAGGTGTACAGGGGCACCAGAAATATCCCTGTGACT
CCTGGCAAGATCATGATGAACCTTTGGCCTGGTATCGGTGTGGATGAGTGGCTTGGTA
GATACGATGGTAGGACTCCTCTGCAGGCTGAGTACGAGTACGTTAAGTACTACCCTAA
CGGCAGATCTGAATTCAAGCTTGTGGTGAATACTCCTTTCGTGGCCGTGTTCAGCAA
CTTCGATTCTAGCCAGTGGGAGAAAGCTGATTGGGCTAACGGTTCTGTGTTCAACTGC
GTGTGGAAGCCTTCTCAGGTGACCTTCTCTAACGGCAAGATGATTCTGACCCTGGACC
GTGAGTATGTTGATTTGCCTATCGGAATTAATATCACTAGATTCCAAACATTGCTTGCT
TTGCATAGGTCATATCTTACACCAGGAGATTCTTCTTCTGGATGGACTGCTGGTGCTGC
TGCTTATTACGTTGGTTACCTTCAACCTAGAACTTTTCTTCTTAAGTACAATGAGAATG
GTACTATTACAGATGCTGTTGATTGTGCTTTGGATCCACTTTCTGAAACTAAGTGCACA
TTGAAGTCTTTTACTGTTGAGAAGGGTATCTATCAAACATCTAACTTCAGAGTTCAACC
TACTGAATCAATCGTTAGGTTCCCAAACATCACAAATCTTTGTCCTTTCGGAGAGGTTT
TTAATGCTACTAGATTCGCTTCTGTTTATGCTTGGAATAGAAAGAGGATTTCTAATTGC
GTTGCTGATTACTCAGTTTTGTACAACTCAGCTTCTTTTTCAACTTTTAAGTGTTACGGT
GTTTCTCCAACAAAGTTGAACGATCTTTGCTTCACTAACGTTTACGCTGATTCATTCGTT
ATCAGAGGAGATGAAGTTAGGCAAATTGCTCCTGGACAAACAGGAAAGATTGCTGATT
ACAACTACAAATTGCCAGATGATTTCACTGGATGTGTTATCGCTTGGAACTCTAATAAC
CTTGATTCAAAGGTTGGAGGTAATTATAACTACTTGTACAGACTTTTTAGGAAGTCTAA
TTTGAAGCCTTTCGAAAGGGATATCTCTACAGAGATCTATCAAGCTGGTTCAACTCCAT
GTAATGGTGTTGAAGGTTTTAATTGCTACTTCCCACTTCAATCATATGGATTTCAACCT
ACAAATGGTGTTGGTTACCAACCATACAGAGTTGTTGTTTTGTCTTTCGAGTTGCTTCA
TGCTCCAGCTACAGTTTGTGGTCCTAAGAAATCAACTAATCTTGTTAAGAATAAGTGCG
TTAACTTCAACTTCAATGGTTTGACTGGAACAGGTGTTCTTACTGAATCTAATAAGAAG
TTCTTGCCATTCCAACAATTCGGTAGAGATATTGCTGATACTACAGATGCTGTTAGGGA
TCCTCAAACTTTGGAGATTCTTGATATTACACCATGTTCTTTTGGAGGTGTTTCAGTTAT
TACACCTGGAACTAACACATCAAACCAAGTTGCTGTTTTGTACCAAGATGTTAATTGCA
CTGAGGTTCCTGTTGCTATTCATGCTGATCAACTTACTCCAACATGGAGAGTTTACTCT
ACTGGTTCAAACGTTTTCCAAACAAGGGCTGGATGTCTTATTGGTGCTGAACATGTTAA
TAACTCTTACGAGTGTGATATTCCTATTGGAGCTGGTATTTGCGCTTCTTATCAAACTC
AAACAAATTCACCAAGAAGGGCTTAGCTCGAGACGAAG
CoV28: Spike 227-684 Loop/762 kDa
(SEQ ID NO: 57)
MGFVLFSQLPSFLLVSTLLLFLVISHSCRAGGSYPYKSGEYRTKSFFGYGYYEVRMKAAKN
VGIVSSFFTYTGPSDNNPWDEIDIEFLGKDTTKVQFNWYKNGVGGNEYLHNLGFDASQDF
HTYGFEWRPDYIDFYVDGKKVYRGTRNIPVTPGKIMMNLWPGIGVDEWLGRYDGRTPLQ
AEYEYVKYYPNGRSVDLPIGINITRFQTLLALHRSYLTPGDSSSGWTAGAAAYYVGYLQPR
TFLLKYNENGTITDAVDCALDPLSETKCTLKSFTVEKGIYQTSNFRVQPTESIVRFPNITNLCP
FGEVFNATRFASVYAWNRKRISNCVADYSVLYNSASFSTFKCYGVSPTKLNDLCFTNVYADSFVIR
GDEVRQIAPGQTGKIADYNYKLPDDFTGCVIAWNSNNLDSKVGGNYNYLYRLFRKSNLKPFERD
ISTEIYQAGSTPCNGVEGFNCYFPLQSYGFQPTNGVGYQPYRVWLSFELLHAPATVCGPKKSTN
LVKNKCVNFNFNGLTGTGVLTESNKKFLPFQQFGRDIADTTDAVRDPQTLEILDITPCSFGG
VSVITPGTNTSNQVAVLYQDVNCTEVPVAIHADQLTPTWRVYSTGSNVFQTRAGCLIGAE
HVNNSYECDIPIGAGICASYQTQTNSPRRAEFKLVVNTPFVAVFSNFDSSQWEKADWANGS
VFNCVWKPSQVTFSNGKMILTLDREY-
(SEQ ID NO: 58)
ACGTCATTAATTAAATGGGATTCGTTTTGTTTTCTCAATTGCCTTCATTTCTTTTGGTTT
CTACTCTTTTGCTTTTTCTTGTTATTTCTCATTCATGTAGAGCTGGCGGCTCTTACCCTTA
TAAGAGCGGTGAGTACCGGACCAAGAGCTTCTTTGGTTACGGTTACTACGAGGTGCGG
ATGAAGGCTGCTAAGAACGTGGGTATCGTGTCCAGCTTCTTTACCTACACCGGGCCAT
CTGATAACAACCCTTGGGATGAGATCGACATCGAGTTCCTTGGTAAGGACACTACCAA
GGTGCAGTTCAACTGGTACAAGAACGGTGTTGGTGGCAACGAGTACCTTCACAACCTT
GGCTTTGATGCCAGCCAGGATTTCCACACTTACGGTTTTGAATGGCGGCCTGACTACAT
CGACTTCTACGTGGACGGTAAGAAGGTGTACAGGGGCACCAGAAATATCCCTGTGACT
CCTGGCAAGATCATGATGAACCTTTGGCCTGGTATCGGTGTGGATGAGTGGCTTGGTA
GATACGATGGTAGGACTCCTCTGCAGGCTGAGTACGAGTACGTTAAGTACTACCCTAA
CGGCAGATCTGTTGATCTTCCTATCGGTATTAATATCACTAGATTCCAAACATTGCTTG
CTTTGCATAGGTCATATCTTACACCTGGAGATTCTTCTTCTGGATGGACTGCTGGTGCT
GCTGCTTATTACGTTGGTTACTTGCAACCAAGAACTTTTCTTCTTAAGTACAATGAGAA
TGGTACTATTACAGATGCTGTTGATTGTGCTTTGGATCCTCTTTCTGAAACTAAGTGCA
CATTGAAGTCTTTTACTGTTGAGAAGGGTATCTATCAAACATCTAACTTCAGAGTTCAA
CCAACTGAATCAATCGTTAGGTTCCCAAACATCACAAATCTTTGTCCTTTCGGAGAGGT
TTTTAATGCTACTAGATTCGCTTCTGTTTATGCTTGGAATAGAAAGAGGATTTCTAATT
GCGTTGCTGATTACTCAGTTCTTTACAACTCAGCTTCTTTTTCAACTTTTAAGTGTTACG
GTGTTTCTCCAACAAAGTTGAACGATCTTTGCTTCACTAACGTTTACGCTGATTCATTC
GTTATCAGAGGAGATGAAGTTAGGCAAATTGCTCCTGGACAAACAGGAAAGATTGCTG
ATTACAACTACAAATTGCCAGATGATTTCACTGGATGTGTTATCGCTTGGAACTCTAAT
AACCTTGATTCAAAGGTTGGAGGTAATTATAACTACTTGTACAGACTTTTTAGGAAGTC
TAATTTGAAGCCTTTCGAAAGGGATATCTCTACAGAGATCTATCAAGCTGGTTCAACTC
CATGTAATGGTGTTGAAGGTTTTAATTGCTACTTCCCACTTCAATCATATGGATTTCAA
CCTACAAATGGTGTTGGTTACCAACCATACAGAGTTGTTGTTTTGTCTTTCGAGTTGCT
TCATGCTCCAGCTACAGTTTGTGGTCCTAAGAAATCAACTAATCTTGTTAAGAATAAGT
GCGTTAACTTCAACTTCAATGGTTTGACTGGAACAGGTGTTCTTACTGAATCTAATAAG
AAGTTCTTGCCTTTCCAACAATTCGGTAGAGATATTGCTGATACTACAGATGCTGTTAG
GGATCCTCAAACTTTGGAGATTCTTGATATTACACCATGTTCTTTTGGAGGTGTTTCAG
TTATTACACCAGGAACTAACACATCAAACCAAGTTGCTGTTTTGTACCAAGATGTTAAT
TGCACTGAAGTTCCTGTTGCTATTCATGCTGATCAACTTACTCCAACATGGAGAGTTTA
CTCTACTGGTTCAAACGTTTTCCAAACAAGGGCTGGATGTTTGATTGGTGCTGAACATG
TTAATAACTCTTACGAGTGTGATATTCCTATTGGAGCTGGTATTTGCGCTTCTTATCAA
ACTCAAACAAATTCACCTAGAAGGGCTGAATTCAAGCTTGTGGTGAATACTCCTTTC
GTGGCCGTGTTCAGCAACTTCGATTCTAGCCAGTGGGAGAAAGCTGATTGGGCTAACG
GTTCTGTGTTCAACTGCGTGTGGAAGCCTTCTCAGGTGACCTTCTCTAACGGCAAGATG
ATTCTGACCCTGGACCGTGAGTATTAGCTCGAGACGAAG
CoV29: Spike 319-684 c-term + T-cell epitope/72.7 kDa
(SEQ ID NO: 59)
MGFVLFSQLPSFLLVSTLLLFLVISHSCRAGGSYPYKSGEYRTKSFFGYGYYEVRMKAAKN
VGIVSSFFTYTGPSDNNPWDEIDIEFLGKDTTKVQFNWYKNGVGGNEYLHNLGFDASQDF
HTYGFEWRPDYIDFYVDGKKVYRGTRNIPVTPGKIMMNLWPGIGVDEWLGRYDGRTPLQ
AEYEYVKYYPNGRSKYEQYIKWPWYIWLGFIAGLIAIVMVTIMLCCMTSCCSCLKGCCS
CGSCCKFDEDDSEPEFKLVVNTPFVAVFSNFDSSQWEKADWANGSVFNCVWKPSQVTFSN
GKMILTLDTREYRVQPTESIVRFPNITNLCPFGEVFNATRFASVYAWNRKRISNCVADYSVLYNSAS
FSTFKCYGVSPTKLNDLCFTNVYADSFVIRGDEVRQIAPGQTGKIADYNYKLPDDFTGCVIAWNS
NNLDSKVGGNYNYLYRLFRKSNLKPFERDISTEIYQAGSTPCNGVEGFNCYFPLQSYGFQPTNGV
GYQPYRVVVLSFELLHAPATVCGPKKSTNLVKNKCVNFNFNGLTGTGVLTESNKKFLPFQQF
GRDIADTTDAVRDPQTLEILDITPCSFGGVSVITPGTNTSNQVAVLYQDVNCTEVPVAIHAD
QLTPTWRVYSTGSNVFQTRAGCLIGAEHVNNSYECDIPIGAGICASYQTQTNSPRRA
(SEQ ID NO: 60)
ACGTCATTAATTAAATGGGATTCGTTTTGTTTTCTCAATTGCCTTCATTTCTTTTGGTTT
CTACTCTTTTGCTTTTTCTTGTTATTTCTCATTCATGTAGAGCTGGCGGCTCTTACCCTTA
TAAGAGCGGTGAGTACCGGACCAAGAGCTTCTTTGGTTACGGTTACTACGAGGTGCGG
ATGAAGGCTGCTAAGAACGTGGGTATCGTGTCCAGCTTCTTTACCTACACCGGGCCAT
CTGATAACAACCCTTGGGATGAGATCGACATCGAGTTCCTTGGTAAGGACACTACCAA
GGTGCAGTTCAACTGGTACAAGAACGGTGTTGGTGGCAACGAGTACCTTCACAACCTT
GGCTTTGATGCCAGCCAGGATTTCCACACTTACGGTTTTGAATGGCGGCCTGACTACAT
CGACTTCTACGTGGACGGTAAGAAGGTGTACAGGGGCACCAGAAATATCCCTGTGACT
CCTGGCAAGATCATGATGAACCTTTGGCCTGGTATCGGTGTGGATGAGTGGCTTGGTA
GATACGATGGTAGGACTCCTCTGCAGGCTGAGTACGAGTACGTTAAGTACTACCCTAA
CGGCAGATCTAAGTACGAGCAATATATTAAGTGGCCTTGGTATATTTGGTTGGGT
TTTATTGCTGGTCTTATCGCTATCGTTATGGTTACTATTATGTTGTGTTGCATGAC
ATCATGTTGCTCTTGTCTTAAGGGATGTTGCTCATGCGGTTCTTGTTGCAAATTTG
ATGAAGATGATTCTGAACCTGAATTCAAGCTTGTGGTGAATACTCCTTTCGTGGCC
GTGTTCAGCAACTTCGATTCTAGCCAGTGGGAGAAAGCTGATTGGGCTAACGGTTCTG
TGTTCAACTGCGTGTGGAAGCCTTCTCAGGTGACCTTCTCTAACGGCAAGATGATTCTG
ACCCTGGACCGTGRGTATAGGGTTCAACCAACTGAGTCAATCGTTAGGTTCCCAAACATCA
CAAATTTGTGTCCTTTCGGTGAAGTTTTTAATGCTACTAGATTCGCTTCTGTTTATGCTTGGAA
TAGAAAGAGGATTTCAAATTGCGTTGCTGATTACTCTGTTCTTTACAACTCTGCTTCTTTTTCTA
CTTTTAAGTGTTACGGTGTTTCACCAACTAAGTTGAACGATCTTTGCTTCACAAACGTTTACGC
TGATTCTTTCGTTATTAGAGGAGATGAGGTTAGGCAAATTGCTCCTGGACAAACTGGAAAGAT
TGCTGATTACAACTACAAATTGCCAGATGATTTCACAGGATGCGTTATCGCTTGGAACTCAAA
TAACCTTGATTCTAAGGTTGGAGGTAATTATAACTACTTGTACAGACTTTTTAGGAAGTCAAAT
TTGAAGCCTTTCGAAAGGGATATCTCAACTGAGATCTATCAAGCTGGTTCTACACCATGTAAT
GGTGTTGAAGGTTTTAATTGCTACTTCCCACTTCAATCTTATGGATTTCAACCTACTAATGGTG
TTGGTTACCAACCATACAGAGTTGTTGTTTTGTCATTCGAGTTGCTTCATGCTCCAGCTACTGT
TTGTGGTCCTAAGAAATCTACAAATCTTGTTAAGAATAAGTGCGTTAACTTCAACTTCAA
TGGTTTGACTGGAACAGGTGTTCTTACAGAATCAAATAAGAAGTTCTTGCCTTTCCAAC
AATTCGGTAGAGATATTGCTGATACTACAGATGCTGTTAGGGATCCTCAAACTTTGGA
GATTCTTGATATTACACCATGTTCATTTGGAGGTGTTTCTGTTATTACTCCAGGAACTA
ACACATCTAACCAAGTTGCTGTTTTGTACCAAGATGTTAATTGCACAGAAGTTCCTGTT
GCTATTCATGCTGATCAACTTACTCCAACATGGAGAGTTTACTCAACTGGATCTAACGT
TTTCCAAACAAGGGCTGGATGTCTTATTGGTGCTGAACATGTTAATAACTCTTACGAGT
GTGATATTCCTATTGGAGCTGGTATTTGCGCTTCATATCAAACTCAAACAAATTCTCCA
AGAAGGGCTTAGCTCGAGACGAAG
CoV30: Spike 319-598 c-term + T-cell epitope 63.5 kDa
(Amplify with PCR from CoV29)
(SEQ ID NO: 61)
MGFVLFSQLPSFLLVSTLLLFLVISHSCRAGGSYPYKSGEYRTKSFFGYGYYEVRMKAAKN
VGIVSSFFTYTGPSDNNPWDEIDIEFLGKDTTKVQFNWYKNGVGGNEYLHNLGFDASQDF
HTYGFEWRPDYIDFYVDGKKVYRGTRNIPVTPGKIMMNLWPGIGVDEWLGRYDGRTPLQ
AEYEYVKYYPNGRSKYEQYIKWPWYIWLGFIAGLIAIVMVTIMLCCMTSCCSCLKGCCS
CGSCCKFDEDDSEPEFKLVVNTPFVAVFSNFDSSQWEKADWANGSVFNCVWKPSQVTFSN
GKMILTLDTREYRVQPTESIVRFPNITNLCPFGEVFNATRFASVYAWNRKRISNCVADYSVLYNSAS
FSTFKCYGVSPTKLNDLCFTNVYADSFVIRGDEVRQIAPGQTGKIADYNYKLPDDFTGCVIAWNS
NNLDSKVGGNYNYLYRLFRKSNLKPFERDISTEIYQAGSTPCNGVEGFNCYFPLQSYGFQPTNGV
GYQPYRVVVLSFELLHAPATVCGPKKSTNLVKNKCVNFNFNGLTGTGVLTESNKKFLPFQQF
GRDIADTTDAVRDPQTLEILDITPCSFGGVSVI
(SEQ ID NO: 62)
ACGTCATTAATTAAATGGGATTCGTTTTGTTTTCTCAATTGCCTTCATTTCTTTTGGTTT
CTACTCTTTTGCTTTTTCTTGTTATTTCTCATTCATGTAGAGCTGGCGGCTCTTACCCTTA
TAAGAGCGGTGAGTACCGGACCAAGAGCTTCTTTGGTTACGGTTACTACGAGGTGCGG
ATGAAGGCTGCTAAGAACGTGGGTATCGTGTCCAGCTTCTTTACCTACACCGGGCCAT
CTGATAACAACCCTTGGGATGAGATCGACATCGAGTTCCTTGGTAAGGACACTACCAA
GGTGCAGTTCAACTGGTACAAGAACGGTGTTGGTGGCAACGAGTACCTTCACAACCTT
GGCTTTGATGCCAGCCAGGATTTCCACACTTACGGTTTTGAATGGCGGCCTGACTACAT
CGACTTCTACGTGGACGGTAAGAAGGTGTACAGGGGCACCAGAAATATCCCTGTGACT
CCTGGCAAGATCATGATGAACCTTTGGCCTGGTATCGGTGTGGATGAGTGGCTTGGTA
GATACGATGGTAGGACTCCTCTGCAGGCTGAGTACGAGTACGTTAAGTACTACCCTAA
CGGCAGATCTAAGTACGAGCAATATATTAAGTGGCCTTGGTATATTTGGTTGGGT
TTTATTGCTGGTCTTATCGCTATCGTTATGGTTACTATTATGTTGTGTTGCATGAC
ATCATGTTGCTCTTGTCTTAAGGGATGTTGCTCATGCGGTTCTTGTTGCAAATTTG
ATGAAGATGATTCTGAACCTGAATTCAAGCTTGTGGTGAATACTCCTTTCGTGGCC
GTGTTCAGCAACTTCGATTCTAGCCAGTGGGAGAAAGCTGATTGGGCTAACGGTTCTG
TGTTCAACTGCGTGTGGAAGCCTTCTCAGGTGACCTTCTCTAACGGCAAGATGATTCTG
ACCCTGGACCGTGAGTATAGGGTTCAACCAACTGAGTCAATCGTTAGGTTCCCAAACA
TCACAAATTTGTGTCCTTTCGGTGAAGTTTTTAATGCTACTAGATTCGCTTCTGTTTATG
CTTGGAATAGAAAGAGGATTTCAAATTGCGTTGCTGATTACTCTGTTCTTTACAACTCT
GCTTCTTTTTCTACTTTTAAGTGTTACGGTGTTTCACCAACTAAGTTGAACGATCTTTGC
TTCACAAACGTTTACGCTGATTCTTTCGTTATTAGAGGAGATGAGGTTAGGCAAATTGC
TCCTGGACAAACTGGAAAGATTGCTGATTACAACTACAAATTGCCAGATGATTTCACA
GGATGCGTTATCGCTTGGAACTCAAATAACCTTGATTCTAAGGTTGGAGGTAATTATA
ACTACTTGTACAGACTTTTTAGGAAGTCAAATTTGAAGCCTTTCGAAAGGGATATCTCA
ACTGAGATCTATCAAGCTGGTTCTACACCATGTAATGGTGTTGAAGGTTTTAATTGCTA
CTTCCCACTTCAATCTTATGGATTTCAACCTACTAATGGTGTTGGTTACCAACCATACA
GAGTTGTTGTTTTGTCATTCGAGTTGCTTCATGCTCCAGCTACTGTTTGTGGTCCTAAGA
AATCTACAAATCTTGTTAAGAATAAGTGCGTTAACTTCAACTTCAATGGTTTGACTGGA
ACAGGTGTTCTTACAGAATCAAATAAGAAGTTCTTGCCTTTCCAACAATTCGGTAGAG
ATATTGCTGATACTACAGATGCTGTTAGGGATCCTCAAACTTTGGAGATTCTTGATATT
ACACCATGTTCATTTGGAGGTGTTTCTGTTATTTAGCTCGAGACGAAG
CoV-31: S1-93/S1-105/S2-78 - 25 kDa
(SEQ ID NO: 63)
MDIDPYKEFGATVELLSFLPSDFFPSVRDLLDTASALYREALESPEHCSPHHTALRQAILCW
GELMTLATWNGANLEDGGGSGGSGGSGGSGGSVLTESNKKFLPFQQFGGGSAIHADQLTPT
WRVYSTGGSDSFKEELDKYFKNHTSGGGSGGSGGSGGSGGSSRDLVVNAVNTNMGLKIRQL
LWFHISCLTFGRETVLEYLVSFGVWIRTPPAYRPPNAPILSTLPETTVV
(SEQ ID NO: 64)
ACGTCATTAATTAAATGGACATCGATCCGTACAAAGAATTTGGCGCGACCGTCGAGCT
GCTGAGCTTCCTGCCGAGCGATTTTTTCCCGAGCGTGCGTGACCTGCTGGACACCGCGA
GCGCACTGTATCGTGAAGCACTGGAAAGCCCAGAGCACTGTAGCCCGCACCACACCGC
CCTGCGCCAGGCGATTCTGTGCTGGGGTGAACTGATGACCCTGGCCACCTGGGTGGGT
GCTAACCTTGAGgaCGGTGGTGGATCAGGAGGAAGTGGCGGCAGTGGAGGTTCAGGGG
GGAGCGTCCTGACTGAGTCGAACAAGAAATTCCTTCCATTTCAGCAGTTTGGTGGGGG
AAGCGCCATACATGCTGATCAACTCACCCCAACTTGGAGAGTATATTCTACGGGTGGA
TCTGATTCATTTAAGGAAGAATTAGACAAATATTTCAAGAATCATACATCTGGTGGTG
GCAGTGGGGGATCGGGTGGAAGTGGAGGTTCCGGAGGCTCTAGCCGTGATCTGGTCGT
CAACTATGTGAATACCAACATGGGTCTGAAAATTCGTCAGCTGCTGTGGTTTCATATTA
GCTGCCTGACCTTCGGTCGTGAAACCGTGCTGGAGTATCTGGTGAGCTTCGGTGTGTGG
ATTCGCACCCCGCCGGCGTATCGTCCGCCGAACGCGCCAATTCTGAGCACGCTGCCGG
AGACCACCGTGGTTTAGCTCGAGACGAAG
CoV-32: Spike 524-598 HBcAg loop - 27 kDa
(SEQ ID NO: 65)
MDIDPYKEFGATVELLSFLPSDFFPSVRDLLDTASALYREALESPEHCSPHHTALRQAILCW
GELMTLATWVGANLEDPGGGSGGSGGSGGSGGSVCGPKKSTNLVKNKCVNFNFNGLTGTG
VLTESNKKFLPFQQFGRDIADTTDAVRDPQTLEILDITPCSFGGVSVIGGGSGGSGGSGGSGGS
ASRDLVVNYVNTNMGLKIRQLLWFHISCLTFGRETVLEYLVSFGVWIRTPPAYRPPNAPILS
TLPETTVV
(SEQ ID NO: 66)
ACGTCATTAATTAAATGGACATCGATCCGTACAAAGAATTTGGCGCGACCGTCGAGCT
GCTGAGCTTCCTGCCGAGCGATTTTTTCCCGAGCGTGCGTGACCTGCTGGACACCGCGA
GCGCACTGTATCGTGAAGCACTGGAAAGCCCAGAGCACTGTAGCCCGCACCACACCGC
CCTGCGCCAGGCGATTCTGTGCTGGGGTGAACTGATGACCCTGGCCACCTGGGTGGGT
GCTAACCTTGAGgaCccTGGTGGTGGATCAGGAGGAAGTGGCGGCAGTGGAGGTTCAGG
GGGGAGCGTTTGTGGTCCTAAGAAAAGTACCAATCTCGTGAAGAACAAATGCGTCAATTTCA
ACTTCAATGGGTTAACTGGAACTGGAGTTCTAACAGAGTCCAATAAGAAATTCCTTCCATTTCA
GCAATTTGGCAGGGATATTGCAGACACAACAGATGCTGTTAGAGATCCACAAACGTTGGAAA
TTCTGGACATAACTCCCTGTTCTTTTGGTGGAGTATCAGTGATCGGTGGTGGCAGTGGGGG
ATCGGGTGGAAGTGGAGGTTCCGGAGGCTCTgctagcCGTGATCTGGTCGTCAACTATGT
GAATACCAACATGGGTCTGAAAATTCGTCAGCTGCTGTGGTTTCATATTAGCTGCCTGA
CCTTCGGTCGTGAAACCGTGCTGGAGTATCTGGTGAGCTTCGGTGTGTGGATTCGCACC
CCGCCGGCGTATCGTCCGCCGAACGCGCCAATTCTGAGCACGCTGCCGGAGACCACCG
TGGTTTAGCTCGAGACGAAG
CoV-33: Spike 601-640 HBcAg loop - 23kDa
(SEQ ID NO: 67)
MDIDPYKEFGATVELLSFLPSDFFPSVRDLLDTASALYREALESPEHCSPHHTALRQAILCW
GELMTLATWVGANLEDPGGGSGGSGGSGGSGGSGTNTSNQVAVLYQDVNCTEVPVAIHADQ
LTPTWRVYSTGSGGGSGGSGGSGGSGGSASRDLVVNYVNTNMGLKIRQLLWFHISCLTFGR
ETVLEYLVSFGVWIRTPPAYRPPNAPILSTLPETTVV
(SEQ ID NO: 68)
ACGTCATTAATTAAATGGACATCGATCCGTACAAAGAATTTGGCGCGACCGTCGAGCT
GCTGAGCTTCCTGCCGAGCGATTTTTTCCCGAGCGTGCGTGACCTGCTGGACACCGCGA
GCGCACTGTATCGTGAAGCACTGGAAAGCCCAGAGCACTGTAGCCCGCACCACACCGC
CCTGCGCCAGGCGATTCTGTGCTGGGGTGAACTGATGACCCTGGCCACCTGGGTGGGT
GCTAACCTTGAGgaCccTGGTGGTGGATCAGGAGGAAGTGGCGGCAGTGGAGGTTCAGG
GGGGAGCGGTACGAATACCTCAAATCAGGTTGCTGTGTTGTATCAAGACGTGAACTGTACTG
AAGTTCCTGTAGCCATTCATGCAGATCAACTTACACCAACTTGGAGAGTCTACTCTACAGGAA
GTGGTGGTGGCAGTGGGGGATCGGGTGGAAGTGGAGGTTCCGGAGGCTCTgctagcCGTG
ATCTGGTCGTCAACTATGTGAATACCAACATGGGTCTGAAAATTCGTCAGCTGCTGTG
GTTTCATATTAGCTGCCTGACCTTCGGTCGTGAAACCGTGCTGGAGTATCTGGTGAGCT
TCGGTGTGTGGATTCGCACCCCGCCGGCGTATCGTCCGCCGAACGCGCCAATTCTGAG
CACGCTGCCGGAGACCACCGTGGTTTAGCTCGAGACGAAG
CoV-34: Spike 530-684 HBcAg dual core - 58 kDa
(SEQ ID NO: 69)
MDIDPYKEFGATVELLSFLPSDFFPSVRDLLDTASALYREALESPEHCSPHHTALRQAILCW
GELMTLATWVGNNLEGSAGGGRDPASRDLVVNYVNTNMGLKIRQLLWFHISCLTFGRET
VLEYLVSFGVWIRTPPAYRPPNAPILSTLPETTVVGGSSGGSGGSGGSGGSGGSGGSTMDID
PYKEFGATVELLSFLPSDFFPSVRDLLDTASALYREALESPEHCSPHHTALRQAILCWGELM
TLATWVGNNLEFGGSSTNLVKNKCVNFNFNGLTGTGVLTESNKKFLPFQQFGRDIADTTDAV
RDPQTLEILDITPCSFGGVSVITPGTNTSNQVAVLYQDVNCTEVPVAIHADQLTPTWRVYSTGSNV
FQTRAGCLIGAEHVNNSYECDIPIGAGICASYQTQTNSPRRASGGASDPASRDLVVNYVNTNM
GLKIRQLLWFHISCLTFGRETVLEYLVSFGVWIRTPPAYRPPNAPILSTLPETTVVRRRDRGR
SPRRRTPSPRRRRSQSPRRRRSQSRESQCLE
(SEQ ID NO: 70)
ttaattaaATGGACATCGATCCGTACAAAGAATTTGGCGCGACCGTCGAGCTGCTGAGCTTC
CTGCCGAGCGATTTTTTCCCGAGCGTGCGTGACCTGCTGGACACCGCGAGCGCACTGT
ATCGTGAAGCACTGGAAAGCCCAGAGCACTGTAGCCCGCACCACACCGCCCTGCGCCA
GGCGATTCTGTGCTGGGGTGAACTGATGACCCTGGCCACCTGGGTGGGTAATAATCTG
GAAGGTTCCGCCGGCGGCGGCCGCGATCCGGCGAGCCGTGATCTGGTCGTCAACTATG
TGAATACCAACATGGGTCTGAAAATTCGTCAGCTGCTGTGGTTTCATATTAGCTGCCTG
ACCTTCGGTCGTGAAACCGTGCTGGAGTATCTGGTGAGCTTCGGTGTGTGGATTCGCAC
CCCGCCGGCGTATCGTCCGCCGAACGCGCCAATTCTGAGCACGCTGCCGGAGACCACC
GTGGTTGGTGGGAGCTCTGGTGGCAGCGGCGGCAGCGGTGGTAGCGGTGGCAGCGGT
GGTAGCGGCGGGTCGACTATGGATATCGACCCATATAAAGAATTTGGCGCGACGGTTG
AGCTGCTGAGCTTTCTGCCAAGCGATTTCTTTCCGAGCGTCCGCGACCTGCTGGATACC
GCCAGCGCACTGTATCGTGAAGCCCTGGAGAGCCCGGAACATTGCAGCCCGCATCATA
CGGCCCTGCGTCAGGCAATCCTGTGCTGGGGCGAACTGATGACCCTGGCAACCTGGGT
CGGCAATAATCTGGAATTCGGCGGATCCAGTACTAATCTTGTGAAAAACAAATGTGTT
AATTTCAACTTCAATGGTTTAACCGGAACTGGTGTTCTGACCGAGAGTAACAAGAAAT
TTCTACCTTTCCAGCAATTTGGGAGGGACATTGCAGATACGACTGATGCTGTTAGAGA
CCCTCAGACATTGGAGATTTTGGATATAACCCCCTGCTCTTTTGGTGGTGTCTCTGTTAT
AACACCTGGCACTAATACTTCAAACCAAGTTGCTGTATTATATCAAGATGTGAATTGTA
CTGAGGTTCCTGTGGCAATTCATGCTGATCAACTTACACCAACATGGAGAGTCTATTCA
ACTGGCAGCAATGTTTTTCAAACAAGAGCTGGATGCTTAATTGGAGCTGAACATGTGA
ACAATTCATATGAATGTGATATCCCAATTGGGGCCGGCATTTGTGCCTCATACCAAACT
CAGACCAATTCTCCAAGAAGGGCCTCCGGAGGCGCTAGCGACCCAGCAAGCCGTGATC
TGGTTGTTAATTACGTGAACACCAACATGGGCCTGAAGATCCGCCAACTGCTGTGGTTT
CATATCAGCTGTCTGACGTTTGGCCGCGAGACGGTGCTGGAATACCTGGTTAGCTTTGG
CGTTTGGATTCGTACGCCACCGGCCTACCGCCCACCAAACGCACCGATTCTGAGCACG
CTGCCGGAAACGACGGTTGTTCGTCGCCGTGATCGCGGCCGTAGCCCGCGCCGCCGTA
CGCCGAGCCCACGTCGTCGCCGCAGCCAGAGCCCGCGCCGCCGTCGCAGCCAGAGCCG
TGAAAGCCAATGTCTGGAGTGActcgag
CoV-35: Spike 437-508 HBcAg dual core [RBM] (PCR amplified
from CoV-2 with CoV35F & R primers)
(SEQ ID NO: 71)
MDIDPYKEFGATVELLSFLPSDFFPSVRDLLDTASALYREALESPEHCSPHHTALRQAILCW
GELMTLATWVGNNLEGSAGGGRDPASRDLVVNYVNTNMGLKIRQLLWFHISCLTFGRET
VLEYLVSFGVWIRTPPAYRPPNAPILSTLPETTVVGGSSGGSGGSGGSGGSGGSGGSTMDID
PYKEFGATVELLSFLPSDFFPSVRDLLDTASALYREALESPEHCSPHHTALRQAILCWGELM
TLATWNGNNLEFGGSNSNNLDSKVGGNYNYLYRLFRKSNLKPFERDISTEIYQAGSTPCNGVEG
FNCYFPLQSYGFQPTNGVGYQPYSGGASDPASRDLVVNYVNTNMGLKIRQLLWFHISCLTF
GRETVLEYLVSFGVWIRTPPAYRPPNAPILSTLPETTVVRRRDRGRSPRRRTPSPRRRRSQSP
RRRRSQSRESQCLE
(SEQ ID NO: 72)
ttaattaaATGGACATCGATCCGTACAAAGAATTTGGCGCGACCGTCGAGCTGCTGAGCTTC
CTGCCGAGCGATTTTTTCCCGAGCGTGCGTGACCTGCTGGACACCGCGAGCGCACTGT
ATCGTGAAGCACTGGAAAGCCCAGAGCACTGTAGCCCGCACCACACCGCCCTGCGCCA
GGCGATTCTGTGCTGGGGTGAACTGATGACCCTGGCCACCTGGGTGGGTAATAATCTG
GAAGGTTCCGCCGGCGGCGGCCGCGATCCGGCGAGCCGTGATCTGGTCGTCAACTATG
TGAATACCAACATGGGTCTGAAAATTCGTCAGCTGCTGTGGTTTCATATTAGCTGCCTG
ACCTTCGGTCGTGAAACCGTGCTGGAGTATCTGGTGAGCTTCGGTGTGTGGATTCGCAC
CCCGCCGGCGTATCGTCCGCCGAACGCGCCAATTCTGAGCACGCTGCCGGAGACCACC
GTGGTTGGTGGGAGCTCTGGTGGCAGCGGCGGCAGCGGTGGTAGCGGTGGCAGCGGT
GGTAGCGGCGGGTCGACTATGGATATCGACCCATATAAAGAATTTGGCGCGACGGTTG
AGCTGCTGAGCTTTCTGCCAAGCGATTTCTTTCCGAGCGTCCGCGACCTGCTGGATACC
GCCAGCGCACTGTATCGTGAAGCCCTGGAGAGCCCGGAACATTGCAGCCCGCATCATA
CGGCCCTGCGTCAGGCAATCCTGTGCTGGGGCGAACTGATGACCCTGGCAACCTGGGT
CGGCAATAATCTGGAATTCGGCGGATCCAACTCTAACAACCTGGACTCTAAGGTTGGCGG
CAACTACAACTACCTCTACAGGCTGTTCCGGAAGTCCAACCTTAAGCCTTTCGAGAGGGATAT
CAGCACCGAGATCTATCAGGCTGGTTCTACTCCTTGCAACGGTGTTGAGGGTTTCAACTGCT
ACTTCCCGCTTCAGTCTTACGGTTTCCAGCCTACTAATGGTGTGGGCTACCAGCCTTATTCCG
GAGGCGCTAGCGACCCAGCAAGCCGTGATCTGGTTGTTAATTACGTGAACACCAACAT
GGGCCTGAAGATCCGCCAACTGCTGTGGTTTCATATCAGCTGTCTGACGTTTGGCCGCG
AGACGGTGCTGGAATACCTGGTTAGCTTTGGCGTTTGGATTCGTACGCCACCGGCCTAC
CGCCCACCAAACGCACCGATTCTGAGCACGCTGCCGGAAACGACGGTTGTTCGTCGCC
GTGATCGCGGCCGTAGCCCGCGCCGCCGTACGCCGAGCCCACGTCGTCGCCGCAGCCA
GAGCCCGCGCCGCCGTCGCAGCCAGAGCCGTGAAAGCCAATGTCTGGAGTGActcgag
In red: HLA Class II profile targeting CD4+ (Th) cells
In bold: HLA class I profile targeting CD8+ (CTL) cells
CoV-36 PR1a-LicKM-N protein T-cell epitope mix no cysteines in loop + RBM -
43.5 kDa
(SEQ ID NO: 73)
MGFVLFSQLPSFLLVSTLLLFLVISHSCRAGGSYPYKSGEYRTKSFFGYGYYEVRMKAAKN
VGIVSSFFTYTGPSDNNPWDEIDIEFLGKDTTKVQFNWYKNGVGGNEYLHNLGFDASQDF
HTYGFEWRPDYIDFYVDGKKVYRGTRNIPVTPGKIMMNLWPGIGVDEWLGRYDGRTPLQ
AEYEYVKYYPNGRSDQIGYYRRATRRIRGGDGKMKDLSPRWYFYYLGTGALALLLLDR
LNQLESKMSAFFGMSRIGMEVADLDDFSKQLQQSMSSADSTEFKLVVNTPFVAVFSNFDS
SQWEKADWANGSVFNCVWKPSQVTFSNGKMILTLDREYNSNNLDSKVGGNYNYLYRLFRKS
NLKPFERDISTEIYQAGSTPCNGVEGFNCYFPLQSYGFQPTNGVGYQPY
(SEQ ID NO: 74)
ACGTCATTAATTAAATGGGATTCGTTTTGTTTTCTCAATTGCCTTCATTTCTTTTGGTTT
CTACTCTTTTGCTTTTTCTTGTTATTTCTCATTCATGTAGAGCTGGCGGCTCTTACCCTTA
TAAGAGCGGTGAGTACCGGACCAAGAGCTTCTTTGGTTACGGTTACTACGAGGTGCGG
ATGAAGGCTGCTAAGAACGTGGGTATCGTGTCCAGCTTCTTTACCTACACCGGGCCAT
CTGATAACAACCCTTGGGATGAGATCGACATCGAGTTCCTTGGTAAGGACACTACCAA
GGTGCAGTTCAACTGGTACAAGAACGGTGTTGGTGGCAACGAGTACCTTCACAACCTT
GGCTTTGATGCCAGCCAGGATTTCCACACTTACGGTTTTGAATGGCGGCCTGACTACAT
CGACTTCTACGTGGACGGTAAGAAGGTGTACAGGGGCACCAGAAATATCCCTGTGACT
CCTGGCAAGATCATGATGAACCTTTGGCCTGGTATCGGTGTGGATGAGTGGCTTGGTA
GATACGATGGTAGGACTCCTCTGCAGGCTGAGTACGAGTACGTTAAGTACTACCCTAA
CGGCAGATCTGACCAAATTGGTTATTATAGAAGAGCAACACGTCGAATAAG
GGGTGGTGATGGAAAAATGAAAGATTTGAGTCCTAGATGGTATTTCTACT
ACTTGGGGACTGGAGCTTTAGCCTTACTGCTACTCGACAGGTTGAATCAA
CTTGAGTCAAAGATGTCTGCTTTCTTTGGCATGAGCCGGATCGGAATGGA
AGTTGCTGATCTTGATGATTTTTCCAAGCAGCTTCAGCAATCGATGTCAA
GTGCAGATTCTACCGAATTCAAGCTTGTGGTGAATACTCCTTTCGTGGCCGTGTTC
AGCAACTTCGATTCTAGCCAGTGGGAGAAAGCTGATTGGGCTAACGGTTCTGTGTTCA
ACTGCGTGTGGAAGCCTTCTCAGGTGACCTTCTCTAACGGCAAGATGATTCTGACCCTG
GACCGTGAGTACAACAGCAACAACCTGGATTCTAAGGTCGGCGGCAACTACAACTACCTCT
ACAGGCTGTTCCGGAAGTCCAACCTTAAGCCTTTCGAGAGGGATATCAGCACCGAGATCTAT
CAGGCTGGTTCTACTCCTTGCAATGGCGTTGAGGGTTTCAACTGCTACTTCCCGCTTCAGTCT
TACGGATTCCAGCCTACTAATGGTGTGGGCTACCAGCCTTATTAGCTCGAGACGAAG
CoV 37 N protein T-cell epitope mix no cysteines in a VLP format -
HB144-CoV-Ntcell: HBcAg core 144 - Linker - N protein
T-cell epitopes (~26 kDa)
(SEQ ID NO: 75)
MDIDPYKEFGATVELLSFLPSDFFPSVRDLLDTASALYREALESPEHCSPHHTALRQAILCW
GELMTLATWVGANLEDPASRDLVVNYVNTNMGLKIRQLLWFHISCLTFGRETVLEYLVSF
GVWIRTPPAYRPPNAPILSTLPSGGSDQIGYYRRATRRIRGGDGKMKDLSPRWYFYYLG
TGALALLLLDRLNQLESKMSAFFGMSRIGMEVADLDDFSKQLQQSMSSADST-
(SEQ ID NO: 76)
ACGTCATTAATTAAATGGACATCGATCCGTACAAAGAATTTGGCGCGACCGTCGAGCT
GCTGAGCTTCCTGCCGAGCGATTTTTTCCCGAGCGTGCGTGACCTGCTGGACACCGCGA
GCGCACTGTATCGTGAAGCACTGGAAAGCCCAGAGCACTGTAGCCCGCACCACACCGC
CCTGCGCCAGGCGATTCTGTGCTGGGGTGAACTGATGACCCTGGCCACCTGGGTGGGT
GCTAACCTTGAGGATCCGGCGAGCCGTGATCTGGTCGTCAACTATGTGAATACCAACA
TGGGTCTGAAAATTCGTCAGCTGCTGTGGTTTCATATTAGCTGCCTGACCTTCGGTCGT
GAAACCGTGCTGGAGTATCTGGTGAGCTTCGGTGTGTGGATTCGCACCCCGCCGGCGT
ATCGTCCGCCGAACGCGCCAATTCTGAGCACGCTGCCGTCCGGAGGTAGCGACCAAA
TTGGTTATTATAGAAGAGCAACACGTCGAATAAGGGGTGGTGATGGAAAAAT
GAAAGATTTGAGTCCTAGATGGTATTTCTACTACTTGGGGACTGGAGCTTTAG
CCTTACTGCTACTCGACAGGTTGAATCAACTTGAGTCAAAGATGTCTGCTTTCT
TTGGCATGAGCCGGATCGGAATGGAAGTTGCTGATCTTGATGATTTTTCCAAG
CAGCTTCAGCAATCGATGTCAAGTGCAGATTCTACCTAGCTCGAGACGAAG
CoV-38 PR1a-LicKM-N protein T-cell epitope mix no cysteines - 35 kDa
(SEQ ID NO: 77)
MGFVLFSQLPSFLLVSTLLLFLVISHSCRAGGSYPYKSGEYRTKSFFGYGYYEVRMKAAKN
VGIVSSFFTYTGPSDNNPWDEIDIEFLGKDTTKVQFNWYKNGVGGNEYLHNLGFDASQDF
HTYGFEWRPDYIDFYVDGKKVYRGTRNIPVTPGKIMMNLWPGIGVDEWLGRYDGRTPLQ
AEYEYVKYYPNGRSEFKLVVNTPFVAVFSNFDSSQWEKADWANGSVFNCVWKPSQVTFS
NGKMILTLDREYQIGYYRRATRRIRGGDGKMKDLSPRWYFYYLGTGALALLLLDRLN
QLESKMSAFFGMSRIGMEVADLDDFSKQLQQSMSSADS
(SEQ ID NO: 78)
ACGTCATTAATTAAATGGGATTCGTTTTGTTTTCTCAATTGCCTTCATTTCTTTTGGTTT
CTACTCTTTTGCTTTTTCTTGTTATTTCTCATTCATGTAGAGCTGGCGGCTCTTACCCTTA
TAAGAGCGGTGAGTACCGGACCAAGAGCTTCTTTGGTTACGGTTACTACGAGGTGCGG
ATGAAGGCTGCTAAGAACGTGGGTATCGTGTCCAGCTTCTTTACCTACACCGGGCCAT
CTGATAACAACCCTTGGGATGAGATCGACATCGAGTTCCTTGGTAAGGACACTACCAA
GGTGCAGTTCAACTGGTACAAGAACGGTGTTGGTGGCAACGAGTACCTTCACAACCTT
GGCTTTGATGCCAGCCAGGATTTCCACACTTACGGTTTTGAATGGCGGCCTGACTACAT
CGACTTCTACGTGGACGGTAAGAAGGTGTACAGGGGCACCAGAAATATCCCTGTGACT
CCTGGCAAGATCATGATGAACCTTTGGCCTGGTATCGGTGTGGATGAGTGGCTTGGTA
GATACGATGGTAGGACTCCTCTGCAGGCTGAGTACGAGTACGTTAAGTACTACCCTAA
CGGCAGATCTGAATTCAAGCTTGTGGTGAATACTCCTTTCGTGGCCGTGTTCAGCAA
CTTCGATTCTAGCCAGTGGGAGAAAGCTGATTGGGCTAACGGTTCTGTGTTCAACTGC
GTGTGGAAGCCTTCTCAGGTGACCTTCTCTAACGGCAAGATGATTCTGACCCTGGACC
GTGAGTATCAAATTGGATACTACAGGCGGGCTACAAGAAGAATCCGTGGTGGTGATGG
AAAAATGAAAGATTTGTCCCCTAGATGGTATTTCTATTATCTTGGGACTGGTGCCTTAG
CATTACTACTCCTGGACCGATTGAATCAACTTGAGAGTAAGATGTCTGCTTTCTTTGGC
ATGAGCAGGATAGGAATGGAAGTTGCAGACCTTGATGATTTTTCAAAGCAATTGCAGC
AGTCGATGTCAAGTGCTGATTCTtgactcgagACGAAG
CoV-39 PR1a-LicKM-N protein T-cell epitope mix no cysteines and RBM at C
terminus - 43.5 kDa
(SEQ ID NO: 79)
MGFVLFSQLPSFLLVSTLLLFLVISHSCRAGGSYPYKSGEYRTKSFFGYGYYEVRMKAAKN
VGIVSSFFTYTGPSDNNPWDEIDIEFLGKDTTKVQFNWYKNGVGGNEYLHNLGFDASQDF
HTYGFEWRPDYIDFYVDGKKVYRGTRNIPVTPGKIMMNLWPGIGVDEWLGRYDGRTPLQ
AEYEYVKYYPNGRSEFKLVVNTPFVAVFSNFDSSQWEKADWANGSVFNCVWKPSQVTFS
NGKMILTLDREYQIGYYRRATRRIRGGDGKMKDLSPRWYFYYLGTGALALLLLDRLN
QLESKMSAFFGMSRIGMEVADLDDFSKQLQQSMSSADSTNSNNLDSKVGGNYNYLYRL
FRKSNLKPFERDISTEIYQAGSTPCNGVEGFNCYFPLQSYGFQPTNGVGYQPY
(SEQ ID NO: 80)
ACGTCATTAATTAAATGGGATTCGTTTTGTTTTCTCAATTGCCTTCATTTCTTTTGGTTTC
TACTCTTTTGCTTTTTCTTGTTATTTCTCATTCATGTAGAGCTGGCGGCTCTTACCCTTAT
AAGAGCGGTGAGTACCGGACCAAGAGCTTCTTTGGTTACGGTTACTACGAGGTGCGGA
TGAAGGCTGCTAAGAACGTGGGTATCGTGTCCAGCTTCTTTACCTACACCGGGCCATCT
GATAACAACCCTTGGGATGAGATCGACATCGAGTTCCTTGGTAAGGACACTACCAAGG
TGCAGTTCAACTGGTACAAGAACGGTGTTGGTGGCAACGAGTACCTTCACAACCTTGG
CTTTGATGCCAGCCAGGATTTCCACACTTACGGTTTTGAATGGCGGCCTGACTACATCG
ACTTCTACGTGGACGGTAAGAAGGTGTACAGGGGCACCAGAAATATCCCTGTGACTCC
TGGCAAGATCATGATGAACCTTTGGCCTGGTATCGGTGTGGATGAGTGGCTTGGTAGAT
ACGATGGTAGGACTCCTCTGCAGGCTGAGTACGAGTACGTTAAGTACTACCCTAACGG
CAGATCTGAATTCAAGCTTGTGGTGAATACTCCTTTCGTGGCCGTGTTCAGCAACTT
CGATTCTAGCCAGTGGGAGAAAGCTGATTGGGCTAACGGTTCTGTGTTCAACTGCGTGT
GGAAGCCTTCTCAGGTGACCTTCTCTAACGGCAAGATGATTCTGACCCTGGACCGTGAG
TATCAAATTGGATACTACAGGCGGGCTACAAGAAGAATCCGTGGTGGTGATGGAAAAA
TGAAAGATTTGTCCCCTAGATGGTATTTCTATTATCTTGGGACTGGTGCCTTAGCATTAC
TACTCCTGGACCGATTGAATCAACTTGAGAGTAAGATGTCTGCTTTCTTTGGCATGAGC
AGGATAGGAATGGAAGTTGCAGACCTTGATGATTTTTCAAAGCAATTGCAGCAGTCGA
TGTCAAGTGCTGATTCTACGAACTCTAACAACCTGGACTCTAAGGTTGGCGGCAACTAC
AACTACCTCTACAGGCTGTTCCGGAAGTCCAACCTTAAGCCTTTCGAGAGGGATATCAG
CACCGAGATCTATCAGGCTGGTTCTACTCCTTGCAACGGTGTTGAGGGTTTCAACTGCT
ACTTCCCGCTTCAGTCTTACGGTTTCCAGCCTACTAATGGTGTGGGCTACCAGCCTTATT
AGctcgagACGAAG
CoV-40 Ext-LicKM-Linker-CoVN 247-365 (39.5 kDa)
(SEQ ID NO: 81)
MGKMASLFATFLVVLVSLSLASESSAGGSYPYKSGEYRTKSFFGYGYYEVRMKAAKNVGI
VSSFFTYTGPSDNNPWDEIDIEFLGKDTTKVQFNWYKNGVGGNEYLHNLGFDASQDFHTY
GFEWRPDYIDFYVDGKKVYRGTRNIPVTPGKIMMNLWPGIGVDEWLGRYDGRTPLQAEY
EYVKYYPNGRSEFKLVVNTPFVAVFSNFDSSQWEKADWANGSVFNCVWKPSQVTFSNGK
MILTLDREYGGGGSGGGGTKKSAAEASKKPRQK GRRGPEQTQGNFGD
QELIRQGTDYKHWPQI [symbol] [symbol] TGAIKLDDKDPNFKDQV
ILLNKHIDAY [symbol]
CoV-40 - Codon-1
(SEQ ID NO: 82)
ACGTCAttaattaaATGGGAAAAATGGCTTCTCTTTTTGCTACTTTCCTTGTTGTGTTGGTTA
GTCTTTCTCTAGCTAGTGAGAGTAGTGCTGGCGGCTCTTACCCTTATAAGAGCGGTGAG
TACCGGACCAAGAGCTTCTTTGGTTACGGTTACTACGAGGTGCGGATGAAGGCTGCTA
AGAACGTGGGTATCGTGTCCAGCTTCTTTACCTACACCGGGCCATCTGATAACAACCCT
TGGGATGAGATCGACATCGAGTTCCTTGGTAAGGACACTACCAAGGTGCAGTTCAACT
GGTACAAGAACGGTGTTGGTGGCAACGAGTACCTTCACAACCTTGGCTTTGATGCCAG
CCAGGATTTCCACACTTACGGTTTTGAATGGCGGCCTGACTACATCGACTTCTACGTGG
ACGGTAAGAAGGTGTACAGGGGCACCAGAAATATCCCTGTGACTCCTGGCAAGATCAT
GATGAACCTTTGGCCTGGTATCGGTGTGGATGAGTGGCTTGGTAGATACGATGGTAGG
ACTCCTCTGCAGGCTGAGTACGAGTACGTTAAGTACTACCCTAACGGCAGATCTGAA
TTCAAGCTTGTGGTGAATACTCCTTTCGTGGCCGTGTTCAGCAACTTCGATTCTAGCC
AGTGGGAGAAAGCTGATTGGGCTAACGGTTCTGTGTTCAACTGCGTGTGGAAGCCTTC
TCAGGTGACCTTCTCTAACGGCAAGATGATTCTGACCCTGGACCGTGAGTATGGCGGG
GGCGGCTCAGGAGGTGGTGGAACGAAGAAGTCTGCTGCTGAAGCTTCAAAGAAACC
ACGACAGAAAAGAACTGCAACAAAAGCATATAATGTAACTCAAGCATTTGGAAGGCG
TGGTCCTGAGCAAACACAAGGAAATTTTGGTGATCAAGAGCTTATAAGGCAAGGTACC
GACTACAAGCATTGGCCTCAGATTGCTCAGTTTGCTCCCAGTGCCTCCGCGTTCTTCGG
CATGTCTAGAATTGGGATGGAAGTTACCCCGAGCGGGACATGGCTCACTTATACTGGA
GCAATCAAGTTGGATGATAAAGATCCAAATTTCAAGGACCAGGTGATACTGTTAAACA
AACACATTGATGCCTATAAAACATTTCCTCCATGActcgagcctaggACGAAG
CoV-40 - Codon-2
(SEQ ID NO:)
ACGTCAttaattaaATGGGAAAAATGGCTTCTCTTTTTGCTACTTTCCTTGTTGTGTTGGTTA
GTCTTTCTCTAGCTAGTGAGAGTAGTGCTGGCGGCTCTTACCCTTATAAGAGCGGTGAG
TACCGGACCAAGAGCTTCTTTGGTTACGGTTACTACGAGGTGCGGATGAAGGCTGCTA
AGAACGTGGGTATCGTGTCCAGCTTCTTTACCTACACCGGGCCATCTGATAACAACCCT
TGGGATGAGATCGACATCGAGTTCCTTGGTAAGGACACTACCAAGGTGCAGTTCAACT
GGTACAAGAACGGTGTTGGTGGCAACGAGTACCTTCACAACCTTGGCTTTGATGCCAG
CCAGGATTTCCACACTTACGGTTTTGAATGGCGGCCTGACTACATCGACTTCTACGTGG
ACGGTAAGAAGGTGTACAGGGGCACCAGAAATATCCCTGTGACTCCTGGCAAGATCAT
GATGAACCTTTGGCCTGGTATCGGTGTGGATGAGTGGCTTGGTAGATACGATGGTAGG
ACTCCTCTGCAGGCTGAGTACGAGTACGTTAAGTACTACCCTAACGGCAGATCTGAA
TTCAAGCTTGTGGTGAATACTCCTTTCGTGGCCGTGTTCAGCAACTTCGATTCTAGCC
AGTGGGAGAAAGCTGATTGGGCTAACGGTTCTGTGTTCAACTGCGTGTGGAAGCCTTC
TCAGGTGACCTTCTCTAACGGCAAGATGATTCTGACCCTGGACCGTGAGTATGGCGGG
GGCGGCTCAGGAGGTGGTGGAACGAAGAAATCCGCGGCCGAGGCATCAAAGAAGC
CAAGGCAGAAAAGAACTGCAACCAAAGCTTATAATGTGACACAAGCATTTGGAAGAC
GAGGCCCTGAACAAACTCAAGGAAATTTTGGTGACCAAGAACTGATCAGGCAAGGTA
CTGATTATAAACATTGGCCTCAGATTGCTCAGTTTGCTCCTTCTGCTTCTGCCTTCTTCG
GGATGAGTCGTATTGGAATGGAGGTAACCCCAAGCGGGACATGGTTGACATATACTGG
TGCAATAAAGCTTGATGATAAAGATCCAAATTTCAAGGACCAGGTTATATTACTCAAC
AAGCACATTGATGCTTACAAAACATTTCCTCCCTGActcgagcctaggACGAAG
CoV-41 Ext-8 HIS-Cov N 247-365 (14.4 kDa)
(SEQ ID NO: 83)
MGKMASLFATFLVVLVSLSLASESSAHHHHHHHHTKKSAAEASKKPRQK [symbol] [symbol]
GRRGPEQTQGNFGDQELIRQGTDYKHWPQI [symbol] [symbol] T
GAIKLDDKDPNFKDQVILLNKHIDAY [symbol]
(SEQ ID NO: 84)
ACGTCAttaattaaATGGGAAAAATGGCTTCTCTTTTTGCTACTTTCCTTGTTGTGTTGGTTA
GTCTTTCTCTAGCTAGTGAGAGTAGTGCTCACCATCATCATCACCATCATCACACCAAG
AAAAGCGCTGCCGAAGCGTCAAAGAAGCCAAGGCAGAAGAGAACTGCTACAAAAGCT
TATAATGTGACTCAAGCATTTGGAAGACGAGGCCCTGAGCAAACACAGGGGAATTTTG
GTGATCAAGAACTGATCAGGCAAGGAACAGATTATAAACATTGGCCTCAAATTGCTCA
GTTTGCTCCTTCTGCCTCCGCATTCTTTGGTATGTCTCGTATTGGAATGGAGGTAACTCC
GAGTGGTACATGGCTTACGTATACTGGCGCAATTAAATTGGATGATAAAGACCCAAAT
TTCAAGGACCAGGTTATACTATTAAACAAGCATATAGATGCATACAAAACCTTCCCAC
CCTGActcgagcctaggACGAAG
CoV-42 Ext-CoV N 247-365 - 8 HIS (14.4 kDa)
(SEQ ID NO: 85)
MGKMASLFATFLVVLVSLSLASESSATKKSAAEASKKPRQK [symbol] GRRGPEQT
QGNFGDQELIRQGTDYKHWPQI [symbol] [symbol] TGAIKLDDKD
PNFKDQVILLNKHIDAY [symbol] HHHHHHHH
(SEQ ID NO: 86)
ACGTCAttaattaaATGGGAAAAATGGCTTCTCTTTTTGCTACTTTCCTTGTTGTGTTGGTTA
GTCTTTCTCTAGCTAGTGAGAGTAGTGCTACAAAGAAATCTGCTGCTGAAGCCAGCAA
GAAGCCAAGACAGAAAAGAACTGCAACAAAAGCATATAATGTTACCCAGGCATTTGG
AAGGCGAGGCCCCGAGCAAACTCAAGGGAATTTTGGTGACCAAGAACTGATAAGGCA
GGGAACTGATTATAAACATTGGCCTCAAATTGCTCAATTTGCTCCGTCAGCGTCAGCCT
TCTTTGGGATGAGTCGTATTGGAATGGAGGTAACGCCATCTGGTACATGGCTTACCTAT
ACTGGTGCAATCAAATTGGATGATAAAGATCCAAATTTCAAGGACCAGGTGATATTAC
TCAACAAGCATATTGATGCTTACAAGACATTCCCTCCTCATCACCATCATCATCACCAC
CATTGActcgagcctaggACGAAG
CoV-43 Ext-8HIS-Cov N 245-365 (14.6 kDa)
(SEQ ID NO: 87)
MGKMASLFATFLVVLVSLSLASESSAHHHHHHHHTVTKKSAAEASKKPRQK [symbol] [symbol]
GRRGPEQTQGNFGDQELIRQGTDYKHWPQI [symbol] [symbol] [symbol]
TGAIKLDDKDPNFKDQVILLNKHIDAY [symbol]
(SEQ ID NO: 88)
ACGTCAttaattaaATGGGAAAAATGGCTTCTCTTTTTGCTACTTTCCTTGTTGTGTTGGTTA
GTCTTTCTCTAGCTAGTGAGAGTAGTGCTCACCATCATCATCACCATCATCACACAGTG
ACCAAGAAAAGCGCTGCCGAAGCGTCAAAGAAGCCAAGGCAGAAGAGAACTGCTACA
AAAGCTTATAATGTGACTCAAGCATTTGGAAGACGAGGCCCTGAGCAAACACAGGGG
AATTTTGGTGATCAAGAACTGATCAGGCAAGGAACAGATTATAAACATTGGCCTCAAA
TTGCTCAGTTTGCTCCTTCTGCCTCCGCATTCTTTGGTATGTCTCGTATTGGAATGGAGG
TAACTCCGAGTGGTACATGGCTTACGTATACTGGCGCAATTAAATTGGATGATAAAGA
CCCAAATTTCAAGGACCAGGTTATACTATTAAACAAGCATATAGATGCATACAAAACC
TTCCCACCCTGActcgagcctaggACGAAG
CoV-44 Ext- CoV N 245-365 - 8HIS (14.6 kDa)
(SEQ ID NO: 89)
MGKMASLFATFLVVLVSLSLASESSATVTKKSAAEASKKPRQK [symbol] GRRGPE
QTQGNFGDQELIRQGTDYKHWPQI [symbol] [symbol] TGAIKLDD
KDPNFKDQVILLNKHIDAY [symbol] HHHHHHHH
(SEQ ID NO: 90)
ACGTCAttaattaaATGGGAAAAATGGCTTCTCTTTTTGCTACTTTCCTTGTTGTGTTGGTTA
GTCTTTCTCTAGCTAGTGAGAGTAGTGCTACAGTGACAAAGAAATCTGCTGCTGAAGC
CAGCAAGAAGCCAAGACAGAAAAGAACTGCAACAAAAGCATATAATGTTACCCAGGC
ATTTGGAAGGCGAGGCCCCGAGCAAACTCAAGGGAATTTTGGTGACCAAGAACTGAT
AAGGCAGGGAACTGATTATAAACATTGGCCTCAAATTGCTCAATTTGCTCCGTCAGCG
TCAGCCTTCTTTGGGATGAGTCGTATTGGAATGGAGGTAACGCCATCTGGTACATGGCT
TACCTATACTGGTGCAATCAAATTGGATGATAAAGATCCAAATTTCAAGGACCAGGTG
ATATTACTCAACAAGCATATTGATGCTTACAAGACATTCCCTCCTCATCACCATCATCA
TCACCACCATTGActcgagcctaggACGAAG
CoV-45 IBIO201 S-NTD (19-310) N-CTD (245-370) Long-Long
(SEQ ID NO: 91)
MGKMASLFATFLVVLVSLSLASESSATTRTQLPPAYTNSFTRGVYYPDKVFRSSVLHSTQD
LFLPFFSNVTWFHAIHVSGTNGTKRFDNPVLPFNDGVYFASTEKSNIIRGWIFGTTLDSKTQS
LLIVNNATNVVIKVCEFQFCNDPFLGVYYHKNNKSWMESEFRVYSSANNCTFEYVSQPFL
MDLEGKQGNFKNLREFVFKNIDGYFKIYSKHTPINLVRDLPQGFSALEPLVDLPIGINITRFQ
TLLALHRSYLTPGDSSSGWTAGAAAYYVGYLQPRTFLLKYNENGTITDAVDCALDPLSET
KCTLKSFTVEKSGGGSTVTKKSAAEASKKPRQKRTATKAYNVTQAFGRRGPEQTQGNFGDQEL
IRQGTDYKHWPQIAQFAPSASAFFGMSRIGMEVTPSGTWLTYTGAIKLDDKDPNFKDQVILLNK
HIDAYKTFPPTEPKK
CoV-45-1
(SEQ ID NO: 92)
ACGTCAttaattaaATGGGAAAAATGGCTTCTCTTTTTGCTACTTTCCTTGTTGTGTTGGTTAG
TCTTTCTCTAGCTAGTGAGAGTAGTGCTACGACCAGGACACAACTTCCCCCTGCTTATA
CAAACTCTTTCACTAGGGGCGTCTATTACCCTGATAAGGTGTTTCGGAGTAGTGTATTG
CATTCAACTCAAGATTTGTTCCTTCCGTTCTTCTCAAATGTGACATGGTTTCATGCCATT
CATGTCTCAGGGACCAATGGTACAAAGAGATTTGATAATCCTGTTCTCCCATTCAATGA
CGGTGTTTATTTTGCTTCAACAGAGAAGAGCAACATTATAAGAGGATGGATTTTTGGAA
CAACGCTTGACTCAAAAACTCAGAGTTTATTGATAGTAAATAATGCAACTAACGTTGTG
ATCAAAGTTTGTGAATTCCAGTTCTGCAATGATCCATTTCTGGGAGTTTACTATCACAA
GAACAACAAGTCTTGGATGGAAAGTGAGTTCAGAGTATATTCTTCTGCAAACAATTGC
ACTTTTGAGTATGTTTCTCAGCCTTTTTTGATGGATCTTGAGGGGAAACAAGGAAATTT
CAAAAATCTACGCGAATTTGTTTTTAAGAATATTGATGGCTACTTCAAGATTTACAGCA
AGCATACTCCAATAAACCTTGTGAGAGACCTGCCACAGGGATTTTCCGCCTTAGAACC
ACTGGTAGACTTACCAATTGGGATCAATATCACACGTTTTCAAACTCTCCTAGCGTTGC
ACAGGAGTTACCTAACTCCTGGTGATTCTTCGAGCGGTTGGACTGCTGGAGCTGCAGCA
TATTATGTGGGTTATTTGCAACCCCGAACATTTCTTCTCAAATATAATGAAAATGGAAC
CATTACTGATGCTGTTGATTGTGCTTTGGATCCTCTTTCTGAAACAAAATGTACCCTGA
AATCATTTACTGTCGAGAAATCCGGTGGTGGCTCCACGGTGACCAAAAAAAGTGCCGC
TGAAGCATCCAAGAAACCGCGACAGAAGAGGACTGCAACAAAAGCTTATAATGTTACT
CAAGCATTTGGGAGACGTGGTCCTGAACAAACTCAGGGAAATTTTGGTGACCAGGAGC
TTATAAGGCAAGGAACTGATTATAAGCATTGGCCTCAAATTGCTCAGTTTGCTCCATCA
GCGTCTGCCTTCTTCGGGATGTCAAGAATTGGAATGGAAGTAACCCCTTCTGGCACATG
GTTAACATATACTGGTGCAATCAAATTGGATGATAAAGACCCCAATTTCAAAGATCAA
GTTATACTCCTGAACAAGCACATTGATGCTTACAAGACATTTCCTCCAACAGAGCCAAA
GAAGTGActcgagTCGACGT
CoV-45-2
(SEQ ID NO: 107)
ACGTCAttaattaaATGGGAAAAATGGCTTCTCTTTTTGCTACTTTCCTTGTTGTGTTGGTTAG
TCTTTCTCTAGCTAGTGAGAGTAGTGCTACGACACGAACTCAGCTACCCCCTGCATATA
CTAACTCATTCACCCGTGGAGTTTACTATCCTGATAAAGTATTTCGCAGCTCAGTGCTT
CATTCGACGCAAGATTTGTTTCTTCCTTTTTTCTCCAATGTTACATGGTTTCATGCAATT
CATGTTAGTGGCACAAATGGAACTAAGAGATTTGATAATCCAGTACTACCGTTCAATG
ATGGGGTCTATTTTGCTTCAACTGAGAAGTCTAACATCATACGGGGTTGGATTTTTGGA
ACAACTTTAGACTCCAAAACTCAAAGCTTGCTGATTGTTAACAATGCCACCAATGTGGT
CATCAAAGTATGTGAATTTCAGTTTTGCAATGACCCTTTCCTTGGCGTGTATTATCACA
AGAACAACAAGTCATGGATGGAATCAGAGTTCAGAGTGTACTCTTCAGCAAATAATTG
TACTTTTGAATATGTTTCTCAGCCATTTTTGATGGATCTTGAAGGAAAGCAAGGGAATT
TTAAGAATTTGAGGGAGTTTGTTTTCAAAAATATTGATGGATATTTCAAGATTTACTCC
AAGCATACGCCAATCAACCTTGTCAGAGACTTACCACAAGGTTTTTCTGCGTTGGAGCC
ATTGGTGGACCTCCCTATTGGAATTAACATAACTAGGTTTCAAACACTTCTTGCACTCC
ACAGAAGTTATCTAACTCCTGGTGATTCTTCTAGTGGATGGACTGCTGGTGCTGCTGCC
TACTACGTAGGTTATTTGCAGCCCAGGACATTCTTACTCAAATATAATGAAAATGGTAC
CATAACTGATGCTGTTGATTGTGCACTGGATCCACTGTCTGAAACAAAATGCACATTAA
AAAGTTTCACAGTTGAGAAATCTGGGGGTGGCAGTACCGTAACGAAGAAGTCAGCTGC
TGAGGCGAGCAAGAAACCAAGGCAGAAAAGAACTGCAACAAAAGCATATAATGTTAC
ACAAGCATTTGGGAGGCGTGGTCCTGAACAAACTCAAGGAAATTTTGGTGATCAAGAA
TTGATACGACAGGGCACTGATTATAAACATTGGCCTCAAATTGCTCAGTTTGCTCCCTC
CGCCTCTGCATTCTTTGGAATGTCAAGAATTGGTATGGAGGTGACCCCGAGTGGGACAT
GGTTAACATATACTGGAGCCATCAAGCTTGATGATAAAGACCCAAATTTCAAGGACCA
GGTTATACTGCTCAACAAGCACATTGATGCTTACAAAACATTCCCTCCTACTGAACCAA
AAAAGTGActcgagTCGACGT
CoV-46 IBIO201 S-NTD (19-310)_N-CTD (248-366) Long-Short
(SEQ ID NO: 93)
MGKMASLFATFLVVLVSLSLASESSATTRTQLPPAYTNSFTRGVYYPDKVFRSSVLHSTQD
LFLPFFSNVTWFHAIHVSGTNGTKRFDNPVLPFNDGVYFASTEKSNIIRGWIFGTTLDSKTQS
LLIVNNATNVVIKVCEFQFCNDPFLGVYYHKNNKSWMESEFRVYSSANNCTFEYVSQPFL
MDLEGKQGNFKNLREFVFKNIDGYFKIYSKHTPINLVRDLPQGFSALEPLVDLPIGINITRFQ
TLLALHRSYLTPGDSSSGWTAGAAAYYVGYLQPRTFLLKYNENGTITDAVDCALDPLSET
KCTLKSFTVEKSGGGSKKSAAEASKKPRQKRTATKAYNVTQAFGRRGPEQTQGNFGDQELIRQ
GTDYKHWPQIAQFAPSASAFFGMSRIGMEVTPSGTWLTYTGAIKLDDKDPNFKDQVILLNKHID
AYKTFPPT
CoV-46-1
(SEQ ID NO: 94)
ACGTCAttaattaaATGGGAAAAATGGCTTCTCTTTTTGCTACTTTCCTTGTTGTGTTGGTTAG
TCTTTCTCTAGCTAGTGAGAGTAGTGCTACGACCAGGACACAACTTCCCCCTGCTTATA
CAAACTCTTTCACTAGGGGCGTCTATTACCCTGATAAGGTGTTTCGGAGTAGTGTATTG
CATTCAACTCAAGATTTGTTCCTTCCGTTCTTCTCAAATGTGACATGGTTTCATGCCATT
CATGTCTCAGGGACCAATGGTACAAAGAGATTTGATAATCCTGTTCTCCCATTCAATGA
CGGTGTTTATTTTGCTTCAACAGAGAAGAGCAACATTATAAGAGGATGGATTTTTGGAA
CAACGCTTGACTCAAAAACTCAGAGTTTATTGATAGTAAATAATGCAACTAACGTTGTG
ATCAAAGTTTGTGAATTCCAGTTCTGCAATGATCCATTTCTGGGAGTTTACTATCACAA
GAACAACAAGTCTTGGATGGAAAGTGAGTTCAGAGTATATTCTTCTGCAAACAATTGC
ACTTTTGAGTATGTTTCTCAGCCTTTTTTGATGGATCTTGAGGGGAAACAAGGAAATTT
CAAAAATCTACGCGAATTTGTTTTTAAGAATATTGATGGCTACTTCAAGATTTACAGCA
AGCATACTCCAATAAACCTTGTGAGAGACCTGCCACAGGGATTTTCCGCCTTAGAACC
ACTGGTAGACTTACCAATTGGGATCAATATCACACGTTTTCAAACTCTCCTAGCGTTGC
ACAGGAGTTACCTAACTCCTGGTGATTCTTCGAGCGGTTGGACTGCTGGAGCTGCAGCA
TATTATGTGGGTTATTTGCAACCCCGAACATTTCTTCTCAAATATAATGAAAATGGAAC
CATTACTGATGCTGTTGATTGTGCTTTGGATCCTCTTTCTGAAACAAAATGTACCCTGA
AATCATTTACTGTCGAGAAATCCGGTGGTGGCTCCAAAAAAAGTGCCGCTGAAGCATC
CAAGAAACCGCGACAGAAGAGGACTGCAACAAAAGCTTATAATGTTACTCAAGCATTT
GGGAGACGTGGTCCTGAACAAACTCAGGGAAATTTTGGTGACCAGGAGCTTATAAGGC
AAGGAACTGATTATAAGCATTGGCCTCAAATTGCTCAGTTTGCTCCATCAGCGTCTGCC
TTCTTCGGGATGTCAAGAATTGGAATGGAAGTAACCCCTTCTGGCACATGGTTAACATA
TACTGGTGCAATCAAATTGGATGATAAAGACCCCAATTTCAAAGATCAAGTTATACTCC
TGAACAAGCACATTGATGCTTACAAGACATTTCCTCCAACATGActcgagTCGACGT
CoV-46-2
(SEQ ID NO: 108)
ACGTCAttaattaaATGGGAAAAATGGCTTCTCTTTTTGCTACTTTCCTTGTTGTGTTGGTTAG
TCTTTCTCTAGCTAGTGAGAGTAGTGCTACGACACGAACTCAGCTACCCCCTGCATATA
CTAACTCATTCACCCGTGGAGTTTACTATCCTGATAAAGTATTTCGCAGCTCAGTGCTT
CATTCGACGCAAGATTTGTTTCTTCCTTTTTTCTCCAATGTTACATGGTTTCATGCAATT
CATGTTAGTGGCACAAATGGAACTAAGAGATTTGATAATCCAGTACTACCGTTCAATG
ATGGGGTCTATTTTGCTTCAACTGAGAAGTCTAACATCATACGGGGTTGGATTTTTGGA
ACAACTTTAGACTCCAAAACTCAAAGCTTGCTGATTGTTAACAATGCCACCAATGTGGT
CATCAAAGTATGTGAATTTCAGTTTTGCAATGACCCTTTCCTTGGCGTGTATTATCACA
AGAACAACAAGTCATGGATGGAATCAGAGTTCAGAGTGTACTCTTCAGCAAATAATTG
TACTTTTGAATATGTTTCTCAGCCATTTTTGATGGATCTTGAAGGAAAGCAAGGGAATT
TTAAGAATTTGAGGGAGTTTGTTTTCAAAAATATTGATGGATATTTCAAGATTTACTCC
AAGCATACGCCAATCAACCTTGTCAGAGACTTACCACAAGGTTTTTCTGCGTTGGAGCC
ATTGGTGGACCTCCCTATTGGAATTAACATAACTAGGTTTCAAACACTTCTTGCACTCC
ACAGAAGTTATCTAACTCCTGGTGATTCTTCTAGTGGATGGACTGCTGGTGCTGCTGCC
TACTACGTAGGTTATTTGCAGCCCAGGACATTCTTACTCAAATATAATGAAAATGGTAC
CATAACTGATGCTGTTGATTGTGCACTGGATCCACTGTCTGAAACAAAATGCACATTAA
AAAGTTTCACAGTTGAGAAATCTGGGGGTGGCAGTAAGAAGTCAGCTGCTGAGGCGAG
CAAGAAACCAAGGCAGAAAAGAACTGCAACAAAAGCATATAATGTTACACAAGCATT
TGGGAGGCGTGGTCCTGAACAAACTCAAGGAAATTTTGGTGATCAAGAATTGATACGA
CAGGGCACTGATTATAAACATTGGCCTCAAATTGCTCAGTTTGCTCCCTCCGCCTCTGC
ATTCTTTGGAATGTCAAGAATTGGTATGGAGGTGACCCCGAGTGGGACATGGTTAACA
TATACTGGAGCCATCAAGCTTGATGATAAAGACCCAAATTTCAAGGACCAGGTTATAC
TGCTCAACAAGCACATTGATGCTTACAAAACATTCCCTCCTACTTGActcgagTCGACGT
CoV-47 IBIO201 S-NTD (19-290)_N-CTD (245-370) Short-Long
(SEQ ID NO: 95)
MGKMASLFATFLVVLVSLSLASESSATTRTQLPPAYTNSFTRGVYYPDKVFRSSVLHSTQD
LFLPFFSNVTWFHAIHVSGTNGTKRFDNPVLPFNDGVYFASTEKSNIIRGWIFGTTLDSKTQS
LLIVNNATNVVIKVCEFQFCNDPFLGVYYHKNNKSWMESEFRVYSSANNCTFEYVSQPFL
MDLEGKQGNFKNLREFVFKNIDGYFKIYSKHTPINLVRDLPQGFSALEPLVDLPIGINITRFQ
TLLALHRSYLTPGDSSSGWTAGAAAYYVGYLQPRTFLLKYNENGTITDAVDSGGGSTVTK
KSAAEASKKPRQKRTATKAYNVTQAFGRRGPEQTQGNFGDQELIRQGTDYKHWPQIAQFAPSAS
AFFGMSRIGMEVTPSGTWLTYTGAIKLDDKDPNFKDQVILLNKHIDAYKTFPPTEPKK
CoV-47-1
(SEQ ID NO: 96)
ACGTCAttaattaaATGGGAAAAATGGCTTCTCTTTTTGCTACTTTCCTTGTTGTGTTGGTTAG
TCTTTCTCTAGCTAGTGAGAGTAGTGCTACGACCAGGACACAACTTCCCCCTGCTTATA
CAAACTCTTTCACTAGGGGCGTCTATTACCCTGATAAGGTGTTTCGGAGTAGTGTATTG
CATTCAACTCAAGATTTGTTCCTTCCGTTCTTCTCAAATGTGACATGGTTTCATGCCATT
CATGTCTCAGGGACCAATGGTACAAAGAGATTTGATAATCCTGTTCTCCCATTCAATGA
CGGTGTTTATTTTGCTTCAACAGAGAAGAGCAACATTATAAGAGGATGGATTTTTGGAA
CAACGCTTGACTCAAAAACTCAGAGTTTATTGATAGTAAATAATGCAACTAACGTTGTG
ATCAAAGTTTGTGAATTCCAGTTCTGCAATGATCCATTTCTGGGAGTTTACTATCACAA
GAACAACAAGTCTTGGATGGAAAGTGAGTTCAGAGTATATTCTTCTGCAAACAATTGC
ACTTTTGAGTATGTTTCTCAGCCTTTTTTGATGGATCTTGAGGGGAAACAAGGAAATTT
CAAAAATCTACGCGAATTTGTTTTTAAGAATATTGATGGCTACTTCAAGATTTACAGCA
AGCATACTCCAATAAACCTTGTGAGAGACCTGCCACAGGGATTTTCCGCCTTAGAACC
ACTGGTAGACTTACCAATTGGGATCAATATCACACGTTTTCAAACTCTCCTAGCGTTGC
ACAGGAGTTACCTAACTCCTGGTGATTCTTCGAGCGGTTGGACTGCTGGAGCTGCAGCA
TATTATGTGGGTTATTTGCAACCCCGAACATTTCTTCTCAAATATAATGAAAATGGAAC
CATTACTGATGCTGTTGATTCCGGTGGTGGCTCCACGGTGACCAAAAAAAGTGCCGCTG
AAGCATCCAAGAAACCGCGACAGAAGAGGACTGCAACAAAAGCTTATAATGTTACTCA
AGCATTTGGGAGACGTGGTCCTGAACAAACTCAGGGAAATTTTGGTGACCAGGAGCTT
ATAAGGCAAGGAACTGATTATAAGCATTGGCCTCAAATTGCTCAGTTTGCTCCATCAGC
GTCTGCCTTCTTCGGGATGTCAAGAATTGGAATGGAAGTAACCCCTTCTGGCACATGGT
TAACATATACTGGTGCAATCAAATTGGATGATAAAGACCCCAATTTCAAAGATCAAGT
TATACTCCTGAACAAGCACATTGATGCTTACAAGACATTTCCTCCAACAGAGCCAAAG
AAGTGActcgagTCGACGT
CoV-47-2
(SEQ ID NO: 109)
ACGTCAttaattaaATGGGAAAAATGGCTTCTCTTTTTGCTACTTTCCTTGTTGTGTTGGTTAG
TCTTTCTCTAGCTAGTGAGAGTAGTGCTACGACACGAACTCAGCTACCCCCTGCATATA
CTAACTCATTCACCCGTGGAGTTTACTATCCTGATAAAGTATTTCGCAGCTCAGTGCTT
CATTCGACGCAAGATTTGTTTCTTCCTTTTTTCTCCAATGTTACATGGTTTCATGCAATT
CATGTTAGTGGCACAAATGGAACTAAGAGATTTGATAATCCAGTACTACCGTTCAATG
ATGGGGTCTATTTTGCTTCAACTGAGAAGTCTAACATCATACGGGGTTGGATTTTTGGA
ACAACTTTAGACTCCAAAACTCAAAGCTTGCTGATTGTTAACAATGCCACCAATGTGGT
CATCAAAGTATGTGAATTTCAGTTTTGCAATGACCCTTTCCTTGGCGTGTATTATCACA
AGAACAACAAGTCATGGATGGAATCAGAGTTCAGAGTGTACTCTTCAGCAAATAATTG
TACTTTTGAATATGTTTCTCAGCCATTTTTGATGGATCTTGAAGGAAAGCAAGGGAATT
TTAAGAATTTGAGGGAGTTTGTTTTCAAAAATATTGATGGATATTTCAAGATTTACTCC
AAGCATACGCCAATCAACCTTGTCAGAGACTTACCACAAGGTTTTTCTGCGTTGGAGCC
ATTGGTGGACCTCCCTATTGGAATTAACATAACTAGGTTTCAAACACTTCTTGCACTCC
ACAGAAGTTATCTAACTCCTGGTGATTCTTCTAGTGGATGGACTGCTGGTGCTGCTGCC
TACTACGTAGGTTATTTGCAGCCCAGGACATTCTTACTCAAATATAATGAAAATGGTAC
CATAACTGATGCTGTTGATTCTGGGGGTGGCAGTACCGTAACGAAGAAGTCAGCTGCT
GAGGCGAGCAAGAAACCAAGGCAGAAAAGAACTGCAACAAAAGCATATAATGTTACA
CAAGCATTTGGGAGGCGTGGTCCTGAACAAACTCAAGGAAATTTTGGTGATCAAGAAT
TGATACGACAGGGCACTGATTATAAACATTGGCCTCAAATTGCTCAGTTTGCTCCCTCC
GCCTCTGCATTCTTTGGAATGTCAAGAATTGGTATGGAGGTGACCCCGAGTGGGACAT
GGTTAACATATACTGGAGCCATCAAGCTTGATGATAAAGACCCAAATTTCAAGGACCA
GGTTATACTGCTCAACAAGCACATTGATGCTTACAAAACATTCCCTCCTACTGAACCAA
AAAAGTGActcgagTCGACGT
CoV-48 IBIO201S-NTD(19-290) - N-CTD(248-366) Short-Short
(SEQ ID NO: 97)
MGKMASLFATFLVVLVSLSLASESSATTRTQLPPAYTNSFTRGVYYPDKVFRSSVLHSTQD
LFLPFFSNVTWFHAIHVSGTNGTKRFDNPVLPFNDGVYFASTEKSNIIRGWIFGTTLDSKTQS
LLIVNNATNVVIKVCEFQFCNDPFLGVYYHKNNKSWMESEFRVYSSANNCTFEYVSQPFL
MDLEGKQGNFKNLREFVFKNIDGYFKIYSKHTPINLVRDLPQGFSALEPLVDLPIGINITRFQ
TLLALHRSYLTPGDSSSGWTAGAAAYYVGYLQPRTFLLKYNENGTITDAVDSGGGSKKSA
AEASKKPRQKRTATKAYNVTQAFGRRGPEQTQGNFGDQELIRQGTDYKHWPQIAQFAPSASAFF
GMSRIGMEVTPSGTWLTYTGAIKLDDKDPNFKDQVILLNKHIDAYKTFPPT
CoV-48-1
(SEQ ID NO: 98)
ACGTCAttaattaaATGGGAAAAATGGCTTCTCTTTTTGCTACTTTCCTTGTTGTGTTGGTTAG
TCTTTCTCTAGCTAGTGAGAGTAGTGCTACGACCAGGACACAACTTCCCCCTGCTTATA
CAAACTCTTTCACTAGGGGCGTCTATTACCCTGATAAGGTGTTTCGGAGTAGTGTATTG
CATTCAACTCAAGATTTGTTCCTTCCGTTCTTCTCAAATGTGACATGGTTTCATGCCATT
CATGTCTCAGGGACCAATGGTACAAAGAGATTTGATAATCCTGTTCTCCCATTCAATGA
CGGTGTTTATTTTGCTTCAACAGAGAAGAGCAACATTATAAGAGGATGGATTTTTGGAA
CAACGCTTGACTCAAAAACTCAGAGTTTATTGATAGTAAATAATGCAACTAACGTTGTG
ATCAAAGTTTGTGAATTCCAGTTCTGCAATGATCCATTTCTGGGAGTTTACTATCACAA
GAACAACAAGTCTTGGATGGAAAGTGAGTTCAGAGTATATTCTTCTGCAAACAATTGC
ACTTTTGAGTATGTTTCTCAGCCTTTTTTGATGGATCTTGAGGGGAAACAAGGAAATTT
CAAAAATCTACGCGAATTTGTTTTTAAGAATATTGATGGCTACTTCAAGATTTACAGCA
AGCATACTCCAATAAACCTTGTGAGAGACCTGCCACAGGGATTTTCCGCCTTAGAACC
ACTGGTAGACTTACCAATTGGGATCAATATCACACGTTTTCAAACTCTCCTAGCGTTGC
ACAGGAGTTACCTAACTCCTGGTGATTCTTCGAGCGGTTGGACTGCTGGAGCTGCAGCA
TATTATGTGGGTTATTTGCAACCCCGAACATTTCTTCTCAAATATAATGAAAATGGAAC
CATTACTGATGCTGTTGATTCCGGTGGTGGCTCCAAAAAAAGTGCCGCTGAAGCATCCA
AGAAACCGCGACAGAAGAGGACTGCAACAAAAGCTTATAATGTTACTCAAGCATTTGG
GAGACGTGGTCCTGAACAAACTCAGGGAAATTTTGGTGACCAGGAGCTTATAAGGCAA
GGAACTGATTATAAGCATTGGCCTCAAATTGCTCAGTTTGCTCCATCAGCGTCTGCCTT
CTTCGGGATGTCAAGAATTGGAATGGAAGTAACCCCTTCTGGCACATGGTTAACATAT
ACTGGTGCAATCAAATTGGATGATAAAGACCCCAATTTCAAAGATCAAGTTATACTCCT
GAACAAGCACATTGATGCTTACAAGACATTTCCTCCAACATGActcgagTCGACGT
CoV-48-2
(SEQ ID NO: 110)
ACGTCAttaattaaATGGGAAAAATGGCTTCTCTTTTTGCTACTTTCCTTGTTGTGTTGGTTAG
TCTTTCTCTAGCTAGTGAGAGTAGTGCTACGACACGAACTCAGCTACCCCCTGCATATA
CTAACTCATTCACCCGTGGAGTTTACTATCCTGATAAAGTATTTCGCAGCTCAGTGCTT
CATTCGACGCAAGATTTGTTTCTTCCTTTTTTCTCCAATGTTACATGGTTTCATGCAATT
CATGTTAGTGGCACAAATGGAACTAAGAGATTTGATAATCCAGTACTACCGTTCAATG
ATGGGGTCTATTTTGCTTCAACTGAGAAGTCTAACATCATACGGGGTTGGATTTTTGGA
ACAACTTTAGACTCCAAAACTCAAAGCTTGCTGATTGTTAACAATGCCACCAATGTGGT
CATCAAAGTATGTGAATTTCAGTTTTGCAATGACCCTTTCCTTGGCGTGTATTATCACA
AGAACAACAAGTCATGGATGGAATCAGAGTTCAGAGTGTACTCTTCAGCAAATAATTG
TACTTTTGAATATGTTTCTCAGCCATTTTTGATGGATCTTGAAGGAAAGCAAGGGAATT
TTAAGAATTTGAGGGAGTTTGTTTTCAAAAATATTGATGGATATTTCAAGATTTACTCC
AAGCATACGCCAATCAACCTTGTCAGAGACTTACCACAAGGTTTTTCTGCGTTGGAGCC
ATTGGTGGACCTCCCTATTGGAATTAACATAACTAGGTTTCAAACACTTCTTGCACTCC
ACAGAAGTTATCTAACTCCTGGTGATTCTTCTAGTGGATGGACTGCTGGTGCTGCTGCC
TACTACGTAGGTTATTTGCAGCCCAGGACATTCTTACTCAAATATAATGAAAATGGTAC
CATAACTGATGCTGTTGATTCTGGGGGTGGCAGTAAGAAGTCAGCTGCTGAGGCGAGC
AAGAAACCAAGGCAGAAAAGAACTGCAACAAAAGCATATAATGTTACACAAGCATTT
GGGAGGCGTGGTCCTGAACAAACTCAAGGAAATTTTGGTGATCAAGAATTGATACGAC
AGGGCACTGATTATAAACATTGGCCTCAAATTGCTCAGTTTGCTCCCTCCGCCTCTGCA
TTCTTTGGAATGTCAAGAATTGGTATGGAGGTGACCCCGAGTGGGACATGGTTAACAT
ATACTGGAGCCATCAAGCTTGATGATAAAGACCCAAATTTCAAGGACCAGGTTATACT
GCTCAACAAGCACATTGATGCTTACAAAACATTCCCTCCTACTTGActcgagTCGACGT
CoV-49 IBIO201S-RBD(319-554) - N-CTD(245-370) Long-Long
(SEQ ID NO: 99)
MGKMASLFATFLVVLVSLSLASESSARVQPTESIVRFPNITNLCPFGEVFNATRFASVYAWN
RKRISNCVADYSVLYNSASFSTFKCYGVSPTKLNDLCFTNVYADSFVIRGDEVRQIAPGQTG
KIADYNYKLPDDFTGCVIAWNSNNLDSKVGGNYNYLYRLFRKSNLKPFERDISTEIYQAGS
TPCNGVEGFNCYFPLQSYGFQPTNGVGYQPYRVVVLSFELLHAPATVCGPKKSTNLVKNK
CVNFNFNGLTGTGVLTESGGGSTVTKKSAAEASKKPRQKRTATKAYNVTQAFGRRGPEQTQG
NFGDQELIRQGTDYKHWPQIAQFAPSASAFFGMSRIGMEVTPSGTWLTYTGAIKLDDKDPNFKD
QVILLNKHIDAYKTFPPTEPKK
CoV-49-1
(SEQ ID NO: 100)
ACGTCAttaattaaATGGGAAAAATGGCTTCTCTTTTTGCTACTTTCCTTGTTGTGTTGGTTAG
TCTTTCTCTAGCTAGTGAGAGTAGTGCTAGGGTTCAGCCTACTGAGTCTATCGTGCGGT
TCCCTAACATCACCAACTTGTGCCCTTTCGGCGAGGTGTTCAATGCTACTAGGTTCGCT
TCTGTGTACGCCTGGAACCGGAAGAGGATTTCTAACTGCGTGGCCGATTACAGCGTGCT
GTACAACTCTGCTAGCTTCAGCACCTTCAAGTGCTACGGTGTGTCTCCTACCAAGCTGA
ACGATCTGTGCTTCACCAACGTGTACGCTGACTCTTTCGTGATCAGGGGTGATGAGGTT
AGGCAGATTGCTCCTGGTCAGACCGGTAAGATCGCTGACTACAACTACAAGCTGCCTG
ATGACTTCACCGGTTGCGTGATCGCTTGGAACTCTAACAACCTGGACTCTAAGGTTGGC
GGCAATTACAACTACCTCTACCGGCTGTTCCGGAAGTCTAACCTTAAGCCTTTCGAGCG
GGATATCAGCACCGAGATCTATCAGGCTGGTTCTACTCCTTGCAATGGCGTTGAGGGTT
TCAACTGCTACTTCCCGCTTCAGTCTTACGGATTCCAGCCTACTAATGGTGTGGGCTAC
CAGCCTTACAGAGTGGTGGTTTTGTCTTTCGAGCTTCTGCATGCTCCTGCTACTGTTTGC
GGTCCTAAGAAGTCTACAAATCTTGTTAAGAATAAGTGCGTTAACTTCAACTTCAATGG
TTTGACTGGAACAGGTGTTCTTACAGAATCCGGTGGTGGCTCCACGGTGACCAAAAAA
AGTGCCGCTGAAGCATCCAAGAAACCGCGACAGAAGAGGACTGCAACAAAAGCTTAT
AATGTTACTCAAGCATTTGGGAGACGTGGTCCTGAACAAACTCAGGGAAATTTTGGTG
ACCAGGAGCTTATAAGGCAAGGAACTGATTATAAGCATTGGCCTCAAATTGCTCAGTT
TGCTCCATCAGCGTCTGCCTTCTTCGGGATGTCAAGAATTGGAATGGAAGTAACCCCTT
CTGGCACATGGTTAACATATACTGGTGCAATCAAATTGGATGATAAAGACCCCAATTTC
AAAGATCAAGTTATACTCCTGAACAAGCACATTGATGCTTACAAGACATTTCCTCCAAC
AGAGCCAAAGAAGTGActcgagTCGACGT
CoV-49-2
(SEQ ID NO: 111)
ACGTCAttaattaaATGGGAAAAATGGCTTCTCTTTTTGCTACTTTCCTTGTTGTGTTGGTTAG
TCTTTCTCTAGCTAGTGAGAGTAGTGCTCGAGTTCAGCCAACAGAGAGTATCGTTAGAT
TCCCTAATATAACCAACCTCTGCCCATTTGGAGAAGTGTTTAACGCCACAAGATTTGCT
TCTGTATATGCTTGGAACCGCAAAAGAATAAGCAATTGTGTGGCAGACTACAGCGTGC
TATACAATTCTGCGAGTTTCTCAACATTCAAGTGCTATGGTGTGTCACCAACTAAGTTA
AATGATCTCTGCTTTACTAATGTGTACGCTGATTCATTTGTCATTAGAGGTGATGAAGT
TCGTCAGATCGCTCCAGGACAAACTGGCAAGATAGCTGACTACAACTATAAGCTTCCG
GATGACTTTACGGGATGTGTTATTGCATGGAATTCCAACAACCTTGATTCTAAAGTTGG
AGGGAATTATAATTACTTGTATCGGCTTTTCAGGAAATCAAATTTGAAACCTTTTGAGA
GGGATATTTCGACCGAAATTTATCAAGCCGGGAGTACTCCATGCAATGGTGTAGAAGG
ATTCAACTGTTACTTTCCTCTGCAATCATATGGATTTCAACCCACAAACGGCGTGGGTT
ATCAGCCCTATAGGGTAGTAGTTCTAAGTTTTGAACTTTTGCATGCACCTGCAACAGTT
TGTGGACCTAAGAAATCCACAAATTTAGTCAAGAATAAATGTGTAAATTTCAATTTCAA
TGGGTTGACCGGCACTGGTGTTCTGACTGAGTCTGGAGGTGGTTCTACCGTAACGAAG
AAGTCAGCTGCTGAGGCGAGCAAGAAACCAAGGCAGAAAAGAACTGCAACAAAAGCA
TATAATGTTACACAAGCATTTGGGAGGCGTGGTCCTGAACAAACTCAAGGAAATTTTG
GTGATCAAGAATTGATACGACAGGGCACTGATTATAAACATTGGCCTCAAATTGCTCA
GTTTGCTCCCTCCGCCTCTGCATTCTTTGGAATGTCAAGAATTGGTATGGAGGTGACCC
CGAGTGGGACATGGTTAACATATACTGGAGCCATCAAGCTTGATGATAAAGACCCAAA
TTTCAAGGACCAGGTTATACTGCTCAACAAGCACATTGATGCTTACAAAACATTCCCTC
CTACTGAACCAAAAAAGTGActcgagTCGACGT
CoV-50 IBIO201S-RBD(319-554) - N-CTD(248-366) Long-Short
(SEQ ID NO: 101)
MGKMASLFATFLVVLVSLSLASESSARVQPTESIVRFPNITNLCPFGEVFNATRFASVYAWN
RKRISNCVADYSVLYNSASFSTFKCYGVSPTKLNDLCFTNVYADSFVIRGDEVRQIAPGQTG
KIADYNYKLPDDFTGCVIAWNSNNLDSKVGGNYNYLYRLFRKSNLKPFERDISTEIYQAGS
TPCNGVEGFNCYFPLQSYGFQPTNGVGYQPYRVVVLSFELLHAPATVCGPKKSTNLVKNK
CVNFNFNGLTGTGVLTESGGGSKKSAAEASKKPRQKRTATKAYNVTQAFGRRGPEQTQGNFG
DQELIRQGTDYKHWPQIAQFAPSASAFFGMSRIGMEVTPSGTWLTYTGAIKLDDKDPNFKDQVI
LLNKHIDAYKTFPPT
CoV-50-1
(SEQ ID NO: 102)
ACGTCAttaattaaATGGGAAAAATGGCTTCTCTTTTTGCTACTTTCCTTGTTGTGTTGGTTAG
TCTTTCTCTAGCTAGTGAGAGTAGTGCTAGGGTTCAGCCTACTGAGTCTATCGTGCGGT
TCCCTAACATCACCAACTTGTGCCCTTTCGGCGAGGTGTTCAATGCTACTAGGTTCGCT
TCTGTGTACGCCTGGAACCGGAAGAGGATTTCTAACTGCGTGGCCGATTACAGCGTGCT
GTACAACTCTGCTAGCTTCAGCACCTTCAAGTGCTACGGTGTGTCTCCTACCAAGCTGA
ACGATCTGTGCTTCACCAACGTGTACGCTGACTCTTTCGTGATCAGGGGTGATGAGGTT
AGGCAGATTGCTCCTGGTCAGACCGGTAAGATCGCTGACTACAACTACAAGCTGCCTG
ATGACTTCACCGGTTGCGTGATCGCTTGGAACTCTAACAACCTGGACTCTAAGGTTGGC
GGCAATTACAACTACCTCTACCGGCTGTTCCGGAAGTCTAACCTTAAGCCTTTCGAGCG
GGATATCAGCACCGAGATCTATCAGGCTGGTTCTACTCCTTGCAATGGCGTTGAGGGTT
TCAACTGCTACTTCCCGCTTCAGTCTTACGGATTCCAGCCTACTAATGGTGTGGGCTAC
CAGCCTTACAGAGTGGTGGTTTTGTCTTTCGAGCTTCTGCATGCTCCTGCTACTGTTTGC
GGTCCTAAGAAGTCTACAAATCTTGTTAAGAATAAGTGCGTTAACTTCAACTTCAATGG
TTTGACTGGAACAGGTGTTCTTACAGAATCCGGTGGTGGCTCCAAAAAAAGTGCCGCT
GAAGCATCCAAGAAACCGCGACAGAAGAGGACTGCAACAAAAGCTTATAATGTTACTC
AAGCATTTGGGAGACGTGGTCCTGAACAAACTCAGGGAAATTTTGGTGACCAGGAGCT
TATAAGGCAAGGAACTGATTATAAGCATTGGCCTCAAATTGCTCAGTTTGCTCCATCAG
CGTCTGCCTTCTTCGGGATGTCAAGAATTGGAATGGAAGTAACCCCTTCTGGCACATGG
TTAACATATACTGGTGCAATCAAATTGGATGATAAAGACCCCAATTTCAAAGATCAAG
TTATACTCCTGAACAAGCACATTGATGCTTACAAGACATTTCCTCCAACATGActcgagTC
GACGT
CoV-50-2
(SEQ ID NO: 112)
ACGTCAttaattaaATGGGAAAAATGGCTTCTCTTTTTGCTACTTTCCTTGTTGTGTTGGTTAG
TCTTTCTCTAGCTAGTGAGAGTAGTGCTCGAGTTCAGCCAACAGAGAGTATCGTTAGAT
TCCCTAATATAACCAACCTCTGCCCATTTGGAGAAGTGTTTAACGCCACAAGATTTGCT
TCTGTATATGCTTGGAACCGCAAAAGAATAAGCAATTGTGTGGCAGACTACAGCGTGC
TATACAATTCTGCGAGTTTCTCAACATTCAAGTGCTATGGTGTGTCACCAACTAAGTTA
AATGATCTCTGCTTTACTAATGTGTACGCTGATTCATTTGTCATTAGAGGTGATGAAGT
TCGTCAGATCGCTCCAGGACAAACTGGCAAGATAGCTGACTACAACTATAAGCTTCCG
GATGACTTTACGGGATGTGTTATTGCATGGAATTCCAACAACCTTGATTCTAAAGTTGG
AGGGAATTATAATTACTTGTATCGGCTTTTCAGGAAATCAAATTTGAAACCTTTTGAGA
GGGATATTTCGACCGAAATTTATCAAGCCGGGAGTACTCCATGCAATGGTGTAGAAGG
ATTCAACTGTTACTTTCCTCTGCAATCATATGGATTTCAACCCACAAACGGCGTGGGTT
ATCAGCCCTATAGGGTAGTAGTTCTAAGTTTTGAACTTTTGCATGCACCTGCAACAGTT
TGTGGACCTAAGAAATCCACAAATTTAGTCAAGAATAAATGTGTAAATTTCAATTTCAA
TGGGTTGACCGGCACTGGTGTTCTGACTGAGTCTGGAGGTGGTTCTAAGAAGTCAGCTG
CTGAGGCGAGCAAGAAACCAAGGCAGAAAAGAACTGCAACAAAAGCATATAATGTTA
CACAAGCATTTGGGAGGCGTGGTCCTGAACAAACTCAAGGAAATTTTGGTGATCAAGA
ATTGATACGACAGGGCACTGATTATAAACATTGGCCTCAAATTGCTCAGTTTGCTCCCT
CCGCCTCTGCATTCTTTGGAATGTCAAGAATTGGTATGGAGGTGACCCCGAGTGGGAC
ATGGTTAACATATACTGGAGCCATCAAGCTTGATGATAAAGACCCAAATTTCAAGGAC
CAGGTTATACTGCTCAACAAGCACATTGATGCTTACAAAACATTCCCTCCTACTTGActcg
agTCGACGT
CoV-51 IBIO201S-RBD(348-523) - N-CTD(245-370) Short-Long
(SEQ ID NO: 103)
MGKMASLFATFLVVLVSLSLASESSAASVYAWNRKRISNCVADYSVLYNSASFSTFKCYG
VSPTKLNDLCFTNVYADSFVIRGDEVRQIAPGQTGKIADYNYKLPDDFTGCVIAWNSNNLD
SKVGGNYNYLYRLFRKSNLKPFERDISTEIYQAGSTPCNGVEGFNCYFPLQSYGFQPTNGV
GYQPYRVVVLSFELLHAPATSGGGSTVTKKSAAEASKKPRQKRTATKAYMTQAFGRRGPEQT
QGNFGDQELIRQGTDYKHWPQIAQFAPSASAFFGMSRIGMEVTPSGTWLTYTGAIKLDDKDPNF
KDQVILLNKHIDAYKTFPPTEPKK
CoV-51-1
(SEQ ID NO: 104)
ACGTCAttaattaaATGGGAAAAATGGCTTCTCTTTTTGCTACTTTCCTTGTTGTGTTGGTTAG
TCTTTCTCTAGCTAGTGAGAGTAGTGCTGCTTCTGTGTACGCCTGGAACCGGAAGAGGA
TTTCTAACTGCGTGGCCGATTACAGCGTGCTGTACAACTCTGCTAGCTTCAGCACCTTC
AAGTGCTACGGTGTGTCTCCTACCAAGCTGAACGATCTGTGCTTCACCAACGTGTACGC
TGACTCTTTCGTGATCAGGGGTGATGAGGTTAGGCAGATTGCTCCTGGTCAGACCGGTA
AGATCGCTGACTACAACTACAAGCTGCCTGATGACTTCACCGGTTGCGTGATCGCTTGG
AACTCTAACAACCTGGACTCTAAGGTTGGCGGCAATTACAACTACCTCTACCGGCTGTT
CCGGAAGTCTAACCTTAAGCCTTTCGAGCGGGATATCAGCACCGAGATCTATCAGGCT
GGTTCTACTCCTTGCAATGGCGTTGAGGGTTTCAACTGCTACTTCCCGCTTCAGTCTTAC
GGATTCCAGCCTACTAATGGTGTGGGCTACCAGCCTTACAGAGTGGTGGTTTTGTCTTT
CGAGCTTCTGCATGCTCCTGCTACTTCTGGAGGTGGTTCTACGGTGACCAAAAAAAGTG
CCGCTGAAGCATCCAAGAAACCGCGACAGAAGAGGACTGCAACAAAAGCTTATAATG
TTACTCAAGCATTTGGGAGACGTGGTCCTGAACAAACTCAGGGAAATTTTGGTGACCA
GGAGCTTATAAGGCAAGGAACTGATTATAAGCATTGGCCTCAAATTGCTCAGTTTGCTC
CATCAGCGTCTGCCTTCTTCGGGATGTCAAGAATTGGAATGGAAGTAACCCCTTCTGGC
ACATGGTTAACATATACTGGTGCAATCAAATTGGATGATAAAGACCCCAATTTCAAAG
ATCAAGTTATACTCCTGAACAAGCACATTGATGCTTACAAGACATTTCCTCCAACAGAG
CCAAAGAAGTGActcgagTCGACGT
CoV-51-2
(SEQ ID NO: 113)
ACGTCAttaattaaATGGGAAAAATGGCTTCTCTTTTTGCTACTTTCCTTGTTGTGTTGGTTAG
TCTTTCTCTAGCTAGTGAGAGTAGTGCTGCTTCTGTATATGCTTGGAACCGCAAAAGAA
TAAGCAATTGTGTGGCAGACTACAGCGTGCTATACAATTCTGCGAGTTTCTCAACATTC
AAGTGCTATGGTGTGTCACCAACTAAGTTAAATGATCTCTGCTTTACTAATGTGTACGC
TGATTCATTTGTCATTAGAGGTGATGAAGTTCGTCAGATCGCTCCAGGACAAACTGGCA
AGATAGCTGACTACAACTATAAGCTTCCGGATGACTTTACGGGATGTGTTATTGCATGG
AATTCCAACAACCTTGATTCTAAAGTTGGAGGGAATTATAATTACTTGTATCGGCTTTT
CAGGAAATCAAATTTGAAACCTTTTGAGAGGGATATTTCGACCGAAATTTATCAAGCC
GGGAGTACTCCATGCAATGGTGTAGAAGGATTCAACTGTTACTTTCCTCTGCAATCATA
TGGATTTCAACCCACAAACGGCGTGGGTTATCAGCCCTATAGGGTAGTAGTTCTAAGTT
TTGAACTTTTGCATGCACCTGCAACATCTGGAGGTGGTTCTACCGTAACGAAGAAGTCA
GCTGCTGAGGCGAGCAAGAAACCAAGGCAGAAAAGAACTGCAACAAAAGCATATAAT
GTTACACAAGCATTTGGGAGGCGTGGTCCTGAACAAACTCAAGGAAATTTTGGTGATC
AAGAATTGATACGACAGGGCACTGATTATAAACATTGGCCTCAAATTGCTCAGTTTGCT
CCCTCCGCCTCTGCATTCTTTGGAATGTCAAGAATTGGTATGGAGGTGACCCCGAGTGG
GACATGGTTAACATATACTGGAGCCATCAAGCTTGATGATAAAGACCCAAATTTCAAG
GACCAGGTTATACTGCTCAACAAGCACATTGATGCTTACAAAACATTCCCTCCTACTGA
ACCAAAAAAGTGActcgagTCGACGT
CoV-52 IBIO201S-RBD(348-523) - N-CTD(248-366) Short-Short
(SEQ ID NO: 105)
MGKMASLFATFLVVLVSLSLASESSAASVYAWNRKRISNCVADYSVLYNSASFSTFKCYG
VSPTKLNDLCFTNVYADSFVIRGDEVRQIAPGQTGKIADYNYKLPDDFTGCVIAWNSNNLD
SKVGGNYNYLYRLFRKSNLKPFERDISTEIYQAGSTPCNGVEGFNCYFPLQSYGFQPTNGV
GYQPYRVVLSFELLHAPATSGGGSKKSAAEASKKPRQKRTATKAYNVTQAFGRRGPEQTQG
NFGDQELIRQGTDYKHWPQIAQFAPSASAFFGMSRIGMEVTPSGTWLTYTGAIKLDDKDPNFKD
QFILLNKHIDAYKTFPPT
CoV-52-1
(SEQ ID NO: 106)
ACGTCAttaattaaATGGGAAAAATGGCTTCTCTTTTTGCTACTTTCCTTGTTGTGTTGGTTAG
TCTTTCTCTAGCTAGTGAGAGTAGTGCTGCTTCTGTGTACGCCTGGAACCGGAAGAGGA
TTTCTAACTGCGTGGCCGATTACAGCGTGCTGTACAACTCTGCTAGCTTCAGCACCTTC
AAGTGCTACGGTGTGTCTCCTACCAAGCTGAACGATCTGTGCTTCACCAACGTGTACGC
TGACTCTTTCGTGATCAGGGGTGATGAGGTTAGGCAGATTGCTCCTGGTCAGACCGGTA
AGATCGCTGACTACAACTACAAGCTGCCTGATGACTTCACCGGTTGCGTGATCGCTTGG
AACTCTAACAACCTGGACTCTAAGGTTGGCGGCAATTACAACTACCTCTACCGGCTGTT
CCGGAAGTCTAACCTTAAGCCTTTCGAGCGGGATATCAGCACCGAGATCTATCAGGCT
GGTTCTACTCCTTGCAATGGCGTTGAGGGTTTCAACTGCTACTTCCCGCTTCAGTCTTAC
GGATTCCAGCCTACTAATGGTGTGGGCTACCAGCCTTACAGAGTGGTGGTTTTGTCTTT
CGAGCTTCTGCATGCTCCTGCTACTTCTGGAGGTGGTTCTAAAAAAAGTGCCGCTGAAG
CATCCAAGAAACCGCGACAGAAGAGGACTGCAACAAAAGCTTATAATGTTACTCAAGC
ATTTGGGAGACGTGGTCCTGAACAAACTCAGGGAAATTTTGGTGACCAGGAGCTTATA
AGGCAAGGAACTGATTATAAGCATTGGCCTCAAATTGCTCAGTTTGCTCCATCAGCGTC
TGCCTTCTTCGGGATGTCAAGAATTGGAATGGAAGTAACCCCTTCTGGCACATGGTTAA
CATATACTGGTGCAATCAAATTGGATGATAAAGACCCCAATTTCAAAGATCAAGTTAT
ACTCCTGAACAAGCACATTGATGCTTACAAGACATTTCCTCCAACATGActcgagTCGACG
T
CoV-52-2
(SEQ ID NO: 114)
ACGTCAttaattaaATGGGAAAAATGGCTTCTCTTTTTGCTACTTTCCTTGTTGTGTTGGTTAG
TCTTTCTCTAGCTAGTGAGAGTAGTGCTGCTTCTGTATATGCTTGGAACCGCAAAAGAA
TAAGCAATTGTGTGGCAGACTACAGCGTGCTATACAATTCTGCGAGTTTCTCAACATTC
AAGTGCTATGGTGTGTCACCAACTAAGTTAAATGATCTCTGCTTTACTAATGTGTACGC
TGATTCATTTGTCATTAGAGGTGATGAAGTTCGTCAGATCGCTCCAGGACAAACTGGCA
AGATAGCTGACTACAACTATAAGCTTCCGGATGACTTTACGGGATGTGTTATTGCATGG
AATTCCAACAACCTTGATTCTAAAGTTGGAGGGAATTATAATTACTTGTATCGGCTTTT
CAGGAAATCAAATTTGAAACCTTTTGAGAGGGATATTTCGACCGAAATTTATCAAGCC
GGGAGTACTCCATGCAATGGTGTAGAAGGATTCAACTGTTACTTTCCTCTGCAATCATA
TGGATTTCAACCCACAAACGGCGTGGGTTATCAGCCCTATAGGGTAGTAGTTCTAAGTT
TTGAACTTTTGCATGCACCTGCAACATCTGGAGGTGGTTCTAAGAAGTCAGCTGCTGAG
GCGAGCAAGAAACCAAGGCAGAAAAGAACTGCAACAAAAGCATATAATGTTACACAA
GCATTTGGGAGGCGTGGTCCTGAACAAACTCAAGGAAATTTTGGTGATCAAGAATTGA
TACGACAGGGCACTGATTATAAACATTGGCCTCAAATTGCTCAGTTTGCTCCCTCCGCC
TCTGCATTCTTTGGAATGTCAAGAATTGGTATGGAGGTGACCCCGAGTGGGACATGGTT
AACATATACTGGAGCCATCAAGCTTGATGATAAAGACCCAAATTTCAAGGACCAGGTT
ATACTGCTCAACAAGCACATTGATGCTTACAAAACATTCCCTCCTACTTGActcgagTCGA
CGT

Identified priority N protein epitopes included:

(SEQ ID NO: 115)
1. QIGYYRRATRRIRGG (83-98);
(SEQ ID NO: 116)
2. QVILLNKHIDAY (349-360);
(SEQ ID NO: 117)
3. LALLLDRLNQLESK (219-233);
(SEQ ID NO: 118)
4. AFFGMSRIGME (313-323);
and
(SEQ ID NO: 119)
5. SKQLQQSMSSADS (404 416).

Most T cell epitopes are selected strictly on predicted immunogenicity. However, a strict immunogenicity analysis fails to take into account manufacturability criteria and epitopes. The present invention takes into account additional structure/function considerations. One such manufacturing consideration is the function of the N protein, which is nucleotide binding. Positively charged amino acids, like Arginine (ARG) are likely involved in binding negatively charged amino acids and would therefore be expected to be facing the interior of the particle and therefore not exposed to neutralizing antibodies. Further, structural analysis of the nucleocapsid N2b domain expresses and dimerizes in E. coli; amino acids 247-365.

Immunogenic peptide 2, QVILLNKHIDAY (349-360) (SEQ ID NO:116) is exposed and highly structured, while AFFGMSRIGME (313-323) (SEQ ID NO:118) looks to be in the center of the beta sheets and is unlikely to be exposed. The present invention also includes constructs designed to express the N2b domain fused to LicKM and “naked” peptides. They also include concatenated N and S epitopes with high immune potential and that are solvent exposed based on respective X-ray structures.

SARS-CoV Spike protein (UniProtKB - P59594 (SPIKE_SARS))
(SEQ ID NO: 120)
MFIFLLFLTLTSGSDLDRCTTFDDVQAPNYTQHTSSMRGVYYPDEIFRSDTLYLTQDLFLPF
YSNVTGFHTINHTFGNPVIPFKDGIYFAATEKSNVVRGWVFGSTMNNKSQSVIIINNSTNVV
IRACNFELCDNPFFAVSKPMGTQTHTMIFDNAFNCTFEYISDAFSLDVSEKSGNFKHLREFV
FKNKDGFLYVYKGYQPIDVVRDLPSGFNTLKPIFKLPLGINITNFRAILTAFSPAQDIWGTSA
AAYFVGYLKPTTFMLKYDENGTITDAVDCSQNPLAELKCSVKSFEIDKGIYQTSNFRVVPS
GDVVRFPNITNLCPFGEVFNATKFPSVYAWERKKISNCVADYSVLYNSTFFSTFKCYGVSA
TKLNDLCFSNVYADSFVVKGDDVRQIAPGQTGVIADYNYKLPDDFMGCVLAWNTRNIDA
TSTGNYNYKYRYLRHGKLRPFERDISNVPFSPDGKPCTPPALNCYWPLNDYGFYTTTGIGY
QPYRVVVLSFELLNAPATVCGPKLSTDLIKNQCVNFNFNGLTGTGVLTPSSKRFQPFQQFG
RDVSDFTDSVRDPKTSEILDISPCSFGGVSVITPGTNASSEVAVLYQDVNCTDVSTAIHADQ
LTPAWRIYSTGNNVFQTQAGCLIGAEHVDTSYECDIPIGAGICASYHTVSLLRSTSQKSIVAYT
MSLGADSSIAYSNNTIAIPTNFSISITTEVMPVSMAKTSVDCNMYICGDSTECANLLLQYGSFCTQL
NRALSGIAAEQDRNTREVFAQVKQMYKTPTLKYFGGFNFSQILPDPLKPTKRSFIEDLLFNKVTL
ADAGFMKQYGECLGDINARDLICAQKFNGLTVLPPLLTDDMIAAYTAALVSGTATAGWTFGAGA
ALQIPFAMQMAYRFNGIGVTQNVLYENQKQIANQFNKAISQIQESLTTTSTALGKLQDWNQNAQ
ALNTLVKQLSSNFGAISSVLNDILSRLDKVEAEVQIDRLITGRLQSLQTYVTQQLIRAAEIRASANLA
ATKMSECVLGQSKRVDFCGKGYHEMSFPQAAPHGWFLHVTYVPSQERNFTTAPAICHEGKAY
FPREGVFVFNGTSWFITQRNFFSPQIITTDNTFVSGNCDWIGIINNTVYDPLQPELDSFKEELDK
YFKNHTSPDVDLGDISGINASWNIQKEIDRLNEVAKNLNESLIDLQELGKYEQYIKWPWYVWLG
FIAGLIAIVMVTILLCCMTSCCSCLKGACSCGSCCKFDEDDSEPVLKGVKLHYT
Spike glycoprotein [Severe acute respiratory syndrome
coronavirus 2] GenBank: QIC53213.1
(SEQ ID NO: 121)
MFVFLVLLPLVSSQCVNLTTRTQLPPAYTNSFTRGVYYPDKVFRSSVLHSTQDLFLPFFSNV
TWFHAIHVSGTNGTKRFDNPVLPFNDGVYFASTEKSNIIRGWIFGTTLDSKTQSLLIVNNAT
NVVIKVCEFQFCNDPFLGVYYHKNNKSWMESEFRVYSSANNCTFEYVSQPFLMDLEGKQG
NFKNLREFVFKNIDGYFKIYSKHTPINLVRDLPQGFSALEPLVDLPIGINITRFQTLLALHRSY
LTPGDSSSGWTAGAAAYYVGYLQPRTFLLKYNENGTITDAVDCALDPLSETKCTLKSFTVE
KGIYQTSNFRVQPTESIVRFPNITNLCPFGEFFNATRFASVYAWNRKRISNCVADYSVLYNSASFST
FKCYGVSPTKLNDLCFTNVYADSFVIRGDEVRQIAPGQTGKIADYNYKLPDDFTGCVIAWNSNNL
DSKVGGNYNYLYRLFRKSNLKPFERDISTEIYQAGSTPCNGVEGFNCYFPLQSYGFQPTNGVGYQ
PYRVVVLSFELLHAPATVCGPKKSTNLVKNKCVNFNFNGLTGTGVLTESNKKFLPFQQFGRDIA
DTTDAVRDPQTLEILDITPCSFGGVSVITPGTNTSNQVAVLYQDVNCTEVPVAIHADQLTPT
WRVYSTGSNVFQTRAGCLIGAEHVNNSYECDIPIGAGICASYQTQTNSPRRARSVASQSIIA
YTMSLGAENSVAYSNNSIAIPTNFTISVTTEILPVSMTKTSVDCTMYICGDSTECSNLLLQYG
SFCTQLNRALTGIAVEQDKNTQEVFAQVKQIYKTPPIKDFGGFNFSQILPDPSKPSKRSFIED
LLFNKVTLADAGFIKQYGDCLGDIAARDLICAQKFNGLTVLPPLLTDEMIAQYTSALLAGTI
TSGWTFGAGAALQIPFAMQMAYRFNGIGVTQNVLYENQKLIANQFNSAIGKIQDSLSSTAS
ALGKLQDVVNQNAQALNTLVKQLSSNFGAISSVLNDILSRLDKVEAEVQIDRLITGRLQSL
QTYVTQQLIRAAEIRASANLAATKMSECVLGQSKRVDFCGKGYHLMSFPQSAPHGVVFLH
VTYVPAQEKNFTTAPAICHDGKAHFPREGVFVSNGTHWFVTQRNFYEPQIITTDNTFVSGN
CDVVIGIVNNTVYDPLQPELDSFKEELDKYFKNHTSPDVDLGDISGINASVVNIQKEIDRLN
EVAKNLNESLIDLQELGKYEQYIKWPWYIWLGFIAGLIAIVMVTIMLCCMTSCCSCLKGCC
SCGSCCKFDEDDSEPVLKGVKLHYT
Italics is the CoV RBD sequence
nCoV RBD seq (RBM 438-510)
(SEQ ID NO: 122)
RVQPTESIVRFPNITNLCPFGEVFNATRFASVYAWNRKRISNCVADYSVLYNSASFSTFKCY
GVSPTKLNDLCFTNVYADSFVIRGDEVRQIAPGQTGKIADYNYKLPDDFTGCVIAWNSNNL
DSKVGGNYNYLYRLFRKSNLKPFERDISTEIYQAGSTPCNGVEGFNCYFPLQSYGFQPTNG
VGYQPYRVVVLSFELLHAPATVCGPKKSTNLVKNKCVNFNFNGLTGTGVLTESNKKFLPF
QQFGRDIADTTDAVRDPQTLE
SARS-CoV Nucleocapsid protein >sp|P59595|1-422
(SEQ ID NO: 123)
MSDNGPQSNQRSAPRITFGGPTDSTDNNQNGGRNGARPKQRRPQGLPNNTASWFTALTQH
GKEELRFPRGQGVPINTNSGPDDQIGYYRRATRRVRGGDGKMKELSPRWYFYYLGTGPEA
SLPYGANKEGIVWVATEGALNTPKDHIGTRNPNNNAATVLQLPQGTTLPKGFYAEGSRGG
SQASSRSSSRSRGNSRNSTPGSSRGNSPARMASGGGETALALLLLDRLNQLESKVSGKGQQ
QQGQTVTKKSAAEASKKPRQKRTATKQYNVTQAFGRRGPEQTQGNFGDQDLIRQGTDYK
HWPQIAQFAPSASAFFGMSRIGMEVTPSGTWLTYHGAIKLDDKDPQFKDNVILLNKHIDAY
KTFPPTEPKKDKKKKTDEAQPLPQRQKKQPTVTLLPAADMDDFSRQLQNSMSGASADSTQ
A
LicB protein sequence
(SEQ ID NO: 124)
MKNRVISLLMASLLLVLSVIVAPFYKAEAATVVNTPFVAVFSNFDSSQWEKADWANGSVF
NCVWKPSQVTFSNGKMILTLDREYGGSYPYKSGEYRTKSFFGYGYYEVRMKAAKNVGIVS
SFFTYTGPSDNNPWDEIDIEFLGKDTTKVQFNWYKNGVGGNEYLHNLGFDASQDFHTYGF
EWRPDYIDFYVDGKKVYRGTRNIPVTPGKIMMNLWPGIGVDEWLGRYDGRTPLQAEYEY
VKYYPNGVPQDNPTPTPTIAPSTPTNPNLPLKGDVNGDGHVNSSDYSLFKRYLLRVIDRFPV
GDQSVADVNRDGRIDSTDLTMLKRYLIRAIPSL
PR1a-LicKM alone (26.7 kDa)
(SEQ ID NO: 125)
MGFVLFSQLPSFLLVSTLLLFLVISHSCRAHHHHHHHHGGSYPYKSGEYRTKSFFGYGYYE
VRMKAAKNVGIVSSFFTYTGPSDNNPWDEIDIEFLGKDTTKVQFNWYKNGVGGNEYLHN
LGFDASQDFHTYGFEWRPDYIDFYVDGKKVYRGTRNIPVTPGKIMMNLWPGIGVDEWLG
RYDGRTPLQAEYEYVKYYPNGRSEFKLVVNTPFVAVFSNFDSSQWEKADWANGSVFNCV
WKPSQVTFSNGKMILTLDREY-
(SEQ ID NO: 126)
ATGGGATTCGTTTTGTTTTCTCAATTGCCTTCATTTCTTTTGGTTTCTACTCTTTTGCTTT
TTCTTGTTATTTCTCATTCATGTAGAGCTCATCACCATCACCACCATCATCATGGCGGCT
CTTACCCTTATAAGAGCGGTGAGTACCGGACCAAGAGCTTCTTTGGTTACGGTTACTAC
GAGGTGCGGATGAAGGCTGCTAAGAACGTGGGTATCGTGTCCAGCTTCTTTACCTACA
CCGGGCCATCTGATAACAACCCTTGGGATGAGATCGACATCGAGTTCCTTGGTAAGGA
CACTACCAAGGTGCAGTTCAACTGGTACAAGAACGGTGTTGGTGGCAACGAGTACCTT
CACAACCTTGGCTTTGATGCCAGCCAGGATTTCCACACTTACGGTTTTGAATGGCGGCC
TGACTACATCGACTTCTACGTGGACGGTAAGAAGGTGTACAGGGGCACCAGAAATATC
CCTGTGACTCCTGGCAAGATCATGATGAACCTTTGGCCTGGTATCGGTGTGGATGAGT
GGCTTGGTAGATACGATGGTAGGACTCCTCTGCAGGCTGAGTACGAGTACGTTAAGTA
CTACCCTAACGGCAGATCTGAATTCAAGCTTGTGGTGAATACTCCTTTCGTGGCCGTGT
TCAGCAACTTCGATTCTAGCCAGTGGGAGAAAGCTGATTGGGCTAACGGTTCTGTGTT
CAACTGCGTGTGGAAGCCTTCTCAGGTGACCTTCTCTAACGGCAAGATGATTCTGACCC
TGGACCGTGAGTATTGA
Cloning strategy for CoV-15 16 & 17 from Cov-2
CoV-2
(SEQ ID NO: 127)
TtaattaaATGGACATCGATCCGTACAAAGAATTTGGCGCGACCGTCGAGCTGCTGAGCTTC
CTGCCGAGCGATTTTTTCCCGAGCGTGCGTGACCTGCTGGACACCGCGAGCGCACTGTA
TCGTGAAGCACTGGAAAGCCCAGAGCACTGTAGCCCGCACCACACCGCCCTGCGCCAG
GCGATTCTGTGCTGGGGTGAACTGATGACCCTGGCCACCTGGGTGGGTGCTAACCTTGA
GGATCCGAACTCTAACAACCTGGACTCTAAGGTTGGCGGCAACTACAACTACCTCTAC
AGGCTGTTCCGGAAGTCCAACCTTAAGCCTTTCGAGAGGGATATCAGCACCGAGATCT
ATCAGGCTGGTTCTACTCCTTGCAACGGTGTTGAGGGTTTCAACTGCTACTTCCCGCTTC
AGTCTTACGGTTTCCAGCCTACTAATGGTGTGGGCTACCAGCCTTATGCTAGCCGTGAT
CTGGTCGTCAACTATGTGAATACCAACATGGGTCTGAAAATTCGTCAGCTGCTGTGGTT
TCATATTAGCTGCCTGACCTTCGGTCGTGAAACCGTGCTGGAGTATCTGGTGAGCTTCG
GTGTGTGGATTCGCACCCCGCCGGCGTATCGTCCGCCGAACGCGCCAATTCTGAGCAC
GCTGCCGGAGACCACCGTGGTTTAGctcgag
BamHI and NheI sites respectively before and after the RBM
For CoV2-F1 - We can use PGR-15 or PGR-10
Primer CoV2-R1 -
GAGTTCGGATCCTCAAGGTTAGCACCCACCCAGG
Primer CoV2-F2 -
GCCTTATGCTAGCCGTGATCTGGTCGTCAAC
For CoV2-R2 -PGR-16 or PGR-2 can be used
Spike glycoprotein [Severe acute respiratory syndrome
coronavirus 2] GenBank: QIC53213.1
(SEQ ID NO: 128)
MFVFLVLLPLVSSQCVNLTTRTQLPPAYTNSFTRGVYYPDKVFRSSVLHSTQDLFLPFFSNV
TWFHAIHVSGTNGTKRFDNPVLPFNDGVYFASTEKSNIIRGWIFGTTLDSKTQSLLIVNNAT
NVVIKVCEFQFCNDPFLGVYYHKNNKSWMESEFRVYSSANNCTFEYVSQPFLMDLEGKQG
NFKNLREFVFKNIDGYFKIYSKHTPINLVRDLPQGFSALEPLVDLPIGINITRFQTLLALHRSY
LTPGDSSSGWTAGAAAYYVGYLQPRTFLLKYNENGTITDAVDCALDPLSETKCTLKSFTVE
KGIYQTSNFRVQPTESIVRFPNITNLCPFGEVFNATRFASVYAWNRKRISNCVADYSVLYNS
ASFSTFKCYGVSPTKLNDLCFTNVYADSFVIRGDEVRQIAPGQTGKIADYNYKLPDDFTGC
VIAWNSNNLDSKVGGNYNYLYRLFRKSNLKPFERDISTEIYQAGSTPCNGVEGFNCYFPLQ
SYGFQPTNGVGYQPYRVVVLSFELLHAPATVCGPKKSTNLVKNKCVNFNFNGLTGTGVLT
ESNKKFLPFQQFGRDIADTTDAVRDPQTLEILDITPCSFGGVSVITPGTNTSNQVAVLYQDV
NCTEVPVAIHADQLTPTWRVYSTGSNVFQTRAGCLIGAEHVNNSYECDIPIGAGICASYQTQ
TNSPRRARSVASQSIIAYTMSLGAENSVAYSNNSIAIPTNFTISVTTEILPVSMTKTSVDCTM
YICGDSTECSNLLLQYGSFCTQLNRALTGIAVEQDKNTQEVFAQVKQIYKTPPIKDFGGFNF
SQILPDPSKPSKRSFIEDLLFNKVTLADAGFIKQYGDCLGDIAARDLICAQKFNGLTVLPPLL
TDEMIAQYTSALLAGTITSGWTFGAGAALQIPFAMQMAYRFNGIGVTQNVLYENQKLIAN
QFNSAIGKIQDSLSSTASALGKLQDVVNQNAQALNTLVKQLSSNFGAISSVLNDILSRLDKV
EAEVQIDRLITGRLQSLQTYVTQQLIRAAEIRASANLAATKMSECVLGQSKRVDFCGKGYH
LMSFPQSAPHGVVFLHVTYVPAQEKNFTTAPAICHDGKAHFPREGVFVSNGTHWFVTQRN
FYEPQIITTDNTFVSGNCDVVIGIVNNTVYDPLQPELDSFKEELDKYFKNHTSPDVDLGDISG
INASVVNIQKEIDRLNEVAKNLNESLIDLQELGKYEQYIKWPWYIWLGFIAGLIAIVMVTIM
LCCMTSCCSCLKGCCSCGSCCKFDEDDSEPVLKGVKLHYT
Epitopes exhibiting potent neutralizing activity.
S1-93:
(SEQ ID NO: 129)
VLTESNKKFLPFQQFG 553-564
S1-105:
(SEQ ID NO: 130)
AIHADQLTPTWRVYST 625-636
S2-78:
(SEQ ID NO: 131)
DSFKEELDKYFKNHTS 1148-1159
Synthetic construct hetero-tandem core protein (CoHe-GFPs) gene,
GenBank: KM396758.1.
(SEQ ID NO: 132)
MDIDPYKEFGATVELLSFLPSDFFPSVRDLLDTASALYREALESPEHCSPHHTALRQAILCW
GELMTLATWVGNNLEGSAGGGRDPASRDLVVNYVNTNMGLKIRQLLWFHISCLTFGRET
VLEYLVSFGVWIRTPPAYRPPNAPILSTLPETTVVGGSSGGSGGSGGSGGSGGSGGSTMDID
PYKEFGATVELLSFLPSDFFPSVRDLLDTASALYREALESPEHCSPHHTALRQAILCWGELM
TLATWVGNNLEFGGSMVSKGEELFTGWPILVELDGDVNGHKFSVSGEGEGDATYGKLTLKFI
CTTGKLPVPWPTLVTTLTYGVQCFSRYPDHMKQHDFFKSAMPEGYVQERTIFFKDDGNYKTRA
EVKFEGDTLVNRIELKGIDFKEDGNILGHKLEYNYNSHNVYIMADKQKNGIKVNFKIRHNIEDGS
VQLADHYQQNTPIGDGPVLLPDNHYLSTQSALSKDPNEKRDHMVLLEFVTAAGITLGMDELYKS
GGASDPASRDLVVNYVNTNMGLKIRQLLWFHISCLTFGRETVLEYLVSFGVWIRTPPAYRP
PNAPILSTLPETTVVRRRDRGRSPRRRTPSPRRRRSQSPRRRRSQSRESQCLE
In underline: HLA Class II profile targeting CD4+ (Th) cells
In bold: HLA class I profile targeting CD8+ (CTL) cells
SARS-CoV-2 Spike glycoprotein Acc# QIC53213.1
(SEQ ID NO: 133)
MFVFLVLLPLVSSQCVNLTTRTQLPPAYTNSFTRGVYYPDKVFRSSVLHSTQDLFLPFFSN
VTWFHAIHVSGTNGTKRFDNPVLPFNDGVYFASTEKSNIIRGWIFGTTLDSKTQSLLIVNNA
TNVVIKVCEFQFCNDPFLGVYYHKNNKSWMESEFRVYSSANNCTFEYVSQPFLMDLEGK
QGNFKNLREFVFKNIDGYFKIYSKHTPINLVRDLPQGFSALEPLVDLPIGINITRFQTLLALHR
SYLTPGDSSSGWTAGAAAYYVGYLQPRTFLLKYNENGTITDAVDCALDPLSETKCTLKS
FTVEKGIYQTSNFRVQPTESIVRFPNITNLCPFGEVFNATRFASVYAWNRKRISNCVADYSV
LYNSASFSTFKCYGVSPTKLNDLCFTNVYADSFVIRGDEVRQIAPGQTGKIADYNYKLPDD
FTGCVIAWNSNNLDSKVGGNYNYLYRLFRKSNLKPFERDISTEIYQAGSTPCNGVEGFNCY
FPLQSYGFQPTNGVGYQPYRVVVLSFELLHAPATVCGPKKSTNLVKNKCVNFNFNGLTG
TGVLTESNKKFLPFQQFGRDIADTTDAVRDPQTLEILDITPCSFGGVSVITPGTNTSNQVAVL
YQDVNCTEVPVAIHADQLTPTWRVYSTGSNVFQTRAGCLIGAEHVNNSYECDIPIGAGICA
SYQTQTNSPRRARSVASQSIIAYTMSLGAENSVAYSNNSIAIPTNFTISVTTEILPVSMTKTS
VDCTMYICGDSTECSNLLLQYGSFCTQLNRALTGIAVEQDKNTQEVFAQVKQIYKTPPIKD
FGGFNFSQILPDPSKPSKRSFIEDLLFNKVTLADAGFIKQYGDCLGDIAARDLICAQKFNGL
TVLPPLLTDEMIAQYTSALLAGTITSGWTFGAGAALQIPFAMQMAYRFNGIGVTQNVLYE
NQKLIANQFNSAIGKIQDSLSSTASALGKLQDVVNQNAQALNTLVKQLSSNFGAISSVLNDI
LSRLDKVEAEVQIDRLITGRLQSLQTYVTQQLIRAAEIRASANLAATKMSECVLGQSKRVD
FCGKGYHLMSFPQSAPHGVVFLHVTYVPAQEKNFTTAPAICHDGKAHFPREGVFVSNGTH
WFVTQRNFYEPQIITTDNTFVSGNCDVVIGIVNNTVYDPLQPELDSFKEELDKYFKNHTSPD
VDLGDISGINASVVNIQKEIDRLNEVAKNLNESLIDLQELGKYEQYIKWPWYIWLGFIAGL
IAIVMVTIMLCCMTSCCSCLKGCCSCGSCCKFDEDDSEPVLKGVKLHYT
In underline: HLA Class II profile targeting CD4+ (Th) cells
In bold: HLA class I profile targeting CD8+ (CTL) cells
SARS-CoV-2 Nucleoprotein Acc# QLC94852.1 (45.6 kDa)
(SEQ ID NO: 134)
MSDNGPQNQRNAPRITFGGPSDSTGSNQNGERSGARSKQRRPQGLPNNTASWFTALTQHG
KEDLKFPRGQGVPINTNSSPDDQIGYYRRATRRIRGGDGKMKDLSPRWYFYYLGTGPEAG
LPYGANKDGIIWVATEGALNTPKDHIGTRNPANNAAIVLQLPQGTTLPKGFYAEGSRGGS
QASSRSSSRSRNSSRNSTPGSSRGTSPARMAGNGGDAALALLLLDRLNQLESKMSGKGQQ
QQGQTVTKKSAAEASKKPRQKRTATKAYNVTQAFGRRGPEQTQGNFGDQELIRQGTDYK
HWPQIAQFAPSASAFFGMSRIGMEVTPSGTWLTYTGAIKLDDKDPNFKDQVILLNKHIDA
YKTFPPTEPKKDKKKKADETQALPQRQKKQQTVTLLPAADLDDFSKQLQQSMSSADSTQ
A
(SEQ ID NO: 135)
DQIGYYRRATRRIRGGDGKMKDLSPRWYFYYLGTGALALLLLDRLNQLESKMSAFFGM
SRIGMEVADLDDFSKQLQQSMSSADST
(SEQ ID NO: 136)
GATCAGATTGGCTATTATCGCCGCGCGACCCGCCGCATTCGCGGCGGCGATGGCAAAA
TGAAAGATCTGAGCCCGCGCTGGTATTTTTATTATCTGGGCACCGGCGCGCTGGCGCTG
CTGCTGCTGGATCGCCTGAACCAGCTGGAAAGCAAAATGAGCGCGTTTTTTGGCATGA
GCCGCATTGGCATGGAAGTGGCGGATCTGGATGATTTTAGCAAACAGCTGCAGCAGAG
CATGAGCAGCGCGGATAGCACC
CoV-53, Ext-CoV N 247-365 (13.4 kDa) (Similar to iBio201
CoV-41 without His tag)
(SEQ ID NO: 137)
MGKMASLFATFLVVLVSLSLASESSATKKSAAEASKKPRQKRTATKAYNVTQAFGRRGP
EQTQGNFGDQELIRQGTDYKHWPQIAQFAPSASAFFGMSRIGMEVTPSGTWLTYTGAIK
LDDKDPNFKDQVILLNKHIDAYKTFPP
(SEQ ID NO: 138)
ACGTCAttaattaaATGGGAAAAATGGCTTCTCTTTTTGCTACTTTCCTTGTTGTGTTGGTTA
GTCTTTCTCTAGCTAGTGAGAGTAGTGCTACCAAGAAAAGCGCTGCCGAAGCGTCAAA
GAAGCCAAGGCAGAAGAGAACTGCTACAAAAGCTTATAATGTGACTCAAGCATTTGG
AAGACGAGGCCCTGAGCAAACACAGGGGAATTTTGGTGATCAAGAACTGATCAGGCA
AGGAACAGATTATAAACATTGGCCTCAAATTGCTCAGTTTGCTCCTTCTGCCTCCGCAT
TCTTTGGTATGTCTCGTATTGGAATGGAGGTAACTCCGAGTGGTACATGGCTTACGTAT
ACTGGCGCAATTAAATTGGATGATAAAGACCCAAATTTCAAGGACCAGGTTATACTAT
TAAACAAGCATATAGATGCATACAAAACCTTCCCACCCTGActcgagcctaggACGAAG
CoV-54 Ext-CoV N 247-365 (13.4 kDa) (Similar to iBio201
CoV-42 without His tag)
(SEQ ID NO: 139)
MGKMASLFATFLVVLVSLSLASESSATKKSAAEASKKPRQKRTATKAYNVTQAFGRRGP
EQTQGNFGDQELIRQGTDYKHWPQIAQFAPSASAFFGMSRIGMEVTPSGTWLTYTGAIK
LDDKDPNFKDQVILLNKHIDAYKTFPP
(SEQ ID NO: 140)
ACGTCAttaattaaATGGGAAAAATGGCTTCTCTTTTTGCTACTTTCCTTGTTGTGTTGGTTA
GTCTTTCTCTAGCTAGTGAGAGTAGTGCTACAAAGAAATCTGCTGCTGAAGCCAGCAA
GAAGCCAAGACAGAAAAGAACTGCAACAAAAGCATATAATGTTACCCAGGCATTTGG
AAGGCGAGGCCCCGAGCAAACTCAAGGGAATTTTGGTGACCAAGAACTGATAAGGCA
GGGAACTGATTATAAACATTGGCCTCAAATTGCTCAATTTGCTCCGTCAGCGTCAGCCT
TCTTTGGGATGAGTCGTATTGGAATGGAGGTAACGCCATCTGGTACATGGCTTACCTAT
ACTGGTGCAATCAAATTGGATGATAAAGATCCAAATTTCAAGGACCAGGTGATATTAC
TCAACAAGCATATTGATGCTTACAAGACATTCCCTCCTTGActcgagcctaggACGAAG
CoV-55 Ext-CoV N 245-365 (13.5 kDa) (Similar to iBio201
CoV-43 without His tag)
(SEQ ID NO: 141)
MGKMASLFATFLVVLVSLSLASESSATVTKKSAAEASKKPRQKRTATKAYNVTQAFGRR
GPEQTQGNFGDQELIRQGTDYKHWPQIAQFAPSASAFFGMSRIGMEVTPSGTWLTYTGA
IKLDDKDPNFKDQVILLNKHIDAYKTFPP
(SEQ ID NO: 142)
ACGTCAttaattaaATGGGAAAAATGGCTTCTCTTTTTGCTACTTTCCTTGTTGTGTTGGTTA
GTCTTTCTCTAGCTAGTGAGAGTAGTGCTACAGTGACCAAGAAAAGCGCTGCCGAAGC
GTCAAAGAAGCCAAGGCAGAAGAGAACTGCTACAAAAGCTTATAATGTGACTCAAGC
ATTTGGAAGACGAGGCCCTGAGCAAACACAGGGGAATTTTGGTGATCAAGAACTGATC
AGGCAAGGAACAGATTATAAACATTGGCCTCAAATTGCTCAGTTTGCTCCTTCTGCCTC
CGCATTCTTTGGTATGTCTCGTATTGGAATGGAGGTAACTCCGAGTGGTACATGGCTTA
CGTATACTGGCGCAATTAAATTGGATGATAAAGACCCAAATTTCAAGGACCAGGTTAT
ACTATTAAACAAGCATATAGATGCATACAAAACCTTCCCACCCTGActcgagcctaggACGA
AG
CoV-56 Ext-CoV N 245-365 (13.5 kDa) (Similar to iBio201
CoV-44 without His tag)
(SEQ ID NO: 143)
MGKMASLFATFLVVLVSLSLASESSATVTKKSAAEASKKPRQKRTATKAYNVTQAFGRR
GPEQTQGNFGDQELIRQGTDYKHWPQIAQFAPSASAFFGMSRIGMEVTPSGTWLTYTGA
IKLDDKDPNFKDQVILLNKHIDAYKTFPP
(SEQ ID NO: 144)
ACGTCAttaattaaATGGGAAAAATGGCTTCTCTTTTTGCTACTTTCCTTGTTGTGTTGGTTA
GTCTTTCTCTAGCTAGTGAGAGTAGTGCTACAGTGACAAAGAAATCTGCTGCTGAAGC
CAGCAAGAAGCCAAGACAGAAAAGAACTGCAACAAAAGCATATAATGTTACCCAGGC
ATTTGGAAGGCGAGGCCCCGAGCAAACTCAAGGGAATTTTGGTGACCAAGAACTGAT
AAGGCAGGGAACTGATTATAAACATTGGCCTCAAATTGCTCAATTTGCTCCGTCAGCG
TCAGCCTTCTTTGGGATGAGTCGTATTGGAATGGAGGTAACGCCATCTGGTACATGGCT
TACCTATACTGGTGCAATCAAATTGGATGATAAAGATCCAAATTTCAAGGACCAGGTG
ATATTACTCAACAAGCATATTGATGCTTACAAGACATTCCCTCCTTGActcgagcctaggACG
AAG
CoV-57 IBIO201S-RBD(319-530)_N-CTD(245-370)Long-Long
(Similar to CoV-49 but shorter RBD)
(38 kDa)
(SEQ ID NO: 145)
MGKMASLFATFLVVLVSLSLASESSARVQPTESIVRFPNITNLCPFGEVFNATRFASVYAWN
RKRISNCVAPYSVLYNSASFSTFKCYGVSPTKLNPLCFTNVYAPSFVIRGPEVRQIAPGQTG
KIAPYNYKLPPPFTGCVIAWNSNNLPSKVGGNYNYLYRLFRKSNLKPFERPISTEIYQAGS
TPCNGVEGFNCYFPLQSYGFQPTNGVGYQPYRVVVLSFELLHAPATVCGPKKSGGGSTVTK
KSAAEASKKPRQKRTATKAYNVTQAFGRRGPEQTQGNFGDQELIRQGTDYKHWPQIAQFAPSAS
AFFGMSRIGMEVTPSGTWLTYTGAIKLDDKDPNFKDQVILLNKHIDAYKTFPPTEPKK
(SEQ ID NO: 146)
ACGTCAttaattaaATGGGAAAAATGGCTTCTCTTTTTGCTACTTTCCTTGTTGTGTTGGTTAG
TCTTTCTCTAGCTAGTGAGAGTAGTGCTAGGGTTCAGCCTACTGAGTCTATCGTGCGGT
TCCCTAACATCACCAACTTGTGCCCTTTCGGCGAGGTGTTCAATGCTACTAGGTTCGCT
TCTGTGTACGCCTGGAACCGGAAGAGGATTTCTAACTGCGTGGCCGATTACAGCGTGCT
GTACAACTCTGCTAGCTTCAGCACCTTCAAGTGCTACGGTGTGTCTCCTACCAAGCTGA
ACGATCTGTGCTTCACCAACGTGTACGCTGACTCTTTCGTGATCAGGGGTGATGAGGTT
AGGCAGATTGCTCCTGGTCAGACCGGTAAGATCGCTGACTACAACTACAAGCTGCCTG
ATGACTTCACCGGTTGCGTGATCGCTTGGAACTCTAACAACCTGGACTCTAAGGTTGGC
GGCAATTACAACTACCTCTACCGGCTGTTCCGGAAGTCTAACCTTAAGCCTTTCGAGCG
GGATATCAGCACCGAGATCTATCAGGCTGGTTCTACTCCTTGCAATGGCGTTGAGGGTT
TCAACTGCTACTTCCCGCTTCAGTCTTACGGATTCCAGCCTACTAATGGTGTGGGCTAC
CAGCCTTACAGAGTGGTGGTTTTGTCTTTCGAGCTTCTGCATGCTCCTGCTACTGTTTGC
GGTCCTAAGAAGTCCGGTGGTGGATCCACGGTGACCAAAAAAAGTGCCGCTGAAGCAT
CCAAGAAACCGCGACAGAAGAGGACTGCAACAAAAGCTTATAATGTTACTCAAGCATT
TGGGAGACGTGGTCCTGAACAAACTCAGGGAAATTTTGGTGACCAGGAGCTTATAAGG
CAAGGAACTGATTATAAGCATTGGCCTCAAATTGCTCAGTTTGCTCCATCAGCGTCTGC
CTTCTTCGGGATGTCAAGAATTGGAATGGAAGTAACCCCTTCTGGCACATGGTTAACAT
ATACTGGTGCAATCAAATTGGATGATAAAGACCCCAATTTCAAAGATCAAGTTATACT
CCTGAACAAGCACATTGATGCTTACAAGACATTTCCTCCAACAGAGCCAAAGAAGTGA
ctcgagTCGACGT
CoV-58 IBIO201S-RBD(319-530)_N-CTD(248-366)Long-Short
(Similar to CoV-50 but shorter RBD) (37.4 kDa)
(SEQ ID NO: 147)
MGKMASLFATFLVVLVSLSLASESSARVQPTESIVRFPNITNLCPFGEVFNATRFASVYAWN
RKRISNCVADYSVLYNSASFSTFKCYGVSPTKLNDLCFTNVYADSFVIRGDEVRQIAPGQTG
KIADYNYKLPDDFTGCVIAWNSNNLDSKVGGNYNYLYRLFRKSNLKPFERDISTEIYQAGS
TPCNGVEGFNCYFPLQSYGFQPTNGVGYQPYRVVVLSFELLHAPATVCGPKKSGGGSKKS
AAEASKKPRQKRTATKAYNVTQAFGRRGPEQTQGNFGDQELIRQGTDYKHWPQIAQFAPSASAF
FGMSRIGMEVTPSGTWLTYTGAIKLDDKDPNFKDQVILLNKHIDAYKTFPPT
(SEQ ID NO: 148)
ACGTCAttaattaaATGGGAAAAATGGCTTCTCTTTTTGCTACTTTCCTTGTTGTGTTGGTTAG
TCTTTCTCTAGCTAGTGAGAGTAGTGCTAGGGTTCAGCCTACTGAGTCTATCGTGCGGT
TCCCTAACATCACCAACTTGTGCCCTTTCGGCGAGGTGTTCAATGCTACTAGGTTCGCT
TCTGTGTACGCCTGGAACCGGAAGAGGATTTCTAACTGCGTGGCCGATTACAGCGTGCT
GTACAACTCTGCTAGCTTCAGCACCTTCAAGTGCTACGGTGTGTCTCCTACCAAGCTGA
ACGATCTGTGCTTCACCAACGTGTACGCTGACTCTTTCGTGATCAGGGGTGATGAGGTT
AGGCAGATTGCTCCTGGTCAGACCGGTAAGATCGCTGACTACAACTACAAGCTGCCTG
ATGACTTCACCGGTTGCGTGATCGCTTGGAACTCTAACAACCTGGACTCTAAGGTTGGC
GGCAATTACAACTACCTCTACCGGCTGTTCCGGAAGTCTAACCTTAAGCCTTTCGAGCG
GGATATCAGCACCGAGATCTATCAGGCTGGTTCTACTCCTTGCAATGGCGTTGAGGGTT
TCAACTGCTACTTCCCGCTTCAGTCTTACGGATTCCAGCCTACTAATGGTGTGGGCTAC
CAGCCTTACAGAGTGGTGGTTTTGTCTTTCGAGCTTCTGCATGCTCCTGCTACTGTTTGC
GGTCCTAAGAAGTCCGGTGGTGGATCCAAAAAAAGTGCCGCTGAAGCATCCAAGAAAC
CGCGACAGAAGAGGACTGCAACAAAAGCTTATAATGTTACTCAAGCATTTGGGAGACG
TGGTCCTGAACAAACTCAGGGAAATTTTGGTGACCAGGAGCTTATAAGGCAAGGAACT
GATTATAAGCATTGGCCTCAAATTGCTCAGTTTGCTCCATCAGCGTCTGCCTTCTTCGG
GATGTCAAGAATTGGAATGGAAGTAACCCCTTCTGGCACATGGTTAACATATACTGGT
GCAATCAAATTGGATGATAAAGACCCCAATTTCAAAGATCAAGTTATACTCCTGAACA
AGCACATTGATGCTTACAAGACATTTCCTCCAACATGActcgagTCGACGT
CoV-59 IBIO201 S-NTD (19-310)_N-CTD (245-370)-8xHis Long-Long
(Similar to CoV-45 with His tag)
(SEQ ID NO: 149)
MGKMASLFATFLVVLVSLSLASESSATTRTQLPPAYTNSFTRGVYYPDKVFRSSVLHSTQD
LFLPFFSNVTWFHAIHVSGTNGTKRFDNPVLPFNDGVYFASTEKSNIIRGWIFGTTLDSKTQS
LLIVNNATNVVIKVCEFQFCNDPFLGVYYHKNNKSWMESEFRVYSSANNCTFEYVSQPFL
MDLEGKQGNFKNLREFVFKNIDGYFKIYSKHTPINLVRDLPQGFSALEPLVDLPIGINITRFQ
TLLALHRSYLTPGDSSSGWTAGAAAYYVGYLQPRTFLLKYNENGTITDAVDCALDPLSET
KCTLKSFTVEKSGGGSTVTKKSAAEASKKPRQKRTATKAYNVTQAFGRRGPEQTQGNFGDQEL
IRQGTDYKHWPQIAQFAPSASAFFGMSRIGMEVTPSGTWLTYTGAIKLDDKDPNFKDQVILLNK
HIDAYKTFPPTEPKKHHHHHHHSA
(SEQ ID NO: 150)
ACGTCAttaattaaATGGGAAAAATGGCTTCTCTTTTTGCTACTTTCCTTGTTGTGTTGGTTAG
TCTTTCTCTAGCTAGTGAGAGTAGTGCTACGACCAGGACACAACTTCCCCCTGCTTATA
CAAACTCTTTCACTAGGGGCGTCTATTACCCTGATAAGGTGTTTCGGAGTAGTGTATTG
CATTCAACTCAAGATTTGTTCCTTCCGTTCTTCTCAAATGTGACATGGTTTCATGCCATT
CATGTCTCAGGGACCAATGGTACAAAGAGATTTGATAATCCTGTTCTCCCATTCAATGA
CGGTGTTTATTTTGCTTCAACAGAGAAGAGCAACATTATAAGAGGATGGATTTTTGGAA
CAACGCTTGACTCAAAAACTCAGAGTTTATTGATAGTAAATAATGCAACTAACGTTGTG
ATCAAAGTTTGTGAATTCCAGTTCTGCAATGATCCATTTCTGGGAGTTTACTATCACAA
GAACAACAAGTCTTGGATGGAAAGTGAGTTCAGAGTATATTCTTCTGCAAACAATTGC
ACTTTTGAGTATGTTTCTCAGCCTTTTTTGATGGATCTTGAGGGGAAACAAGGAAATTT
CAAAAATCTACGCGAATTTGTTTTTAAGAATATTGATGGCTACTTCAAGATTTACAGCA
AGCATACTCCAATAAACCTTGTGAGAGACCTGCCACAGGGATTTTCCGCCTTAGAACC
ACTGGTAGACTTACCAATTGGGATCAATATCACACGTTTTCAAACTCTCCTAGCGTTGC
ACAGGAGTTACCTAACTCCTGGTGATTCTTCGAGCGGTTGGACTGCTGGAGCTGCAGCA
TATTATGTGGGTTATTTGCAACCCCGAACATTTCTTCTCAAATATAATGAAAATGGAAC
CATTACTGATGCTGTTGATTGTGCTTTGGATCCTCTTTCTGAAACAAAATGTACCCTGA
AATCATTTACTGTCGAGAAATCCGGTGGTGGCTCCACGGTGACCAAAAAAAGTGCCGC
TGAAGCATCCAAGAAACCGCGACAGAAGAGGACTGCAACAAAAGCTTATAATGTTACT
CAAGCATTTGGGAGACGTGGTCCTGAACAAACTCAGGGAAATTTTGGTGACCAGGAGC
TTATAAGGCAAGGAACTGATTATAAGCATTGGCCTCAAATTGCTCAGTTTGCTCCATCA
GCGTCTGCCTTCTTCGGGATGTCAAGAATTGGAATGGAAGTAACCCCTTCTGGCACATG
GTTAACATATACTGGTGCAATCAAATTGGATGATAAAGACCCCAATTTCAAAGATCAA
GTTATACTCCTGAACAAGCACATTGATGCTTACAAGACATTTCCTCCAACAGAGCCAAA
GAAGCATCACCATCATCATCACCACCATTGActcgagACGAAG
CoV-60 IBIO201 S-NTD (19-310)_N-CTD (248-366)-8xHis Long-Short
(Similar to CoV-46 with His tag)
(SEQ ID NO: 151)
MGKMASLFATFLVVLVSLSLASESSATTRTQLPPAYTNSFTRGVYYPDKVFRSSVLHSTQD
LFLPFFSNVTWFHAIHVSGTNGTKRFDNPVLPFNDGVYFASTEKSNIIRGWIFGTTLDSKTQS
LLIVNNATNVVIKVCEFQFCNDPFLGVYYHKNNKSWMESEFRVYSSANNCTFEYVSQPFL
MDLEGKQGNFKNLREFVFKNIDGYFKIYSKHTPINLVRDLPQGFSALEPLVDLPIGINITRFQ
TLLALHRSYLTPGDSSSGWTAGAAAYYVGYLQPRTFLLKYNENGTITDAVDCALDPLSET
KCTLKSFTVEKSGGGSKKSAAEASKKPRQKRTATKAYNVTQAFGRRGPEQTQGNFGDQELIRQ
GTDYKHWPQIAQFAPSASAFFGMSRIGMEVTPSGTWLTYTGAIKLDDKDPNFKDQVILLNKHID
AYKTFPPTHHHHHHHH
(SEQ ID NO: 152)
ACGTCAttaattaaATGGGAAAAATGGCTTCTCTTTTTGCTACTTTCCTTGTTGTGTTGGTTAG
TCTTTCTCTAGCTAGTGAGAGTAGTGCTACGACCAGGACACAACTTCCCCCTGCTTATA
CAAACTCTTTCACTAGGGGCGTCTATTACCCTGATAAGGTGTTTCGGAGTAGTGTATTG
CATTCAACTCAAGATTTGTTCCTTCCGTTCTTCTCAAATGTGACATGGTTTCATGCCATT
CATGTCTCAGGGACCAATGGTACAAAGAGATTTGATAATCCTGTTCTCCCATTCAATGA
CGGTGTTTATTTTGCTTCAACAGAGAAGAGCAACATTATAAGAGGATGGATTTTTGGAA
CAACGCTTGACTCAAAAACTCAGAGTTTATTGATAGTAAATAATGCAACTAACGTTGTG
ATCAAAGTTTGTGAATTCCAGTTCTGCAATGATCCATTTCTGGGAGTTTACTATCACAA
GAACAACAAGTCTTGGATGGAAAGTGAGTTCAGAGTATATTCTTCTGCAAACAATTGC
ACTTTTGAGTATGTTTCTCAGCCTTTTTTGATGGATCTTGAGGGGAAACAAGGAAATTT
CAAAAATCTACGCGAATTTGTTTTTAAGAATATTGATGGCTACTTCAAGATTTACAGCA
AGCATACTCCAATAAACCTTGTGAGAGACCTGCCACAGGGATTTTCCGCCTTAGAACC
ACTGGTAGACTTACCAATTGGGATCAATATCACACGTTTTCAAACTCTCCTAGCGTTGC
ACAGGAGTTACCTAACTCCTGGTGATTCTTCGAGCGGTTGGACTGCTGGAGCTGCAGCA
TATTATGTGGGTTATTTGCAACCCCGAACATTTCTTCTCAAATATAATGAAAATGGAAC
CATTACTGATGCTGTTGATTGTGCTTTGGATCCTCTTTCTGAAACAAAATGTACCCTGA
AATCATTTACTGTCGAGAAATCCGGTGGTGGCTCCAAAAAAAGTGCCGCTGAAGCATC
CAAGAAACCGCGACAGAAGAGGACTGCAACAAAAGCTTATAATGTTACTCAAGCATTT
GGGAGACGTGGTCCTGAACAAACTCAGGGAAATTTTGGTGACCAGGAGCTTATAAGGC
AAGGAACTGATTATAAGCATTGGCCTCAAATTGCTCAGTTTGCTCCATCAGCGTCTGCC
TTCTTCGGGATGTCAAGAATTGGAATGGAAGTAACCCCTTCTGGCACATGGTTAACATA
TACTGGTGCAATCAAATTGGATGATAAAGACCCCAATTTCAAAGATCAAGTTATACTCC
TGAACAAGCACATTGATGCTTACAAGACATTTCCTCCAACACATCACCATCATCATCAC
CACCATTGActcgagACGAAG
CoV-61 IBIO201 S-NTD (19-290)_N-CTD (245-370)-8xHis Short-Long
(Similar to CoV-47 with His tag)
(SEQ ID NO: 153)
MGKMASLFATFLVVLVSLSLASESSATTRTQLPPAYTNSFTRGVYYPDKVFRSSVLHSTQD
LFLPFFSNVTWFHAIHVSGTNGTKRFDNPVLPFNDGVYFASTEKSNIIRGWIFGTTLDSKTQS
LLIVNNATNVVIKVCEFQFCNDPFLGVYYHKNNKSWMESEFRVYSSANNCTFEYVSQPFL
MDLEGKQGNFKNLREFVFKNIDGYFKIYSKHTPINLVRDLPQGFSALEPLVDLPIGINITRFQ
TLLALHRSYLTPGDSSSGWTAGAAAYYVGYLQPRTFLLKYNENGTITDAVDSGGGSTVTK
KSAAEASKKPRQKRTATKAYNVTQAFGRRGPEQTQGNFGDQELIRQGTDYKHWPQIAQFAPSAS
AFFGMSRIGMEVTPSGTWLTYTGAIKLDDKDPNFKDQVILLNKHIDAYKTFPPTEPKKHHHHHH
HH
(SEQ ID NO: 154)
ACGTCAttaattaaATGGGAAAAATGGCTTCTCTTTTTGCTACTTTCCTTGTTGTGTTGGTTAG
TCTTTCTCTAGCTAGTGAGAGTAGTGCTACGACCAGGACACAACTTCCCCCTGCTTATA
CAAACTCTTTCACTAGGGGCGTCTATTACCCTGATAAGGTGTTTCGGAGTAGTGTATTG
CATTCAACTCAAGATTTGTTCCTTCCGTTCTTCTCAAATGTGACATGGTTTCATGCCATT
CATGTCTCAGGGACCAATGGTACAAAGAGATTTGATAATCCTGTTCTCCCATTCAATGA
CGGTGTTTATTTTGCTTCAACAGAGAAGAGCAACATTATAAGAGGATGGATTTTTGGAA
CAACGCTTGACTCAAAAACTCAGAGTTTATTGATAGTAAATAATGCAACTAACGTTGTG
ATCAAAGTTTGTGAATTCCAGTTCTGCAATGATCCATTTCTGGGAGTTTACTATCACAA
GAACAACAAGTCTTGGATGGAAAGTGAGTTCAGAGTATATTCTTCTGCAAACAATTGC
ACTTTTGAGTATGTTTCTCAGCCTTTTTTGATGGATCTTGAGGGGAAACAAGGAAATTT
CAAAAATCTACGCGAATTTGTTTTTAAGAATATTGATGGCTACTTCAAGATTTACAGCA
AGCATACTCCAATAAACCTTGTGAGAGACCTGCCACAGGGATTTTCCGCCTTAGAACC
ACTGGTAGACTTACCAATTGGGATCAATATCACACGTTTTCAAACTCTCCTAGCGTTGC
ACAGGAGTTACCTAACTCCTGGTGATTCTTCGAGCGGTTGGACTGCTGGAGCTGCAGCA
TATTATGTGGGTTATTTGCAACCCCGAACATTTCTTCTCAAATATAATGAAAATGGAAC
CATTACTGATGCTGTTGATTCCGGTGGTGGCTCCACGGTGACCAAAAAAAGTGCCGCTG
AAGCATCCAAGAAACCGCGACAGAAGAGGACTGCAACAAAAGCTTATAATGTTACTCA
AGCATTTGGGAGACGTGGTCCTGAACAAACTCAGGGAAATTTTGGTGACCAGGAGCTT
ATAAGGCAAGGAACTGATTATAAGCATTGGCCTCAAATTGCTCAGTTTGCTCCATCAGC
GTCTGCCTTCTTCGGGATGTCAAGAATTGGAATGGAAGTAACCCCTTCTGGCACATGGT
TAACATATACTGGTGCAATCAAATTGGATGATAAAGACCCCAATTTCAAAGATCAAGT
TATACTCCTGAACAAGCACATTGATGCTTACAAGACATTTCCTCCAACAGAGCCAAAG
AAGCATCACCATCATCATCACCACCATTGActcgagACGAAG
CoV-62 IBIQ201S-NTD(19-290)_N-CTD(248-366)-8xHis Short-Short
(Similar to CoV-48 with His tag)
(SEQ ID NO: 155)
MGKMASLFATFLVVLVSLSLASESSATTRTQLPPAYTNSFTRGVYYPDKVFRSSVLHSTQD
LFLPFFSNVTWFHAIHVSGTNGTKRFDNPVLPFNDGVYFASTEKSNIIRGWIFGTTLDSKTQS
LLIVNNATNVVIKVCEFQFCNDPFLGVYYHKNNKSWMESEFRVYSSANNCTFEYVSQPFL
MDLEGKQGNFKNLREFVFKNIDGYFKIYSKHTPINLVRDLPQGFSALEPLVDLPIGINITRFQ
TLLALHRSYLTPGDSSSGWTAGAAAYYVGYLQPRTFLLKYNENGTITDAVDSGGGSKKSA
AEASKKPRQKRTATKAYNVTQAFGRRGPEQTQGNFGDQELIRQGTDYKHWPQIAQFAPSASAFF
GMSRIGMEVTPSGTWLTYTGAIKLDDKDPNFKDQVILLNKHIDAYKTFPPTHHHHHHHH
(SEQ ID NO: 156)
ACGTCAttaattaaATGGGAAAAATGGCTTCTCTTTTTGCTACTTTCCTTGTTGTGTTGGTTAG
TCTTTCTCTAGCTAGTGAGAGTAGTGCTACGACCAGGACACAACTTCCCCCTGCTTATA
CAAACTCTTTCACTAGGGGCGTCTATTACCCTGATAAGGTGTTTCGGAGTAGTGTATTG
CATTCAACTCAAGATTTGTTCCTTCCGTTCTTCTCAAATGTGACATGGTTTCATGCCATT
CATGTCTCAGGGACCAATGGTACAAAGAGATTTGATAATCCTGTTCTCCCATTCAATGA
CGGTGTTTATTTTGCTTCAACAGAGAAGAGCAACATTATAAGAGGATGGATTTTTGGAA
CAACGCTTGACTCAAAAACTCAGAGTTTATTGATAGTAAATAATGCAACTAACGTTGTG
ATCAAAGTTTGTGAATTCCAGTTCTGCAATGATCCATTTCTGGGAGTTTACTATCACAA
GAACAACAAGTCTTGGATGGAAAGTGAGTTCAGAGTATATTCTTCTGCAAACAATTGC
ACTTTTGAGTATGTTTCTCAGCCTTTTTTGATGGATCTTGAGGGGAAACAAGGAAATTT
CAAAAATCTACGCGAATTTGTTTTTAAGAATATTGATGGCTACTTCAAGATTTACAGCA
AGCATACTCCAATAAACCTTGTGAGAGACCTGCCACAGGGATTTTCCGCCTTAGAACC
ACTGGTAGACTTACCAATTGGGATCAATATCACACGTTTTCAAACTCTCCTAGCGTTGC
ACAGGAGTTACCTAACTCCTGGTGATTCTTCGAGCGGTTGGACTGCTGGAGCTGCAGCA
TATTATGTGGGTTATTTGCAACCCCGAACATTTCTTCTCAAATATAATGAAAATGGAAC
CATTACTGATGCTGTTGATTCCGGTGGTGGCTCCAAAAAAAGTGCCGCTGAAGCATCCA
AGAAACCGCGACAGAAGAGGACTGCAACAAAAGCTTATAATGTTACTCAAGCATTTGG
GAGACGTGGTCCTGAACAAACTCAGGGAAATTTTGGTGACCAGGAGCTTATAAGGCAA
GGAACTGATTATAAGCATTGGCCTCAAATTGCTCAGTTTGCTCCATCAGCGTCTGCCTT
CTTCGGGATGTCAAGAATTGGAATGGAAGTAACCCCTTCTGGCACATGGTTAACATAT
ACTGGTGCAATCAAATTGGATGATAAAGACCCCAATTTCAAAGATCAAGTTATACTCCT
GAACAAGCACATTGATGCTTACAAGACATTTCCTCCAACACATCACCATCATCATCACC
ACCATTGActcgagACGAAG
N + S Designs-N CTD + S RBD
N-CTD (248-366) + S-RBD (319-554)
(SEP ID NO: 157)
KKS AAEASKKPRQ KRTATKAYNV TQAFGRRGPE QTQGNFGDQE LIRQGTDYKH
WPQIAQFAPS ASAFFGMSRI GMEYTPSGTW LTYTGAIKLD DKDPNFKDQV ILLNKHIDAY
KTFPPT SGGGS TTRTQLPPAY TNSFTRGVYY PDKVFRSSVL HSTQDLFLPF
FSNVTWFHAI HVSGTNGTKR FDNPVLPFND GVYFASTEKS NIIRGWIFGT TLDSKTQSLL
IVNNATNVVI KVCEFQFCND PFLGVYYHKN NKSWMESEFR VYSSANNCTF
EYVSQPFLMD LEGKQGNEKN LREFVFKNID GYFKIYSKHT PINLVRDLPQ GFSALEPLVD
LPIGINITRF QTLLALHRSY LTPGDSSSGW TAGAAAYYVG YLQPRTFLLK YNENGTIDA
VDCALDPLSE TKCTLKSFTV EK
N-CTD (245-370) + S-RBD (319-554)
(SEQ ID NO: 158)
TVTKKSAAE ASKKPRQKRT ATKAYNVTQA FGRRGPEQTQ GNFGDQEIJR QGTDYKHWPQ
IAQFAPSASA FFGMSRIGME VTPSGTWLTY IGAIKLDDKD PNFKDQVILL NKHIDAYKTF
PPTEPKK SGGGS RVQPTESIVR FPNITNLCPF GEVFNATRFA SVYAWNRKRI
SNCVADYSVL YNSASFSTFK CYGVSPTKLN DLCFTNVYAD SFVIRGDEVR QIAPGQTGKI
ADYNYKLPDD FTGCVIAWNS NNLDSKVGGN YNYLYRLFRK SNLKPFERDI
STEIYQAGST PCNGVEGFNC YFPLQSYGFQ PTNGVGYQPY RVVVLSFELL
HAPATVCGPK KSTNLVKNKC VNFNFNGLTG TGVLTE
N-CTD (248-366) + S-RBD (319-554)
(SEQ ID NO: 159)
KKSAAEASK KPRQKRTATK AYNVTQAFGR RGPEQTQGRF GDQELIRQGT DYKHWPQIAQ
FAPSASAFFG MSRIGMEFTP SGTWLTYTGA IKLDDKDPNF KDQVILLNKH IDAYKTFPPT
SGGGS RVQPTESIVR FPNITNLCPF GEVFNATRFA SVYAWNRKRI SNCVADYSVL
YNSASFSTFK CYGVSPTKLN DLCFTNVYAD SFVIRGDEVR QIAPGQTGKI
ADYNYKLPDD FTGCVIAWNS NNLDSKVGGN YNYLYRLFRK SNLKPFERDI
STEIYQAGST PCNGVEGFNC YFPLQSYGFQ PTNGVGYQPY RVVVLSFELL
HAPATVCGPK KSTNLVKNKC VNFNFNGLTG TGVLTE
N-CTD (245-370) + S-RBD (348-523)
(SEP ID NO: 160)
TVTKKSAAE ASKKPRQKRT ATKAYNVTQA FGRRGPEQTQ GNFGDQEIJR QGTDYKHWPQ
IAQFAPSASA FFGMSRIGME VTPSGTWLTY TGAIKLDDKD PNFKDQVILL NKHIDAYKTF
PPTEPKK SGGGS ASVYAWNRKR ISNCVADYSV LYNSASFSTF KCYGVSPTKL
NDLCFTNVYA DSFVIRGDEV RQIAPGQTGK IADYNYKLPD DFTGCVIAWN
SNNLDSKVGG NYNYLYRLFR KSNLKPFERD ISTEIYQAGS TPCNGVEGFN CYFPLQSYGF
QPTNGVGYQP YRVVVLSFEL LHAPAT
N-CTD (248-366) + S-RBD (319-525)
(SEQ ID NO: 161)
KKSAAEASK KPRQKRTATK AYNVTQAFGR RGPEQTQGNF GDQELIRQGT DYKHWPQIAQ
FAPSASAFFG MSRIGMEVTP SGTWLTYTGA PKEDDKDPNF KDQVILLNKH IDAYKTFPPT
SGGGS
RVQPTESIVRFPNITNLCPFGEVFNATRFASVYAWNRKRISNCVADYSVLYNSASFSTFKCY
GVSPTKLNDLCFTNVYADSFVIRGDEVRQIAPGQTGKIADYNYKLPDDFTGCVIAWNSNNL
DSKVGGNYNYLYRLFRKSNLKPFERDISTEIYQAGSTPCNGVEGFNCYFPLQSYGFQPTNG
VGYQPYRVVVLSFELLHAPATVCGPKK

Mouse Immunization Plan Overview.

10 ug SARS-CoV-2 Nucleocapsid c-terminal domain (CoV-41) as antigen, 7 intramuscular arms (1-7), 7 intranasal arms (8-14).

Regimen. Prime-boost strategy with boost at D21 and sac at D42. Titers at D28, D35 and D42, ELIspot on spleens, FACS on spleens.

Adjuvant strategy. Portfolio of adjuvants to include; Th-1 skewing and Th-2 skewing.

### Group Codes

1/8 CoV41, unadjuvanted
2/9 CoV41+TQL1055 (Adjuvance, US)
3/10 CoV41+SE (IDRI, US)
4/11 CoV41+GLA/SE (IDRI, US)
5/12 CoV41+3M-052/Alum (IDRI, US)
6/13 CoV41+3M-052/SE (IDRI, US)
7/14 CoV41+1 ug CpG oligo (Sigma, US)

FIG. 5 is a graph that shows the results from mouse immunizations with CoV-41 and the immunological skew at day 42 (D42) with the different groups. Intramuscular injection, prime/boost. Seven arms, including unadjuvanted. N-specific IgG2/1 titers by ELISA.

Key Findings. Unadjuvanted N only protein does not lead to a significant titer. Inflammatory Th-2 response has IgG1>IgG2. Th-1 response has IgG1<IgG2. N+GLA/SE (Group 4) has a 50/50 ratio. N+CpG DNA (Group 7) has a 2.5/1 ratio favoring IgG2c. N+SE (Group 3) has a 1/5 ratio, shows Th-2 skew and high likelihood for immune pathology.

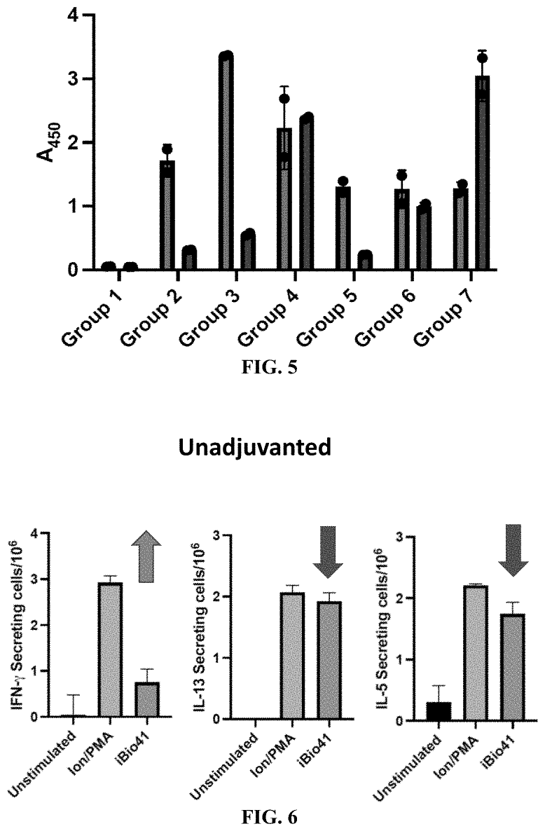

FIG. 6 are graphs that show the results from mouse immunizations with CoV-41 and show the T cell priming by ELIspot. Intramuscular injection, prime/boost. Seven arms, including unadjuvanted. Spleen cell response (mixed immune cells).

Key Findings. Re-exposure to N protein does not lead to robust IFN response in mice immunized without adjuvant. Unadjuvanted N only protein does lead to robust IL-5 and IL-13 responses, consistent with an inflammatory response to the antigen. Low IFN/High IL-13/5 memory response is contra-indicated in the context of a COVID vaccine, as this is an inflammatory response.

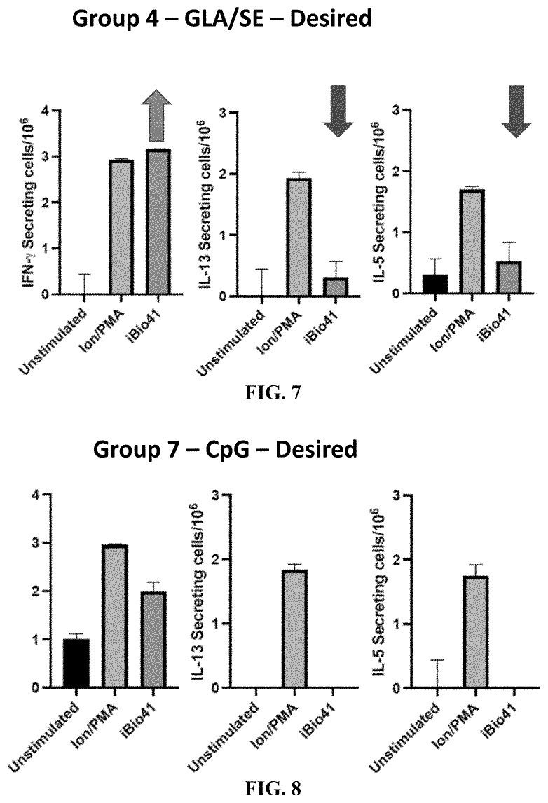

FIG. 7 are graphs that show the results from mouse immunizations with CoV-41 and show the T cell priming by ELIspot. Intramuscular injection, prime/boost. Spleen cell response (mixed immune cells).

Key Findings, Group 4. Re-exposure to N protein does lead to robust IFN response in mice immunized with N+GLA/SE. N+GLA/SE does not lead to robust IL-5 and IL-13 responses. High IFN/Low IL-13/5 is desirable in the context of COVID vaccine, as this is a Th-1 skew, not an inflammatory response.

FIG. 8 are graphs that show the results from mouse immunizations with CoV-41 and show the T cell priming by ELIspot. Intramuscular injection, prime/boost. Spleen cell response (mixed immune cells).

Key Findings, Group 7. Re-exposure to N protein does lead to increased IFN response in mice immunized with N+CpG. N+CpG does not lead to robust IL-5 and IL-13 responses.

Increased IFN/Low IL-13/5 is desirable in the context of COVID vaccine, as this is a Th-1 skew, not an inflammatory response.

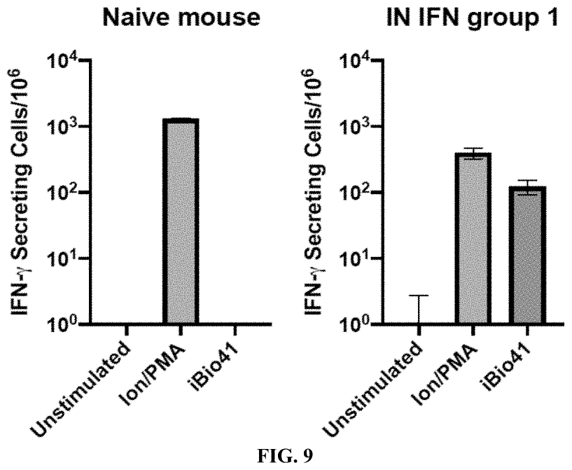

FIG. 9 are graphs that show the results from mouse immunizations with CoV-41 and show the T cell priming by ELIspot. Intranasal (IN) administration, prime/boost. Seven arms, including unadjuvanted. Spleen cell response (mixed immune cells). IFN responses were tested in apparently non-responsive mice based on IgG titers.

Key Findings. Unadjuvanted N protein does lead to increased IFN release in mice immunized without adjuvant, which was not observed for intramuscular arm. To verify the surprising result of IFN release from mice without substantial anti-N IgG titers, a naïve mouse's spleen cells were evaluated for response to N; no stimulation was observed.

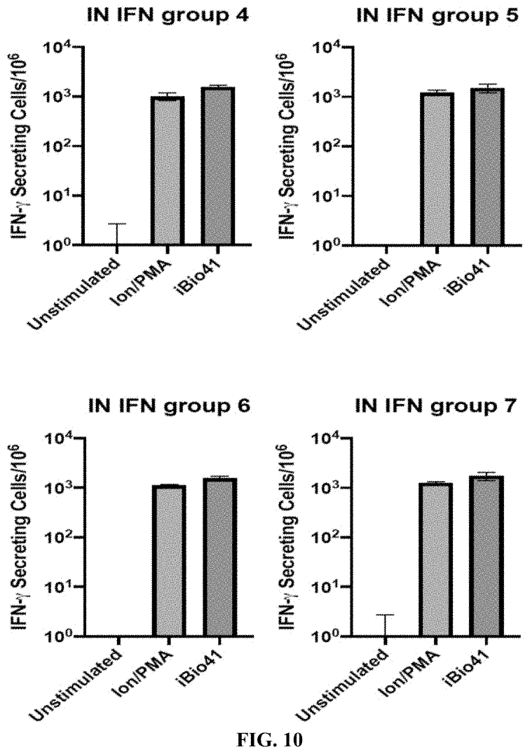

FIG. 10 are graphs that show the results from mouse immunizations with CoV-41 and show the T cell priming by ELIspot. Intranasal (IN) administration, prime/boost. Seven arms, including unadjuvanted. Spleen cell response (mixed immune cells).

Key Findings/Summary. Strong IFN release was observed in every group of mice immunized intranasally, for every adjuvant. As examples, Groups 5 and 6 did not show IFN release of this magnitude in intramuscular arms. Prevalence and magnitude of the response may allow for less antigen to be used.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present.

The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

What is claimed is:

1. An immunogenic protein comprising at least 90% amino acid identity to an amino acid sequence of at least one antigenic peptide selected from: a coronavirus Receptor Binding Domain (RBD), coronavirus a Receptor Binding Motif (RBM) of a coronavirus spike protein, a coronavirus spike protein N-terminus, a nucleocapsid protein, one or more T cell epitopes from a coronavirus spike protein, or one or more T cell epitopes from a coronavirus nucleocapsid protein, or combination thereof.

2. The immunogenic protein of claim 1, further comprising at least one of:
a carrier protein or peptide tag, wherein the at least one antigenic peptide is positioned at, at least one of, an N-terminus, a C-terminus, or in a loop region of the carrier protein,
a carrier protein selected from a modified thermostable lichenase (LicKM), a human hepatitis core antigen (HBcAg), or a truncated woodchuck hepatitis core antigen (WHcAg);
the immunogenic protein is formulated into an immunization;
further comprising an adjuvant selected from at least one of alum, aluminum hydroxide, aluminum phosphate, calcium phosphate hydroxide, cytosine-guanosine oligonucleotide (CpG-ODN) sequence, granulocyte macrophage colony stimulating factor (GM-CSF), monophosphoryl lipid A (MPL), poly(I:C), MF59, Quil A, N-acetyl muramyl-L-alanyl-D-isoglutamine (MDP), FIA, montanide, poly (DL-lactide-coglycolide), squalene, glucopyranosyl lipid adjuvant (GLA), GLA-Alum, 3M-052, a glucopyranosyl lipid adjuvant GLA emulsion with squalene (GLA-SE), virosome, AS03, ASO4, IL-1, IL-2, IL-3, IL-4, IL-5, IL-6, IL-7, IL-8, IL-10, IL-12, IL-15, IL-17, IL-18, STING, CD40L, pathogen-associated molecular patterns (PAMPs), damage-associated molecular pattern molecules (DAMPs), Freund's complete adjuvant, Freund's incomplete adjuvant, transforming growth factor (TGF)-beta antibody or antagonists, A2aR antagonists, lipopolysaccharides (LPS), Fas ligand, Trail, lymphotactin, Mannan (M-FP), APG-2, Hsp70 and Hsp90, pattern recognition receptor ligands, TLR3 ligands, TLR4 ligands, TLR5 ligands, TLR7/8 ligands, or TLR9 ligands; or
the immunogenic protein is further modified to include one or more engineered glycosylation sites, or less disulfide forming residues.

3. The immunogenic protein of claim 1, wherein the at least one antigenic peptide is a fusion protein is selected from at least one of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 79, 81, 83, 85, 87, 91, 93, 95, 97, 99, 101, 103, 105, 137, 139, 141, 43, 145, 147, 149, 151, 153, 155, 157, 158, 159, 160, or 161.

4. The immunogenic protein of claim 1, wherein the immunogenic protein is encoded by a nucleic acid selected from at least one of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 107, 108, 109, 110, 111, 112, 113, 114, 138, 140, 142, 144, 146, 148, 150, 152, 154, or 156.

5. The immunogenic protein of claim 1, wherein the coronavirus is SARS, MERS, 229E (alpha), NL63 (alpha), OC43 (beta), HKU1 (beta), or SARS-CoV-2 variants including the Wuhan parental sequence with or without the D614G mutation, Alpha (B.1.1.7 and Q lineages), Beta (B.1.351 and descendent lineages), Gamma (P.1 and descendent lineages), Epsilon (B.1.427 and B.1.429), Eta (B.1.525), Iota (B.1.526), Kappa (B.1.617.1), Mu (B.1.621, B.1.621.1), Zeta (P.2), Delta (B.1.617.2 and AY lineages), and Omicron (B.1.1.529) at least one of variants BA.1, BA.2, or BA.3.

6. A method of stimulating an immune response in an animal comprising administering to the animal a composition comprising an immunogenic protein that has at least 90% amino acid identity to at least one antigenic peptide selected from: a coronavirus Receptor Binding Domain (RBD), coronavirus a Receptor Binding Motif (RBM) of a coronavirus spike protein, a coronavirus spike protein N-terminus, a nucleocapsid protein, one or more T cell epitopes from a coronavirus spike protein, or one or more T cell epitopes from a coronavirus nucleocapsid protein, or combination thereof.

7. The method of claim 6, further comprising at least one of:
wherein a carrier protein or peptide tag, wherein the at least one antigenic peptide is positioned at, at least one of, an N-terminus, a C-terminus, or in a loop region of the carrier protein,
wherein a carrier protein selected from a modified thermostable lichenase (LicKM), a human hepatitis core antigen (HBcAg), or a truncated woodchuck hepatitis core antigen (WHcAg);
wherein the immunogenic protein is formulated into an immunization;
further comprising an adjuvant selected from at least one of alum, aluminum hydroxide, aluminum phosphate, calcium phosphate hydroxide, cytosine-guanosine oligonucleotide (CpG-ODN) sequence, granulocyte macrophage colony stimulating factor (GM-CSF), monophosphoryl lipid A (MPL), poly(I:C), MF59, Quil A, N-acetyl muramyl-L-alanyl-D-isoglutamine (MDP), FIA, montanide, poly (DL-lactide-coglycolide), squalene, glucopyranosyl lipid adjuvant (GLA), GLA-Alum, 3M-052, a glucopyranosyl lipid adjuvant GLA emulsion with squalene (GLA-SE), virosome, AS03, ASO4, IL-1, IL-2, IL-3, IL-4, IL-5, IL-6, IL-7, IL-8, IL-10, IL-12, IL-15, IL-17, IL-18, STING, CD40L, pathogen-associated molecular patterns (PAMPs), damage-associated molecular pattern molecules (DAMPs), Freund's complete adjuvant, Freund's incomplete adjuvant, transforming growth factor (TGF)-beta antibody or antagonists, A2aR antagonists, lipopolysaccharides (LPS), Fas ligand, Trail, lymphotactin, Mannan (M-FP), APG-2, Hsp70 and Hsp90, pattern recognition receptor ligands, TLR3 ligands, TLR4 ligands, TLR5 ligands, TLR7/8 ligands, or TLR9 ligands;
wherein the immunogenic protein is further modified to include one or more engineered glycosylation sites, or less disulfide forming residues; or
wherein the immune response is at least one of: a humoral immune response, a cellular immune response, or an innate immune response.

8. The method of claim 6, wherein the at least one antigenic peptide is a fusion protein is selected from at least one of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 79, 81, 83, 85, 87, 91, 93, 95, 97, 99, 101, 103, 105, 137, 139, 141, 43, 145, 147, 149, 151, 153, 155, 157, 158, 159, 160, or 161.

9. The method of claim 6, wherein the immunogenic protein is encoded by a nucleic acid selected from at least one of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 107, 108, 109, 110, 111, 112, 113, 114, 138, 140, 142, 144, 146, 148, 150, 152, 154, or 156.

10. The method of claim 6, wherein the coronavirus is SARS, MERS, 229E (alpha), NL63 (alpha), OC43 (beta), HKU1 (beta), or SARS-CoV-2 variants including the Wuhan parental sequence with or without the D614G mutation, Alpha (B.1.1.7 and Q lineages), Beta (B.1.351 and descendent lineages), Gamma (P.1 and descendent lineages), Epsilon (B.1.427 and B.1.429), Eta (B.1.525), Iota (B.1.526), Kappa (B.1.617.1), Mu (B.1.621, B.1.621.1), Zeta (P.2), Delta (B.1.617.2 and AY lineages), and Omicron (B.1.1.529) at least one of variants BA.1, BA.2, or BA.3.

11. A method for production of a carrier protein in a plant comprising:
(a) providing a plant containing an expression cassette having a nucleic acid encoding an immunogenic protein that has at least 90% amino acid identity to at least one antigenic peptide selected from: a coronavirus Receptor Binding Domain (RBD), coronavirus a Receptor Binding Motif (RBM) of a coronavirus spike protein, a coronavirus spike protein N-terminus, a nucleocapsid protein, one or more T cell epitopes from a coronavirus spike protein, or one or more T cell epitopes from a coronavirus nucleocapsid protein, or combination thereof; and
(b) growing the plant under conditions in which the nucleic acid is expressed and the immunogenic protein is produced.

12. The method of claim 11, further comprising at least one of:
a carrier protein or peptide tag, wherein the at least one antigenic peptide is positioned at, at least one of, an N-terminus, a C-terminus, or in a loop region of the carrier protein, a carrier protein selected from a modified thermostable lichenase (LicKM), a human hepatitis core antigen (HBcAg), or a truncated woodchuck hepatitis core antigen (WHcAg);
the immunogenic protein is formulated into an immunization;
further comprising an adjuvant selected from at least one of alum, aluminum hydroxide, aluminum phosphate, calcium phosphate hydroxide, cytosine-guanosine oligonucleotide (CpG-ODN) sequence, granulocyte macrophage colony stimulating factor (GM-CSF), monophosphoryl lipid A (MPL), poly(I:C), MF59, Quil A, N-acetyl muramyl-L-alanyl-D-isoglutamine (MDP), FIA, montanide, poly (DL-lactide-coglycolide), squalene, glucopyranosyl lipid adjuvant (GLA), GLA-Alum, 3M-052, a glucopyranosyl lipid adjuvant GLA emulsion with squalene (GLA-SE), virosome, AS03, ASO4, IL-1, IL-2, IL-3, IL-4, IL-5, IL-6, IL-7, IL-8, IL-10, IL-12, IL-15, IL-17, IL-18, STING, CD40L, pathogen-associated molecular patterns (PAMPs), damage-associated molecular pattern molecules (DAMPs), Freund's complete adjuvant, Freund's incomplete adjuvant, transforming growth factor (TGF)-beta antibody or antagonists, A2aR antagonists, lipopolysaccharides (LPS), Fas ligand, Trail, lymphotactin, Mannan (M-FP), APG-2, Hsp70 and Hsp90, pattern recognition receptor ligands, TLR3 ligands, TLR4 ligands, TLR5 ligands, TLR7/8 ligands, or TLR9 ligands; or
the immunogenic protein is further modified to include one or more engineered glycosylation sites, or less disulfide forming residues.

13. The method of claim 11, further comprising at least one of:
recovering the immunogenic protein;
wherein a promoter is selected from the group consisting of plant constitutive promoters and plant tissue specific promoters;
wherein the immunogenic protein is expressed in leaf, root, fruit, tubercle or seed of a plant;
wherein a plant is a Nicotiana sp. plant; or
wherein the immunogenic protein is formulated into an immunization.

14. The method of claim 11, wherein the at least one antigenic peptide is a fusion protein is selected from at least one of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 79, 81, 83, 85, 87, 91, 93, 95, 97, 99, 101, 103, 105, 137, 139, 141, 43, 145, 147, 149, 151, 153, 155, 157, 158, 159, 160, or 161.

15. The method of claim 11, wherein the immunogenic protein is encoded by a nucleic acid selected from at least one of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 107, 108, 109, 110, 111, 112, 113, 114, 138, 140, 142, 144, 146, 148, 150, 152, 154, or 156.

16. The method of claim 11, wherein the coronavirus is SARS, MERS, 229E (alpha), NL63 (alpha), OC43 (beta), HKU1 (beta), or SARS-CoV-2 variants including the Wuhan parental sequence with or without the D614G mutation, Alpha (B.1.1.7 and Q lineages), Beta (B.1.351 and descendent lineages), Gamma (P.1 and descendent lineages), Epsilon (B.1.427 and B.1.429), Eta (B.1.525), Iota (B.1.526), Kappa (B.1.617.1), Mu (B.1.621, B.1.621.1), Zeta (P.2), Delta (B.1.617.2 and AY lineages), and Omicron (B.1.1.529) at least one of variants BA.1, BA.2, or BA.3.

17. The method of claim 11, wherein the antigen is administered intranasally and only triggers a T cell response.

18. A nucleic acid encoding a protein comprising:
an immunogenic protein that has at least 90% amino acid identity to at least one antigenic peptide selected from: a coronavirus Receptor Binding Domain (RBD), coronavirus a Receptor Binding Motif (RBM) of a coronavirus spike protein, a coronavirus spike protein N-terminus, a nucleocapsid protein, one or more T cell epitopes from a coronavirus spike protein, or one or more T cell epitopes from a coronavirus nucleocapsid protein, or combination thereof.

19. The nucleic acid of claim 18, further comprising at least one of:
a carrier protein or peptide tag, wherein the at least one immunogenic protein is positioned at, at least one of, an N-terminus, a C-terminus, or in a loop region of the carrier protein or peptide tag;
wherein the nucleic acid further comprises a promoter for plant cell expression;
wherein the nucleic acid further comprises a plant promoter selected from one or more plant constitutive promoters, and one or more plant tissue specific promoters;
wherein the at least one antigenic peptide is expressed in a leaf, root, fruit, tubercle or seed of a plant;
wherein the at least one antigenic peptide is inserted into a recombinant RNA viral vector has a recombinant genomic component of a tobamovirus, an alfalfa mosaic virus, an ilarvirus, a cucumovirus or a closterovirus; or
wherein a host plant is a dicotyledon or a monocotyledon.

20. The nucleic acid of claim 18, wherein the coronavirus is wherein the coronavirus is SARS, MERS, 229E (alpha), NL63 (alpha), OC43 (beta), HKU1 (beta), or SARS-CoV-2 variants including the Wuhan parental sequence with or without the D614G mutation, Alpha (B.1.1.7 and Q lineages), Beta (B.1.351 and descendent lineages), Gamma (P.1 and descendent lineages), Epsilon (B.1.427 and B.1.429), Eta (B.1.525), Iota (B.1.526), Kappa (B.1.617.1), Mu (B.1.621, B.1.621.1), Zeta (P.2), Delta (B.1.617.2 and AY lineages), and Omicron (B.1.1.529) at least one of variants BA.1, BA.2, or BA.3.

21. The nucleic acid of claim 18, wherein the nucleic acid is selected from at least one of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 107, 108, 109, 110, 111, 112, 113, 114, 138, 140, 142, 144, 146, 148, 150, 152, 154, or 156.

22. The nucleic acid of claim 18, wherein the nucleic acid encodes a protein selected from at least one of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 79, 81, 83, 85, 87, 91, 93, 95, 97, 99, 101, 103, 105, 137, 139, 141, 43, 145, 147, 149, 151, 153, 155, 157, 158, 159, 160, or 161.

23. A vector that comprises a nucleic acid that encodes an immunogenic protein that has at least 90% amino acid identity to at least one antigenic peptide selected from: a coronavirus Receptor Binding Domain (RBD), coronavirus a Receptor Binding Motif (RBM) of a coronavirus spike protein, a coronavirus spike protein N-terminus, a nucleocapsid protein, one or more T cell epitopes from a coronavirus spike protein, or one or more T cell epitopes from a coronavirus nucleocapsid protein, or combination thereof, and optionally, wherein the at least one an immunogenic protein is positioned at, at least one of, the N-terminus, the C-terminus, or in a loop region of a carrier protein or peptide tag.

24. A host cell that comprises a vector that expresses an immunogenic protein that has at least 90% amino acid identity to at least one antigenic peptide selected from: a coronavirus Receptor Binding Domain (RBD), coronavirus a Receptor Binding Motif (RBM) of a coronavirus spike protein, a coronavirus spike protein N-terminus, a nucleocapsid protein, one or more T cell epitopes from a coronavirus spike protein, or one or more T cell epitopes from a coronavirus nucleocapsid protein, or combination thereof, and optionally, wherein the at least one immunogenic protein is positioned at, at least one of, an N-terminus, a C-terminus, or in a loop region of the carrier protein or peptide tag.

25. A pan-coronavirus booster comprising:
an immunogenic protein comprising at least 90% amino acid identity to an amino acid sequence of a coronavirus nucleocapsid protein and adjuvant that triggers a Th1 immune response.

26. The pan-coronavirus booster of claim 25, wherein at least one of:
the pan-coronavirus booster is adapted for intramuscular or intranasal administration;
the pan-coronavirus booster triggers a Th1 immune response;
the Th1 immune response shows a high secretion of IFN and low secretion of IL-13, IL-5, or both when compared to a non-immunized subject or a subject with a TH2 immune response;
the coronavirus is SARS, MERS, 229E (alpha), NL63 (alpha), OC43 (beta), HKU1 (beta), or SARS-CoV-2 variants including the Wuhan parental sequence with or without the D614G mutation, Alpha (B.1.1.7 and Q lineages), Beta (B.1.351 and descendent lineages), Gamma (P.1 and descendent lineages), Epsilon (B.1.427 and B.1.429), Eta (B.1.525), Iota (B.1.526), Kappa (B.1.617.1), Mu (B.1.621, B.1.621.1), Zeta (P.2), Delta (B.1.617.2 and AY lineages), and Omicron (B.1.1.529) at least one of variants BA.1, BA.2, or BA.3;
wherein the immunogenic protein only triggers a T cell response when administered intranasally without an adjuvant;
wherein the immunogenic protein is administered intramuscularly with an adjuvant and intranasally without an adjuvant;
wherein the immunogenic protein is administered with an adjuvant that triggers a Th1 immune response; or
wherein the immunogenic protein is administered to a subject previously immunized with a coronavirus vaccine.


SEQUENCE LISTING (141 sequences)

SEQ ID NO: 1 - protein, 220 aa, Artificial Sequence
  Synthetic
MDIDPYKEFGATVELLSFLPSDFFPSVRDLLDTASALYREALESPEHCSPHHTALRQAILCWGELMTLATWVGANLEDPASRDLVVNYVNTNMGLKIRQLLWFHISCLTFGRETVLEYLVSFGVWIRTPPAYRPPNAPILSTLPSGGSNSNNLDSKVGGNYNYLYRLFRKSNLKPFERDISTEIYQAGSTPCNGVEGFNCYFPLQSYGFQPTNGVGYQPY

SEQ ID NO: 2 - DNA, 677 bp, Artificial Sequence
  Synthetic
ttaattaaatggacatcgatccgtacaaagaatttggcgcgaccgtcgagctgctgagcttcctgccgagcgattttttcccgagcgtgcgtgacctgctggacaccgcgagcgcactgtatcgtgaagcactggaaagcccagagcactgtagcccgcaccacaccgccctgcgccaggcgattctgtgctggggtgaactgatgaccctggccacctgggtgggtgctaaccttgaggatccggcgagccgtgatctggtcgtcaactatgtgaataccaacatgggtctgaaaattcgtcagctgctgtggtttcatattagctgcctgaccttcggtcgtgaaaccgtgctggagtatctggtgagcttcggtgtgtggattcgcaccccgccggcgtatcgtccgccgaacgcgccaattctgagcacgctgccgtccggaggtagcaactctaacaacctggactctaaggttggcggcaactacaactacctctacaggctgttccggaagtccaaccttaagcctttcgagagggatatcagcaccgagatctatcaggctggttctactccttgcaacggtgttgagggtttcaactgctacttcccgcttcagtcttacggtttccagcctactaatggtgtgggctaccagccttattagctcgag

SEQ ID NO: 3 - protein, 221 aa, Artificial Sequence
  Synthetic
MDIDPYKEFGATVELLSFLPSDFFPSVRDLLDTASALYREALESPEHCSPHHTALRQAILCWGELMTLATWVGANLEDPNSNNLDSKVGGNYNYLYRLFRKSNLKPFERDISTEIYQAGSTPCNGVEGFNCYFPLQSYGFQPTNGVGYQPYASRDLVVNYVNTNMGLKIRQLLWFHISCLTFGRETVLEYLVSFGVWIRTPPAYRPPNAPILSTLPETTVV

SEQ ID NO: 4 - DNA, 680 bp, Artificial Sequence
  Synthetic
ttaattaaatggacatcgatccgtacaaagaatttggcgcgaccgtcgagctgctgagcttcctgccgagcgattttttcccgagcgtgcgtgacctgctggacaccgcgagcgcactgtatcgtgaagcactggaaagcccagagcactgtagcccgcaccacaccgccctgcgccaggcgattctgtgctggggtgaactgatgaccctggccacctgggtgggtgctaaccttgaggatccgaactctaacaacctggactctaaggttggcggcaactacaactacctctacaggctgttccggaagtccaaccttaagcctttcgagagggatatcagcaccgagatctatcaggctggttctactccttgcaacggtgttgagggtttcaactgctacttcccgcttcagtcttacggtttccagcctactaatggtgtgggctaccagccttatgctagccgtgatctggtcgtcaactatgtgaataccaacatgggtctgaaaattcgtcagctgctgtggtttcatattagctgcctgaccttcggtcgtgaaaccgtgctggagtatctggtgagcttcggtgtgtggattcgcaccccgccggcgtatcgtccgccgaacgcgccaattctgagcacgctgccggagaccaccgtggtttagctcgag

SEQ ID NO: 5 - protein, 250 aa, Artificial Sequence
  Synthetic
MGKMASLFATFLVVLVSLSLASESSADIDPYKEFGSSYQLLNFLPLDFFPDLNALVDTATALYEEELTGREHCSPHHTAIRQALVCWDELTKLIAWMSSNITSEQVRTIIVNHVNDTWGLKVRQSLWFHLSCLTFGQHTVQEFLVSFGVWIRTPAPYRPPNAPILSTLPEHTVISGGSNSNNLDSKVGGNYNYLYRLFRKSNLKPFERDISTEIYQAGSTPCNGVEGFNCYFPLQSYGFQPTNGVGYQPY

SEQ ID NO: 6 - DNA, 767 bp, Artificial Sequence
  Synthetic
ttaattaaatgggaaaaatggcttctctttttgctactttccttgttgtgttggttagtctttctctagctagtgagagtagtgctgatatcgatccttataaggaattcggttcttcataccaacttttgaattttcttcctttggatttctttccagatcttaatgctttggttgatactgctacagctctttatgaagaggaattgactggaagagagcattgttctccacatcatacagctattaggcaagctttggtttgctgggatgagcttactaagttgattgcttggatgtcttcaaacatcacttcagaacaagttagaacaattattgttaaccatgttaatgatacatggggtcttaaagttaggcaatctttgtggttccatctttcatgtttgacttttggacaacatacagttcaagagtttcttgtttcttttggtgtttggattagaactccagctccttataggccacctaatgctcctattctttctactttgccagaacatacagttatttccggaggtagcaactctaacaacctggactctaaggttggcggcaactacaactacctctacaggctgttccggaagtccaaccttaagcctttcgagagggatatcagcaccgagatctatcaggctggttctactccttgcaacggtgttgagggtttcaactgctacttcccgcttcagtcttacggtttccagcctactaatggtgtgggctaccagccttattagctcgag

SEQ ID NO: 7 - protein, 250 aa, Artificial Sequence
  Synthetic
MGKMASLFATFLVVLVSLSLASESSADIDPYKEFGSSYQLLNFLPLDFFPDLNALVDTATALYEEELTGREHCSPHHTAIRQALVCWDELTKLIAWMSSNITSEENSNNLDSKVGGNYNYLYRLFRKSNLKPFERDISTEIYQAGSTPCNGVEGFNCYFPLQSYGFQPTNGVGYQPYEEEQVRTIIVNHVNDTWGLKVRQSLWFHLSCLTFGQHTVQEFLVSFGVWIRTPAPYRPPNAPILSTLPEHTVI

SEQ ID NO: 8 - DNA, 767 bp, Artificial Sequence
  Synthetic
ttaattaaatgggaaaaatggcttctctttttgctactttccttgttgtgttggttagtctttctctagctagtgagagtagtgctgatatcgatccttataaggaattcggttcttcataccaacttttgaattttcttcctttggatttctttccagatcttaatgctttggttgatactgctacagctctttatgaagaggaattgactggaagagagcattgttctccacatcatacagctattaggcaagctttggtttgctgggatgagcttactaagttgattgcttggatgtcttcaaacatcacttcagaagagaactctaacaacctggactctaaggttggcggcaactacaactacctctacaggctgttccggaagtccaaccttaagcctttcgagagggatatcagcaccgagatctatcaggctggttctactccttgcaacggtgttgagggtttcaactgctacttcccgcttcagtcttacggtttccagcctactaatggtgtgggctaccagccttatgaagaggaacaagttagaacaattattgttaaccatgttaatgatacatggggtcttaaagttaggcaatctttgtggttccatctttcatgtttgacttttggacaacatacagttcaagagtttcttgtttcttttggtgtttggattagaactccagctccttataggccacctaatgctcctattctttctactttgccagaacatacagttatttagctcgag

SEQ ID NO: 9 - protein, 246 aa, Artificial Sequence
  Synthetic
MGKMASLFATFLVVLVSLSLASESSADIDPYKEFGSSYQLLNFLPLDFFPDLNALVDTATALYEEELTGREHCSPHHTAIRQALVCWDELTKLIAWMSSNITSNSNNLDSKVGGNYNYLYRLFRKSNLKPFERDISTEIYQAGSTPCNGVEGFNCYFPLQSYGFQPTNGVGYQPYEQVRTIIVNHVNDTWGLKVRQSLWFHLSCLTFGQHTVQEFLVSFGVWIRTPAPYRPPNAPILSTLPEHTVI

SEQ ID NO: 10 - DNA, 755 bp, Artificial Sequence
  Synthetic
ttaattaaatgggaaaaatggcttctctttttgctactttccttgttgtgttggttagtctttctctagctagtgagagtagtgctgatatcgatccttataaggaattcggttcttcataccaacttttgaattttcttcctttggatttctttccagatcttaatgctttggttgatactgctacagctctttatgaagaggaattgactggaagagagcattgttctccacatcatacagctattaggcaagctttggtttgctgggatgagcttactaagttgattgcttggatgtcttcaaacatcacttcaaactctaacaacctggactctaaggttggcggcaactacaactacctctacaggctgttccggaagtccaaccttaagcctttcgagagggatatcagcaccgagatctatcaggctggttctactccttgcaacggtgttgagggtttcaactgctacttcccgcttcagtcttacggtttccagcctactaatggtgtgggctaccagccttatgaacaagttagaacaattattgttaaccatgttaatgatacatggggtcttaaagttaggcaatctttgtggttccatctttcatgtttgacttttggacaacatacagttcaagagtttcttgtttcttttggtgtttggattagaactccagctccttataggccacctaatgctcctattctttctactttgccagaacatacagttatttagctcgag

SEQ ID NO: 11 - protein, 250 aa, Artificial Sequence
  Synthetic
MGKMASLFATFLVVLVSLSLASESSADIDPYKEFGSSYQLLNFLPLDFFPDLNALVDTATALYEEELTGREHCSPHHTAIRQALVCWDELTKLIAWMSSEENSNNLDSKVGGNYNYLYRLFRKSNLKPFERDISTEIYQAGSTPCNGVEGFNCYFPLQSYGFQPTNGVGYQPYEENITSEQVRTIIVNHVNDTWGLKVRQSLWFHLSCLTFGQHTVQEFLVSFGVWIRTPAPYRPPNAPILSTLPEHTVI

SEQ ID NO: 12 - DNA, 767 bp, Artificial Sequence
  Synthetic
ttaattaaatgggaaaaatggcttctctttttgctactttccttgttgtgttggttagtctttctctagctagtgagagtagtgctgatatcgatccttataaggaattcggttcttcataccaacttttgaattttcttcctttggatttctttccagatcttaatgctttggttgatactgctacagctctttatgaagaggaattgactggaagagagcattgttctccacatcatacagctattaggcaagctttggtttgctgggatgagcttactaagttgattgcttggatgtcttcagaagagaactctaacaacctggactctaaggttggcggcaactacaactacctctacaggctgttccggaagtccaaccttaagcctttcgagagggatatcagcaccgagatctatcaggctggttctactccttgcaacggtgttgagggtttcaactgctacttcccgcttcagtcttacggtttccagcctactaatggtgtgggctaccagccttatgaagagaacatcacttcagaacaagttagaacaattattgttaaccatgttaatgatacatggggtcttaaagttaggcaatctttgtggttccatctttcatgtttgacttttggacaacatacagttcaagagtttcttgtttcttttggtgtttggattagaactccagctccttataggccacctaatgctcctattctttctactttgccagaacatacagttatttagctcgag

SEQ ID NO: 13 - protein, 440 aa, Artificial Sequence
  Synthetic
MHHHHHHHHGGSYPYKSGEYRTKSFFGYGYYEVRMKAAKNVGIVSSFFTYTGPSDNNPWDEIDIEFLGKDTTKVQFNWYKNGVGGNEYLHNLGFDASQDFHTYGFEWRPDYIDFYVDGKKVYRGTRNIPVTPGKIMMNLWPGIGVDEWLGRYDGRTPLQAEYEYVKYYPNGRSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKFKLVVNTPFVAVFSNFDSSQWEKADWANGSVFNCVWKPSQVTFSNGKMILTLDREY

SEQ ID NO: 14 - DNA, 1337 bp, Artificial Sequence
  Synthetic
ttaattaaatgcatcaccatcaccaccatcatcatggcggctcttacccttataagagcggtgagtaccggaccaagagcttctttggttacggttactacgaggtgcggatgaaggctgctaagaacgtgggtatcgtgtccagcttctttacctacaccgggccatctgataacaacccttgggatgagatcgacatcgagttccttggtaaggacactaccaaggtgcagttcaactggtacaagaacggtgttggtggcaacgagtaccttcacaaccttggctttgatgccagccaggatttccacacttacggttttgaatggcggcctgactacatcgacttctacgtggacggtaagaaggtgtacaggggcaccagaaatatccctgtgactcctggcaagatcatgatgaacctttggcctggtatcggtgtggatgagtggcttggtagatacgatggtaggactcctctgcaggctgagtacgagtacgttaagtactaccctaacggcagatctagggttcagcctactgagtctattgtgcggttcccgaacatcaccaacttgtgcccttttggcgaggtgttcaatgctaccaggttcgcttctgtgtacgcctggaatcggaagcggatttctaactgcgtggccgattacagcgtgctgtacaactctgctagcttcagcaccttcaagtgctacggtgtgtctcctaccaagctgaacgatctgtgcttcaccaacgtgtacgctgactctttcgtgatcaggggtgatgaggttaggcagattgctcctggtcagaccggtaagatcgctgactacaactacaagctgcctgatgacttcaccggttgcgtgatcgcttggaactctaacaacctggactctaaggttggcggcaattacaactacctctaccggctgttccggaagtctaaccttaagcctttcgagcgggatatcagcaccgagatctatcaggctggttctactccttgcaatggcgttgagggtttcaactgctacttcccgcttcagtcttacggattccagcctactaatggtgtgggctaccagccttacagagtggtggttttgtctttcgagcttctgcatgctcctgctactgtttgcggtccgaagaagttcaagcttgtcgttaatacccctttcgtggccgtgttcagcaacttcgattctagccagtgggagaaagctgattgggctaacggttctgtgttcaactgcgtgtggaagccttctcaggtgaccttctctaacggcaagatgattctgaccctggaccgtgagtattagctcgag

SEQ ID NO: 15 - protein, 301 aa, Artificial Sequence
  Synthetic
MHHHHHHHHGGSYPYKSGEYRTKSFFGYGYYEVRMKAAKNVGIVSSFFTYTGPSDNNPWDEIDIEFLGKDTTKVQFNWYKNGVGGNEYLHNLGFDASQDFHTYGFEWRPDYIDFYVDGKKVYRGTRNIPVTPGKIMMNLWPGIGVDEWLGRYDGRTPLQAEYEYVKYYPNGRSNSNNLDSKVGGNYNYLYRLFRKSNLKPFERDISTEIYQAGSTPCNGVEGFNCYFPLQSYGFQPTNGVGYQPYFKLVVNTPFVAVFSNFDSSQWEKADWANGSVFNCVWKPSQVTFSNGKMILTLDREY

SEQ ID NO: 16 - DNA, 920 bp, Artificial Sequence
  Synthetic
ttaattaaatgcatcaccatcaccaccatcatcatggcggctcttacccttataagagcggtgagtaccggaccaagagcttctttggttacggttactacgaggtgcggatgaaggctgctaagaacgtgggtatcgtgtccagcttctttacctacaccgggccatctgataacaacccttgggatgagatcgacatcgagttccttggtaaggacactaccaaggtgcagttcaactggtacaagaacggtgttggtggcaacgagtaccttcacaaccttggctttgatgccagccaggatttccacacttacggttttgaatggcggcctgactacatcgacttctacgtggacggtaagaaggtgtacaggggcaccagaaatatccctgtgactcctggcaagatcatgatgaacctttggcctggtatcggtgtggatgagtggcttggtagatacgatggtaggactcctctgcaggctgagtacgagtacgttaagtactaccctaacggcagatctaacagcaacaacctggattctaaggttggcggcaactacaactacctctacaggctgttccggaagtccaaccttaagcctttcgagagggatatcagcaccgagatctatcaggctggttctactccttgcaatggcgttgagggtttcaactgctacttcccgcttcagtcttacggattccagcctactaatggtgttggctaccagccgtacttcaagcttgtggtgaatacccctttcgtggccgtgttcagcaacttcgattctagccagtgggagaaagctgattgggctaacggttctgtgttcaactgcgtgtggaagccttctcaggtgaccttctctaacggcaagatgattctgaccctggaccgtgagtattagctcgag

SEQ ID NO: 17 - protein, 441 aa, Artificial Sequence
  Synthetic
MHHHHHHHHGGSYPYKSGEYRTKSFFGYGYYEVRMKAAKNVGIVSSFFTYTGPSDNNPWDEIDIEFLGKDTTKVQFNWYKNGVGGNEYLHNLGFDASQDFHTYGFEWRPDYIDFYVDGKKVYRGTRNIPVTPGKIMMNLWPGIGVDEWLGRYDGRTPLQAEYEYVKYYPNGRSEFKLVVNTPFVAVFSNFDSSQWEKADWANGSVFNCVWKPSQVTFSNGKMILTLDREYRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKK

SEQ ID NO: 18 - DNA, 1340 bp, Artificial Sequence
  Synthetic
ttaattaaatgcatcaccatcaccaccatcatcatggcggctcttacccttataagagcggtgagtaccggaccaagagcttctttggttacggttactacgaggtgcggatgaaggctgctaagaacgtgggtatcgtgtccagcttctttacctacaccgggccatctgataacaacccttgggatgagatcgacatcgagttccttggtaaggacactaccaaggtgcagttcaactggtacaagaacggtgttggtggcaacgagtaccttcacaaccttggctttgatgccagccaggatttccacacttacggttttgaatggcggcctgactacatcgacttctacgtggacggtaagaaggtgtacaggggcaccagaaatatccctgtgactcctggcaagatcatgatgaacctttggcctggtatcggtgtggatgagtggcttggtagatacgatggtaggactcctctgcaggctgagtacgagtacgttaagtactaccctaacggcagatctgaattcaagcttgtggtgaatactcctttcgtggccgtgttcagcaacttcgattctagccagtgggagaaagctgattgggctaacggttctgtgttcaactgcgtgtggaagccttctcaggtgaccttctctaacggcaagatgattctgaccctggaccgtgagtatagggttcagcctactgagtctatcgtgcggttccctaacatcaccaacttgtgccctttcggcgaggtgttcaatgctactaggttcgcttctgtgtacgcctggaaccggaagaggatttctaactgcgtggccgattacagcgtgctgtacaactctgctagcttcagcaccttcaagtgctacggtgtgtctcctaccaagctgaacgatctgtgcttcaccaacgtgtacgctgactctttcgtgatcaggggtgatgaggttaggcagattgctcctggtcagaccggtaagatcgctgactacaactacaagctgcctgatgacttcaccggttgcgtgatcgcttggaactctaacaacctggactctaaggttggcggcaattacaactacctctaccggctgttccggaagtctaaccttaagcctttcgagcgggatatcagcaccgagatctatcaggctggttctactccttgcaatggcgttgagggtttcaactgctacttcccgcttcagtcttacggattccagcctactaatggtgtgggctaccagccttacagagtggtggttttgtctttcgagcttctgcatgctcctgctactgtttgcggtcctaagaagtagctcgag

SEQ ID NO: 19 - protein, 302 aa, Artificial Sequence
  Synthetic
MHHHHHHHHGGSYPYKSGEYRTKSFFGYGYYEVRMKAAKNVGIVSSFFTYTGPSDNNPWDEIDIEFLGKDTTKVQFNWYKNGVGGNEYLHNLGFDASQDFHTYGFEWRPDYIDFYVDGKKVYRGTRNIPVTPGKIMMNLWPGIGVDEWLGRYDGRTPLQAEYEYVKYYPNGRSEFKLVVNTPFVAVFSNFDSSQWEKADWANGSVFNCVWKPSQVTFSNGKMILTLDREYNSNNLDSKVGGNYNYLYRLFRKSNLKPFERDISTEIYQAGSTPCNGVEGFNCYFPLQSYGFQPTNGVGYQPY

SEQ ID NO: 20 - DNA, 923 bp, Artificial Sequence
  Synthetic
ttaattaaatgcatcaccatcaccaccatcatcatggcggctcttacccttataagagcggtgagtaccggaccaagagcttctttggttacggttactacgaggtgcggatgaaggctgctaagaacgtgggtatcgtgtccagcttctttacctacaccgggccatctgataacaacccttgggatgagatcgacatcgagttccttggtaaggacactaccaaggtgcagttcaactggtacaagaacggtgttggtggcaacgagtaccttcacaaccttggctttgatgccagccaggatttccacacttacggttttgaatggcggcctgactacatcgacttctacgtggacggtaagaaggtgtacaggggcaccagaaatatccctgtgactcctggcaagatcatgatgaacctttggcctggtatcggtgtggatgagtggcttggtagatacgatggtaggactcctctgcaggctgagtacgagtacgttaagtactaccctaacggcagatctgaattcaagcttgtggtgaatactcctttcgtggccgtgttcagcaacttcgattctagccagtgggagaaagctgattgggctaacggttctgtgttcaactgcgtgtggaagccttctcaggtgaccttctctaacggcaagatgattctgaccctggaccgtgagtacaacagcaacaacctggattctaaggtcggcggcaactacaactacctctacaggctgttccggaagtccaaccttaagcctttcgagagggatatcagcaccgagatctatcaggctggttctactccttgcaatggcgttgagggtttcaactgctacttcccgcttcagtcttacggattccagcctactaatggtgtgggctaccagccttattagctcgag

SEQ ID NO: 21 - protein, 469 aa, Artificial Sequence
  Synthetic
MGFVLFSQLPSFLLVSTLLLFLVISHSCRAHHHHHHHHGGSYPYKSGEYRTKSFFGYGYYEVRMKAAKNVGIVSSFFTYTGPSDNNPWDEIDIEFLGKDTTKVQFNWYKNGVGGNEYLHNLGFDASQDFHTYGFEWRPDYIDFYVDGKKVYRGTRNIPVTPGKIMMNLWPGIGVDEWLGRYDGRTPLQAEYEYVKYYPNGRSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKFKLVVNTPFVAVFSNFDSSQWEKADWANGSVFNCVWKPSQVTFSNGKMILTLDREY

SEQ ID NO: 22 - DNA, 1436 bp, Artificial Sequence
  Synthetic
acgtcattaattaaatgggattcgttttgttttctcaattgccttcatttcttttggtttctactcttttgctttttcttgttatttctcattcatgtagagctcatcaccatcaccaccatcatcatggcggctcttacccttataagagcggtgagtaccggaccaagagcttctttggttacggttactacgaggtgcggatgaaggctgctaagaacgtgggtatcgtgtccagcttctttacctacaccgggccatctgataacaacccttgggatgagatcgacatcgagttccttggtaaggacactaccaaggtgcagttcaactggtacaagaacggtgttggtggcaacgagtaccttcacaaccttggctttgatgccagccaggatttccacacttacggttttgaatggcggcctgactacatcgacttctacgtggacggtaagaaggtgtacaggggcaccagaaatatccctgtgactcctggcaagatcatgatgaacctttggcctggtatcggtgtggatgagtggcttggtagatacgatggtaggactcctctgcaggctgagtacgagtacgttaagtactaccctaacggcagatctagggttcagcctactgagtctattgtgcggttcccgaacatcaccaacttgtgcccttttggcgaggtgttcaatgctaccaggttcgcttctgtgtacgcctggaatcggaagcggatttctaactgcgtggccgattacagcgtgctgtacaactctgctagcttcagcaccttcaagtgctacggtgtgtctcctaccaagctgaacgatctgtgcttcaccaacgtgtacgctgactctttcgtgatcaggggtgatgaggttaggcagattgctcctggtcagaccggtaagatcgctgactacaactacaagctgcctgatgacttcaccggttgcgtgatcgcttggaactctaacaacctggactctaaggttggcggcaattacaactacctctaccggctgttccggaagtctaaccttaagcctttcgagcgggatatcagcaccgagatctatcaggctggttctactccttgcaatggcgttgagggtttcaactgctacttcccgcttcagtcttacggattccagcctactaatggtgtgggctaccagccttacagagtggtggttttgtctttcgagcttctgcatgctcctgctactgtttgcggtccgaagaagttcaagcttgtcgttaatacccctttcgtggccgtgttcagcaacttcgattctagccagtgggagaaagctgattgggctaacggttctgtgttcaactgcgtgtggaagccttctcaggtgaccttctctaacggcaagatgattctgaccctggaccgtgagtattagctcgagacgaag

SEQ ID NO: 23 - protein, 330 aa, Artificial Sequence
  Synthetic
MGFVLFSQLPSFLLVSTLLLFLVISHSCRAHHHHHHHHGGSYPYKSGEYRTKSFFGYGYYEVRMKAAKNVGIVSSFFTYTGPSDNNPWDEIDIEFLGKDTTKVQFNWYKNGVGGNEYLHNLGFDASQDFHTYGFEWRPDYIDFYVDGKKVYRGTRNIPVTPGKIMMNLWPGIGVDEWLGRYDGRTPLQAEYEYVKYYPNGRSNSNNLDSKVGGNYNYLYRLFRKSNLKPFERDISTEIYQAGSTPCNGVEGFNCYFPLQSYGFQPTNGVGYQPYFKLVVNTPFVAVFSNFDSSQWEKADWANGSVFNCVWKPSQVTFSNGKMILTLDREY

SEQ ID NO: 24 - DNA, 1019 bp, Artificial Sequence
  Synthetic
acgtcattaattaaatgggattcgttttgttttctcaattgccttcatttcttttggtttctactcttttgctttttcttgttatttctcattcatgtagagctcatcaccatcaccaccatcatcatggcggctcttacccttataagagcggtgagtaccggaccaagagcttctttggttacggttactacgaggtgcggatgaaggctgctaagaacgtgggtatcgtgtccagcttctttacctacaccgggccatctgataacaacccttgggatgagatcgacatcgagttccttggtaaggacactaccaaggtgcagttcaactggtacaagaacggtgttggtggcaacgagtaccttcacaaccttggctttgatgccagccaggatttccacacttacggttttgaatggcggcctgactacatcgacttctacgtggacggtaagaaggtgtacaggggcaccagaaatatccctgtgactcctggcaagatcatgatgaacctttggcctggtatcggtgtggatgagtggcttggtagatacgatggtaggactcctctgcaggctgagtacgagtacgttaagtactaccctaacggcagatctaacagcaacaacctggattctaaggttggcggcaactacaactacctctacaggctgttccggaagtccaaccttaagcctttcgagagggatatcagcaccgagatctatcaggctggttctactccttgcaatggcgttgagggtttcaactgctacttcccgcttcagtcttacggattccagcctactaatggtgttggctaccagccgtacttcaagcttgtggtgaatacccctttcgtggccgtgttcagcaacttcgattctagccagtgggagaaagctgattgggctaacggttctgtgttcaactgcgtgtggaagccttctcaggtgaccttctctaacggcaagatgattctgaccctggaccgtgagtattagctcgagacgaag

SEQ ID NO: 25 - protein, 470 aa, Artificial Sequence
  Synthetic
MGFVLFSQLPSFLLVSTLLLFLVISHSCRAHHHHHHHHGGSYPYKSGEYRTKSFFGYGYYEVRMKAAKNVGIVSSFFTYTGPSDNNPWDEIDIEFLGKDTTKVQFNWYKNGVGGNEYLHNLGFDASQDFHTYGFEWRPDYIDFYVDGKKVYRGTRNIPVTPGKIMMNLWPGIGVDEWLGRYDGRTPLQAEYEYVKYYPNGRSEFKLVVNTPFVAVFSNFDSSQWEKADWANGSVFNCVWKPSQVTFSNGKMILTLDREYRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKK

SEQ ID NO: 26 - DNA, 1439 bp, Artificial Sequence
  Synthetic
acgtcattaattaaatgggattcgttttgttttctcaattgccttcatttcttttggtttctactcttttgctttttcttgttatttctcattcatgtagagctcatcaccatcaccaccatcatcatggcggctcttacccttataagagcggtgagtaccggaccaagagcttctttggttacggttactacgaggtgcggatgaaggctgctaagaacgtgggtatcgtgtccagcttctttacctacaccgggccatctgataacaacccttgggatgagatcgacatcgagttccttggtaaggacactaccaaggtgcagttcaactggtacaagaacggtgttggtggcaacgagtaccttcacaaccttggctttgatgccagccaggatttccacacttacggttttgaatggcggcctgactacatcgacttctacgtggacggtaagaaggtgtacaggggcaccagaaatatccctgtgactcctggcaagatcatgatgaacctttggcctggtatcggtgtggatgagtggcttggtagatacgatggtaggactcctctgcaggctgagtacgagtacgttaagtactaccctaacggcagatctgaattcaagcttgtggtgaatactcctttcgtggccgtgttcagcaacttcgattctagccagtgggagaaagctgattgggctaacggttctgtgttcaactgcgtgtggaagccttctcaggtgaccttctctaacggcaagatgattctgaccctggaccgtgagtatagggttcagcctactgagtctatcgtgcggttccctaacatcaccaacttgtgccctttcggcgaggtgttcaatgctactaggttcgcttctgtgtacgcctggaaccggaagaggatttctaactgcgtggccgattacagcgtgctgtacaactctgctagcttcagcaccttcaagtgctacggtgtgtctcctaccaagctgaacgatctgtgcttcaccaacgtgtacgctgactctttcgtgatcaggggtgatgaggttaggcagattgctcctggtcagaccggtaagatcgctgactacaactacaagctgcctgatgacttcaccggttgcgtgatcgcttggaactctaacaacctggactctaaggttggcggcaattacaactacctctaccggctgttccggaagtctaaccttaagcctttcgagcgggatatcagcaccgagatctatcaggctggttctactccttgcaatggcgttgagggtttcaactgctacttcccgcttcagtcttacggattccagcctactaatggtgtgggctaccagccttacagagtggtggttttgtctttcgagcttctgcatgctcctgctactgtttgcggtcctaagaagtagctcgagacgaag

SEQ ID NO: 27 - protein, 331 aa, Artificial Sequence
  Synthetic
MGFVLFSQLPSFLLVSTLLLFLVISHSCRAHHHHHHHHGGSYPYKSGEYRTKSFFGYGYYEVRMKAAKNVGIVSSFFTYTGPSDNNPWDEIDIEFLGKDTTKVQFNWYKNGVGGNEYLHNLGFDASQDFHTYGFEWRPDYIDFYVDGKKVYRGTRNIPVTPGKIMMNLWPGIGVDEWLGRYDGRTPLQAEYEYVKYYPNGRSEFKLVVNTPFVAVFSNFDSSQWEKADWANGSVFNCVWKPSQVTFSNGKMILTLDREYNSNNLDSKVGGNYNYLYRLFRKSNLKPFERDISTEIYQAGSTPCNGVEGFNCYFPLQSYGFQPTNGVGYQPY

SEQ ID NO: 28 - DNA, 1022 bp, Artificial Sequence
  Synthetic
acgtcattaattaaatgggattcgttttgttttctcaattgccttcatttcttttggtttctactcttttgctttttcttgttatttctcattcatgtagagctcatcaccatcaccaccatcatcatggcggctcttacccttataagagcggtgagtaccggaccaagagcttctttggttacggttactacgaggtgcggatgaaggctgctaagaacgtgggtatcgtgtccagcttctttacctacaccgggccatctgataacaacccttgggatgagatcgacatcgagttccttggtaaggacactaccaaggtgcagttcaactggtacaagaacggtgttggtggcaacgagtaccttcacaaccttggctttgatgccagccaggatttccacacttacggttttgaatggcggcctgactacatcgacttctacgtggacggtaagaaggtgtacaggggcaccagaaatatccctgtgactcctggcaagatcatgatgaacctttggcctggtatcggtgtggatgagtggcttggtagatacgatggtaggactcctctgcaggctgagtacgagtacgttaagtactaccctaacggcagatctgaattcaagcttgtggtgaatactcctttcgtggccgtgttcagcaacttcgattctagccagtgggagaaagctgattgggctaacggttctgtgttcaactgcgtgtggaagccttctcaggtgaccttctctaacggcaagatgattctgaccctggaccgtgagtacaacagcaacaacctggattctaaggtcggcggcaactacaactacctctacaggctgttccggaagtccaaccttaagcctttcgagagggatatcagcaccgagatctatcaggctggttctactccttgcaatggcgttgagggtttcaactgctacttcccgcttcagtcttacggattccagcctactaatggtgtgggctaccagccttattagctcgagacgaag

SEQ ID NO: 29 - protein, 232 aa, Artificial Sequence
  Synthetic
MDIDPYKEFGATVELLSFLPSDFFPSVRDLLDTASALYREALESPEHCSPHHTALRQAILCWGELMTLATWVGANLEDPSGGSVCGPKKSTNLVKNKCVNFNFNGLTGTGVLTESNKKFLPFQQFGRDIADTTDAVRDPQTLEILDITPCSFGGVSVISGGSASRDLVVNYVNTNMGLKIRQLLWFHISCLTFGRETVLEYLVSFGVWIRTPPAYRPPNAPILSTLPETTVV

SEQ ID NO: 30 - DNA, 699 bp, Artificial Sequence
  Synthetic
atggacatcgatccgtacaaagaatttggcgcgaccgtcgagctgctgagcttcctgccgagcgattttttcccgagcgtgcgtgacctgctggacaccgcgagcgcactgtatcgtgaagcactggaaagcccagagcactgtagcccgcaccacaccgccctgcgccaggcgattctgtgctggggtgaactgatgaccctggccacctgggtgggtgctaaccttgaggatccgtccggaggtagcgtttgtggtcctaagaaaagtaccaatctcgtgaagaacaaatgcgtcaatttcaacttcaatgggttaactggaactggagttctaacagagtccaataagaaattccttccatttcagcaatttggcagggatattgcagacacaacagatgctgttagagatccacaaacgttggaaattctggacataactccctgttcttttggtggagtatcagtgatctccggaggtagcgctagccgtgatctggtcgtcaactatgtgaataccaacatgggtctgaaaattcgtcagctgctgtggtttcatattagctgcctgaccttcggtcgtgaaaccgtgctggagtatctggtgagcttcggtgtgtggattcgcaccccgccggcgtatcgtccgccgaacgcgccaattctgagcacgctgccggagaccaccgtggtttag

SEQ ID NO: 31 - protein, 197 aa, Artificial Sequence
  Synthetic
MDIDPYKEFGATVELLSFLPSDFFPSVRDLLDTASALYREALESPEHCSPHHTALRQAILCWGELMTLATWVGANLEDPSGGSGTNTSNQVAVLYQDVNCTEVPVAIHADQLTPTWRVYSTGSSGGSASRDLVVNYVNTNMGLKIRQLLWFHISCLTFGRETVLEYLVSFGVWIRTPPAYRPPNAPILSTLPETTVV

SEQ ID NO: 32 - DNA, 594 bp, Artificial Sequence
  Synthetic
atggacatcgatccgtacaaagaatttggcgcgaccgtcgagctgctgagcttcctgccgagcgattttttcccgagcgtgcgtgacctgctggacaccgcgagcgcactgtatcgtgaagcactggaaagcccagagcactgtagcccgcaccacaccgccctgcgccaggcgattctgtgctggggtgaactgatgaccctggccacctgggtgggtgctaaccttgaggatccgtccggaggtagcggtacgaatacctcaaatcaggttgctgtgttgtatcaagacgtgaactgtactgaagttcctgtagccattcatgcagatcaacttacaccaacttggagagtctactctacaggaagttccggaggtagcgctagccgtgatctggtcgtcaactatgtgaataccaacatgggtctgaaaattcgtcagctgctgtggtttcatattagctgcctgaccttcggtcgtgaaaccgtgctggagtatctggtgagcttcggtgtgtggattcgcaccccgccggcgtatcgtccgccgaacgcgccaattctgagcacgctgccggagaccaccgtggtttag

SEQ ID NO: 33 - protein, 274 aa, Artificial Sequence
  Synthetic
MDIDPYKEFGATVELLSFLPSDFFPSVRDLLDTASALYREALESPEHCSPHHTALRQAILCWGELMTLATWVGANLEDPSGGSVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSSGGSASRDLVVNYVNTNMGLKIRQLLWFHISCLTFGRETVLEYLVSFGVWIRTPPAYRPPNAPILSTLPETTVV

SEQ ID NO: 34 - DNA, 825 bp, Artificial Sequence
  Synthetic
atggacatcgatccgtacaaagaatttggcgcgaccgtcgagctgctgagcttcctgccgagcgattttttcccgagcgtgcgtgacctgctggacaccgcgagcgcactgtatcgtgaagcactggaaagcccagagcactgtagcccgcaccacaccgccctgcgccaggcgattctgtgctggggtgaactgatgaccctggccacctgggtgggtgctaaccttgaggatccgtccggaggtagcgtctgtggtcccaagaaaagcaccaatctggtgaagaacaaatgcgtgaatttcaacttcaatggcttaacaggaacaggtgttctaacggagtctaacaagaaattccttccgtttcaacagtttggcagagacattgctgataccacagatgcagttcgtgatcctcaaactctcgaaatcttggacattactccatgtagttttggaggtgtttctgtgataactccaggaactaacacctccaatcaagttgctgtgttgtatcaggatgtcaattgcactgaagtacctgttgccattcatgcagatcagcttactccaacatggagggtatactcaacagggtcatccggaggtagcgctagccgtgatctggtcgtcaactatgtgaataccaacatgggtctgaaaattcgtcagctgctgtggtttcatattagctgcctgaccttcggtcgtgaaaccgtgctggagtatctggtgagcttcggtgtgtggattcgcaccccgccggcgtatcgtccgccgaacgcgccaattctgagcacgctgccggagaccaccgtggtttag

SEQ ID NO: 35 - protein, 462 aa, Artificial Sequence
  Synthetic
MGFVLFSQLPSFLLVSTLLLFLVISHSCRAGGSYPYKSGEYRTKSFFGYGYYEVRMKAAKNVGIVSSFFTYTGPSDNNPWDEIDIEFLGKDTTKVQFNWYKNGVGGNEYLHNLGFDASQDFHTYGFEWRPDYIDFYVDGKKVYRGTRNIPVTPGKIMMNLWPGIGVDEWLGRYDGRTPLQAEYEYVKYYPNGRSEFKLVVNTPFVAVFSNFDSSQWEKADWANGSVFNCVWKPSQVTFSNGKMILTLDREYRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKK

SEQ ID NO: 36 - DNA, 1415 bp, Artificial Sequence
  Synthetic
acgtcattaattaaatgggattcgttttgttttctcaattgccttcatttcttttggtttctactcttttgctttttcttgttatttctcattcatgtagagctggcggctcttacccttataagagcggtgagtaccggaccaagagcttctttggttacggttactacgaggtgcggatgaaggctgctaagaacgtgggtatcgtgtccagcttctttacctacaccgggccatctgataacaacccttgggatgagatcgacatcgagttccttggtaaggacactaccaaggtgcagttcaactggtacaagaacggtgttggtggcaacgagtaccttcacaaccttggctttgatgccagccaggatttccacacttacggttttgaatggcggcctgactacatcgacttctacgtggacggtaagaaggtgtacaggggcaccagaaatatccctgtgactcctggcaagatcatgatgaacctttggcctggtatcggtgtggatgagtggcttggtagatacgatggtaggactcctctgcaggctgagtacgagtacgttaagtactaccctaacggcagatctgaattcaagcttgtggtgaatactcctttcgtggccgtgttcagcaacttcgattctagccagtgggagaaagctgattgggctaacggttctgtgttcaactgcgtgtggaagccttctcaggtgaccttctctaacggcaagatgattctgaccctggaccgtgagtatagggttcagcctactgagtctatcgtgcggttccctaacatcaccaacttgtgccctttcggcgaggtgttcaatgctactaggttcgcttctgtgtacgcctggaaccggaagaggatttctaactgcgtggccgattacagcgtgctgtacaactctgctagcttcagcaccttcaagtgctacggtgtgtctcctaccaagctgaacgatctgtgcttcaccaacgtgtacgctgactctttcgtgatcaggggtgatgaggttaggcagattgctcctggtcagaccggtaagatcgctgactacaactacaagctgcctgatgacttcaccggttgcgtgatcgcttggaactctaacaacctggactctaaggttggcggcaattacaactacctctaccggctgttccggaagtctaaccttaagcctttcgagcgggatatcagcaccgagatctatcaggctggttctactccttgcaatggcgttgagggtttcaactgctacttcccgcttcagtcttacggattccagcctactaatggtgtgggctaccagccttacagagtggtggttttgtctttcgagcttctgcatgctcctgctactgtttgcggtcctaagaagtagctcgagacgaag

SEQ ID NO: 37 - protein, 221 aa, Artificial Sequence; identical to SEQ ID NO: 3
  Synthetic

SEQ ID NO: 38 - protein, 360 aa, Artificial Sequence
  Synthetic
MDIDPYKEFGATVELLSFLPSDFFPSVRDLLDTASALYREALESPEHCSPHHTALRQAILCWGELMTLATWVGANLEDPRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKASRDLVVNYVNTNMGLKIRQLLWFHISCLTFGRETVLEYLVSFGVWIRTPPAYRPPNAPILSTLPETTVV

SEQ ID NO: 39 - protein, 238 aa, Artificial Sequence
  Synthetic
MDIDPYKEFGATVELLSFLPSDFFPSVRDLLDTASALYREALESPEHCSPHHTALRQAILCWGELMTLATWVGANLEDGGGGSGGGGTNSNNLDSKVGGNYNYLYRLFRKSNLKPFERDISTEIYQAGSTPCNGVEGFNCYFPLQSYGFQPTNGVGYQPYGGGGSGGGGSRDLVVNYVNTNMGLKIRQLLWFHISCLTFGRETVLEYLVSFGVWIRTPPAYRPPNAPILSTLPETTVV

SEQ ID NO: 40 - DNA, 717 bp, Artificial Sequence
  Synthetic
atggatattgatccatacaaggaattcggtgcaactgtggagcttctgtcattcttgccaagcgactttttcccgtcagttcgagatctgcttgatacggcttctgcactttacagggaagcgttggagagtccagagcattgctctcctcaccatactgcactaagacaagcaatactttgttggggtgaattaatgacactcgctacatgggttggagccaacctcgaagatggaggaggcggctctggtgggggcggaactaattccaataatttggactctaaagtaggtggtaactataattacctgtatcgtttatttagaaaatcgaatctcaaaccatttgagagagacatctcaactgagatataccaagctggatcaaccccatgcaacggtgtggaagggttcaattgttattttccgttacaatcctatggttttcaacctacaaatggtgttggttatcagccttacggcggcggagggagtggaggtggagggagtagagatttggttgtcaactatgtcaataccaacatgggattgaagattaggcagcttttatggtttcatatttcttgtttgacattcgggcgggaaacagtacttgaatatctagtaagctttggagtgtggatccgcaccccccctgcttataggcccccaaatgctcctattctttcaactcttcctgaaactacagttgtttga

SEQ ID NO: 41 - protein, 385 aa, Artificial Sequence
  Synthetic
MDIDPYKEFGATVELLSFLPSDFFPSVRDLLDTASALYREALESPEHCSPHHTALRQAILCWGELMTLATWVGANLEDGGGGSGGGGTRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKGGGGSGGGGSRDLVVNYVNTNMGLKIRQLLWFHISCLTFGRETVLEYLVSFGVWIRTPPAYRPPNAPILSTLPETTVV

SEQ ID NO: 42 - DNA, 1158 bp, Artificial Sequence
  Synthetic
atggatattgatccttataaagaatttggggctaccgtcgaactactatctttcttgccgtctgattttttcccatccgtcagagaccttcttgacactgcatctgctttatacagagaggctctggaatctcctgagcattgttcaccacaccacacagctctgagacaagcgatattgtgttggggtgaattgatgacacttgctacatgggttggagcaaatctggaagatggagggggtggttcaggtggtgggggcactcgggttcaaccaacagagagcattgttaggttccctaatattaccaacttgtgcccttttggagaggttttcaatgcaacccgtttcgccagcgtctatgcatggaaccgcaagaggatctccaactgtgtagcagattattctgtactatacaattccgccagtttttcaactttcaaatgttatggtgtaagtcccactaagcttaatgatttatgttttacgaatgtgtatgctgattcgtttgtaatcagaggtgatgaagtgagacagatagcacctgggcaaacaggaaaaatagcagactacaattacaagttgcctgatgatttcactggttgtgttattgcttggaatagtaacaaccttgatagtaaggtaggtggaaactacaactatttgtacaggctgtttcgtaaatccaatttaaagccgtttgagagagatattagcacagagatttatcaagctggatcaactccttgcaatggcgtagaaggatttaattgctattttcccttgcagtcatacggttttcaacctactaacggagtcggctaccagccatatagggtggttgtgctctctttcgagttgcttcatgcaccagcgactgtttgtggaccaaagaaatcgacaaatctagtgaagaataaaggaggaggcggctcagggggtggcgggtcacgagacttggttgtgaattatgttaatacaaacatgggactgaaaatccggcagctattatggtttcatattagttgcctcactttcggaagggaaactgttttagaatatcttgtctcttttggtgtttggattagaacgcctcccgcctatcgaccaccaaatgctccaatactttctactctccctgaaacaaccgttgtgtga

SEQ ID NO: 43 - protein, 446 aa, Artificial Sequence
  Synthetic
MDIDPYKEFGATVELLSFLPSDFFPSVRDLLDTASALYREALESPEHCSPHHTALRQAILCWGELMTLATWVGANLEDGGGGSGGGGTRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVIGGGGSGGGGSRDLVVNYVNTNMGLKIRQLLWFHISCLTFGRETVLEYLVSFGVWIRTPPAYRPPNAPILSTLPETTVV

SEQ ID NO: 44 - DNA, 1341 bp, Artificial Sequence
  Synthetic
atggatattgatccttacaaagaatttggtgctaccgtggagttactcagtttcctaccctctgattttttccctagtgtcagggacctattggacaccgcctccgctctttaccgagaagctttagagtctccagaacactgttctcctcatcatacagccttgagacaagctattttgtgttggggagagctgatgactcttgccacatgggttggggcaaatcttgaagatggtggaggcggctcaggtggtggaggtacccgtgttcaaccaactgagtcaattgttaggtttcctaatattacaaatttatgcccatttggcgaagtttttaatgcaacgagatttgctagcgtgtatgcctggaatcggaagagaatcagcaattgtgtagctgattattcagtgctctataattcggcgtccttttctacgttcaagtgctatggcgtgtcgccaacaaaacttaatgatctttgcttcactaatgtttatgcagactcttttgtgatcagaggagatgaagtgcgacagatagccccgggacaaactgggaaaattgcagattacaattacaagttgcctgatgatttcacaggttgtgtaatagcatggaactcgaataacttggattcaaaagttggtggtaactacaactatttatatcgtctcttcaggaaatccaatctgaagccttttgaaagagatatctcaactgaaatatatcaagcagggtctactccatgcaatggagttgaaggatttaactgttattttccactgcaatcttatggttttcaacctacgaacggggtaggatatcagccctacagagttgtggtactaagttttgagcttttacacgctcctgcaactgtttgtggtccaaagaaatcaaccaacctagtgaaaaataaatgtgtcaacttcaatttcaatggtttgactggaactggtgtcctgacagagagcaacaagaagtttctgccctttcagcaattcgggagagatatagctgacactactgatgctgttcgagacccacagaccttggagattctagacataacaccttgtagtttcgggggagtaagtgttatcggcggtggaggcagtggagggggcgggtcccgtgatctcgttgtcaattatgttaatacaaacatgggattgaagattaggcagcttctttggtttcatatttcttgcttaacattcggtcgggaaactgtcttggaatatttggtttcatttggagtttggattcgcaccccaccggcatacaggcctccaaatgctccaattctttcaactttacctgagacaacagttgtatga

SEQ ID NO: 45 - protein, 488 aa, Artificial Sequence
  Synthetic
MDIDPYKEFGATVELLSFLPSDFFPSVRDLLDTASALYREALESPEHCSPHHTALRQAILCWGELMTLATWVGANLEDGGGGSGGGGTRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSGGGGSGGGGSRDLVVNYVNTNMGLKIRQLLWFHISCLTFGRETVLEYLVSFGVWIRTPPAYRPPNAPILSTLPETTVV

SEQ ID NO: 46 - DNA, 1467 bp, Artificial Sequence
  Synthetic
atggatatcgatccttacaaagaatttggtgcaaccgtcgagcttctatcctttcttccttctgattttttcccgagtgtacgtgacctacttgacactgcttcagctctttacagggaagcattagagtctcccgagcattgtagccctcatcatacagctcttaggcaagcaatactctgttggggtgagttgatgacccttgcaacttgggtaggagcgaatttggaagatggtggaggaggcagtggtggaggtggcaccagagttcaaccaacagaaagcatcgttcggttccccaatattactaacctctgcccattcggagaagtttttaatgctactaggtttgcttctgtatatgcttggaacagaaaaagaatcagcaactgtgtcgccgattatagtgttttatacaactctgcctccttctccactttcaagtgctatggagtttcgcccactaaattaaatgatttatgcttcacaaatgtttatgcagattcttttgtgatacgaggtgatgaagtacggcaaattgcccctggtcaaactggaaagattgctgactacaattacaagctgcctgatgatttcactggttgtgttatagcatggaattctaataatcttgattccaaagtcggaggaaattataactatttgtaccgactctttaggaagtctaatctaaaaccatttgagagggacattagtactgaaatatatcaggctggtagtacaccttgcaatggtgttgaaggatttaattgttattttccattgcagagttatgggtttcagccaactaatggggttggttaccagccttatcgtgtggtggtactaagctttgaattattgcacgcgcccgcaactgtgtgtggaccaaagaagtctacaaatcttgtgaagaataaatgcgtgaatttcaacttcaacgggcttacgggtacaggtgtcttaaccgagtcaaacaagaaatttctcccgtttcaacaatttggcagagatatagccgacacaactgatgctgtacgcgatccacaaacattggagattttggacattacgccatgttcattcggaggcgtgtctgtaatcactcctgggaccaacacatccaatcaagttgctgttctgtatcaagatgttaactgcacagaggttccagtcgcgattcatgctgatcagttgaccccaacatggagagtctattctacaggatcgggtggcggtgggtcaggcggcggggggtctagggatttggtagtgaattatgttaatactaacatgggactgaaaattcgtcagctcttatggtttcacatttcatgtttgacatttgggagagaaacagtgcttgaatatctggtctcattcggagtttggattagaactccacctgcatacagacctcctaatgcaccgatcttatcaacgctgcctgagactactgtggtttga

SEQ ID NO: 47 - protein, 373 aa, Artificial Sequence
  Synthetic
MDIDPYKEFGATVELLSFLPSDFFPSVRDLLDTASALYREALESPEHCSPHHTALRQAILCWGELMTLATWVGANLEDPASRDLVVNYVNTNMGLKIRQLLWFHISCLTFGRETVLEYLVSFGVWIRTPPAYRPPNAPILSTLPGGGGSGGGGTRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNK

SEQ ID NO: 48 - DNA, 1122 bp, Artificial Sequence
  Synthetic
atggatattgatccatacaaggaatttggtgcaactgtggagctgttaagtttcttaccttctgatttcttccctagtgtgcgtgatttgctggacactgcgtcggctctttatcgtgaggctctggagagtcctgaacattgcagtccacaccacacagctttgagacaggcaattttgtgttggggtgaacttatgactcttgctacctgggttggagctaacttagaagatcctgcatcgagagacctcgttgttaattatgtgaatactaacatgggactcaaaatacgacaacttttgtggtttcatattagctgtctaacatttgggcgggagacagtgcttgagtacctagtatcatttggagtttggattcgtactcctccagcctataggccaccaaatgctcctatcctttctaccctacccggtggaggaggttctgggggcggcggtacgagggttcaacccacagaatctattgtgcgcttcccaaatatcactaatctctgcccctttggggaagttttcaatgcaacaagatttgcctcagtttatgcatggaatagaaaaagaatatctaattgtgttgcagactactccgtcctttacaactcagcctccttctctactttcaagtgttatggtgtttcaccaactaaattgaatgatctttgctttaccaatgtatatgctgacagctttgttatccggggagatgaggtgaggcaaatagcaccaggacagacgggtaagatagcagattacaattacaaactgcctgatgattttacagggtgcgtcattgcttggaacagtaataatttggacagcaaggttggtggcaactataactacttgtatagattgttcaggaaatcaaacttgaagccttttgaaagggatatttcaactgaaatttatcaagctggctccacaccctgtaatggtgtagaagggtttaattgctattttcctttacagtcatatggattccaaccaacaaatggagtgggttatcagccttatagagtcgtagtattatcttttgagctccttcatgctccggccactgtttgtgggccgaagaaaagtaccaacctagtcaagaataaatag

SEQ ID NO: 49 - protein, 433 aa, Artificial Sequence
  Synthetic
MGGSYPYKSGEYRTKSFFGYGYYEVRMKAAKNVGIVSSFFTYTGPSDNNPWDEIDIEFLGKDTTKVQFNWYKNGVGGNEYLHNLGFDASQDFHTYGFEWRPDYIDFYVDGKKVYRGTRNIPVTPGKIMMNLWPGIGVDEWLGRYDGRTPLQAEYEYVKYYPNGRSEFKLVVNTPFVAVFSNFDSSQWEKADWANGSVFNCVWKPSQVTFSNGKMILTLDREYRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKK

SEQ ID NO: 50 - DNA, 1328 bp, Artificial Sequence
  Synthetic
acgtcattaattaaatgggcggctcttacccttataagagcggtgagtaccggaccaagagcttctttggttacggttactacgaggtgcggatgaaggctgctaagaacgtgggtatcgtgtccagcttctttacctacaccgggccatctgataacaacccttgggatgagatcgacatcgagttccttggtaaggacactaccaaggtgcagttcaactggtacaagaacggtgttggtggcaacgagtaccttcacaaccttggctttgatgccagccaggatttccacacttacggttttgaatggcggcctgactacatcgacttctacgtggacggtaagaaggtgtacaggggcaccagaaatatccctgtgactcctggcaagatcatgatgaacctttggcctggtatcggtgtggatgagtggcttggtagatacgatggtaggactcctctgcaggctgagtacgagtacgttaagtactaccctaacggcagatctgaattcaagcttgtggtgaatactcctttcgtggccgtgttcagcaacttcgattctagccagtgggagaaagctgattgggctaacggttctgtgttcaactgcgtgtggaagccttctcaggtgaccttctctaacggcaagatgattctgaccctggaccgtgagtatagggttcagcctactgagtctatcgtgcggttccctaacatcaccaacttgtgccctttcggcgaggtgttcaatgctactaggttcgcttctgtgtacgcctggaaccggaagaggatttctaactgcgtggccgattacagcgtgctgtacaactctgctagcttcagcaccttcaagtgctacggtgtgtctcctaccaagctgaacgatctgtgcttcaccaacgtgtacgctgactctttcgtgatcaggggtgatgaggttaggcagattgctcctggtcagaccggtaagatcgctgactacaactacaagctgcctgatgacttcaccggttgcgtgatcgcttggaactctaacaacctggactctaaggttggcggcaattacaactacctctaccggctgttccggaagtctaaccttaagcctttcgagcgggatatcagcaccgagatctatcaggctggttctactccttgcaatggcgttgagggtttcaactgctacttcccgcttcagtcttacggattccagcctactaatggtgtgggctaccagccttacagagtggtggttttgtctttcgagcttctgcatgctcctgctactgtttgcggtcctaagaagtagctcgagacgaag

SEQ ID NO: 51 - protein, 617 aa, Artificial Sequence
  Synthetic
MGFVLFSQLPSFLLVSTLLLFLVISHSCRAGGSYPYKSGEYRTKSFFGYGYYEVRMKAAKNVGIVSSFFTYTGPSDNNPWDEIDIEFLGKDTTKVQFNWYKNGVGGNEYLHNLGFDASQDFHTYGFEWRPDYIDFYVDGKKVYRGTRNIPVTPGKIMMNLWPGIGVDEWLGRYDGRTPLQAEYEYVKYYPNGRSEFKLVVNTPFVAVFSNFDSSQWEKADWANGSVFNCVWKPSQVTFSNGKMILTLDREYRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRA

SEQ ID NO: 52 - DNA, 1880 bp, Artificial Sequence
  Synthetic
acgtcattaattaaatgggattcgttttgttttctcaattgccttcatttcttttggtttctactcttttgctttttcttgttatttctcattcatgtagagctggcggctcttacccttataagagcggtgagtaccggaccaagagcttctttggttacggttactacgaggtgcggatgaaggctgctaagaacgtgggtatcgtgtccagcttctttacctacaccgggccatctgataacaacccttgggatgagatcgacatcgagttccttggtaaggacactaccaaggtgcagttcaactggtacaagaacggtgttggtggcaacgagtaccttcacaaccttggctttgatgccagccaggatttccacacttacggttttgaatggcggcctgactacatcgacttctacgtggacggtaagaaggtgtacaggggcaccagaaatatccctgtgactcctggcaagatcatgatgaacctttggcctggtatcggtgtggatgagtggcttggtagatacgatggtaggactcctctgcaggctgagtacgagtacgttaagtactaccctaacggcagatctgaattcaagcttgtggtgaatactcctttcgtggccgtgttcagcaacttcgattctagccagtgggagaaagctgattgggctaacggttctgtgttcaactgcgtgtggaagccttctcaggtgaccttctctaacggcaagatgattctgaccctggaccgtgagtatagggttcaaccaactgagtcaatcgttaggttcccaaacatcacaaatttgtgtcctttcggtgaagtttttaatgctactagattcgcttctgtttacgcttggaacagaaaaaggatctcaaattgcgttgctgattactctgttctttacaactctgcttctttttctacttttaagtgttacggtgtttcaccaactaagttgaacgatctttgcttcacaaacgtttacgctgattctttcgttattagaggagatgaggttaggcaaattgctcctggacaaactggaaagattgctgattacaactacaaattgccagatgatttcacaggatgcgttatcgcttggaactcaaataaccttgattctaaggttggaggtaattataactacttgtacagactttttaggaagtcaaatttgaagcctttcgaaagggatatctcaactgagatctatcaagctggttctacaccatgtaatggtgttgaaggttttaattgctacttcccacttcaatcttatggatttcaacctactaatggtgttggttaccaaccatacagagttgttgttttgtcattcgagttgcttcatgctccagctactgtttgtggtcctaagaaatctacaaatcttgttaagaataagtgcgttaacttcaacttcaatggtttgactggaacaggtgttcttactgaatcaaataagaagttcttgcctttccaacaattcggtagagatattgctgatactacagatgctgttagggatcctcaaactttggagattcttgatattacaccatgttcatttggaggtgtttctgttattactccaggaactaacacatctaaccaagttgctgttttgtaccaagatgttaattgcacagaagttcctgttgctattcatgctgatcaacttactccaacatggagagtttactcaactggatctaacgttttccaaacaagggctggatgtcttattggtgctgaacatgttaataactcttacgagtgtgatattcctattggagctggtatttgcgcttcatatcaaactcaaacaaattctccaagaagggcttagctcgagacgaag

SEQ ID NO: 53 - protein, 617 aa, Artificial Sequence
  Synthetic
MGFVLFSQLPSFLLVSTLLLFLVISHSCRAGGSYPYKSGEYRTKSFFGYGYYEVRMKAAKNVGIVSSFFTYTGPSDNNPWDEIDIEFLGKDTTKVQFNWYKNGVGGNEYLHNLGFDASQDFHTYGFEWRPDYIDFYVDGKKVYRGTRNIPVTPGKIMMNLWPGIGVDEWLGRYDGRTPLQAEYEYVKYYPNGRSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAEFKLVVNTPFVAVFSNFDSSQWEKADWANGSVFNCVWKPSQVTFSNGKMILTLDREY

SEQ ID NO: 54 - DNA, 1880 bp, Artificial Sequence
  Synthetic
acgtcattaattaaatgggattcgttttgttttctcaattgccttcatttcttttggtttctactcttttgctttttcttgttatttctcattcatgtagagctggcggctcttacccttataagagcggtgagtaccggaccaagagcttctttggttacggttactacgaggtgcggatgaaggctgctaagaacgtgggtatcgtgtccagcttctttacctacaccgggccatctgataacaacccttgggatgagatcgacatcgagttccttggtaaggacactaccaaggtgcagttcaactggtacaagaacggtgttggtggcaacgagtaccttcacaaccttggctttgatgccagccaggatttccacacttacggttttgaatggcggcctgactacatcgacttctacgtggacggtaagaaggtgtacaggggcaccagaaatatccctgtgactcctggcaagatcatgatgaacctttggcctggtatcggtgtggatgagtggcttggtagatacgatggtaggactcctctgcaggctgagtacgagtacgttaagtactaccctaacggcagatctagggttcaacctactgaatctatcgttaggttcccaaacatcacaaatttgtgtcctttcggagaggtttttaatgctactagattcgcttctgtttatgcttggaatagaaagaggatttcaaattgcgttgctgattactctgttctttacaactctgcttctttttctacttttaagtgttacggtgtttcaccaactaagttgaacgatctttgcttcacaaacgtttacgctgattctttcgttattagaggagatgaggttaggcaaattgctcctggacaaactggaaagattgctgattacaactacaaattgccagatgatttcacaggttgtgttatcgcttggaactcaaataaccttgattctaaggttggaggtaattataactacttgtacagactttttaggaagtcaaatttgaagcctttcgaaagggatatctcaactgagatctatcaagctggttctacaccatgtaatggtgttgaaggttttaattgctacttcccacttcaatcttatggatttcaacctactaatggtgttggttaccaaccatacagagttgttgttttgtcattcgagttgcttcatgctccagctactgtttgtggtcctaagaaatctacaaatcttgttaagaataagtgcgttaacttcaacttcaatggtttgactggaacaggtgttcttacagaatcaaataagaagttccttcctttccaacaattcggtagagatattgctgatactacagatgctgttagggatcctcaaactttggagattcttgatattacaccatgttcatttggaggtgtttctgttattactccaggaactaacacatctaaccaagttgctgttttgtaccaagatgttaattgcacagaagttcctgttgctattcatgctgatcaacttactccaacatggagagtttactcaactggatctaacgttttccaaacaagggctggatgtttgattggtgctgaacatgttaataactcttacgagtgtgatattcctattggagctggtatttgcgcttcatatcaaactcaaacaaattctcctagaagggctgaattcaagcttgtggtgaatactcctttcgtggccgtgttcagcaacttcgattctagccagtgggagaaagctgattgggctaacggttctgtgttcaactgcgtgtggaagccttctcaggtgaccttctctaacggcaagatgattctgaccctggaccgtgagtattagctcgagacgaag

SEQ ID NO: 55 - protein, 709 aa, Artificial Sequence
  Synthetic
MGFVLFSQLPSFLLVSTLLLFLVISHSCRAGGSYPYKSGEYRTKSFFGYGYYEVRMKAAKNVGIVSSFFTYTGPSDNNPWDEIDIEFLGKDTTKVQFNWYKNGVGGNEYLHNLGFDASQDFHTYGFEWRPDYIDFYVDGKKVYRGTRNIPVTPGKIMMNLWPGIGVDEWLGRYDGRTPLQAEYEYVKYYPNGRSEFKLVVNTPFVAVFSNFDSSQWEKADWANGSVFNCVWKPSQVTFSNGKMILTLDREYVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRA

SEQ ID NO: 56 - DNA, 2156 bp, Artificial Sequence
  Synthetic
acgtcattaattaaatgggattcgttttgttttctcaattgccttcatttcttttggtttctactcttttgctttttcttgttatttctcattcatgtagagctggcggctcttacccttataagagcggtgagtaccggaccaagagcttctttggttacggttactacgaggtgcggatgaaggctgctaagaacgtgggtatcgtgtccagcttctttacctacaccgggccatctgataacaacccttgggatgagatcgacatcgagttccttggtaaggacactaccaaggtgcagttcaactggtacaagaacggtgttggtggcaacgagtaccttcacaaccttggctttgatgccagccaggatttccacacttacggttttgaatggcggcctgactacatcgacttctacgtggacggtaagaaggtgtacaggggcaccagaaatatccctgtgactcctggcaagatcatgatgaacctttggcctggtatcggtgtggatgagtggcttggtagatacgatggtaggactcctctgcaggctgagtacgagtacgttaagtactaccctaacggcagatctgaattcaagcttgtggtgaatactcctttcgtggccgtgttcagcaacttcgattctagccagtgggagaaagctgattgggctaacggttctgtgttcaactgcgtgtggaagccttctcaggtgaccttctctaacggcaagatgattctgaccctggaccgtgagtatgttgatttgcctatcggaattaatatcactagattccaaacattgcttgctttgcataggtcatatcttacaccaggagattcttcttctggatggactgctggtgctgctgcttattacgttggttaccttcaacctagaacttttcttcttaagtacaatgagaatggtactattacagatgctgttgattgtgctttggatccactttctgaaactaagtgcacattgaagtcttttactgttgagaagggtatctatcaaacatctaacttcagagttcaacctactgaatcaatcgttaggttcccaaacatcacaaatctttgtcctttcggagaggtttttaatgctactagattcgcttctgtttatgcttggaatagaaagaggatttctaattgcgttgctgattactcagttttgtacaactcagcttctttttcaacttttaagtgttacggtgtttctccaacaaagttgaacgatctttgcttcactaacgtttacgctgattcattcgttatcagaggagatgaagttaggcaaattgctcctggacaaacaggaaagattgctgattacaactacaaattgccagatgatttcactggatgtgttatcgcttggaactctaataaccttgattcaaaggttggaggtaattataactacttgtacagactttttaggaagtctaatttgaagcctttcgaaagggatatctctacagagatctatcaagctggttcaactccatgtaatggtgttgaaggttttaattgctacttcccacttcaatcatatggatttcaacctacaaatggtgttggttaccaaccatacagagttgttgttttgtctttcgagttgcttcatgctccagctacagtttgtggtcctaagaaatcaactaatcttgttaagaataagtgcgttaacttcaacttcaatggtttgactggaacaggtgttcttactgaatctaataagaagttcttgccattccaacaattcggtagagatattgctgatactacagatgctgttagggatcctcaaactttggagattcttgatattacaccatgttcttttggaggtgtttcagttattacacctggaactaacacatcaaaccaagttgctgttttgtaccaagatgttaattgcactgaggttcctgttgctattcatgctgatcaacttactccaacatggagagtttactctactggttcaaacgttttccaaacaagggctggatgtcttattggtgctgaacatgttaataactcttacgagtgtgatattcctattggagctggtatttgcgcttcttatcaaactcaaacaaattcaccaagaagggcttagctcgagacgaag

SEQ ID NO: 57 - protein, 709 aa, Artificial Sequence
  Synthetic
MGFVLFSQLPSFLLVSTLLLFLVISHSCRAGGSYPYKSGEYRTKSFFGYGYYEVRMKAAKNVGIVSSFFTYTGPSDNNPWDEIDIEFLGKDTTKVQFNWYKNGVGGNEYLHNLGFDASQDFHTYGFEWRPDYIDFYVDGKKVYRGTRNIPVTPGKIMMNLWPGIGVDEWLGRYDGRTPLQAEYEYVKYYPNGRSVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAEFKLVVNTPFVAVFSNFDSSQWEKADWANGSVFNCVWKPSQVTFSNGKMILTLDREY

SEQ ID NO: 58 - DNA, 2156 bp, Artificial Sequence
  Synthetic
acgtcattaattaaatgggattcgttttgttttctcaattgccttcatttcttttggtttctactcttttgctttttcttgttatttctcattcatgtagagctggcggctcttacccttataagagcggtgagtaccggaccaagagcttctttggttacggttactacgaggtgcggatgaaggctgctaagaacgtgggtatcgtgtccagcttctttacctacaccgggccatctgataacaacccttgggatgagatcgacatcgagttccttggtaaggacactaccaaggtgcagttcaactggtacaagaacggtgttggtggcaacgagtaccttcacaaccttggctttgatgccagccaggatttccacacttacggttttgaatggcggcctgactacatcgacttctacgtggacggtaagaaggtgtacaggggcaccagaaatatccctgtgactcctggcaagatcatgatgaacctttggcctggtatcggtgtggatgagtggcttggtagatacgatggtaggactcctctgcaggctgagtacgagtacgttaagtactaccctaacggcagatctgttgatcttcctatcggtattaatatcactagattccaaacattgcttgctttgcataggtcatatcttacacctggagattcttcttctggatggactgctggtgctgctgcttattacgttggttacttgcaaccaagaacttttcttcttaagtacaatgagaatggtactattacagatgctgttgattgtgctttggatcctctttctgaaactaagtgcacattgaagtcttttactgttgagaagggtatctatcaaacatctaacttcagagttcaaccaactgaatcaatcgttaggttcccaaacatcacaaatctttgtcctttcggagaggtttttaatgctactagattcgcttctgtttatgcttggaatagaaagaggatttctaattgcgttgctgattactcagttctttacaactcagcttctttttcaacttttaagtgttacggtgtttctccaacaaagttgaacgatctttgcttcactaacgtttacgctgattcattcgttatcagaggagatgaagttaggcaaattgctcctggacaaacaggaaagattgctgattacaactacaaattgccagatgatttcactggatgtgttatcgcttggaactctaataaccttgattcaaaggttggaggtaattataactacttgtacagactttttaggaagtctaatttgaagcctttcgaaagggatatctctacagagatctatcaagctggttcaactccatgtaatggtgttgaaggttttaattgctacttcccacttcaatcatatggatttcaacctacaaatggtgttggttaccaaccatacagagttgttgttttgtctttcgagttgcttcatgctccagctacagtttgtggtcctaagaaatcaactaatcttgttaagaataagtgcgttaacttcaacttcaatggtttgactggaacaggtgttcttactgaatctaataagaagttcttgcctttccaacaattcggtagagatattgctgatactacagatgctgttagggatcctcaaactttggagattcttgatattacaccatgttcttttggaggtgtttcagttattacaccaggaactaacacatcaaaccaagttgctgttttgtaccaagatgttaattgcactgaagttcctgttgctattcatgctgatcaacttactccaacatggagagtttactctactggttcaaacgttttccaaacaagggctggatgtttgattggtgctgaacatgttaataactcttacgagtgtgatattcctattggagctggtatttgcgcttcttatcaaactcaaacaaattcacctagaagggctgaattcaagcttgtggtgaatactcctttcgtggccgtgttcagcaacttcgattctagccagtgggagaaagctgattgggctaacggttctgtgttcaactgcgtgtggaagccttctcaggtgaccttctctaacggcaagatgattctgaccctggaccgtgagtattagctcgagacgaag

SEQ ID NO: 59 - protein, 676 aa, Artificial Sequence
  Synthetic
MGFVLFSQLPSFLLVSTLLLFLVISHSCRAGGSYPYKSGEYRTKSFFGYGYYEVRMKAAKNVGIVSSFFTYTGPSDNNPWDEIDIEFLGKDTTKVQFNWYKNGVGGNEYLHNLGFDASQDFHTYGFEWRPDYIDFYVDGKKVYRGTRNIPVTPGKIMMNLWPGIGVDEWLGRYDGRTPLQAEYEYVKYYPNGRSKYEQYIKWPWYIWLGFIAGLIAIVMVTIMLCCMTSCCSCLKGCCSCGSCCKFDEDDSEPEFKLVVNTPFVAVFSNFDSSQWEKADWANGSVFNCVWKPSQVTFSNGKMILTLDREYRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRA

SEQ ID NO: 60 - DNA, 2057 bp, Artificial Sequence
  Synthetic
acgtcattaattaaatgggattcgttttgttttctcaattgccttcatttcttttggtttctactcttttgctttttcttgttatttctcattcatgtagagctggcggctcttacccttataagagcggtgagtaccggaccaagagcttctttggttacggttactacgaggtgcggatgaaggctgctaagaacgtgggtatcgtgtccagcttctttacctacaccgggccatctgataacaacccttgggatgagatcgacatcgagttccttggtaaggacactaccaaggtgcagttcaactggtacaagaacggtgttggtggcaacgagtaccttcacaaccttggctttgatgccagccaggatttccacacttacggttttgaatggcggcctgactacatcgacttctacgtggacggtaagaaggtgtacaggggcaccagaaatatccctgtgactcctggcaagatcatgatgaacctttggcctggtatcggtgtggatgagtggcttggtagatacgatggtaggactcctctgcaggctgagtacgagtacgttaagtactaccctaacggcagatctaagtacgagcaatatattaagtggccttggtatatttggttgggttttattgctggtcttatcgctatcgttatggttactattatgttgtgttgcatgacatcatgttgctcttgtcttaagggatgttgctcatgcggttcttgttgcaaatttgatgaagatgattctgaacctgaattcaagcttgtggtgaatactcctttcgtggccgtgttcagcaacttcgattctagccagtgggagaaagctgattgggctaacggttctgtgttcaactgcgtgtggaagccttctcaggtgaccttctctaacggcaagatgattctgaccctggaccgtgagtatagggttcaaccaactgagtcaatcgttaggttcccaaacatcacaaatttgtgtcctttcggtgaagtttttaatgctactagattcgcttctgtttatgcttggaatagaaagaggatttcaaattgcgttgctgattactctgttctttacaactctgcttctttttctacttttaagtgttacggtgtttcaccaactaagttgaacgatctttgcttcacaaacgtttacgctgattctttcgttattagaggagatgaggttaggcaaattgctcctggacaaactggaaagattgctgattacaactacaaattgccagatgatttcacaggatgcgttatcgcttggaactcaaataaccttgattctaaggttggaggtaattataactacttgtacagactttttaggaagtcaaatttgaagcctttcgaaagggatatctcaactgagatctatcaagctggttctacaccatgtaatggtgttgaaggttttaattgctacttcccacttcaatcttatggatttcaacctactaatggtgttggttaccaaccatacagagttgttgttttgtcattcgagttgcttcatgctccagctactgtttgtggtcctaagaaatctacaaatcttgttaagaataagtgcgttaacttcaacttcaatggtttgactggaacaggtgttcttacagaatcaaataagaagttcttgcctttccaacaattcggtagagatattgctgatactacagatgctgttagggatcctcaaactttggagattcttgatattacaccatgttcatttggaggtgtttctgttattactccaggaactaacacatctaaccaagttgctgttttgtaccaagatgttaattgcacagaagttcctgttgctattcatgctgatcaacttactccaacatggagagtttactcaactggatctaacgttttccaaacaagggctggatgtcttattggtgctgaacatgttaataactcttacgagtgtgatattcctattggagctggtatttgcgcttcatatcaaactcaaacaaattctccaagaagggcttagctcgagacgaag

SEQ ID NO: 61 - protein, 590 aa, Artificial Sequence
  Synthetic
MGFVLFSQLPSFLLVSTLLLFLVISHSCRAGGSYPYKSGEYRTKSFFGYGYYEVRMKAAKNVGIVSSFFTYTGPSDNNPWDEIDIEFLGKDTTKVQFNWYKNGVGGNEYLHNLGFDASQDFHTYGFEWRPDYIDFYVDGKKVYRGTRNIPVTPGKIMMNLWPGIGVDEWLGRYDGRTPLQAEYEYVKYYPNGRSKYEQYIKWPWYIWLGFIAGLIAIVMVTIMLCCMTSCCSCLKGCCSCGSCCKFDEDDSEPEFKLVVNTPFVAVFSNFDSSQWEKADWANGSVFNCVWKPSQVTFSNGKMILTLDREYRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVI

SEQ ID NO: 62 - DNA, 1799 bp, Artificial Sequence
  Synthetic
acgtcattaattaaatgggattcgttttgttttctcaattgccttcatttcttttggtttctactcttttgctttttcttgttatttctcattcatgtagagctggcggctcttacccttataagagcggtgagtaccggaccaagagcttctttggttacggttactacgaggtgcggatgaaggctgctaagaacgtgggtatcgtgtccagcttctttacctacaccgggccatctgataacaacccttgggatgagatcgacatcgagttccttggtaaggacactaccaaggtgcagttcaactggtacaagaacggtgttggtggcaacgagtaccttcacaaccttggctttgatgccagccaggatttccacacttacggttttgaatggcggcctgactacatcgacttctacgtggacggtaagaaggtgtacaggggcaccagaaatatccctgtgactcctggcaagatcatgatgaacctttggcctggtatcggtgtggatgagtggcttggtagatacgatggtaggactcctctgcaggctgagtacgagtacgttaagtactaccctaacggcagatctaagtacgagcaatatattaagtggccttggtatatttggttgggttttattgctggtcttatcgctatcgttatggttactattatgttgtgttgcatgacatcatgttgctcttgtcttaagggatgttgctcatgcggttcttgttgcaaatttgatgaagatgattctgaacctgaattcaagcttgtggtgaatactcctttcgtggccgtgttcagcaacttcgattctagccagtgggagaaagctgattgggctaacggttctgtgttcaactgcgtgtggaagccttctcaggtgaccttctctaacggcaagatgattctgaccctggaccgtgagtatagggttcaaccaactgagtcaatcgttaggttcccaaacatcacaaatttgtgtcctttcggtgaagtttttaatgctactagattcgcttctgtttatgcttggaatagaaagaggatttcaaattgcgttgctgattactctgttctttacaactctgcttctttttctacttttaagtgttacggtgtttcaccaactaagttgaacgatctttgcttcacaaacgtttacgctgattctttcgttattagaggagatgaggttaggcaaattgctcctggacaaactggaaagattgctgattacaactacaaattgccagatgatttcacaggatgcgttatcgcttggaactcaaataaccttgattctaaggttggaggtaattataactacttgtacagactttttaggaagtcaaatttgaagcctttcgaaagggatatctcaactgagatctatcaagctggttctacaccatgtaatggtgttgaaggttttaattgctacttcccacttcaatcttatggatttcaacctactaatggtgttggttaccaaccatacagagttgttgttttgtcattcgagttgcttcatgctccagctactgtttgtggtcctaagaaatctacaaatcttgttaagaataagtgcgttaacttcaacttcaatggtttgactggaacaggtgttcttacagaatcaaataagaagttcttgcctttccaacaattcggtagagatattgctgatactacagatgctgttagggatcctcaaactttggagattcttgatattacaccatgttcatttggaggtgtttctgttatttagctcgagacgaag

SEQ ID NO: 63 - protein, 233 aa, Artificial Sequence
  Synthetic
MDIDPYKEFGATVELLSFLPSDFFPSVRDLLDTASALYREALESPEHCSPHHTALRQAILCWGELMTLATWVGANLEDGGGSGGSGGSGGSGGSVLTESNKKFLPFQQFGGGSAIHADQLTPTWRVYSTGGSDSFKEELDKYFKNHTSGGGSGGSGGSGGSGGSSRDLVVNYVNTNMGLKIRQLLWFHISCLTFGRETVLEYLVSFGVWIRTPPAYRPPNAPILSTLPETTVV

SEQ ID NO: 64 - DNA, 728 bp, Artificial Sequence
  Synthetic
acgtcattaattaaatggacatcgatccgtacaaagaatttggcgcgaccgtcgagctgctgagcttcctgccgagcgattttttcccgagcgtgcgtgacctgctggacaccgcgagcgcactgtatcgtgaagcactggaaagcccagagcactgtagcccgcaccacaccgccctgcgccaggcgattctgtgctggggtgaactgatgaccctggccacctgggtgggtgctaaccttgaggacggtggtggatcaggaggaagtggcggcagtggaggttcaggggggagcgtcctgactgagtcgaacaagaaattccttccatttcagcagtttggtgggggaagcgccatacatgctgatcaactcaccccaacttggagagtatattctacgggtggatctgattcatttaaggaagaattagacaaatatttcaagaatcatacatctggtggtggcagtgggggatcgggtggaagtggaggttccggaggctctagccgtgatctggtcgtcaactatgtgaataccaacatgggtctgaaaattcgtcagctgctgtggtttcatattagctgcctgaccttcggtcgtgaaaccgtgctggagtatctggtgagcttcggtgtgtggattcgcaccccgccggcgtatcgtccgccgaacgcgccaattctgagcacgctgccggagaccaccgtggtttagctcgagacgaag

SEQ ID NO: 65 - protein, 256 aa, Artificial Sequence
  Synthetic
MDIDPYKEFGATVELLSFLPSDFFPSVRDLLDTASALYREALESPEHCSPHHTALRQAILCWGELMTLATWVGANLEDPGGGSGGSGGSGGSGGSVCGPKKSTNLVKNKCVNFNFNGLTGTGVLTESNKKFLPFQQFGRDIADTTDAVRDPQTLEILDITPCSFGGVSVIGGGSGGSGGSGGSGGSASRDLVVNYVNTNMGLKIRQLLWFHISCLTFGRETVLEYLVSFGVWIRTPPAYRPPNAPILSTLPETTVV

SEQ ID NO: 66 - DNA, 797 bp, Artificial Sequence
  Synthetic
acgtcattaattaaatggacatcgatccgtacaaagaatttggcgcgaccgtcgagctgctgagcttcctgccgagcgattttttcccgagcgtgcgtgacctgctggacaccgcgagcgcactgtatcgtgaagcactggaaagcccagagcactgtagcccgcaccacaccgccctgcgccaggcgattctgtgctggggtgaactgatgaccctggccacctgggtgggtgctaaccttgaggaccctggtggtggatcaggaggaagtggcggcagtggaggttcaggggggagcgtttgtggtcctaagaaaagtaccaatctcgtgaagaacaaatgcgtcaatttcaacttcaatgggttaactggaactggagttctaacagagtccaataagaaattccttccatttcagcaatttggcagggatattgcagacacaacagatgctgttagagatccacaaacgttggaaattctggacataactccctgttcttttggtggagtatcagtgatcggtggtggcagtgggggatcgggtggaagtggaggttccggaggctctgctagccgtgatctggtcgtcaactatgtgaataccaacatgggtctgaaaattcgtcagctgctgtggtttcatattagctgcctgaccttcggtcgtgaaaccgtgctggagtatctggtgagcttcggtgtgtggattcgcaccccgccggcgtatcgtccgccgaacgcgccaattctgagcacgctgccggagaccaccgtggtttagctcgagacgaag

SEQ ID NO: 67 - protein, 221 aa, Artificial Sequence
  Synthetic
MDIDPYKEFGATVELLSFLPSDFFPSVRDLLDTASALYREALESPEHCSPHHTALRQAILCWGELMTLATWVGANLEDPGGGSGGSGGSGGSGGSGTNTSNQVAVLYQDVNCTEVPVAIHADQLTPTWRVYSTGSGGGSGGSGGSGGSGGSASRDLVVNYVNTNMGLKIRQLLWFHISCLTFGRETVLEYLVSFGVWIRTPPAYRPPNAPILSTLPETTVV

SEQ ID NO: 68 - DNA, 692 bp, Artificial Sequence
  Synthetic
acgtcattaattaaatggacatcgatccgtacaaagaatttggcgcgaccgtcgagctgctgagcttcctgccgagcgattttttcccgagcgtgcgtgacctgctggacaccgcgagcgcactgtatcgtgaagcactggaaagcccagagcactgtagcccgcaccacaccgccctgcgccaggcgattctgtgctggggtgaactgatgaccctggccacctgggtgggtgctaaccttgaggaccctggtggtggatcaggaggaagtggcggcagtggaggttcaggggggagcggtacgaatacctcaaatcaggttgctgtgttgtatcaagacgtgaactgtactgaagttcctgtagccattcatgcagatcaacttacaccaacttggagagtctactctacaggaagtggtggtggcagtgggggatcgggtggaagtggaggttccggaggctctgctagccgtgatctggtcgtcaactatgtgaataccaacatgggtctgaaaattcgtcagctgctgtggtttcatattagctgcctgaccttcggtcgtgaaaccgtgctggagtatctggtgagcttcggtgtgtggattcgcaccccgccggcgtatcgtccgccgaacgcgccaattctgagcacgctgccggagaccaccgtggtttagctcgagacgaag

SEQ ID NO: 69 - protein, 530 aa, Artificial Sequence
  Synthetic
MDIDPYKEFGATVELLSFLPSDFFPSVRDLLDTASALYREALESPEHCSPHHTALRQAILCWGELMTLATWVGNNLEGSAGGGRDPASRDLVVNYVNTNMGLKIRQLLWFHISCLTFGRETVLEYLVSFGVWIRTPPAYRPPNAPILSTLPETTVVGGSSGGSGGSGGSGGSGGSGGSTMDIDPYKEFGATVELLSFLPSDFFPSVRDLLDTASALYREALESPEHCSPHHTALRQAILCWGELMTLATWVGNNLEFGGSSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRASGGASDPASRDLVVNYVNTNMGLKIRQLLWFHISCLTFGRETVLEYLVSFGVWIRTPPAYRPPNAPILSTLPETTVVRRRDRGRSPRRRTPSPRRRRSQSPRRRRSQSRESQCLE

SEQ ID NO: 70 - DNA, 1607 bp, Artificial Sequence
  Synthetic
ttaattaaatggacatcgatccgtacaaagaatttggcgcgaccgtcgagctgctgagcttcctgccgagcgattttttcccgagcgtgcgtgacctgctggacaccgcgagcgcactgtatcgtgaagcactggaaagcccagagcactgtagcccgcaccacaccgccctgcgccaggcgattctgtgctggggtgaactgatgaccctggccacctgggtgggtaataatctggaaggttccgccggcggcggccgcgatccggcgagccgtgatctggtcgtcaactatgtgaataccaacatgggtctgaaaattcgtcagctgctgtggtttcatattagctgcctgaccttcggtcgtgaaaccgtgctggagtatctggtgagcttcggtgtgtggattcgcaccccgccggcgtatcgtccgccgaacgcgccaattctgagcacgctgccggagaccaccgtggttggtgggagctctggtggcagcggcggcagcggtggtagcggtggcagcggtggtagcggcgggtcgactatggatatcgacccatataaagaatttggcgcgacggttgagctgctgagctttctgccaagcgatttctttccgagcgtccgcgacctgctggataccgccagcgcactgtatcgtgaagccctggagagcccggaacattgcagcccgcatcatacggccctgcgtcaggcaatcctgtgctggggcgaactgatgaccctggcaacctgggtcggcaataatctggaattcggcggatccagtactaatcttgtgaaaaacaaatgtgttaatttcaacttcaatggtttaaccggaactggtgttctgaccgagagtaacaagaaatttctacctttccagcaatttgggagggacattgcagatacgactgatgctgttagagaccctcagacattggagattttggatataaccccctgctcttttggtggtgtctctgttataacacctggcactaatacttcaaaccaagttgctgtattatatcaagatgtgaattgtactgaggttcctgtggcaattcatgctgatcaacttacaccaacatggagagtctattcaactggcagcaatgtttttcaaacaagagctggatgcttaattggagctgaacatgtgaacaattcatatgaatgtgatatcccaattggggccggcatttgtgcctcataccaaactcagaccaattctccaagaagggcctccggaggcgctagcgacccagcaagccgtgatctggttgttaattacgtgaacaccaacatgggcctgaagatccgccaactgctgtggtttcatatcagctgtctgacgtttggccgcgagacggtgctggaatacctggttagctttggcgtttggattcgtacgccaccggcctaccgcccaccaaacgcaccgattctgagcacgctgccggaaacgacggttgttcgtcgccgtgatcgcggccgtagcccgcgccgccgtacgccgagcccacgtcgtcgccgcagccagagcccgcgccgccgtcgcagccagagccgtgaaagccaatgtctggagtgactcgag

SEQ ID NO: 71 - protein, 447 aa, Artificial Sequence
  Synthetic
MDIDPYKEFGATVELLSFLPSDFFPSVRDLLDTASALYREALESPEHCSPHHTALRQAILCWGELMTLATWVGNNLEGSAGGGRDPASRDLVVNYVNTNMGLKIRQLLWFHISCLTFGRETVLEYLVSFGVWIRTPPAYRPPNAPILSTLPETTVVGGSSGGSGGSGGSGGSGGSGGSTMDIDPYKEFGATVELLSFLPSDFFPSVRDLLDTASALYREALESPEHCSPHHTALRQAILCWGELMTLATWVGNNLEFGGSNSNNLDSKVGGNYNYLYRLFRKSNLKPFERDISTEIYQAGSTPCNGVEGFNCYFPLQSYGFQPTNGVGYQPYSGGASDPASRDLVVNYVNTNMGLKIRQLLWFHISCLTFGRETVLEYLVSFGVWIRTPPAYRPPNAPILSTLPETTVVRRRDRGRSPRRRTPSPRRRRSQSPRRRRSQSRESQCLE

SEQ ID NO: 72 - DNA, 1358 bp, Artificial Sequence
  Synthetic
ttaattaaatggacatcgatccgtacaaagaatttggcgcgaccgtcgagctgctgagcttcctgccgagcgattttttcccgagcgtgcgtgacctgctggacaccgcgagcgcactgtatcgtgaagcactggaaagcccagagcactgtagcccgcaccacaccgccctgcgccaggcgattctgtgctggggtgaactgatgaccctggccacctgggtgggtaataatctggaaggttccgccggcggcggccgcgatccggcgagccgtgatctggtcgtcaactatgtgaataccaacatgggtctgaaaattcgtcagctgctgtggtttcatattagctgcctgaccttcggtcgtgaaaccgtgctggagtatctggtgagcttcggtgtgtggattcgcaccccgccggcgtatcgtccgccgaacgcgccaattctgagcacgctgccggagaccaccgtggttggtgggagctctggtggcagcggcggcagcggtggtagcggtggcagcggtggtagcggcgggtcgactatggatatcgacccatataaagaatttggcgcgacggttgagctgctgagctttctgccaagcgatttctttccgagcgtccgcgacctgctggataccgccagcgcactgtatcgtgaagccctggagagcccggaacattgcagcccgcatcatacggccctgcgtcaggcaatcctgtgctggggcgaactgatgaccctggcaacctgggtcggcaataatctggaattcggcggatccaactctaacaacctggactctaaggttggcggcaactacaactacctctacaggctgttccggaagtccaaccttaagcctttcgagagggatatcagcaccgagatctatcaggctggttctactccttgcaacggtgttgagggtttcaactgctacttcccgcttcagtcttacggtttccagcctactaatggtgtgggctaccagccttattccggaggcgctagcgacccagcaagccgtgatctggttgttaattacgtgaacaccaacatgggcctgaagatccgccaactgctgtggtttcatatcagctgtctgacgtttggccgcgagacggtgctggaatacctggttagctttggcgtttggattcgtacgccaccggcctaccgcccaccaaacgcaccgattctgagcacgctgccggaaacgacggttgttcgtcgccgtgatcgcggccgtagcccgcgccgccgtacgccgagcccacgtcgtcgccgcagccagagcccgcgccgccgtcgcagccagagccgtgaaagccaatgtctggagtgactcgag

SEQ ID NO: 73 - protein, 408 aa, Artificial Sequence
  Synthetic
MGFVLFSQLPSFLLVSTLLLFLVISHSCRAGGSYPYKSGEYRTKSFFGYGYYEVRMKAAKNVGIVSSFFTYTGPSDNNPWDEIDIEFLGKDTTKVQFNWYKNGVGGNEYLHNLGFDASQDFHTYGFEWRPDYIDFYVDGKKVYRGTRNIPVTPGKIMMNLWPGIGVDEWLGRYDGRTPLQAEYEYVKYYPNGRSDQIGYYRRATRRIRGGDGKMKDLSPRWYFYYLGTGALALLLLDRLNQLESKMSAFFGMSRIGMEVADLDDFSKQLQQSMSSADSTEFKLVVNTPFVAVFSNFDSSQWEKADWANGSVFNCVWKPSQVTFSNGKMILTLDREYNSNNLDSKVGGNYNYLYRLFRKSNLKPFERDISTEIYQAGSTPCNGVEGFNCYFPLQSYGFQPTNGVGYQPY

SEQ ID NO: 74 - DNA, 1253 bp, Artificial Sequence
  Synthetic
acgtcattaattaaatgggattcgttttgttttctcaattgccttcatttcttttggtttctactcttttgctttttcttgttatttctcattcatgtagagctggcggctcttacccttataagagcggtgagtaccggaccaagagcttctttggttacggttactacgaggtgcggatgaaggctgctaagaacgtgggtatcgtgtccagcttctttacctacaccgggccatctgataacaacccttgggatgagatcgacatcgagttccttggtaaggacactaccaaggtgcagttcaactggtacaagaacggtgttggtggcaacgagtaccttcacaaccttggctttgatgccagccaggatttccacacttacggttttgaatggcggcctgactacatcgacttctacgtggacggtaagaaggtgtacaggggcaccagaaatatccctgtgactcctggcaagatcatgatgaacctttggcctggtatcggtgtggatgagtggcttggtagatacgatggtaggactcctctgcaggctgagtacgagtacgttaagtactaccctaacggcagatctgaccaaattggttattatagaagagcaacacgtcgaataaggggtggtgatggaaaaatgaaagatttgagtcctagatggtatttctactacttggggactggagctttagccttactgctactcgacaggttgaatcaacttgagtcaaagatgtctgctttctttggcatgagccggatcggaatggaagttgctgatcttgatgatttttccaagcagcttcagcaatcgatgtcaagtgcagattctaccgaattcaagcttgtggtgaatactcctttcgtggccgtgttcagcaacttcgattctagccagtgggagaaagctgattgggctaacggttctgtgttcaactgcgtgtggaagccttctcaggtgaccttctctaacggcaagatgattctgaccctggaccgtgagtacaacagcaacaacctggattctaaggtcggcggcaactacaactacctctacaggctgttccggaagtccaaccttaagcctttcgagagggatatcagcaccgagatctatcaggctggttctactccttgcaatggcgttgagggtttcaactgctacttcccgcttcagtcttacggattccagcctactaatggtgtgggctaccagccttattagctcgagacgaag

SEQ ID NO: 75 - protein, 233 aa, Artificial Sequence
  Synthetic
MDIDPYKEFGATVELLSFLPSDFFPSVRDLLDTASALYREALESPEHCSPHHTALRQAILCWGELMTLATWVGANLEDPASRDLVVNYVNTNMGLKIRQLLWFHISCLTFGRETVLEYLVSFGVWIRTPPAYRPPNAPILSTLPSGGSDQIGYYRRATRRIRGGDGKMKDLSPRWYFYYLGTGALALLLLDRLNQLESKMSAFFGMSRIGMEVADLDDFSKQLQQSMSSADST

SEQ ID NO: 76 - DNA, 728 bp, Artificial Sequence
  Synthetic
acgtcattaattaaatggacatcgatccgtacaaagaatttggcgcgaccgtcgagctgctgagcttcctgccgagcgattttttcccgagcgtgcgtgacctgctggacaccgcgagcgcactgtatcgtgaagcactggaaagcccagagcactgtagcccgcaccacaccgccctgcgccaggcgattctgtgctggggtgaactgatgaccctggccacctgggtgggtgctaaccttgaggatccggcgagccgtgatctggtcgtcaactatgtgaataccaacatgggtctgaaaattcgtcagctgctgtggtttcatattagctgcctgaccttcggtcgtgaaaccgtgctggagtatctggtgagcttcggtgtgtggattcgcaccccgccggcgtatcgtccgccgaacgcgccaattctgagcacgctgccgtccggaggtagcgaccaaattggttattatagaagagcaacacgtcgaataaggggtggtgatggaaaaatgaaagatttgagtcctagatggtatttctactacttggggactggagctttagccttactgctactcgacaggttgaatcaacttgagtcaaagatgtctgctttctttggcatgagccggatcggaatggaagttgctgatcttgatgatttttccaagcagcttcagcaatcgatgtcaagtgcagattctacctagctcgagacgaag

SEQ ID NO: 77 - protein, 334 aa, Artificial Sequence
  Synthetic
MGFVLFSQLPSFLLVSTLLLFLVISHSCRAGGSYPYKSGEYRTKSFFGYGYYEVRMKAAKNVGIVSSFFTYTGPSDNNPWDEIDIEFLGKDTTKVQFNWYKNGVGGNEYLHNLGFDASQDFHTYGFEWRPDYIDFYVDGKKVYRGTRNIPVTPGKIMMNLWPGIGVDEWLGRYDGRTPLQAEYEYVKYYPNGRSEFKLVVNTPFVAVFSNFDSSQWEKADWANGSVFNCVWKPSQVTFSNGKMILTLDREYQIGYYRRATRRIRGGDGKMKDLSPRWYFYYLGTGALALLLLDRLNQLESKMSAFFGMSRIGMEVADLDDFSKQLQQSMSSADS

SEQ ID NO: 78 - DNA, 1031 bp, Artificial Sequence
  Synthetic
acgtcattaattaaatgggattcgttttgttttctcaattgccttcatttcttttggtttctactcttttgctttttcttgttatttctcattcatgtagagctggcggctcttacccttataagagcggtgagtaccggaccaagagcttctttggttacggttactacgaggtgcggatgaaggctgctaagaacgtgggtatcgtgtccagcttctttacctacaccgggccatctgataacaacccttgggatgagatcgacatcgagttccttggtaaggacactaccaaggtgcagttcaactggtacaagaacggtgttggtggcaacgagtaccttcacaaccttggctttgatgccagccaggatttccacacttacggttttgaatggcggcctgactacatcgacttctacgtggacggtaagaaggtgtacaggggcaccagaaatatccctgtgactcctggcaagatcatgatgaacctttggcctggtatcggtgtggatgagtggcttggtagatacgatggtaggactcctctgcaggctgagtacgagtacgttaagtactaccctaacggcagatctgaattcaagcttgtggtgaatactcctttcgtggccgtgttcagcaacttcgattctagccagtgggagaaagctgattgggctaacggttctgtgttcaactgcgtgtggaagccttctcaggtgaccttctctaacggcaagatgattctgaccctggaccgtgagtatcaaattggatactacaggcgggctacaagaagaatccgtggtggtgatggaaaaatgaaagatttgtcccctagatggtatttctattatcttgggactggtgccttagcattactactcctggaccgattgaatcaacttgagagtaagatgtctgctttctttggcatgagcaggataggaatggaagttgcagaccttgatgatttttcaaagcaattgcagcagtcgatgtcaagtgctgattcttgactcgagacgaag

SEQ ID NO: 79 - protein, 407 aa, Artificial Sequence
  Synthetic
MGFVLFSQLPSFLLVSTLLLFLVISHSCRAGGSYPYKSGEYRTKSFFGYGYYEVRMKAAKNVGIVSSFFTYTGPSDNNPWDEIDIEFLGKDTTKVQFNWYKNGVGGNEYLHNLGFDASQDFHTYGFEWRPDYIDFYVDGKKVYRGTRNIPVTPGKIMMNLWPGIGVDEWLGRYDGRTPLQAEYEYVKYYPNGRSEFKLVVNTPFVAVFSNFDSSQWEKADWANGSVFNCVWKPSQVTFSNGKMILTLDREYQIGYYRRATRRIRGGDGKMKDLSPRWYFYYLGTGALALLLLDRLNQLESKMSAFFGMSRIGMEVADLDDFSKQLQQSMSSADSTNSNNLDSKVGGNYNYLYRLFRKSNLKPFERDISTEIYQAGSTPCNGVEGFNCYFPLQSYGFQPTNGVGYQPY

SEQ ID NO: 80 - DNA, 1250 bp, Artificial Sequence
  Synthetic
acgtcattaattaaatgggattcgttttgttttctcaattgccttcatttcttttggtttctactcttttgctttttcttgttatttctcattcatgtagagctggcggctcttacccttataagagcggtgagtaccggaccaagagcttctttggttacggttactacgaggtgcggatgaaggctgctaagaacgtgggtatcgtgtccagcttctttacctacaccgggccatctgataacaacccttgggatgagatcgacatcgagttccttggtaaggacactaccaaggtgcagttcaactggtacaagaacggtgttggtggcaacgagtaccttcacaaccttggctttgatgccagccaggatttccacacttacggttttgaatggcggcctgactacatcgacttctacgtggacggtaagaaggtgtacaggggcaccagaaatatccctgtgactcctggcaagatcatgatgaacctttggcctggtatcggtgtggatgagtggcttggtagatacgatggtaggactcctctgcaggctgagtacgagtacgttaagtactaccctaacggcagatctgaattcaagcttgtggtgaatactcctttcgtggccgtgttcagcaacttcgattctagccagtgggagaaagctgattgggctaacggttctgtgttcaactgcgtgtggaagccttctcaggtgaccttctctaacggcaagatgattctgaccctggaccgtgagtatcaaattggatactacaggcgggctacaagaagaatccgtggtggtgatggaaaaatgaaagatttgtcccctagatggtatttctattatcttgggactggtgccttagcattactactcctggaccgattgaatcaacttgagagtaagatgtctgctttctttggcatgagcaggataggaatggaagttgcagaccttgatgatttttcaaagcaattgcagcagtcgatgtcaagtgctgattctacgaactctaacaacctggactctaaggttggcggcaactacaactacctctacaggctgttccggaagtccaaccttaagcctttcgagagggatatcagcaccgagatctatcaggctggttctactccttgcaacggtgttgagggtttcaactgctacttcccgcttcagtcttacggtttccagcctactaatggtgtgggctaccagccttattagctcgagacgaag

SEQ ID NO: 81 - protein, 375 aa, Artificial Sequence
  Synthetic
MGKMASLFATFLVVLVSLSLASESSAGGSYPYKSGEYRTKSFFGYGYYEVRMKAAKNVGIVSSFFTYTGPSDNNPWDEIDIEFLGKDTTKVQFNWYKNGVGGNEYLHNLGFDASQDFHTYGFEWRPDYIDFYVDGKKVYRGTRNIPVTPGKIMMNLWPGIGVDEWLGRYDGRTPLQAEYEYVKYYPNGRSEFKLVVNTPFVAVFSNFDSSQWEKADWANGSVFNCVWKPSQVTFSNGKMILTLDREYGGGGSGGGGTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPP

SEQ ID NO: 82 - DNA, 1160 bp, Artificial Sequence
  Synthetic
acgtcattaattaaatgggaaaaatggcttctctttttgctactttccttgttgtgttggttagtctttctctagctagtgagagtagtgctggcggctcttacccttataagagcggtgagtaccggaccaagagcttctttggttacggttactacgaggtgcggatgaaggctgctaagaacgtgggtatcgtgtccagcttctttacctacaccgggccatctgataacaacccttgggatgagatcgacatcgagttccttggtaaggacactaccaaggtgcagttcaactggtacaagaacggtgttggtggcaacgagtaccttcacaaccttggctttgatgccagccaggatttccacacttacggttttgaatggcggcctgactacatcgacttctacgtggacggtaagaaggtgtacaggggcaccagaaatatccctgtgactcctggcaagatcatgatgaacctttggcctggtatcggtgtggatgagtggcttggtagatacgatggtaggactcctctgcaggctgagtacgagtacgttaagtactaccctaacggcagatctgaattcaagcttgtggtgaatactcctttcgtggccgtgttcagcaacttcgattctagccagtgggagaaagctgattgggctaacggttctgtgttcaactgcgtgtggaagccttctcaggtgaccttctctaacggcaagatgattctgaccctggaccgtgagtatggcgggggcggctcaggaggtggtggaacgaagaagtctgctgctgaagcttcaaagaaaccacgacagaaaagaactgcaacaaaagcatataatgtaactcaagcatttggaaggcgtggtcctgagcaaacacaaggaaattttggtgatcaagagcttataaggcaaggtaccgactacaagcattggcctcagattgctcagtttgctcccagtgcctccgcgttcttcggcatgtctagaattgggatggaagttaccccgagcgggacatggctcacttatactggagcaatcaagttggatgataaagatccaaatttcaaggaccaggtgatactgttaaacaaacacattgatgcctataaaacatttcctccatgactcgagcctaggacgaag

SEQ ID NO: 83 - protein, 153 aa, Artificial Sequence
  Synthetic
MGKMASLFATFLVVLVSLSLASESSAHHHHHHHHTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPP

SEQ ID NO: 84 - DNA, 494 bp, Artificial Sequence
  Synthetic
acgtcattaattaaatgggaaaaatggcttctctttttgctactttccttgttgtgttggttagtctttctctagctagtgagagtagtgctcaccatcatcatcaccatcatcacaccaagaaaagcgctgccgaagcgtcaaagaagccaaggcagaagagaactgctacaaaagcttataatgtgactcaagcatttggaagacgaggccctgagcaaacacaggggaattttggtgatcaagaactgatcaggcaaggaacagattataaacattggcctcaaattgctcagtttgctccttctgcctccgcattctttggtatgtctcgtattggaatggaggtaactccgagtggtacatggcttacgtatactggcgcaattaaattggatgataaagacccaaatttcaaggaccaggttatactattaaacaagcatatagatgcatacaaaaccttcccaccctgactcgagcctaggacgaag

SEQ ID NO: 85 - protein, 153 aa, Artificial Sequence
  Synthetic
MGKMASLFATFLVVLVSLSLASESSATKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPHHHHHHHH

SEQ ID NO: 86 - DNA, 494 bp, Artificial Sequence
  Synthetic
acgtcattaattaaatgggaaaaatggcttctctttttgctactttccttgttgtgttggttagtctttctctagctagtgagagtagtgctacaaagaaatctgctgctgaagccagcaagaagccaagacagaaaagaactgcaacaaaagcatataatgttacccaggcatttggaaggcgaggccccgagcaaactcaagggaattttggtgaccaagaactgataaggcagggaactgattataaacattggcctcaaattgctcaatttgctccgtcagcgtcagccttctttgggatgagtcgtattggaatggaggtaacgccatctggtacatggcttacctatactggtgcaatcaaattggatgataaagatccaaatttcaaggaccaggtgatattactcaacaagcatattgatgcttacaagacattccctcctcatcaccatcatcatcaccaccattgactcgagcctaggacgaag

SEQ ID NO: 87 - protein, 155 aa, Artificial Sequence
  Synthetic
MGKMASLFATFLVVLVSLSLASESSAHHHHHHHHTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPP

SEQ ID NO: 88 - DNA, 500 bp, Artificial Sequence
  Synthetic
acgtcattaattaaatgggaaaaatggcttctctttttgctactttccttgttgtgttggttagtctttctctagctagtgagagtagtgctcaccatcatcatcaccatcatcacacagtgaccaagaaaagcgctgccgaagcgtcaaagaagccaaggcagaagagaactgctacaaaagcttataatgtgactcaagcatttggaagacgaggccctgagcaaacacaggggaattttggtgatcaagaactgatcaggcaaggaacagattataaacattggcctcaaattgctcagtttgctccttctgcctccgcattctttggtatgtctcgtattggaatggaggtaactccgagtggtacatggcttacgtatactggcgcaattaaattggatgataaagacccaaatttcaaggaccaggttatactattaaacaagcatatagatgcatacaaaaccttcccaccctgactcgagcctaggacgaag

SEQ ID NO: 89 - protein, 155 aa, Artificial Sequence
  Synthetic
MGKMASLFATFLVVLVSLSLASESSATVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPHHHHHHHH

SEQ ID NO: 90 - DNA, 500 bp, Artificial Sequence
  Synthetic
acgtcattaattaaatgggaaaaatggcttctctttttgctactttccttgttgtgttggttagtctttctctagctagtgagagtagtgctacagtgacaaagaaatctgctgctgaagccagcaagaagccaagacagaaaagaactgcaacaaaagcatataatgttacccaggcatttggaaggcgaggccccgagcaaactcaagggaattttggtgaccaagaactgataaggcagggaactgattataaacattggcctcaaattgctcaatttgctccgtcagcgtcagccttctttgggatgagtcgtattggaatggaggtaacgccatctggtacatggcttacctatactggtgcaatcaaattggatgataaagatccaaatttcaaggaccaggtgatattactcaacaagcatattgatgcttacaagacattccctcctcatcaccatcatcatcaccaccattgactcgagcctaggacgaag

SEQ ID NO: 91 - protein, 449 aa, Artificial Sequence
  Synthetic
MGKMASLFATFLVVLVSLSLASESSATTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKSGGGSTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKK

SEQ ID NO: 92 - DNA, 1377 bp, Artificial Sequence
  Synthetic
acgtcattaattaaatgggaaaaatggcttctctttttgctactttccttgttgtgttggttagtctttctctagctagtgagagtagtgctacgaccaggacacaacttccccctgcttatacaaactctttcactaggggcgtctattaccctgataaggtgtttcggagtagtgtattgcattcaactcaagatttgttccttccgttcttctcaaatgtgacatggtttcatgccattcatgtctcagggaccaatggtacaaagagatttgataatcctgttctcccattcaatgacggtgtttattttgcttcaacagagaagagcaacattataagaggatggatttttggaacaacgcttgactcaaaaactcagagtttattgatagtaaataatgcaactaacgttgtgatcaaagtttgtgaattccagttctgcaatgatccatttctgggagtttactatcacaagaacaacaagtcttggatggaaagtgagttcagagtatattcttctgcaaacaattgcacttttgagtatgtttctcagccttttttgatggatcttgaggggaaacaaggaaatttcaaaaatctacgcgaatttgtttttaagaatattgatggctacttcaagatttacagcaagcatactccaataaaccttgtgagagacctgccacagggattttccgccttagaaccactggtagacttaccaattgggatcaatatcacacgttttcaaactctcctagcgttgcacaggagttacctaactcctggtgattcttcgagcggttggactgctggagctgcagcatattatgtgggttatttgcaaccccgaacatttcttctcaaatataatgaaaatggaaccattactgatgctgttgattgtgctttggatcctctttctgaaacaaaatgtaccctgaaatcatttactgtcgagaaatccggtggtggctccacggtgaccaaaaaaagtgccgctgaagcatccaagaaaccgcgacagaagaggactgcaacaaaagcttataatgttactcaagcatttgggagacgtggtcctgaacaaactcagggaaattttggtgaccaggagcttataaggcaaggaactgattataagcattggcctcaaattgctcagtttgctccatcagcgtctgccttcttcgggatgtcaagaattggaatggaagtaaccccttctggcacatggttaacatatactggtgcaatcaaattggatgataaagaccccaatttcaaagatcaagttatactcctgaacaagcacattgatgcttacaagacatttcctccaacagagccaaagaagtgactcgagtcgacgt

SEQ ID NO: 93 - protein, 442 aa, Artificial Sequence
  Synthetic
MGKMASLFATFLVVLVSLSLASESSATTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKSGGGSKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPT

SEQ ID NO: 94 - DNA, 1356 bp, Artificial Sequence
  Synthetic
acgtcattaattaaatgggaaaaatggcttctctttttgctactttccttgttgtgttggttagtctttctctagctagtgagagtagtgctacgaccaggacacaacttccccctgcttatacaaactctttcactaggggcgtctattaccctgataaggtgtttcggagtagtgtattgcattcaactcaagatttgttccttccgttcttctcaaatgtgacatggtttcatgccattcatgtctcagggaccaatggtacaaagagatttgataatcctgttctcccattcaatgacggtgtttattttgcttcaacagagaagagcaacattataagaggatggatttttggaacaacgcttgactcaaaaactcagagtttattgatagtaaataatgcaactaacgttgtgatcaaagtttgtgaattccagttctgcaatgatccatttctgggagtttactatcacaagaacaacaagtcttggatggaaagtgagttcagagtatattcttctgcaaacaattgcacttttgagtatgtttctcagccttttttgatggatcttgaggggaaacaaggaaatttcaaaaatctacgcgaatttgtttttaagaatattgatggctacttcaagatttacagcaagcatactccaataaaccttgtgagagacctgccacagggattttccgccttagaaccactggtagacttaccaattgggatcaatatcacacgttttcaaactctcctagcgttgcacaggagttacctaactcctggtgattcttcgagcggttggactgctggagctgcagcatattatgtgggttatttgcaaccccgaacatttcttctcaaatataatgaaaatggaaccattactgatgctgttgattgtgctttggatcctctttctgaaacaaaatgtaccctgaaatcatttactgtcgagaaatccggtggtggctccaaaaaaagtgccgctgaagcatccaagaaaccgcgacagaagaggactgcaacaaaagcttataatgttactcaagcatttgggagacgtggtcctgaacaaactcagggaaattttggtgaccaggagcttataaggcaaggaactgattataagcattggcctcaaattgctcagtttgctccatcagcgtctgccttcttcgggatgtcaagaattggaatggaagtaaccccttctggcacatggttaacatatactggtgcaatcaaattggatgataaagaccccaatttcaaagatcaagttatactcctgaacaagcacattgatgcttacaagacatttcctccaacatgactcgagtcgacgt

SEQ ID NO: 95 - protein, 429 aa, Artificial Sequence
  Synthetic
MGKMASLFATFLVVLVSLSLASESSATTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDSGGGSTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKK

SEQ ID NO: 96 - DNA, 1317 bp, Artificial Sequence
  Synthetic
acgtcattaattaaatgggaaaaatggcttctctttttgctactttccttgttgtgttggttagtctttctctagctagtgagagtagtgctacgaccaggacacaacttccccctgcttatacaaactctttcactaggggcgtctattaccctgataaggtgtttcggagtagtgtattgcattcaactcaagatttgttccttccgttcttctcaaatgtgacatggtttcatgccattcatgtctcagggaccaatggtacaaagagatttgataatcctgttctcccattcaatgacggtgtttattttgcttcaacagagaagagcaacattataagaggatggatttttggaacaacgcttgactcaaaaactcagagtttattgatagtaaataatgcaactaacgttgtgatcaaagtttgtgaattccagttctgcaatgatccatttctgggagtttactatcacaagaacaacaagtcttggatggaaagtgagttcagagtatattcttctgcaaacaattgcacttttgagtatgtttctcagccttttttgatggatcttgaggggaaacaaggaaatttcaaaaatctacgcgaatttgtttttaagaatattgatggctacttcaagatttacagcaagcatactccaataaaccttgtgagagacctgccacagggattttccgccttagaaccactggtagacttaccaattgggatcaatatcacacgttttcaaactctcctagcgttgcacaggagttacctaactcctggtgattcttcgagcggttggactgctggagctgcagcatattatgtgggttatttgcaaccccgaacatttcttctcaaatataatgaaaatggaaccattactgatgctgttgattccggtggtggctccacggtgaccaaaaaaagtgccgctgaagcatccaagaaaccgcgacagaagaggactgcaacaaaagcttataatgttactcaagcatttgggagacgtggtcctgaacaaactcagggaaattttggtgaccaggagcttataaggcaaggaactgattataagcattggcctcaaattgctcagtttgctccatcagcgtctgccttcttcgggatgtcaagaattggaatggaagtaaccccttctggcacatggttaacatatactggtgcaatcaaattggatgataaagaccccaatttcaaagatcaagttatactcctgaacaagcacattgatgcttacaagacatttcctccaacagagccaaagaagtgactcgagtcgacgt

SEQ ID NO: 97 - protein, 422 aa, Artificial Sequence
  Synthetic
MGKMASLFATFLVVLVSLSLASESSATTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDSGGGSKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPT

SEQ ID NO: 98 - DNA, 1296 bp, Artificial Sequence
  Synthetic
acgtcattaattaaatgggaaaaatggcttctctttttgctactttccttgttgtgttggttagtctttctctagctagtgagagtagtgctacgaccaggacacaacttccccctgcttatacaaactctttcactaggggcgtctattaccctgataaggtgtttcggagtagtgtattgcattcaactcaagatttgttccttccgttcttctcaaatgtgacatggtttcatgccattcatgtctcagggaccaatggtacaaagagatttgataatcctgttctcccattcaatgacggtgtttattttgcttcaacagagaagagcaacattataagaggatggatttttggaacaacgcttgactcaaaaactcagagtttattgatagtaaataatgcaactaacgttgtgatcaaagtttgtgaattccagttctgcaatgatccatttctgggagtttactatcacaagaacaacaagtcttggatggaaagtgagttcagagtatattcttctgcaaacaattgcacttttgagtatgtttctcagccttttttgatggatcttgaggggaaacaaggaaatttcaaaaatctacgcgaatttgtttttaagaatattgatggctacttcaagatttacagcaagcatactccaataaaccttgtgagagacctgccacagggattttccgccttagaaccactggtagacttaccaattgggatcaatatcacacgttttcaaactctcctagcgttgcacaggagttacctaactcctggtgattcttcgagcggttggactgctggagctgcagcatattatgtgggttatttgcaaccccgaacatttcttctcaaatataatgaaaatggaaccattactgatgctgttgattccggtggtggctccaaaaaaagtgccgctgaagcatccaagaaaccgcgacagaagaggactgcaacaaaagcttataatgttactcaagcatttgggagacgtggtcctgaacaaactcagggaaattttggtgaccaggagcttataaggcaaggaactgattataagcattggcctcaaattgctcagtttgctccatcagcgtctgccttcttcgggatgtcaagaattggaatggaagtaaccccttctggcacatggttaacatatactggtgcaatcaaattggatgataaagaccccaatttcaaagatcaagttatactcctgaacaagcacattgatgcttacaagacatttcctccaacatgactcgagtcgacgt

SEQ ID NO: 99 - protein, 393 aa, Artificial Sequence
  Synthetic
MGKMASLFATFLVVLVSLSLASESSARVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESGGGSTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKK

SEQ ID NO: 100 - DNA, 1209 bp, Artificial Sequence
  Synthetic
acgtcattaattaaatgggaaaaatggcttctctttttgctactttccttgttgtgttggttagtctttctctagctagtgagagtagtgctagggttcagcctactgagtctatcgtgcggttccctaacatcaccaacttgtgccctttcggcgaggtgttcaatgctactaggttcgcttctgtgtacgcctggaaccggaagaggatttctaactgcgtggccgattacagcgtgctgtacaactctgctagcttcagcaccttcaagtgctacggtgtgtctcctaccaagctgaacgatctgtgcttcaccaacgtgtacgctgactctttcgtgatcaggggtgatgaggttaggcagattgctcctggtcagaccggtaagatcgctgactacaactacaagctgcctgatgacttcaccggttgcgtgatcgcttggaactctaacaacctggactctaaggttggcggcaattacaactacctctaccggctgttccggaagtctaaccttaagcctttcgagcgggatatcagcaccgagatctatcaggctggttctactccttgcaatggcgttgagggtttcaactgctacttcccgcttcagtcttacggattccagcctactaatggtgtgggctaccagccttacagagtggtggttttgtctttcgagcttctgcatgctcctgctactgtttgcggtcctaagaagtctacaaatcttgttaagaataagtgcgttaacttcaacttcaatggtttgactggaacaggtgttcttacagaatccggtggtggctccacggtgaccaaaaaaagtgccgctgaagcatccaagaaaccgcgacagaagaggactgcaacaaaagcttataatgttactcaagcatttgggagacgtggtcctgaacaaactcagggaaattttggtgaccaggagcttataaggcaaggaactgattataagcattggcctcaaattgctcagtttgctccatcagcgtctgccttcttcgggatgtcaagaattggaatggaagtaaccccttctggcacatggttaacatatactggtgcaatcaaattggatgataaagaccccaatttcaaagatcaagttatactcctgaacaagcacattgatgcttacaagacatttcctccaacagagccaaagaagtgactcgagtcgacgt

SEQ ID NO: 101 - protein, 386 aa, Artificial Sequence
  Synthetic
MGKMASLFATFLVVLVSLSLASESSARVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESGGGSKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPT

SEQ ID NO: 102 - DNA, 1188 bp, Artificial Sequence
  Synthetic
acgtcattaattaaatgggaaaaatggcttctctttttgctactttccttgttgtgttggttagtctttctctagctagtgagagtagtgctagggttcagcctactgagtctatcgtgcggttccctaacatcaccaacttgtgccctttcggcgaggtgttcaatgctactaggttcgcttctgtgtacgcctggaaccggaagaggatttctaactgcgtggccgattacagcgtgctgtacaactctgctagcttcagcaccttcaagtgctacggtgtgtctcctaccaagctgaacgatctgtgcttcaccaacgtgtacgctgactctttcgtgatcaggggtgatgaggttaggcagattgctcctggtcagaccggtaagatcgctgactacaactacaagctgcctgatgacttcaccggttgcgtgatcgcttggaactctaacaacctggactctaaggttggcggcaattacaactacctctaccggctgttccggaagtctaaccttaagcctttcgagcgggatatcagcaccgagatctatcaggctggttctactccttgcaatggcgttgagggtttcaactgctacttcccgcttcagtcttacggattccagcctactaatggtgtgggctaccagccttacagagtggtggttttgtctttcgagcttctgcatgctcctgctactgtttgcggtcctaagaagtctacaaatcttgttaagaataagtgcgttaacttcaacttcaatggtttgactggaacaggtgttcttacagaatccggtggtggctccaaaaaaagtgccgctgaagcatccaagaaaccgcgacagaagaggactgcaacaaaagcttataatgttactcaagcatttgggagacgtggtcctgaacaaactcagggaaattttggtgaccaggagcttataaggcaaggaactgattataagcattggcctcaaattgctcagtttgctccatcagcgtctgccttcttcgggatgtcaagaattggaatggaagtaaccccttctggcacatggttaacatatactggtgcaatcaaattggatgataaagaccccaatttcaaagatcaagttatactcctgaacaagcacattgatgcttacaagacatttcctccaacatgactcgagtcgacgt

SEQ ID NO: 103 - protein, 333 aa, Artificial Sequence
  Synthetic
MGKMASLFATFLVVLVSLSLASESSAASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATSGGGSTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKK

SEQ ID NO: 104 - DNA, 1029 bp, Artificial Sequence
  Synthetic
acgtcattaattaaatgggaaaaatggcttctctttttgctactttccttgttgtgttggttagtctttctctagctagtgagagtagtgctgcttctgtgtacgcctggaaccggaagaggatttctaactgcgtggccgattacagcgtgctgtacaactctgctagcttcagcaccttcaagtgctacggtgtgtctcctaccaagctgaacgatctgtgcttcaccaacgtgtacgctgactctttcgtgatcaggggtgatgaggttaggcagattgctcctggtcagaccggtaagatcgctgactacaactacaagctgcctgatgacttcaccggttgcgtgatcgcttggaactctaacaacctggactctaaggttggcggcaattacaactacctctaccggctgttccggaagtctaaccttaagcctttcgagcgggatatcagcaccgagatctatcaggctggttctactccttgcaatggcgttgagggtttcaactgctacttcccgcttcagtcttacggattccagcctactaatggtgtgggctaccagccttacagagtggtggttttgtctttcgagcttctgcatgctcctgctacttctggaggtggttctacggtgaccaaaaaaagtgccgctgaagcatccaagaaaccgcgacagaagaggactgcaacaaaagcttataatgttactcaagcatttgggagacgtggtcctgaacaaactcagggaaattttggtgaccaggagcttataaggcaaggaactgattataagcattggcctcaaattgctcagtttgctccatcagcgtctgccttcttcgggatgtcaagaattggaatggaagtaaccccttctggcacatggttaacatatactggtgcaatcaaattggatgataaagaccccaatttcaaagatcaagttatactcctgaacaagcacattgatgcttacaagacatttcctccaacagagccaaagaagtgactcgagtcgacgt

SEQ ID NO: 105 - protein, 326 aa, Artificial Sequence
  Synthetic
MGKMASLFATFLVVLVSLSLASESSAASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATSGGGSKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPT

SEQ ID NO: 106 - DNA, 1008 bp, Artificial Sequence
  Synthetic
acgtcattaattaaatgggaaaaatggcttctctttttgctactttccttgttgtgttggttagtctttctctagctagtgagagtagtgctgcttctgtgtacgcctggaaccggaagaggatttctaactgcgtggccgattacagcgtgctgtacaactctgctagcttcagcaccttcaagtgctacggtgtgtctcctaccaagctgaacgatctgtgcttcaccaacgtgtacgctgactctttcgtgatcaggggtgatgaggttaggcagattgctcctggtcagaccggtaagatcgctgactacaactacaagctgcctgatgacttcaccggttgcgtgatcgcttggaactctaacaacctggactctaaggttggcggcaattacaactacctctaccggctgttccggaagtctaaccttaagcctttcgagcgggatatcagcaccgagatctatcaggctggttctactccttgcaatggcgttgagggtttcaactgctacttcccgcttcagtcttacggattccagcctactaatggtgtgggctaccagccttacagagtggtggttttgtctttcgagcttctgcatgctcctgctacttctggaggtggttctaaaaaaagtgccgctgaagcatccaagaaaccgcgacagaagaggactgcaacaaaagcttataatgttactcaagcatttgggagacgtggtcctgaacaaactcagggaaattttggtgaccaggagcttataaggcaaggaactgattataagcattggcctcaaattgctcagtttgctccatcagcgtctgccttcttcgggatgtcaagaattggaatggaagtaaccccttctggcacatggttaacatatactggtgcaatcaaattggatgataaagaccccaatttcaaagatcaagttatactcctgaacaagcacattgatgcttacaagacatttcctccaacatgactcgagtcgacgt

SEQ ID NO: 107 - DNA, 1377 bp, Artificial Sequence
  Synthetic
acgtcattaattaaatgggaaaaatggcttctctttttgctactttccttgttgtgttggttagtctttctctagctagtgagagtagtgctacgacacgaactcagctaccccctgcatatactaactcattcacccgtggagtttactatcctgataaagtatttcgcagctcagtgcttcattcgacgcaagatttgtttcttccttttttctccaatgttacatggtttcatgcaattcatgttagtggcacaaatggaactaagagatttgataatccagtactaccgttcaatgatggggtctattttgcttcaactgagaagtctaacatcatacggggttggatttttggaacaactttagactccaaaactcaaagcttgctgattgttaacaatgccaccaatgtggtcatcaaagtatgtgaatttcagttttgcaatgaccctttccttggcgtgtattatcacaagaacaacaagtcatggatggaatcagagttcagagtgtactcttcagcaaataattgtacttttgaatatgtttctcagccatttttgatggatcttgaaggaaagcaagggaattttaagaatttgagggagtttgttttcaaaaatattgatggatatttcaagatttactccaagcatacgccaatcaaccttgtcagagacttaccacaaggtttttctgcgttggagccattggtggacctccctattggaattaacataactaggtttcaaacacttcttgcactccacagaagttatctaactcctggtgattcttctagtggatggactgctggtgctgctgcctactacgtaggttatttgcagcccaggacattcttactcaaatataatgaaaatggtaccataactgatgctgttgattgtgcactggatccactgtctgaaacaaaatgcacattaaaaagtttcacagttgagaaatctgggggtggcagtaccgtaacgaagaagtcagctgctgaggcgagcaagaaaccaaggcagaaaagaactgcaacaaaagcatataatgttacacaagcatttgggaggcgtggtcctgaacaaactcaaggaaattttggtgatcaagaattgatacgacagggcactgattataaacattggcctcaaattgctcagtttgctccctccgcctctgcattctttggaatgtcaagaattggtatggaggtgaccccgagtgggacatggttaacatatactggagccatcaagcttgatgataaagacccaaatttcaaggaccaggttatactgctcaacaagcacattgatgcttacaaaacattccctcctactgaaccaaaaaagtgactcgagtcgacgt

SEQ ID NO: 108 - DNA, 1356 bp, Artificial Sequence
  Synthetic
acgtcattaattaaatgggaaaaatggcttctctttttgctactttccttgttgtgttggttagtctttctctagctagtgagagtagtgctacgacacgaactcagctaccccctgcatatactaactcattcacccgtggagtttactatcctgataaagtatttcgcagctcagtgcttcattcgacgcaagatttgtttcttccttttttctccaatgttacatggtttcatgcaattcatgttagtggcacaaatggaactaagagatttgataatccagtactaccgttcaatgatggggtctattttgcttcaactgagaagtctaacatcatacggggttggatttttggaacaactttagactccaaaactcaaagcttgctgattgttaacaatgccaccaatgtggtcatcaaagtatgtgaatttcagttttgcaatgaccctttccttggcgtgtattatcacaagaacaacaagtcatggatggaatcagagttcagagtgtactcttcagcaaataattgtacttttgaatatgtttctcagccatttttgatggatcttgaaggaaagcaagggaattttaagaatttgagggagtttgttttcaaaaatattgatggatatttcaagatttactccaagcatacgccaatcaaccttgtcagagacttaccacaaggtttttctgcgttggagccattggtggacctccctattggaattaacataactaggtttcaaacacttcttgcactccacagaagttatctaactcctggtgattcttctagtggatggactgctggtgctgctgcctactacgtaggttatttgcagcccaggacattcttactcaaatataatgaaaatggtaccataactgatgctgttgattgtgcactggatccactgtctgaaacaaaatgcacattaaaaagtttcacagttgagaaatctgggggtggcagtaagaagtcagctgctgaggcgagcaagaaaccaaggcagaaaagaactgcaacaaaagcatataatgttacacaagcatttgggaggcgtggtcctgaacaaactcaaggaaattttggtgatcaagaattgatacgacagggcactgattataaacattggcctcaaattgctcagtttgctccctccgcctctgcattctttggaatgtcaagaattggtatggaggtgaccccgagtgggacatggttaacatatactggagccatcaagcttgatgataaagacccaaatttcaaggaccaggttatactgctcaacaagcacattgatgcttacaaaacattccctcctacttgactcgagtcgacgt

SEQ ID NO: 109 - DNA, 1317 bp, Artificial Sequence
  Synthetic
acgtcattaattaaatgggaaaaatggcttctctttttgctactttccttgttgtgttggttagtctttctctagctagtgagagtagtgctacgacacgaactcagctaccccctgcatatactaactcattcacccgtggagtttactatcctgataaagtatttcgcagctcagtgcttcattcgacgcaagatttgtttcttccttttttctccaatgttacatggtttcatgcaattcatgttagtggcacaaatggaactaagagatttgataatccagtactaccgttcaatgatggggtctattttgcttcaactgagaagtctaacatcatacggggttggatttttggaacaactttagactccaaaactcaaagcttgctgattgttaacaatgccaccaatgtggtcatcaaagtatgtgaatttcagttttgcaatgaccctttccttggcgtgtattatcacaagaacaacaagtcatggatggaatcagagttcagagtgtactcttcagcaaataattgtacttttgaatatgtttctcagccatttttgatggatcttgaaggaaagcaagggaattttaagaatttgagggagtttgttttcaaaaatattgatggatatttcaagatttactccaagcatacgccaatcaaccttgtcagagacttaccacaaggtttttctgcgttggagccattggtggacctccctattggaattaacataactaggtttcaaacacttcttgcactccacagaagttatctaactcctggtgattcttctagtggatggactgctggtgctgctgcctactacgtaggttatttgcagcccaggacattcttactcaaatataatgaaaatggtaccataactgatgctgttgattctgggggtggcagtaccgtaacgaagaagtcagctgctgaggcgagcaagaaaccaaggcagaaaagaactgcaacaaaagcatataatgttacacaagcatttgggaggcgtggtcctgaacaaactcaaggaaattttggtgatcaagaattgatacgacagggcactgattataaacattggcctcaaattgctcagtttgctccctccgcctctgcattctttggaatgtcaagaattggtatggaggtgaccccgagtgggacatggttaacatatactggagccatcaagcttgatgataaagacccaaatttcaaggaccaggttatactgctcaacaagcacattgatgcttacaaaacattccctcctactgaaccaaaaaagtgactcgagtcgacgt

SEQ ID NO: 110 - DNA, 1296 bp, Artificial Sequence
  Synthetic
acgtcattaattaaatgggaaaaatggcttctctttttgctactttccttgttgtgttggttagtctttctctagctagtgagagtagtgctacgacacgaactcagctaccccctgcatatactaactcattcacccgtggagtttactatcctgataaagtatttcgcagctcagtgcttcattcgacgcaagatttgtttcttccttttttctccaatgttacatggtttcatgcaattcatgttagtggcacaaatggaactaagagatttgataatccagtactaccgttcaatgatggggtctattttgcttcaactgagaagtctaacatcatacggggttggatttttggaacaactttagactccaaaactcaaagcttgctgattgttaacaatgccaccaatgtggtcatcaaagtatgtgaatttcagttttgcaatgaccctttccttggcgtgtattatcacaagaacaacaagtcatggatggaatcagagttcagagtgtactcttcagcaaataattgtacttttgaatatgtttctcagccatttttgatggatcttgaaggaaagcaagggaattttaagaatttgagggagtttgttttcaaaaatattgatggatatttcaagatttactccaagcatacgccaatcaaccttgtcagagacttaccacaaggtttttctgcgttggagccattggtggacctccctattggaattaacataactaggtttcaaacacttcttgcactccacagaagttatctaactcctggtgattcttctagtggatggactgctggtgctgctgcctactacgtaggttatttgcagcccaggacattcttactcaaatataatgaaaatggtaccataactgatgctgttgattctgggggtggcagtaagaagtcagctgctgaggcgagcaagaaaccaaggcagaaaagaactgcaacaaaagcatataatgttacacaagcatttgggaggcgtggtcctgaacaaactcaaggaaattttggtgatcaagaattgatacgacagggcactgattataaacattggcctcaaattgctcagtttgctccctccgcctctgcattctttggaatgtcaagaattggtatggaggtgaccccgagtgggacatggttaacatatactggagccatcaagcttgatgataaagacccaaatttcaaggaccaggttatactgctcaacaagcacattgatgcttacaaaacattccctcctacttgactcgagtcgacgt

SEQ ID NO: 111 - DNA, 1209 bp, Artificial Sequence
  Synthetic
acgtcattaattaaatgggaaaaatggcttctctttttgctactttccttgttgtgttggttagtctttctctagctagtgagagtagtgctcgagttcagccaacagagagtatcgttagattccctaatataaccaacctctgcccatttggagaagtgtttaacgccacaagatttgcttctgtatatgcttggaaccgcaaaagaataagcaattgtgtggcagactacagcgtgctatacaattctgcgagtttctcaacattcaagtgctatggtgtgtcaccaactaagttaaatgatctctgctttactaatgtgtacgctgattcatttgtcattagaggtgatgaagttcgtcagatcgctccaggacaaactggcaagatagctgactacaactataagcttccggatgactttacgggatgtgttattgcatggaattccaacaaccttgattctaaagttggagggaattataattacttgtatcggcttttcaggaaatcaaatttgaaaccttttgagagggatatttcgaccgaaatttatcaagccgggagtactccatgcaatggtgtagaaggattcaactgttactttcctctgcaatcatatggatttcaacccacaaacggcgtgggttatcagccctatagggtagtagttctaagttttgaacttttgcatgcacctgcaacagtttgtggacctaagaaatccacaaatttagtcaagaataaatgtgtaaatttcaatttcaatgggttgaccggcactggtgttctgactgagtctggaggtggttctaccgtaacgaagaagtcagctgctgaggcgagcaagaaaccaaggcagaaaagaactgcaacaaaagcatataatgttacacaagcatttgggaggcgtggtcctgaacaaactcaaggaaattttggtgatcaagaattgatacgacagggcactgattataaacattggcctcaaattgctcagtttgctccctccgcctctgcattctttggaatgtcaagaattggtatggaggtgaccccgagtgggacatggttaacatatactggagccatcaagcttgatgataaagacccaaatttcaaggaccaggttatactgctcaacaagcacattgatgcttacaaaacattccctcctactgaaccaaaaaagtgactcgagtcgacgt

SEQ ID NO: 112 - DNA, 1188 bp, Artificial Sequence
  Synthetic
acgtcattaattaaatgggaaaaatggcttctctttttgctactttccttgttgtgttggttagtctttctctagctagtgagagtagtgctcgagttcagccaacagagagtatcgttagattccctaatataaccaacctctgcccatttggagaagtgtttaacgccacaagatttgcttctgtatatgcttggaaccgcaaaagaataagcaattgtgtggcagactacagcgtgctatacaattctgcgagtttctcaacattcaagtgctatggtgtgtcaccaactaagttaaatgatctctgctttactaatgtgtacgctgattcatttgtcattagaggtgatgaagttcgtcagatcgctccaggacaaactggcaagatagctgactacaactataagcttccggatgactttacgggatgtgttattgcatggaattccaacaaccttgattctaaagttggagggaattataattacttgtatcggcttttcaggaaatcaaatttgaaaccttttgagagggatatttcgaccgaaatttatcaagccgggagtactccatgcaatggtgtagaaggattcaactgttactttcctctgcaatcatatggatttcaacccacaaacggcgtgggttatcagccctatagggtagtagttctaagttttgaacttttgcatgcacctgcaacagtttgtggacctaagaaatccacaaatttagtcaagaataaatgtgtaaatttcaatttcaatgggttgaccggcactggtgttctgactgagtctggaggtggttctaagaagtcagctgctgaggcgagcaagaaaccaaggcagaaaagaactgcaacaaaagcatataatgttacacaagcatttgggaggcgtggtcctgaacaaactcaaggaaattttggtgatcaagaattgatacgacagggcactgattataaacattggcctcaaattgctcagtttgctccctccgcctctgcattctttggaatgtcaagaattggtatggaggtgaccccgagtgggacatggttaacatatactggagccatcaagcttgatgataaagacccaaatttcaaggaccaggttatactgctcaacaagcacattgatgcttacaaaacattccctcctacttgactcgagtcgacgt

SEQ ID NO: 113 - DNA, 1029 bp, Artificial Sequence
  Synthetic
acgtcattaattaaatgggaaaaatggcttctctttttgctactttccttgttgtgttggttagtctttctctagctagtgagagtagtgctgcttctgtatatgcttggaaccgcaaaagaataagcaattgtgtggcagactacagcgtgctatacaattctgcgagtttctcaacattcaagtgctatggtgtgtcaccaactaagttaaatgatctctgctttactaatgtgtacgctgattcatttgtcattagaggtgatgaagttcgtcagatcgctccaggacaaactggcaagatagctgactacaactataagcttccggatgactttacgggatgtgttattgcatggaattccaacaaccttgattctaaagttggagggaattataattacttgtatcggcttttcaggaaatcaaatttgaaaccttttgagagggatatttcgaccgaaatttatcaagccgggagtactccatgcaatggtgtagaaggattcaactgttactttcctctgcaatcatatggatttcaacccacaaacggcgtgggttatcagccctatagggtagtagttctaagttttgaacttttgcatgcacctgcaacatctggaggtggttctaccgtaacgaagaagtcagctgctgaggcgagcaagaaaccaaggcagaaaagaactgcaacaaaagcatataatgttacacaagcatttgggaggcgtggtcctgaacaaactcaaggaaattttggtgatcaagaattgatacgacagggcactgattataaacattggcctcaaattgctcagtttgctccctccgcctctgcattctttggaatgtcaagaattggtatggaggtgaccccgagtgggacatggttaacatatactggagccatcaagcttgatgataaagacccaaatttcaaggaccaggttatactgctcaacaagcacattgatgcttacaaaacattccctcctactgaaccaaaaaagtgactcgagtcgacgt

SEQ ID NO: 114 - DNA, 1008 bp, Artificial Sequence
  Synthetic
acgtcattaattaaatgggaaaaatggcttctctttttgctactttccttgttgtgttggttagtctttctctagctagtgagagtagtgctgcttctgtatatgcttggaaccgcaaaagaataagcaattgtgtggcagactacagcgtgctatacaattctgcgagtttctcaacattcaagtgctatggtgtgtcaccaactaagttaaatgatctctgctttactaatgtgtacgctgattcatttgtcattagaggtgatgaagttcgtcagatcgctccaggacaaactggcaagatagctgactacaactataagcttccggatgactttacgggatgtgttattgcatggaattccaacaaccttgattctaaagttggagggaattataattacttgtatcggcttttcaggaaatcaaatttgaaaccttttgagagggatatttcgaccgaaatttatcaagccgggagtactccatgcaatggtgtagaaggattcaactgttactttcctctgcaatcatatggatttcaacccacaaacggcgtgggttatcagccctatagggtagtagttctaagttttgaacttttgcatgcacctgcaacatctggaggtggttctaagaagtcagctgctgaggcgagcaagaaaccaaggcagaaaagaactgcaacaaaagcatataatgttacacaagcatttgggaggcgtggtcctgaacaaactcaaggaaattttggtgatcaagaattgatacgacagggcactgattataaacattggcctcaaattgctcagtttgctccctccgcctctgcattctttggaatgtcaagaattggtatggaggtgaccccgagtgggacatggttaacatatactggagccatcaagcttgatgataaagacccaaatttcaaggaccaggttatactgctcaacaagcacattgatgcttacaaaacattccctcctacttgactcgagtcgacgt

SEQ ID NO: 115 - protein, 15 aa, Artificial Sequence
  Synthetic
QIGYYRRATRRIRGG

SEQ ID NO: 116 - protein, 12 aa, Artificial Sequence
  Synthetic
QVILLNKHIDAY

SEQ ID NO: 117 - protein, 14 aa, Artificial Sequence
  Synthetic
LALLLDRLNQLESK

SEQ ID NO: 118 - protein, 11 aa, Artificial Sequence
  Synthetic
AFFGMSRIGME

SEQ ID NO: 119 - protein, 13 aa, Artificial Sequence
  Synthetic
SKQLQQSMSSADS

SEQ ID NO: 120 - protein, 1255 aa, Severe acute respiratory syndrome coronavirus 2
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 121 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 122 - protein, 265 aa, Artificial Sequence
  Synthetic
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLE

SEQ ID NO: 123 - protein, 422 aa, Artificial Sequence
  Synthetic
MSDNGPQSNQRSAPRITFGGPTDSTDNNQNGGRNGARPKQRRPQGLPNNTASWFTALTQHGKEELRFPRGQGVPINTNSGPDDQIGYYRRATRRVRGGDGKMKELSPRWYFYYLGTGPEASLPYGANKEGIVWVATEGALNTPKDHIGTRNPNNNAATVLQLPQGTTLPKGFYAEGSRGGSQASSRSSSRSRGNSRNSTPGSSRGNSPARMASGGGETALALLLLDRLNQLESKVSGKGQQQQGQTVTKKSAAEASKKPRQKRTATKQYNVTQAFGRRGPEQTQGNFGDQDLIRQGTDYKHWPQIAQFAPSASAFFGMSRIGMEVTPSGTWLTYHGAIKLDDKDPQFKDNVILLNKHIDAYKTFPPTEPKKDKKKKTDEAQPLPQRQKKQPTVTLLPAADMDDFSRQLQNSMSGASADSTQA

SEQ ID NO: 124 - protein, 334 aa, Artificial Sequence
  Synthetic
MKNRVISLLMASLLLVLSVIVAPFYKAEAATVVNTPFVAVFSNFDSSQWEKADWANGSVFNCVWKPSQVTFSNGKMILTLDREYGGSYPYKSGEYRTKSFFGYGYYEVRMKAAKNVGIVSSFFTYTGPSDNNPWDEIDIEFLGKDTTKVQFNWYKNGVGGNEYLHNLGFDASQDFHTYGFEWRPDYIDFYVDGKKVYRGTRNIPVTPGKIMMNLWPGIGVDEWLGRYDGRTPLQAEYEYVKYYPNGVPQDNPTPTPTIAPSTPTNPNLPLKGDVNGDGHVNSSDYSLFKRYLLRVIDRFPVGDQSVADVNRDGRIDSTDLTMLKRYLIRAIPSL

SEQ ID NO: 125 - protein, 259 aa, Artificial Sequence
  Synthetic
MGFVLFSQLPSFLLVSTLLLFLVISHSCRAHHHHHHHHGGSYPYKSGEYRTKSFFGYGYYEVRMKAAKNVGIVSSFFTYTGPSDNNPWDEIDIEFLGKDTTKVQFNWYKNGVGGNEYLHNLGFDASQDFHTYGFEWRPDYIDFYVDGKKVYRGTRNIPVTPGKIMMNLWPGIGVDEWLGRYDGRTPLQAEYEYVKYYPNGRSEFKLVVNTPFVAVFSNFDSSQWEKADWANGSVFNCVWKPSQVTFSNGKMILTLDREY

SEQ ID NO: 126 - DNA, 780 bp, Artificial Sequence
  Synthetic
atgggattcgttttgttttctcaattgccttcatttcttttggtttctactcttttgctttttcttgttatttctcattcatgtagagctcatcaccatcaccaccatcatcatggcggctcttacccttataagagcggtgagtaccggaccaagagcttctttggttacggttactacgaggtgcggatgaaggctgctaagaacgtgggtatcgtgtccagcttctttacctacaccgggccatctgataacaacccttgggatgagatcgacatcgagttccttggtaaggacactaccaaggtgcagttcaactggtacaagaacggtgttggtggcaacgagtaccttcacaaccttggctttgatgccagccaggatttccacacttacggttttgaatggcggcctgactacatcgacttctacgtggacggtaagaaggtgtacaggggcaccagaaatatccctgtgactcctggcaagatcatgatgaacctttggcctggtatcggtgtggatgagtggcttggtagatacgatggtaggactcctctgcaggctgagtacgagtacgttaagtactaccctaacggcagatctgaattcaagcttgtggtgaatactcctttcgtggccgtgttcagcaacttcgattctagccagtgggagaaagctgattgggctaacggttctgtgttcaactgcgtgtggaagccttctcaggtgaccttctctaacggcaagatgattctgaccctggaccgtgagtattga

SEQ ID NO: 127 - DNA, 680 bp, Severe acute respiratory syndrome coronavirus 2; identical to SEQ ID NO: 4

SEQ ID NO: 128 - protein, 1273 aa, Severe acute respiratory syndrome coronavirus 2; identical to SEQ ID NO: 121

SEQ ID NO: 129 - protein, 16 aa, Artificial Sequence
  Synthetic
VLTESNKKFLPFQQFG

SEQ ID NO: 130 - protein, 16 aa, Artificial Sequence
  Synthetic
AIHADQLTPTWRVYST

SEQ ID NO: 131 - protein, 16 aa, Artificial Sequence
  Synthetic
DSFKEELDKYFKNHTS

SEQ ID NO: 132 - protein, 614 aa, Artificial Sequence
  Synthetic
MDIDPYKEFGATVELLSFLPSDFFPSVRDLLDTASALYREALESPEHCSPHHTALRQAILCWGELMTLATWVGNNLEGSAGGGRDPASRDLVVNYVNTNMGLKIRQLLWFHISCLTFGRETVLEYLVSFGVWIRTPPAYRPPNAPILSTLPETTVVGGSSGGSGGSGGSGGSGGSGGSTMDIDPYKEFGATVELLSFLPSDFFPSVRDLLDTASALYREALESPEHCSPHHTALRQAILCWGELMTLATWVGNNLEFGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKSGGASDPASRDLVVNYVNTNMGLKIRQLLWFHISCLTFGRETVLEYLVSFGVWIRTPPAYRPPNAPILSTLPETTVVRRRDRGRSPRRRTPSPRRRRSQSPRRRRSQSRESQCLE

SEQ ID NO: 133 - protein, 1273 aa, Severe acute respiratory syndrome coronavirus 2; identical to SEQ ID NO: 121

SEQ ID NO: 134 - protein, 419 aa, Artificial Sequence
  Synthetic
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 135 - protein, 85 aa, Artificial Sequence
  Synthetic
DQIGYYRRATRRIRGGDGKMKDLSPRWYFYYLGTGALALLLLDRLNQLESKMSAFFGMSRIGMEVADLDDFSKQLQQSMSSADST

SEQ ID NO: 136 - DNA, 255 bp, Artificial Sequence
  Synthetic
gatcagattggctattatcgccgcgcgacccgccgcattcgcggcggcgatggcaaaatgaaagatctgagcccgcgctggtatttttattatctgggcaccggcgcgctggcgctgctgctgctggatcgcctgaaccagctggaaagcaaaatgagcgcgttttttggcatgagccgcattggcatggaagtggcggatctggatgattttagcaaacagctgcagcagagcatgagcagcgcggatagcacc

SEQ ID NO: 137 - protein, 145 aa, Artificial Sequence
  Synthetic
MGKMASLFATFLVVLVSLSLASESSATKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPP

SEQ ID NO: 138 - DNA, 470 bp, Artificial Sequence
  Synthetic
acgtcattaattaaatgggaaaaatggcttctctttttgctactttccttgttgtgttggttagtctttctctagctagtgagagtagtgctaccaagaaaagcgctgccgaagcgtcaaagaagccaaggcagaagagaactgctacaaaagcttataatgtgactcaagcatttggaagacgaggccctgagcaaacacaggggaattttggtgatcaagaactgatcaggcaaggaacagattataaacattggcctcaaattgctcagtttgctccttctgcctccgcattctttggtatgtctcgtattggaatggaggtaactccgagtggtacatggcttacgtatactggcgcaattaaattggatgataaagacccaaatttcaaggaccaggttatactattaaacaagcatatagatgcatacaaaaccttcccaccctgactcgagcctaggacgaag

SEQ ID NO: 139 - DNA, 1160 bp, Artificial Sequence
  Synthetic
acgtcattaattaaatgggaaaaatggcttctctttttgctactttccttgttgtgttggttagtctttctctagctagtgagagtagtgctggcggctcttacccttataagagcggtgagtaccggaccaagagcttctttggttacggttactacgaggtgcggatgaaggctgctaagaacgtgggtatcgtgtccagcttctttacctacaccgggccatctgataacaacccttgggatgagatcgacatcgagttccttggtaaggacactaccaaggtgcagttcaactggtacaagaacggtgttggtggcaacgagtaccttcacaaccttggctttgatgccagccaggatttccacacttacggttttgaatggcggcctgactacatcgacttctacgtggacggtaagaaggtgtacaggggcaccagaaatatccctgtgactcctggcaagatcatgatgaacctttggcctggtatcggtgtggatgagtggcttggtagatacgatggtaggactcctctgcaggctgagtacgagtacgttaagtactaccctaacggcagatctgaattcaagcttgtggtgaatactcctttcgtggccgtgttcagcaacttcgattctagccagtgggagaaagctgattgggctaacggttctgtgttcaactgcgtgtggaagccttctcaggtgaccttctctaacggcaagatgattctgaccctggaccgtgagtatggcgggggcggctcaggaggtggtggaacgaagaaatccgcggccgaggcatcaaagaagccaaggcagaaaagaactgcaaccaaagcttataatgtgacacaagcatttggaagacgaggccctgaacaaactcaaggaaattttggtgaccaagaactgatcaggcaaggtactgattataaacattggcctcagattgctcagtttgctccttctgcttctgccttcttcgggatgagtcgtattggaatggaggtaaccccaagcgggacatggttgacatatactggtgcaataaagcttgatgataaagatccaaatttcaaggaccaggttatattactcaacaagcacattgatgcttacaaaacatttcctccctgactcgagcctaggacgaag

SEQ ID NO: 140 - DNA, 34 bp, Artificial Sequence
  Synthetic
gagttcggatcctcaaggttagcacccacccagg

SEQ ID NO: 141 - DNA, 31 bp, Artificial Sequence
  Synthetic
gccttatgctagccgtgatctggtcgtcaac